(19) United States
(12) Patent Application Publication
(10) Pub. No.: US 2010/0209427 A1
(43) Pub. Date: Aug. 19, 2010

(54) LYSINE ACETYLATION SITES

(76) Inventors: YU LI, ANDOVER, MA (US); TING-LEI GU, WOBURN, MA (US); DAVID LOMBARD, Ann Arbor, MI (US)

Correspondence Address: Nancy Chiu Wilker, Ph.D.;Chief Intellectual Property Counsel, CELL SIGNALING TECHNOLOGY, INC., 3 Trask Lane, Danvers, MA, 01923, US

(21) Appl. No.: 12/566,523

(22) Filed: Sep. 24, 2009

Related U.S. Application Data
(60) Provisional application No. 61/194,097, filed on Sep. 24, 2008.

(51) Int. Cl. A61K 39/395; C07K 16/00; A61P 3/00; G01N 33/566

(52) U.S. Cl. 424/139.1; 530/387.9; 530/387.3; 530/391.7; 424/183.1; 436/501

(57) ABSTRACT

The invention discloses 1302 novel acetylation sites peptides (including AQUA peptides) comprising an acetylation site of the invention, antibodies specifically bind to a novel acetylation site of the invention, and diagnostic and therapeutic uses of the above. The acetylation sites identified provide important information regarding metabolism in energy signaling pathways.

### RELATED APPLICATIONS

Pursuant to 35 U.S.C. §119(e) this application claims the benefit of, and priority to, provisional application U.S. Ser. No. 61/194,097, filed Sep. 24, 2008, the contents of which is incorporated herein, in its entirety, by reference.

### FIELD OF THE INVENTION

The invention relates generally to novel lysine acetylation sites, methods and compositions for detecting, quantitating and modulating same.

### BACKGROUND OF THE INVENTION

The activation of proteins by post-translational modification is an important cellular mechanism for regulating most aspects of biological organization and control, including growth, development, homeostasis, and cellular communication. Protein acetylation, for example, plays a critical role in the etiology of many pathological conditions and diseases, including to mention but a few: metabolic disorders, cancer, developmental disorders, autoimmune diseases, and diabetes. Yet, in spite of the importance of protein modification, it is not yet well understood at the molecular level, due to the extraordinary complexity of signaling pathways, and the slow development of technology necessary to unravel it.

Protein acetylation plays a complex and critical role in the regulation of biological processes and may prove to be important to diagnostic or therapeutic targets for molecular medicine. Protein acetylation on lysine residues is a dynamic, reversible and highly regulated chemical modification. Historically, histones were perceived as the most important substrate of acetylation, if not the sole substrate. It was proposed 40 years ago that structural modification of histones by acetylation plays an important role in chromatin remodeling and gene expression. Two groups of enzymes, histone deacetylases (HDACs) and histone acetyltransferases (HATs), are responsible for deacetylating and acetylating the histones.

Recent studies have revealed that HDACs are involved in a much broader assay of biological processes. For example, HDAC6 has been implicated in the regulation of microtubules, growth factor-induced chemotaxis and misfolded protein stress response. See Cohen et al., Science, vol 245:42 (2004). Consistent with these non-histone functions, HDAC6 is mainly located to the cytoplasm.

A growing list of acetylated proteins is currently available. It shows that both cytoplasmic and nuclear proteins can undergo reversible acetylation, and protein acetylation can have the following effects on its function: 1) Protein stability. Both acetylation and ubiquitylation often occur on the same lysine, competition between these two modifications affects the protein stability. It has been shown that HDACs can decrease the half-life of some proteins by exposing the lysine for ubiquitylation. 2) Protein-protein interactions. It has been shown that acetylation induces STAT3 dimerization and subsequently nuclear translocation. In the case of nuclear DNA-damage-response protein Ku70, the deacetylated form of Ku70 sequesters BAX, the pro-apoptotic protein, in the cytoplasm and protects cells from apoptosis. In response to apoptotic stimuli, Ku70 becomes acetylated and subsequently releases Bax from its sequestration, leading to translocation of BAX to the mitochondria and activation of apoptotic cascade. 3) Protein translocation. As described for STAT3 and BAX, reversible acetylation affects the subcellular localization. In the case of STAT3, its nuclear localization signal contains lysine residues that favor nuclear retention when acetylated. 4) DNA binding. It have been shown that acetylation of p53 regulates its stability, its DNA binding and its transcriptional activity. Similarly, the DNA binding affinity of NF-kB and its transcriptional activation are also regulated by HATs and HDACs. See Minucci et al., Nature Cancer Reviews, 6: 38-51 (2005).

HATs and HDACs have been linked to pathogenesis of cancer. Specific HATs (p300 and CBP) are targets of viral oncoproteins (adenoviral E1A, human papilloma virus E6 and SV40 T antigen). See Eckner, R. et al., Cold Spring Harb. Symp. Quant. Biol., 59: 85-95 (1994). Structural alterations in HATs, including translocation, amplifications, deletions and point mutations have been found in various human cancers. See Iyer, N G. et al., Oncogene, 23: 4225-4231 (2004). For HDACs, increased expression of HDAC1 has been detected in gastric cancers, esophageal squamous cell carcinoma, and prostate cancer. See Halkidou, K. et al., Prostate 59: 177-189 (2004). Increased expression of HDAC2 has been detected in colon cancer and has been shown to interact functionally with Wnt pathway. Knockdown of HDAC2 by siRNA in colon cancer cells resulted in cell death. See Zhu, P. et al., Cancer Cell, 5: 455-463 (2004). Increased expression of HDAC6 has been linked to better survival in breast cancer, See Zhang, Z. et al., Clin. Cancer Res., 10: 6962-6968 (2004), while reduced expression of HDAC5 and 10 have been associated with poor prognosis in lung cancer patients. See Osada, H. et al., Cancer, 112: 26-32 (2004).

HDAC inhibitors (HDACi) are promising new targeted anti-cancer agents, and first-generation HDACi in several clinical trials show significant activity against a spectrum of both hematological and solid tumors at doses that are well tolerated by the patients. See Drummond, D C. et al., Annu. Rev. Pharmacol. Toxicol., 45: 495-528 (2005). However, the relationship between the toxicity of HDACi and their pharmacokinetic properties is still largely unknown, which makes it difficult to optimize HDACi treatment. More importantly the key targets for HDACi action are unknown. This makes it difficult to select patients who are most likely to respond to HDACi. Proposed surrogate markers, like measuring the level of acetylated histone from blood cells before and after treatment, should be serve as indicators of effectiveness, but these need to be validated clinically yet and do not always correlated with pharmacokinetic profile. Therefore, to identify the entire spectrum of acetylated proteins deserves a much more systematic experimental strategy which would optimally involve a dynamic map of the acetylated proteins and their functions.

Despite the identification of a few key molecules involved in protein acetylation signaling pathways, the vast majority of signaling protein changes underlying these pathways remains unknown. There is, therefore, relatively scarce information about acetylation-driven signaling pathways and acetylation sites relevant to the pathogenesis of cancer and other human diseases. This has hampered a complete and accurate understanding of how protein activation within signaling pathways may be driving different human diseases, including cancer.

Presently, diagnosis of cancer and metabolic disorders is made by tissue biopsy and detection of different cell surface markers. However, misdiagnosis can occur since some cell types (e.g., cancerous cells) cases can be negative for certain markers and because these markers may not indicate which genes or protein kinases may be deregulated. Although the genetic translocations and/or mutations characteristic of a particular form of disease (e.g., cancer) can be sometimes detected, it is clear that other downstream effectors of constitutively active kinases having potential diagnostic, predictive, or therapeutic value, remain to be elucidated.

The sirtuins are a family of seven human homologs of the yeast Sir2 (silent information regulator 2) gene that play a role in regulating gene expression in a variety of organisms through the deacetylation of modified lysine residues on histones, transcription factors and other proteins. For example, human SIRT1 regulates a number of transcription factors that modulate endocrine signaling including PPARγ, forkhead-box transcription factors, p53, or PPARγ coactivator 1α.

SIRT3 has been shown to be localized in the mitochondria and regulates mitochondrial function and thermogenesis in brown adipocytes. These genes may also play a role in the mediation of the metabolic effects of caloric restriction in an animal, which effects have been associated with increased longevity. As such, it would be beneficial to have methods to determine what effects, if any, treatment with therapeutic modalities, caloric restriction, genetics, aging, etc., may have on energy metabolism via changes in protein acetylation in signaling pathways associated with mitochondrial function. See, for example, Guarente, L. and Picard, F. Cell 120: 473-482, 2005; Yang, T., et al., Trends Endocrinol Metab., 17(5):186-91, 2006; Shi, T., et al., J. Biol. Chem., 250(14): 13560-13567, 2005; Onyango, P., et al., PNAS 99(21): 13653-13658, 2002; and Schwer, B., et al., PNAS 103(27): 10224-10229, 2006.

Accordingly, identification of downstream signaling molecules and acetylation sites involved in different types of diseases including for example, diseases resulting from metabolic disorders, and the development of new reagents to detect and quantify these sites and proteins may lead to improved diagnostic/prognostic markers, as well as novel drug targets, for the detection and treatment of many diseases.

### SUMMARY OF THE INVENTION

The present invention provides in one aspect novel lysine acetylation sites (Table 1) identified in signal transduction proteins and pathways relevant to protein acetylation signaling. The novel sites occur in proteins such as: adaptor/scaffold proteins, apoptosis proteins, calcium-binding proteins, cell cycle regulation proteins, chaperone proteins, chromatin or DNA binding/repair/replication proteins, metabolic proteins, mitochondrial proteins, cytoskeletal proteins, endoplasmic reticulum or golgi proteins, enzyme proteins, G proteins or regulator proteins, lipid binding proteins, mitochondrial proteins, motor or contractile proteins, proteases, protein kinases, receptor/channel/transporter/cell surface proteins, RNA binding proteins, transcriptional regulators, translational regulators, ubiquitan conjugating system, and proteins of unknown function.

In another aspect, the invention provides peptides comprising the novel acetylation sites of the invention, and proteins and peptides that are mutated to eliminate the novel acetylation sites.

In another aspect, the invention provides modulators that modulate lysine acetylation at a novel acetylation site of the invention, including small molecules, peptides comprising a novel acetylation site, and binding molecules that specifically bind at a novel acetylation site, including but not limited to antibodies or antigen-binding fragments thereof.

In another aspect, the invention provides compositions for detecting, quantitating or modulating a novel acetylation site of the invention, including peptides comprising a novel acetylation site and antibodies or antigen-binding fragments thereof that specifically bind at a novel acetylation site. In certain embodiments, the compositions for detecting, quantitating or modulating a novel acetylation site of the invention are Heavy-Isotope Labeled Peptides (AQUA peptides) comprising a novel acetylation site.

In another aspect, the invention discloses acetylation site specific antibodies or antigen-binding fragments thereof. In one embodiment, the antibodies specifically bind to an amino acid sequence comprising a acetylation site identified in Table 1 when the lysine identified in Column D is acetylated, and do not significantly bind when the lysine is not acetylated. In another embodiment, the antibodies specifically bind to an amino acid sequence comprising an acetylation site when the lysine is not acetylated, and do not significantly bind when the lysine is acetylated.

In another aspect, the invention provides a method for making acetylation site-specific antibodies.

In another aspect, the invention provides compositions comprising a peptide, protein, or antibody of the invention, including pharmaceutical compositions.

In a further aspect, the invention provides methods of treating or preventing a metabolic disorder in a subject, wherein the metabolic disorder is associated with the acetylation state of a novel acetylation site in Table 1, whether acetylated or deacetylated. In certain embodiments, the methods comprise administering to a subject a therapeutically effective amount of a peptide comprising a novel acetylation site of the invention. In certain embodiments, the methods comprise administering to a subject a therapeutically effective amount of an antibody or antigen-binding fragment thereof that specifically binds at a novel acetylation site of the invention.

In a further aspect, the invention provides methods for detecting and quantitating acetylation at a novel lysine acetylation site of the invention.

In another aspect, the invention provides a method for identifying an agent that modulates lysine acetylation at a novel acetylation site of the invention, comprising: contacting a peptide or protein comprising a novel acetylation site of the invention with a candidate agent, and determining the acetylation state or level at the novel acetylation site. A change in the acetylation state or level at the specified lysine in the presence of the test agent, as compared to a control, indicates that the candidate agent potentially modulates lysine acetylation at a novel acetylation site of the invention.

In another aspect, the invention discloses immunoassays for binding, purifying, quantifying and otherwise generally detecting the acetylation of a protein or peptide at a novel acetylation site of the invention.

Also provided are pharmaceutical compositions and kits comprising one or more antibodies or peptides of the invention and methods of using them.

A further aspect of the invention provides a method for measuring changes in acetylation of proteins in signaling pathways associated with mitochondrial function in a mammal. The method comprises the steps of: (a) collecting and processing a sample from the mammal; (b) treating the processed sample from step (a) with an antibody to a site according to Table 1; and (c) identifying and quantitating changes in acetylation patterns.

### BRIEF DESCRIPTION OF THE DRAWINGS

FIG. 1 is a diagram depicting the immuno-affinity isolation and mass-spectrometric characterization methodology (IAP) used in the Examples to identify the novel acetylation sites disclosed herein.

FIG. 2 is a table (corresponding to Table 1) summarizing the 1302 novel acetylation sites of the invention: Column A=the parent proteins from which the acetylation sites are derived; Column B=the SwissProt accession number for the human homologue of the identified parent proteins; Column C=the protein type/classification; Column D=the lysine residues at which acetylation occurs (each number refers to the amino acid residue position of the lysine in the parent human protein, according to the published sequence retrieved by the SwissProt accession number); Column E=flanking sequences of the acetylatable lysine residues; sequences (SEQ ID NOs) were identified using Trypsin digestion of the parent proteins; in each sequence, the lysine (see corresponding rows in Column D) appears in lowercase; Column F=the cell type(s)/Tissue/Patient Sample in which each of the acetylation site was discovered; and Column G=the SEQ ID NOs of the trypsin-digested peptides identified in Column E.

FIGS. 3A and 3B are scanned images of Western blotting analysis of mitochondrial preparations made from wild-type or SIRT3 knock-out mice using (i.e., blotting with) representative, non-limiting antibodies of the invention, namely rabbit polyclonal antibodies that specifically bind to the acetylated lysine residue at position 221 within SEQ ID NO: 887.

FIG. 4 is a scanned image of Western blotting analysis of mitochondrial preparations made from wild-type or SIRT3 knock-out mice using (i.e., blotting with) representative, non-limiting antibodies of the invention, namely rabbit polyclonal antibodies that specifically bind to the acetylated lysine residue at position 455 (also referred as position 454) within SEQ ID NO: 708.

FIG. 5 is a scanned image of Western blotting analysis of mitochondrial preparations made from wild-type or SIRT3 knock-out mice using (i.e., blotting with) representative, non-limiting antibodies of the invention, namely rabbit polyclonal antibodies that specifically bind to the acetylated lysine residue at position 111 within SEQ ID NO: 788.

FIG. 6 is an exemplary mass spectrograph depicting the detection and quantitation of the acetylation of murine CPS1 during caloric restriction of the animals.

FIG. 7 is an exemplary mass spectrograph depicting the detection and quantitation of the acetylation of murine GOT2 during caloric restriction of the animals.

FIG. 8 is an exemplary mass spectrograph depicting the detection and quantitation of the acetylation of murine HADHA during caloric restriction of the animals.

### DETAILED DESCRIPTION OF THE INVENTION

The inventors have discovered and disclosed herein novel lysine acetylation sites in signaling proteins. The newly discovered acetylation sites significantly extend our knowledge of HDAC substrates and of the proteins in which the novel sites occur. The disclosure herein of the novel acetylation sites and reagents including peptides and antibodies specific for the sites add important new tools for the elucidation of signaling pathways that are associate with a host of biological processes including cell division, growth, differentiation, developmental changes and disease. Their discovery provides and focuses further elucidation on various multiparametric processes. And, the novel sites provide additional diagnostic and therapeutic targets.

### 1. Novel Acetylation Sites

In one aspect, the invention provides 1302 novel lysine acetylation sites in signaling proteins from cellular extracts from a variety cell lines and tissue samples (as further described below in Examples), identified using the techniques described in “Immunoaffinity Isolation of Modified Peptides From Complex Mixtures,” U.S. Patent Publication No. 20030044848, Rush et al., using Table 1 summarizes the identified novel acetylation sites.

These acetylation sites thus occur in proteins found principally in adipose and liver tissues and in selected cell lines. The sequences of the human homologues are publicly available in SwissProt database and their Accession numbers listed in Column B of Table 1. The novel sites occur in proteins such as: adaptor/scaffold proteins, apoptosis proteins, calcium-binding proteins, cell cycle regulation proteins, cell surface proteins, chromatin or DNA binding/repair/replication proteins, metabolic proteins, cytoskeletal proteins, enzyme proteins, g proteins or regulator proteins, proteases, phosphatases, receptor/channel/transporter/cell surface proteins, mitochondrial proteins, RNA binding proteins, transcriptional regulators, translational regulators, ubiquitan conjugating system, vesicle proteins and proteins of unknown function. (see Column C of Table 1).

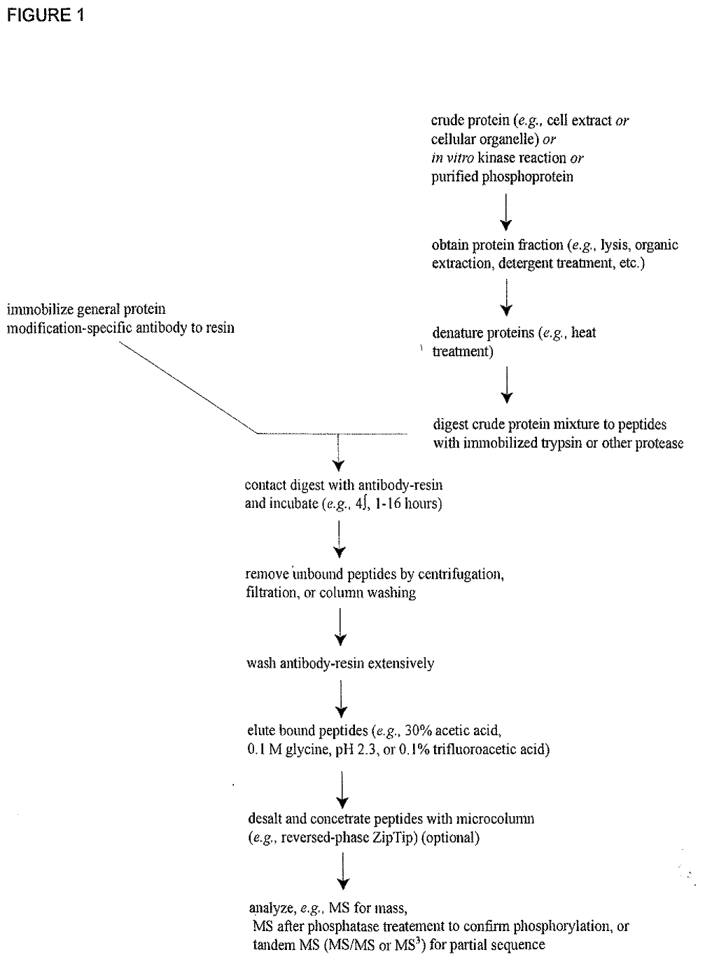

The novel acetylation sites of the invention were identified according to the methods described by Rush et al., U.S. Patent Publication No. 20030044848, which are herein incorporated by reference in its entirety. Briefly, acetylation sites were isolated and characterized by immunoaffinity isolation and mass-spectrometric characterization (IAP) (FIG. 1), using cell lines and/or tissue samples. In addition to the newly discovered acetylation sites (all having an acetylatable lysine), many known acetylation sites were also identified.

The immunoaffinity/mass spectrometric technique described in Rush et al, i.e., the “IAP” method, is described in detail in the Examples and briefly summarized below.

The IAP method generally comprises the following steps: (a) a proteinaceous preparation (e.g., a digested cell extract) comprising acetyl peptides from two or more different proteins is obtained from an organism; (b) the preparation is contacted with at least one immobilized general acetylated-lysine-specific antibody; (c) at least one acetyl peptide specifically bound by the immobilized antibody in step (b) is isolated; and (d) the modified peptide isolated in step (c) is characterized by mass spectrometry (MS) and/or tandem mass spectrometry (MS-MS). Subsequently, (e) a search program (e.g., Sequest) may be utilized to substantially match the spectra obtained for the isolated, modified peptide during the characterization of step (d) with the spectra for a known peptide sequence. A quantification step, e.g., using SILAC or AQUA, may also be used to quantify isolated peptides in order to compare peptide levels in a sample to a baseline.

In the IAP method as disclosed herein, a general acetylated lysine-specific monoclonal antibody (commercially available from Cell Signaling Technology, Inc., Beverly, Mass., Cat #9681) may be used in the immunoaffinity step to isolate the widest possible number of acetyl-lysine containing peptides from the cell extracts.

As described in more detail in the Examples, lysates may be prepared from various cell lines or tissue samples and digested with trypsin after treatment with DTT and iodoacetamide to alkylate cysteine residues. Before the immunoaffinity step, peptides may be pre-fractionated (e.g., by reversed-phase solid phase extraction using Sep-Pak C₁₈ columns) to separate peptides from other cellular components. The solid phase extraction cartridges may then be eluted (e.g., with acetonitrile). Each lyophilized peptide fraction can be redissolved and treated with acetyl-lysine specific antibody (e.g., CST Catalogue #8691) immobilized on protein Agarose. Immunoaffinity-purified peptides can be eluted and a portion of this fraction may be concentrated (e.g., with Stage or Zip tips) and analyzed by LC-MS/MS (e.g., using a ThermoFinnigan LCQ Deca XP Plus ion trap mass spectrometer or LTQ). MS/MS spectra can be evaluated using, e.g., the program Sequest with the NCBI human protein database.

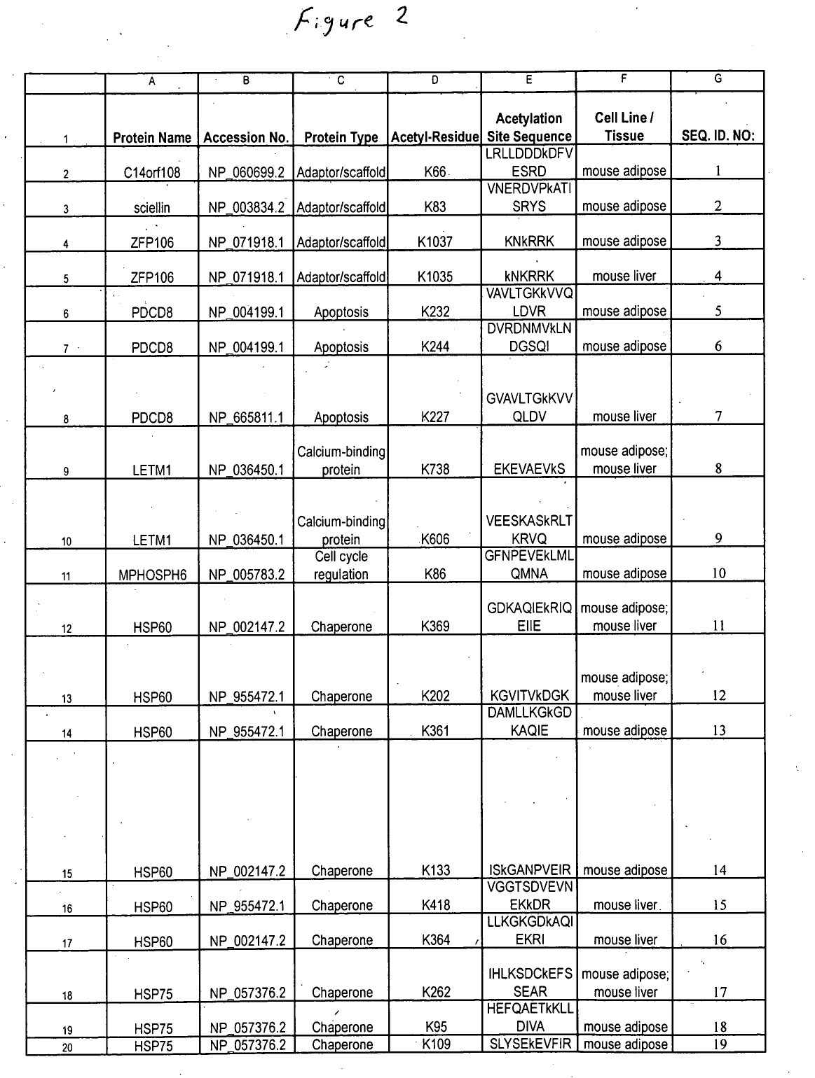

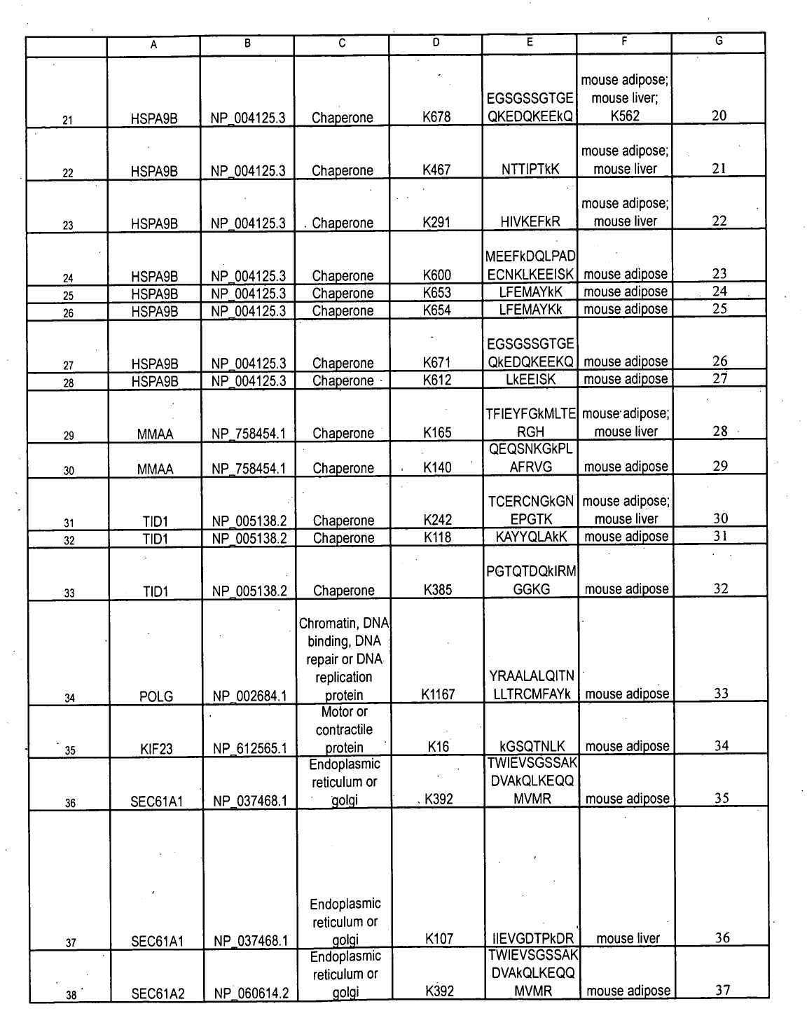

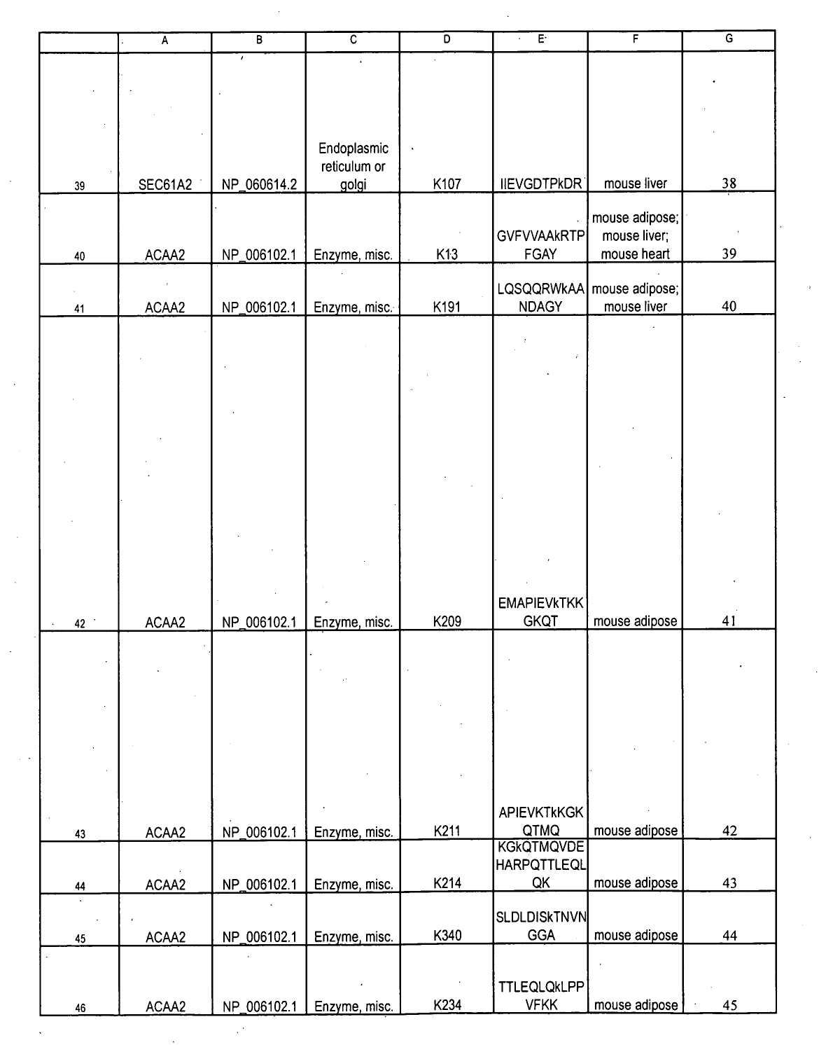

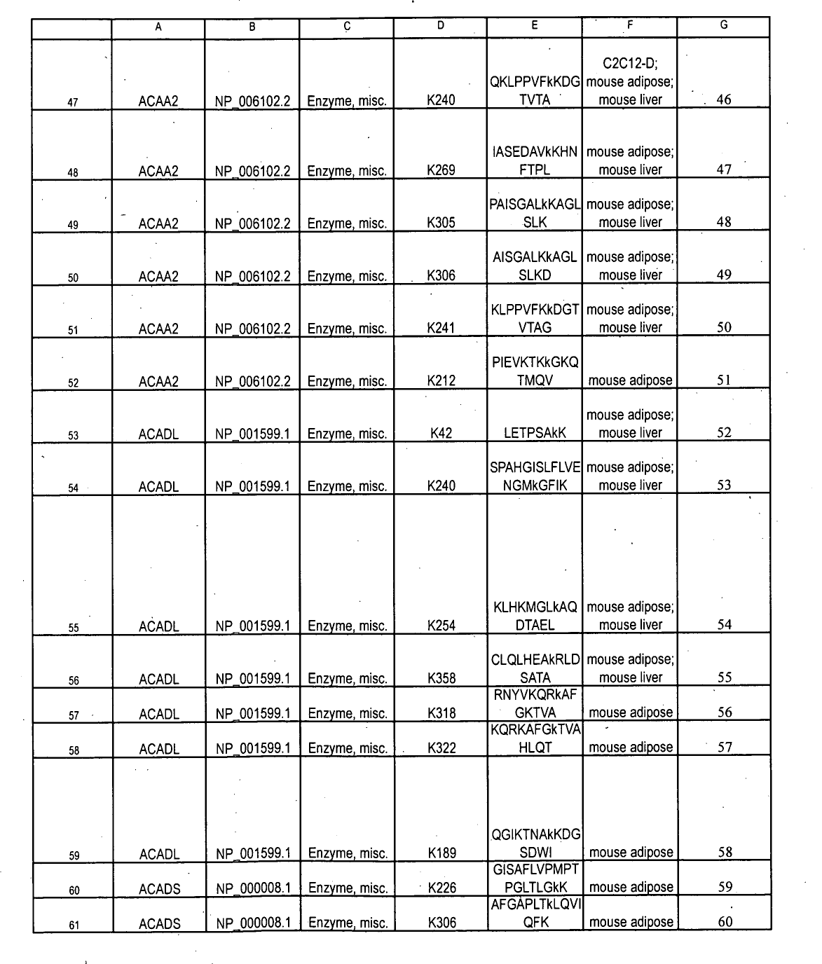

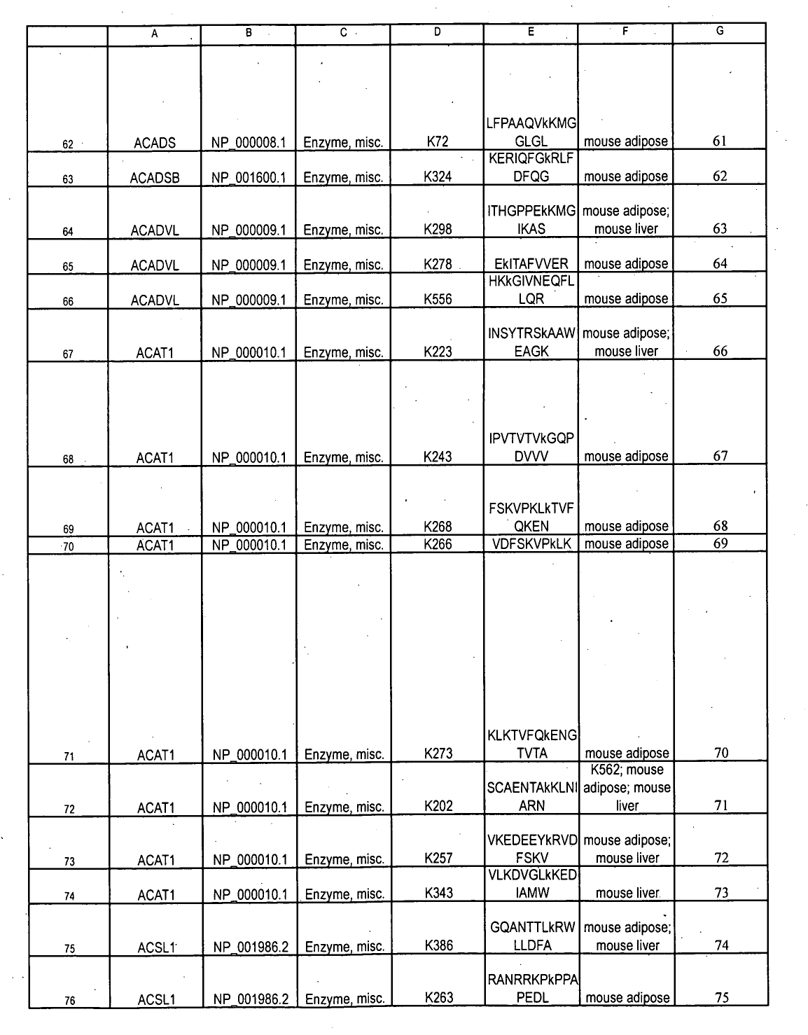

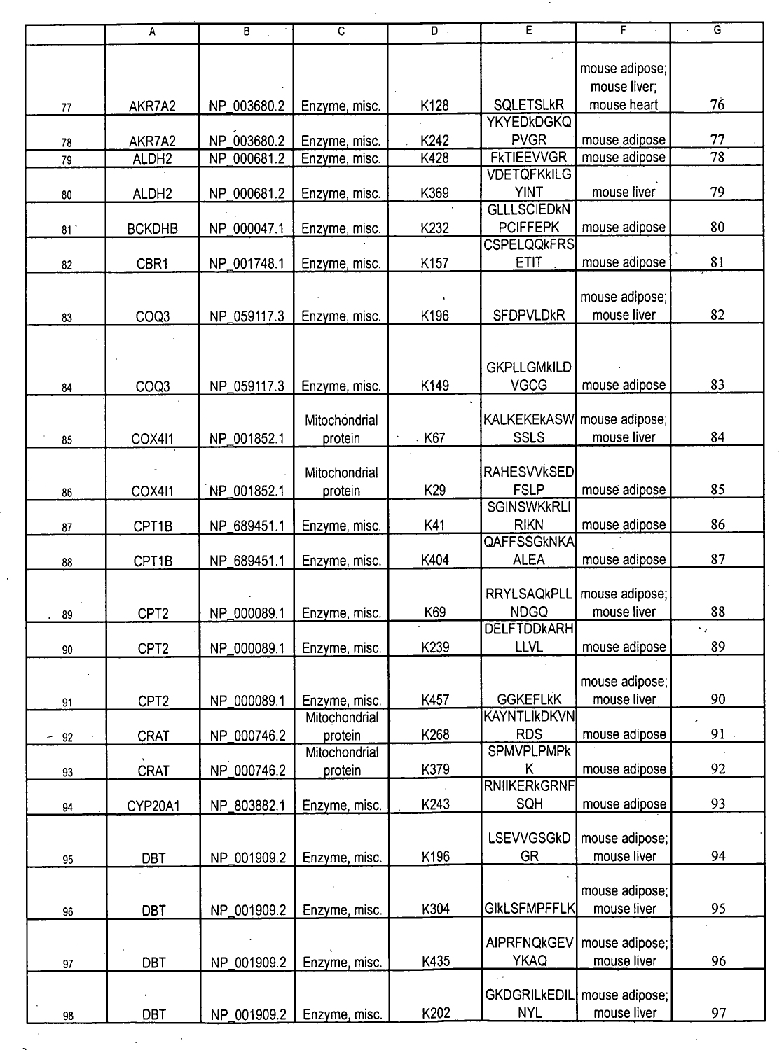

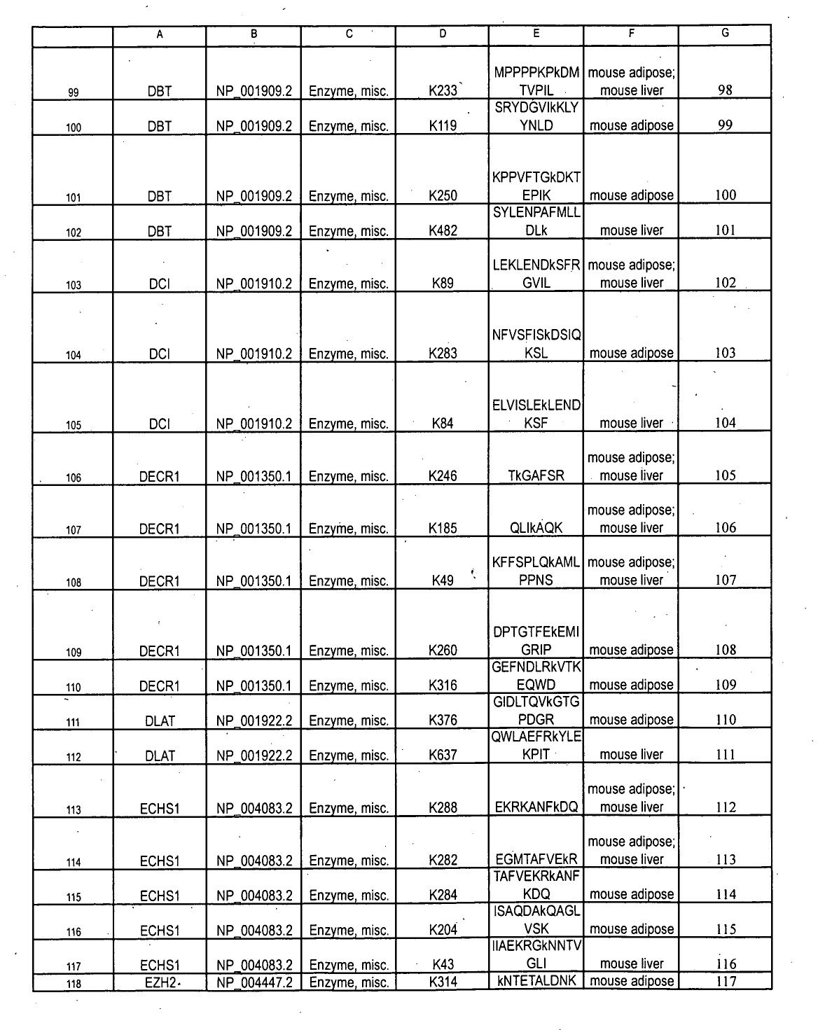

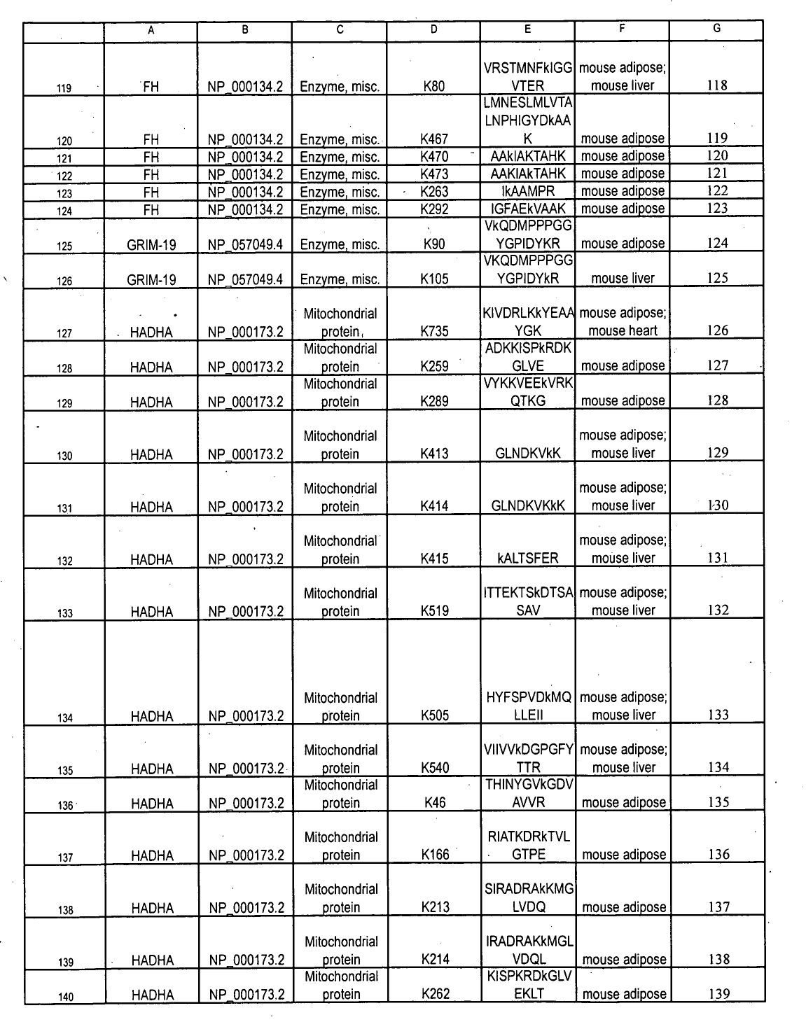

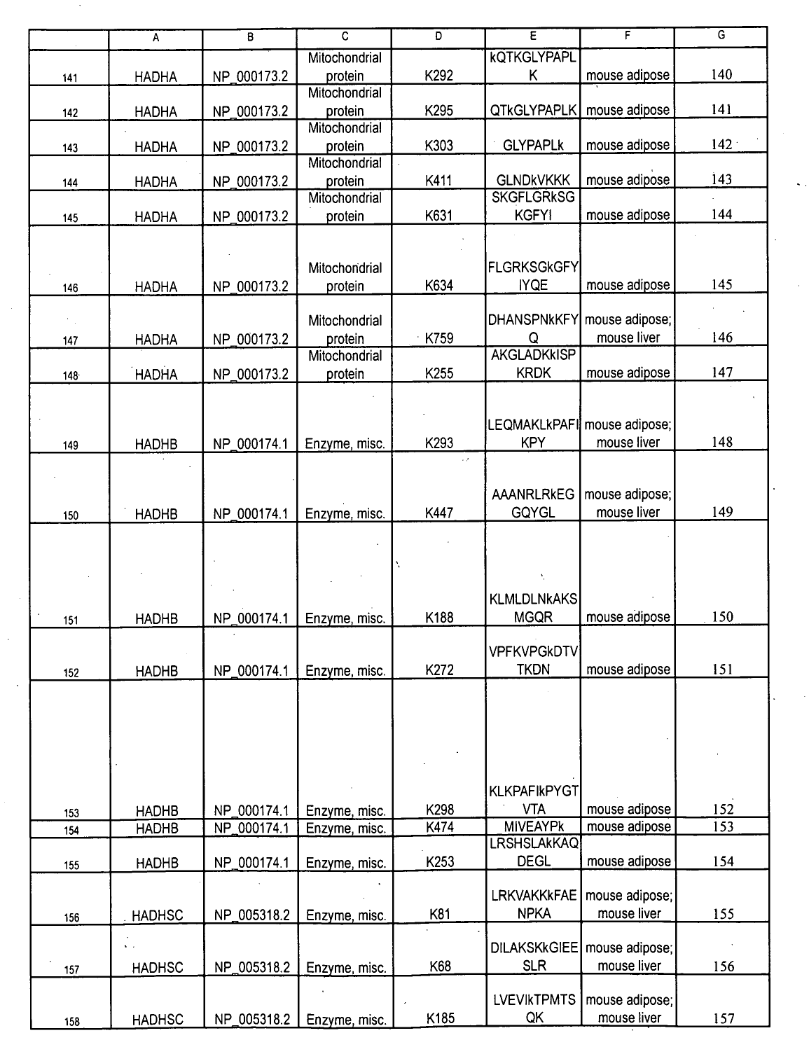

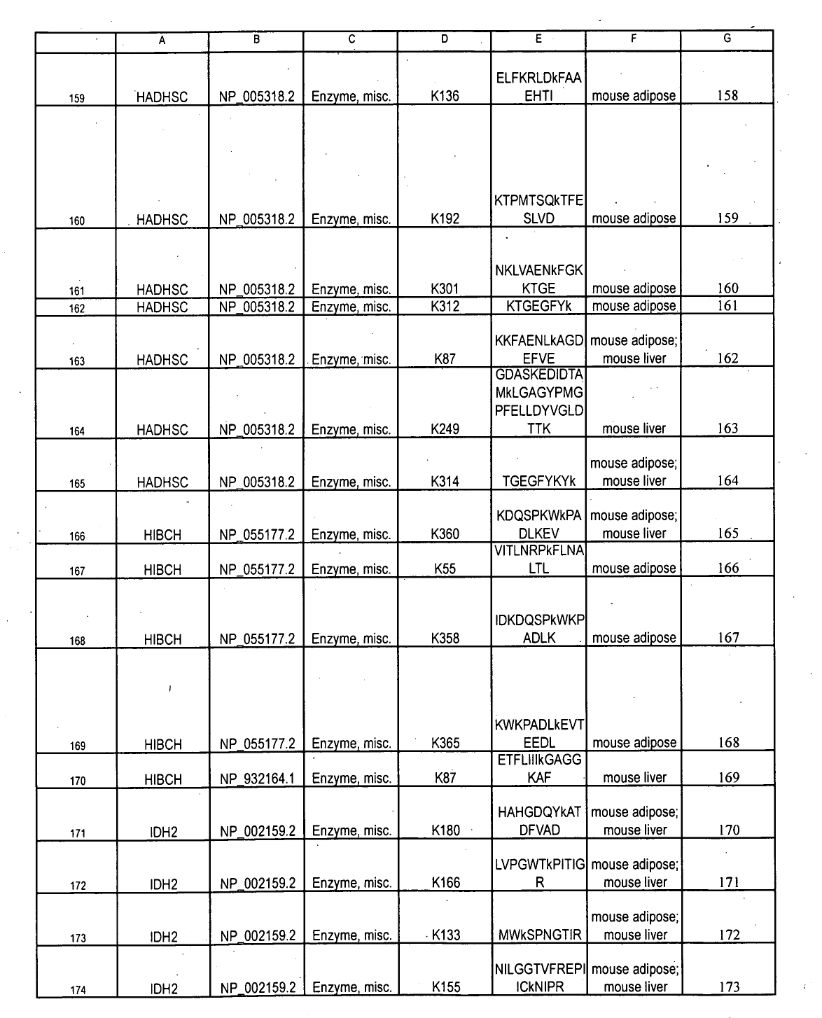

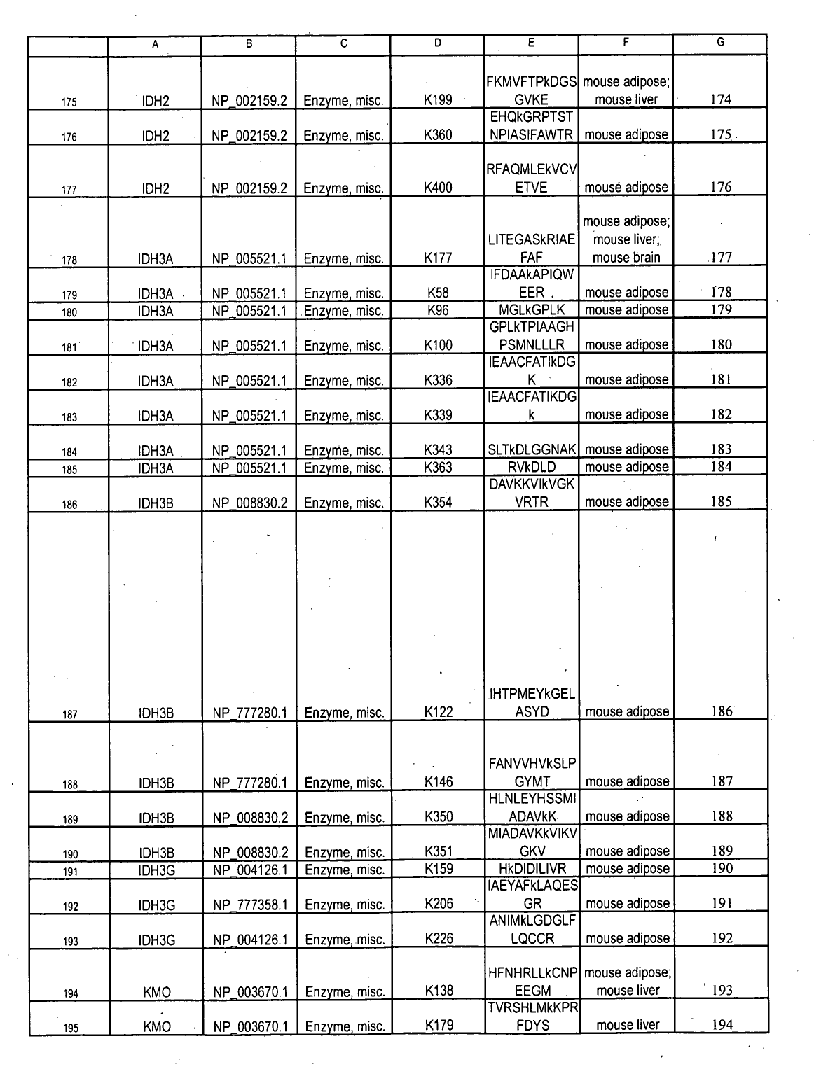

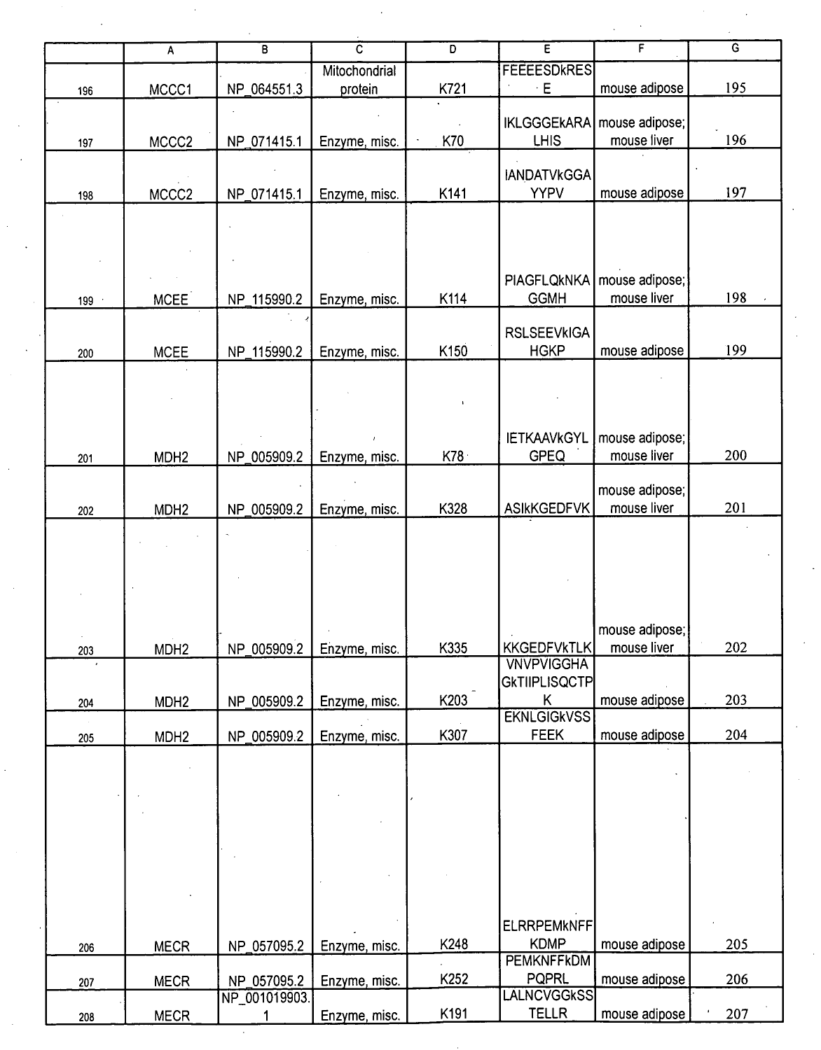

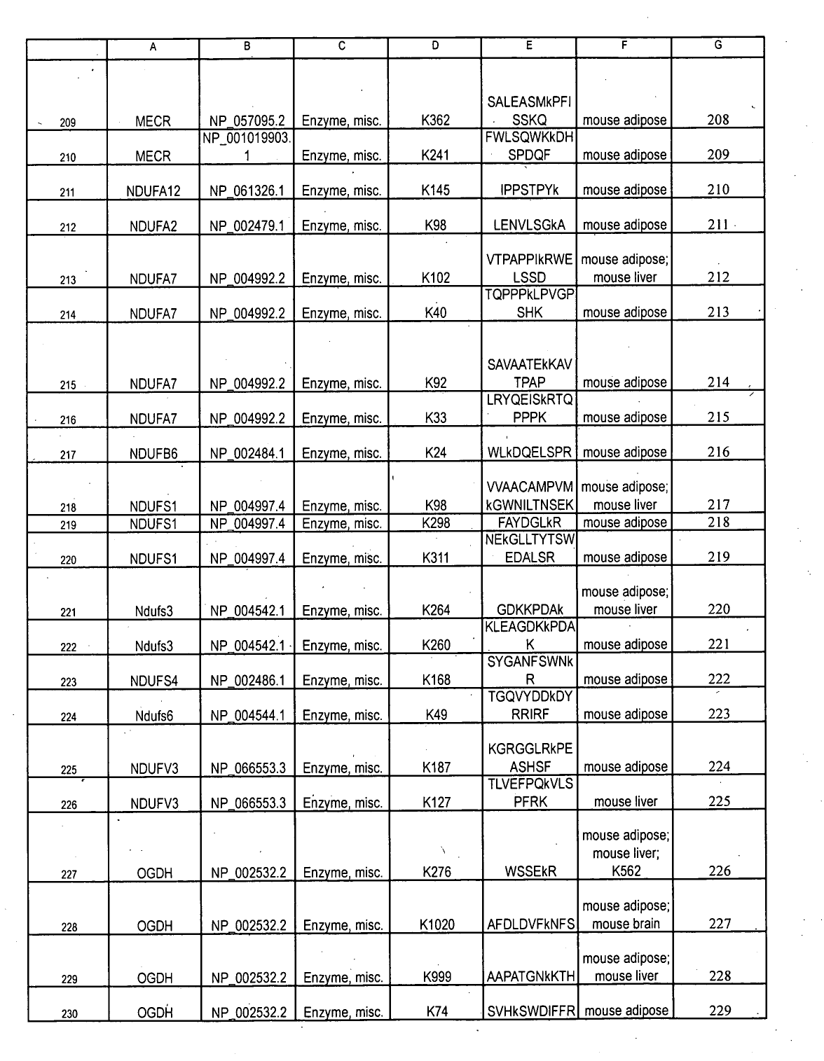

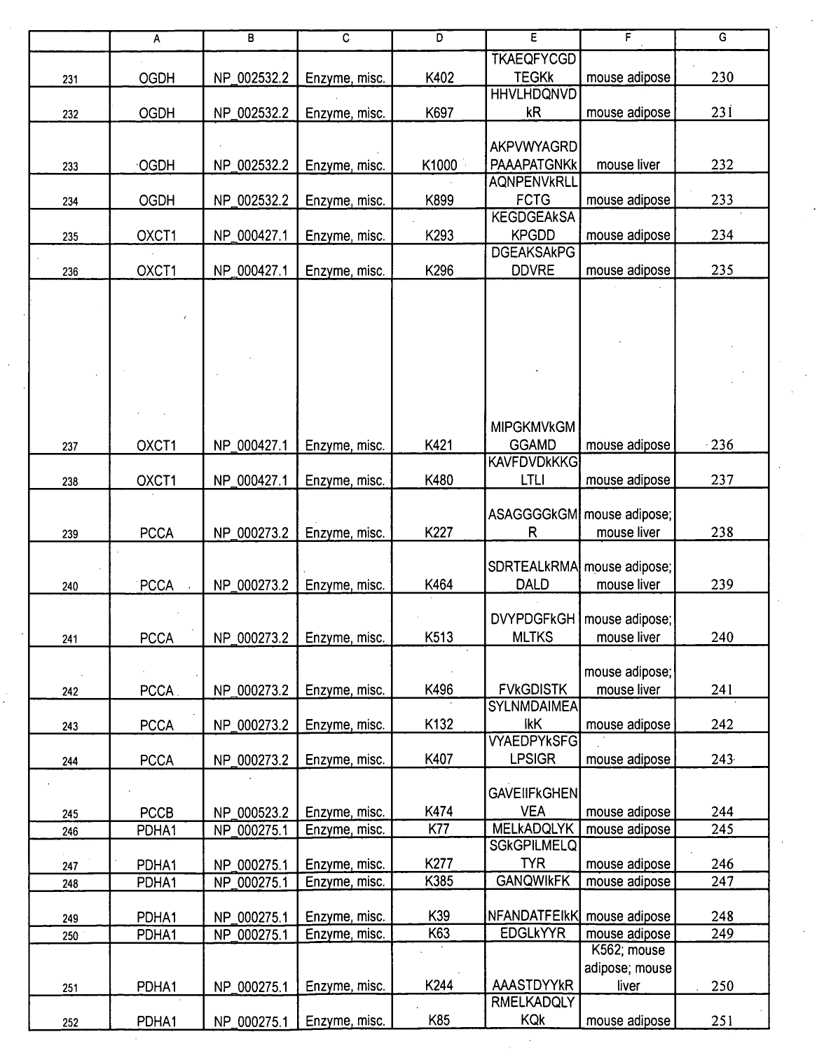

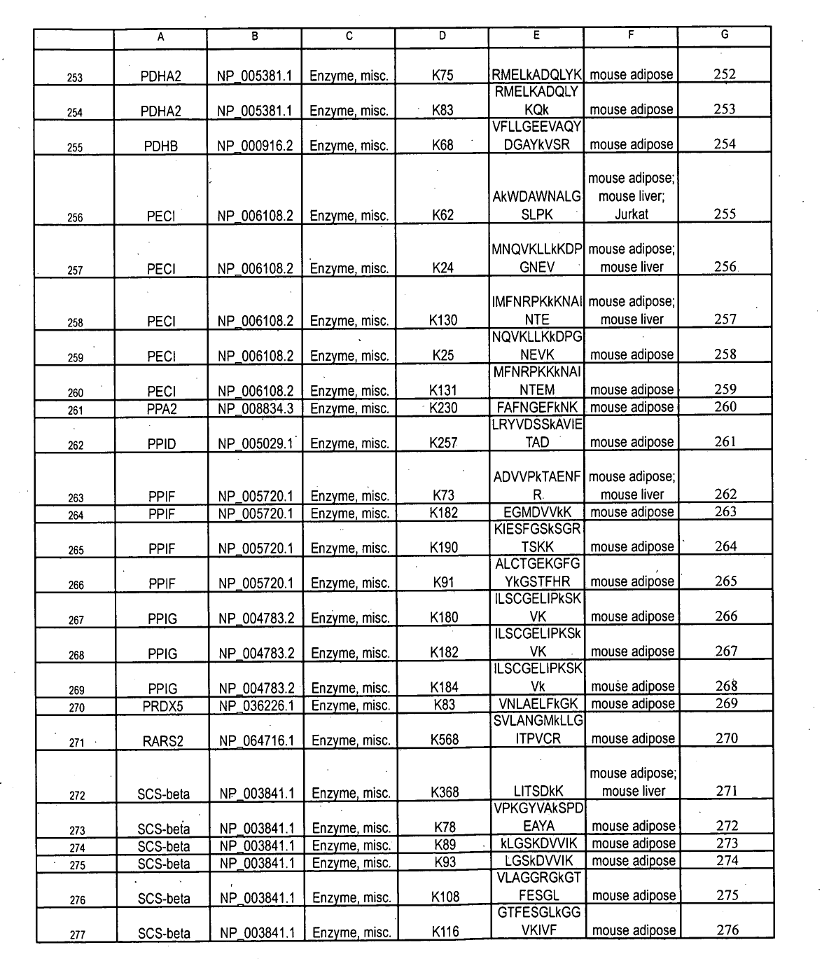

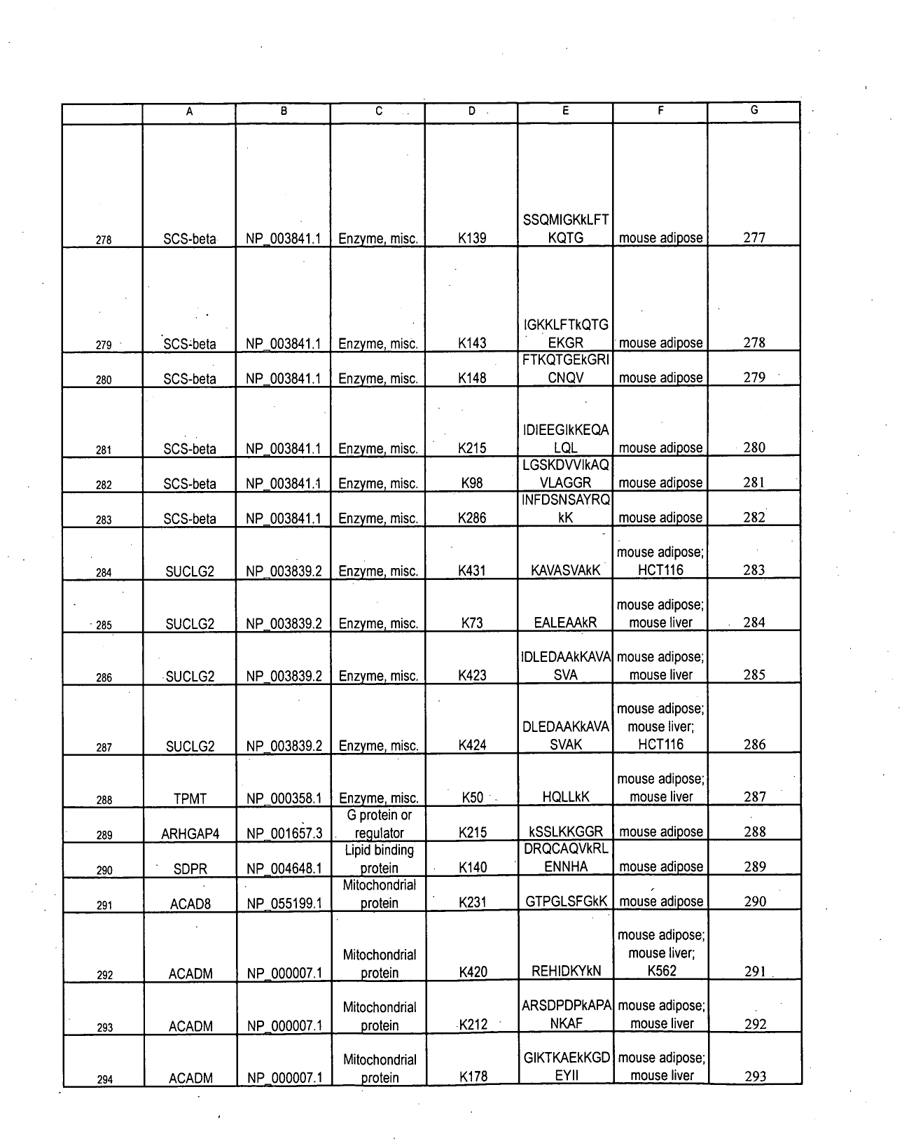

The novel acetylation sites identified are summarized in Table 1/FIG. 2. Column A lists the parent (signaling) protein in which the acetylation site occurs. Column D identifies the lysine residue at which acetylation occurs (each number refers to the amino acid residue position of the lysine in the parent human protein, according to the published sequence retrieved by the SwissProt accession number). Column E shows flanking sequences of the identified lysine residues (which are the sequences of trypsin-digested peptides).

TABLE 1
Novel Acetylation Sites.

| | | | | | | H |
|---|---|---|---|---|---|---|
| | A | | | D | E | SEQ. |
| | Protein | B | C | Acetyl- | Acetylation Site | ID. |
| 1 | Name | Accession No. | Protein Type | Residue | Sequence | NO: |

| | | | | | | |
|---|---|---|---|---|---|---|
| 2 | C14orf108 | NP_060699.2 | Adaptor/scaffold | K66 | LRLLDDDkDFVESRD | 1 |
| 3 | sciellin | NP_003834.2 | Adaptor/scaffold | K83 | VNERDVPkATISRYS | 2 |
| 4 | ZFP106 | NP_071918.1 | Adaptor/scaffold | K1037 | KNkRRK | 3 |
| 5 | ZFP106 | NP_071918.1 | Adaptor/scaffold | K1035 | kNKRRK | 4 |
| 6 | PDCD8 | NP_004199.1 | Apoptosis | K232 | VAVLTGKkVVQLDVR | 5 |
| 7 | PDCD8 | NP_004199.1 | Apoptosis | K244 | DVRDNMVkLNDGSQI | 6 |
| 8 | PDCD8 | NP_665811.1 | Apoptosis | K227 | GVAVLTGkKVVQLDV | 7 |
| 9 | LETM1 | NP_036450.1 | Calcium-binding protein | K738 | EKEVAEVkS | 8 |
| 10 | LETM1 | NP_036450.1 | Calcium-binding protein | K606 | VEESKASkRLTKRVQ | 9 |
| 11 | MPHOSPH6 | NP_005783.2 | Cell cycle regulation | K86 | GFNPEVEkLMLQMNA | 10 |
| 12 | HSP60 | NP_002147.2 | Chaperone | K369 | GDKAQIEkRIQEIIE | 11 |
| 13 | HSP60 | NP_955472.1 | Chaperone | K202 | KGVITVkDGK | 12 |
| 14 | HSP60 | NP_955472.1 | Chaperone | K361 | DAMLLKGkGDKAQIE | 13 |
| 15 | HSP60 | NP_002147.2 | Chaperone | K133 | ISkGANPVEIR | 14 |
| 16 | HSP60 | NP_955472.1 | Chaperone | K418 | VGGTSDVEVNEKkDR | 15 |
| 17 | HSP60 | NP_002147.2 | Chaperone | K364 | LLKGKGDkAQIEKRI | 16 |
| 18 | HSP75 | NP_057376.2 | Chaperone | K262 | IHLKSDCkEFSSEAR | 17 |
| 19 | HSP75 | NP_057376.2 | Chaperone | K95 | HEFQAETkKLLDIVA | 18 |
| 20 | HSP75 | NP_057376.2 | Chaperone | K109 | SLYSEkEVFIR | 19 |
| 21 | HSPA9B | NP_004125.3 | Chaperone | K678 | EGSGSSGTGEQKEDQKEEkQ | 20 |
| 22 | HSPA9B | NP_004125.3 | Chaperone | K467 | NTTIPTkK | 21 |
| 23 | HSPA9B | NP_004125.3 | Chaperone | K291 | HIVKEFkR | 22 |
| 24 | HSPA9B | NP_004125.3 | Chaperone | K600 | MEEFkDQLPADECNKLKEEISK | 23 |
| 25 | HSPA9B | NP_004125.3 | Chaperone | K653 | LFEMAYkK | 24 |
| 26 | HSPA9B | NP_004125.3 | Chaperone | K654 | LFEMAYKk | 25 |
| 27 | HSPA9B | NP_004125.3 | Chaperone | K671 | EGSGSSGTGEQkEDQKEEKQ | 26 |
| 28 | HSPA9B | NP_004125.3 | Chaperone | K612 | LkEEISK | 27 |
| 29 | MMAA | NP_758454.1 | Chaperone | K165 | TFIEYFGkMLTERGH | 28 |
| 30 | MMAA | NP_758454.1 | Chaperone | K140 | QEQSNKGkPLAFRVG | 29 |
| 31 | TID1 | NP_005138.2 | Chaperone | K242 | TCERCNGkGNEPGTK | 30 |
| 32 | TID1 | NP_005138.2 | Chaperone | K118 | KAYYQLAkK | 31 |
| 33 | TID1 | NP_005138.2 | Chaperone | K385 | PGTQTDQkIRMGGKG | 32 |
| 34 | POLG | NP_002684.1 | Chromatin, DNA-binding, | K1167 | YRAALALQITNLLTRCMFAYk | 33 |
| | | | DNA repair or DNA | | | |
| | | | replication protein | | | |
| 35 | KIF23 | NP_612565.1 | Motor or contractile | K16 | kGSQTNLK | 34 |
| | | | protein | | | |
| 36 | SEC61A1 | NP_037468.1 | Endoplasmic reticulum or | K392 | TWIEVSGSSAKDVAkQLKEQQ | 35 |
| | | | golgi | | MVMR | |
| 37 | SEC61A1 | NP_037468.1 | Endoplasmic reticulum or | K107 | IIEVGDTPkDR | 36 |
| | | | golgi | | | |
| 38 | SEC61A2 | NP_060614.2 | Endoplasmic reticulum or | K392 | TWIEVSGSSAKDVAkQLKEQQ | 37 |
| | | | golgi | | MVMR | |
| 39 | SEC61A2 | NP_060614.2 | Endoplasmic reticulum or | K107 | IIEVGDTPkDR | 38 |
| | | | golgi | | | |
| 40 | ACAA2 | NP_006102.1 | Enzyme, misc. | K13 | GVFVVAAkRTPFGAY | 39 |
| 41 | ACAA2 | NP_006102.1 | Enzyme, misc. | K191 | LQSQQRWkAANDAGY | 40 |
| 42 | ACAA2 | NP_006102.1 | Enzyme, misc. | K209 | EMAPIEVkTKKGKQT | 41 |
| 43 | ACAA2 | NP_006102.1 | Enzyme, misc. | K211 | APIEVKTkKGKQTMQ | 42 |
| 44 | ACAA2 | NP_006102.1 | Enzyme, misc. | K214 | KGkQTMQVDEHARPQTTLEQ | 43 |
| | | | | | LQK | |
| 45 | ACAA2 | NP_006102.1 | Enzyme, misc. | K340 | SLDLDISkTNVNGGA | 44 |
| 46 | ACAA2 | NP_006102.1 | Enzyme, misc. | K234 | TTLEQLQkLPPVFKK | 45 |
| 47 | ACAA2 | NP_006102.2 | Enzyme, misc. | K240 | QKLPPVFkKDGTVTA | 46 |
| 48 | ACAA2 | NP_006102.2 | Enzyme, misc. | K269 | IASEDAVkKHNFTPL | 47 |
| 49 | ACAA2 | NP_006102.2 | Enzyme, misc. | K305 | PAISGALkKAGLSLK | 48 |
| 50 | ACAA2 | NP_006102.2 | Enzyme, misc. | K306 | AISGALKkAGLSLKD | 49 |
| 51 | ACAA2 | NP_006102.2 | Enzyme, misc. | K241 | KLPPVFKkDGTVTAG | 50 |
| 52 | ACAA2 | NP_006102.2 | Enzyme, misc. | K212 | PIEVKTKkGKQTMQV | 51 |
| 53 | ACADL | NP_001599.1 | Enzyme, misc. | K42 | LETPSAkK | 52 |
| 54 | ACADL | NP_001599.1 | Enzyme, misc. | K240 | SPAHGISLFLVENGMkGFIK | 53 |
| 55 | ACADL | NP_001599.1 | Enzyme, misc. | K254 | KLHKMGLkAQDTAEL | 54 |
| 56 | ACADL | NP_001599.1 | Enzyme, misc. | K358 | CLQLHEAkRLDSATA | 55 |
| 57 | ACADL | NP_001599.1 | Enzyme, misc. | K318 | RNYVKQRkAFGKTVA | 56 |
| 58 | ACADL | NP_001599.1 | Enzyme, misc. | K322 | KQRKAFGkTVAHLQT | 57 |
| 59 | ACADL | NP_001599.1 | Enzyme, misc. | K189 | QGIKTNAkKDGSDWI | 58 |
| 60 | ACADS | NP_000008.1 | Enzyme, misc. | K226 | GISAFLVPMPTPGLTLGkK | 59 |
| 61 | ACADS | NP_000008.1 | Enzyme, misc. | K306 | AFGAPLTkLQVIQFK | 60 |
| 62 | ACADS | NP_000008.1 | Enzyme, misc. | K72 | LFPAAQVkKMGGLGL | 61 |
| 63 | ACADSB | NP_001600.1 | Enzyme, misc. | K324 | KERIQFGkRLFDFQG | 62 |
| 64 | ACADVL | NP_000009.1 | Enzyme, misc. | K298 | ITHGPPEkKMGIKAS | 63 |
| 65 | ACADVL | NP_000009.1 | Enzyme, misc. | K278 | EkITAFVVER | 64 |
| 66 | ACADVL | NP_000009.1 | Enzyme, misc. | K556 | HKkGIVNEQFLLQR | 65 |
| 67 | ACAT1 | NP_000010.1 | Enzyme, misc. | K223 | INSYTRSkAAWEAGK | 66 |
| 68 | ACAT1 | NP_000010.1 | Enzyme, misc. | K243 | IPVTVTVkGQPDVVV | 67 |
| 69 | ACAT1 | NP_000010.1 | Enzyme, misc. | K268 | FSKVPKLkTVFQKEN | 68 |
| 70 | ACAT1 | NP_000010.1 | Enzyme, misc. | K266 | VDFSKVPkLK | 69 |
| 71 | ACAT1 | NP_000010.1 | Enzyme, misc. | K273 | KLKTVFQkENGTVTA | 70 |
| 72 | ACAT1 | NP_000010.1 | Enzyme, misc. | K202 | SCAENTAkKLNIARN | 71 |
| 73 | ACAT1 | NP_000010.1 | Enzyme, misc. | K257 | VKEDEEYkRVDFSKV | 72 |
| 74 | ACAT1 | NP_000010.1 | Enzyme, misc. | K343 | VLKDVGLkKEDIAMW | 73 |
| 75 | ACSL1 | NP_001986.2 | Enzyme, misc. | K386 | GQANTTLkRWLLDFA | 74 |
| 76 | ACSL1 | NP_001986.2 | Enzyme, misc. | K263 | RANRRKPkPPAPEDL | 75 |
| 77 | AKR7A2 | NP_003680.2 | Enzyme, misc. | K128 | SQLETSLkR | 76 |
| 78 | AKR7A2 | NP_003680.2 | Enzyme, misc. | K242 | YKYEDkDGKQPVGR | 77 |
| 79 | ALDH2 | NP_000681.2 | Enzyme, misc. | K428 | FkTIEEVVGR | 78 |
| 80 | ALDH2 | NP_000681.2 | Enzyme, misc. | K369 | VDETQFKkILGYINT | 79 |
| 81 | BCKDHB | NP_000047.1 | Enzyme, misc. | K232 | GLLLSCIEDkNPCIFFEPK | 80 |
| 82 | CBR1 | NP_001748.1 | Enzyme, misc. | K157 | CSPELQQkFRSETIT | 81 |
| 83 | COQ3 | NP_059117.3 | Enzyme, misc. | K196 | SFDPVLDkR | 82 |
| 84 | COQ3 | NP_059117.3 | Enzyme, misc. | K149 | GKPLLGMkILDVGCG | 83 |
| 85 | COX4I1 | NP_001852.1 | Mitochondrial protein | K67 | KALKEKEkASWSSLS | 84 |
| 86 | COX4I1 | NP_001852.1 | Mitochondrial protein | K29 | RAHESVVkSEDFSLP | 85 |
| 87 | CPT1B | NP_689451.1 | Enzyme, misc. | K41 | SGINSWKkRLIRIKN | 86 |
| 88 | CPT1B | NP_689451.1 | Enzyme, misc. | K404 | QAFFSSGkNKAALEA | 87 |
| 89 | CPT2 | NP_000089.1 | Enzyme, misc. | K69 | RRYLSAQkPLLNDGQ | 88 |
| 90 | CPT2 | NP_000089.1 | Enzyme, misc. | K239 | DELFTDDkARHLLVL | 89 |
| 91 | CPT2 | NP_000089.1 | Enzyme, misc. | K457 | GGKEFLkK | 90 |
| 92 | CRAT | NP_000746.2 | Mitochondrial protein | K268 | KAYNTLIkDKVNRDS | 91 |
| 93 | CRAT | NP_000746.2 | Mitochondrial protein | K379 | SPMVPLPMPkK | 92 |
| 94 | CYP20A1 | NP_803882.1 | Enzyme, misc. | K243 | RNIIKERkGRNFSQH | 93 |
| 95 | DBT | NP_001909.2 | Enzyme, misc. | K196 | LSEVVGSGkDGR | 94 |
| 96 | DBT | NP_001909.2 | Enzyme, misc. | K304 | GIkLSFMPFFLK | 95 |
| 97 | DBT | NP_001909.2 | Enzyme, misc. | K435 | AIPRFNQkGEVYKAQ | 96 |
| 98 | DBT | NP_001909.2 | Enzyme, misc. | K202 | GKDGRILkEDILNYL | 97 |
| 99 | DBT | NP_001909.2 | Enzyme, misc. | K233 | MPPPPKPkDMTVPIL | 98 |
| 100 | DBT | NP_001909.2 | Enzyme, misc. | K119 | SRYDGVIkKLYYNLD | 99 |
| 101 | DBT | NP_001909.2 | Enzyme, misc. | K250 | KPPVFTGkDKTEPIK | 100 |
| 102 | DBT | NP_001909.2 | Enzyme, misc. | K482 | SYLENPAFMLLDLk | 101 |
| 103 | DCI | NP_001910.2 | Enzyme, misc. | K89 | LEKLENDkSFRGVIL | 102 |
| 104 | DCI | NP_001910.2 | Enzyme, misc. | K283 | NFVSFISkDSIQKSL | 103 |
| 105 | DCI | NP_001910.2 | Enzyme, misc. | K84 | ELVISLEkLENDKSF | 104 |
| 106 | DECR1 | NP_001350.1 | Enzyme, misc. | K246 | TkGAFSR | 105 |
| 107 | DECR1 | NP_001350.1 | Enzyme, misc. | K185 | QLIkAQK | 106 |
| 108 | DECR1 | NP_001350.1 | Enzyme, misc. | K49 | KFFSPLQkAMLPPNS | 107 |
| 109 | DECR1 | NP_001350.1 | Enzyme, misc. | K260 | DPTGTFEkEMIGRIP | 108 |
| 110 | DECR1 | NP_001350.1 | Enzyme, misc. | K316 | GEFNDLRkVTKEQWD | 109 |
| 111 | DLAT | NP_001922.2 | Enzyme, misc. | K376 | GIDLTQVkGTGPDGR | 110 |
| 112 | DLAT | NP_001922.2 | Enzyme, misc. | K637 | QWLAEFRkYLEKPIT | 111 |
| 113 | ECHS1 | NP_004083.2 | Enzyme, misc. | K288 | EKRKANFkDQ | 112 |
| 114 | ECHS1 | NP_004083.2 | Enzyme, misc. | K282 | EGMTAFVEkR | 113 |
| 115 | ECHS1 | NP_004083.2 | Enzyme, misc. | K284 | TAFVEKRkANFKDQ | 114 |
| 116 | ECHS1 | NP_004083.2 | Enzyme, misc. | K204 | ISAQDAkQAGLVSK | 115 |
| 117 | ECHS1 | NP_004083.2 | Enzyme, misc. | K43 | IIAEKRGkNNTVGLI | 116 |
| 118 | EZH2 | NP_004447.2 | Enzyme, misc. | K314 | kNTETALDNK | 117 |
| 119 | FH | NP_000134.2 | Enzyme, misc. | K80 | VRSTMNFkIGGVTER | 118 |
| 120 | FH | NP_000134.2 | Enzyme, misc. | K467 | LMNESLMLVTALNPHIGYDkA | 119 |
| | | | | | AK | |
| 121 | FH | NP_000134.2 | Enzyme, misc. | K470 | AAkIAKTAHK | 120 |
| 122 | FH | NP_000134.2 | Enzyme, misc. | K473 | AAKIAkTAHK | 121 |
| 123 | FH | NP_000134.2 | Enzyme, misc. | K263 | IkAAMPR | 122 |
| 124 | FH | NP_000134.2 | Enzyme, misc. | K292 | IGFAEkVAAK | 123 |
| 125 | GRIM-19 | NP_057049.4 | Enzyme, misc. | K90 | VkQDMPPPGGYGPIDYKR | 124 |
| 126 | GRIM-19 | NP_057049.4 | Enzyme, misc. | K105 | VKQDMPPPGGYGPIDYkR | 125 |
| 127 | HADHA | NP_000173.2 | Mitochondrial protein | K735 | KIVDRLKkYEAAYGK | 126 |
| 128 | HADHA | NP_000173.2 | Mitochondrial protein | K259 | ADKKISPkRDKGLVE | 127 |
| 129 | HADHA | NP_000173.2 | Mitochondrial protein | K289 | VYKKVEEkVRKQTKG | 128 |
| 130 | HADHA | NP_000173.2 | Mitochondrial protein | K413 | GLNDKVkK | 129 |
| 131 | HADHA | NP_000173.2 | Mitochondrial protein | K414 | GLNDKVKkK | 130 |
| 132 | HADHA | NP_000173.2 | Mitochondrial protein | K415 | kALTSFER | 131 |
| 133 | HADHA | NP_000173.2 | Mitochondrial protein | K519 | ITTEKTSkDTSASAV | 132 |
| 134 | HADHA | NP_000173.2 | Mitochondrial protein | K505 | HYFSPVDkMQLLEII | 133 |
| 135 | HADHA | NP_000173.2 | Mitochondrial protein | K540 | VIIVVkDGPGFYTTR | 134 |
| 136 | HADHA | NP_000173.2 | Mitochondrial protein | K46 | THINYGVkGDVAVVR | 135 |
| 137 | HADHA | NP_000173.2 | Mitochondrial protein | K166 | RIATKDRkTVLGTPE | 136 |
| 138 | HADHA | NP_000173.2 | Mitochondrial protein | K213 | SIRADRAkKMGLVDQ | 137 |
| 139 | HADHA | NP_000173.2 | Mitochondrial protein | K214 | IRADRAKkMGLVDQL | 138 |
| 140 | HADHA | NP_000173.2 | Mitochondrial protein | K262 | KISPKRDkGLVEKLT | 139 |
| 141 | HADHA | NP_000173.2 | Mitochondrial protein | K292 | kQTKGLYPAPLK | 140 |
| 142 | HADHA | NP_000173.2 | Mitochondrial protein | K295 | QTkGLYPAPLK | 141 |
| 143 | HADHA | NP_000173.2 | Mitochondrial protein | K303 | GLYPAPLk | 142 |
| 144 | HADHA | NP_000173.2 | Mitochondrial protein | K411 | GLNDkVKKK | 143 |
| 145 | HADHA | NP_000173.2 | Mitochondrial protein | K631 | SKGFLGRkSGKGFYI | 144 |
| 146 | HADHA | NP_000173.2 | Mitochondrial protein | K634 | FLGRKSGkGFYIYQE | 145 |
| 147 | HADHA | NP_000173.2 | Mitochondrial protein | K759 | DHANSPNkKFYQ | 146 |
| 148 | HADHA | NP_000173.2 | Mitochondrial protein | K255 | AKGLADKkISPKRDK | 147 |
| 149 | HADHB | NP_000174.1 | Enzyme, misc. | K293 | LEQMAKLkPAFIKPY | 148 |
| 150 | HADHB | NP_000174.1 | Enzyme, misc. | K447 | AAANRLRkEGGQYGL | 149 |
| 151 | HADHB | NP_000174.1 | Enzyme, misc. | K188 | KLMLDLNkAKSMGQR | 150 |
| 152 | HADHB | NP_000174.1 | Enzyme, misc. | K272 | VPFKVPGkDTVTKDN | 151 |
| 153 | HADHB | NP_000174.1 | Enzyme, misc. | K298 | KLKPAFIkPYGTVTA | 152 |
| 154 | HADHB | NP_000174.1 | Enzyme, misc. | K474 | MIVEAYPk | 153 |
| 155 | HADHB | NP_000174.1 | Enzyme, misc. | K253 | LRSHSLAkKAQDEGL | 154 |
| 156 | HADHSC | NP_005318.2 | Enzyme, misc. | K81 | LRKVAKKkFAENPKA | 155 |
| 157 | HADHSC | NP_005318.2 | Enzyme, misc. | K68 | DILAKSKkGIEESLR | 156 |
| 158 | HADHSC | NP_005318.2 | Enzyme, misc. | K185 | LVEVIkTPMTSQK | 157 |
| 159 | HADHSC | NP_005318.2 | Enzyme, misc. | K136 | ELFKRLDkFAAEHTI | 158 |
| 160 | HADHSC | NP_005318.2 | Enzyme, misc. | K192 | KTPMTSQkTFESLVD | 159 |
| 161 | HADHSC | NP_005318.2 | Enzyme, misc. | K301 | NKLVAENkFGKKTGE | 160 |
| 162 | HADHSC | NP_005318.2 | Enzyme, misc. | K312 | KTGEGFYk | 161 |
| 163 | HADHSC | NP_005318.2 | Enzyme, misc. | K87 | KKFAENLkAGDEFVE | 162 |
| 164 | HADHSC | NP_005318.2 | Enzyme, misc. | K249 | GDASKEDIDTAMkLGAGYPMG | 163 |
| | | | | | PFELLDYVGLDTTK | |
| 165 | HADHSC | NP_005318.2 | Enzyme, misc. | K314 | TGEGFYKYk | 164 |
| 166 | HIBCH | NP_055177.2 | Enzyme, misc. | K360 | KDQSPKWkPADLKEV | 165 |
| 167 | HIBCH | NP_055177.2 | Enzyme, misc. | K55 | VITLNRPkFLNALTL | 166 |
| 168 | HIBCH | NP_055177.2 | Enzyme, misc. | K358 | IDKDQSPkWKPADLK | 167 |
| 169 | HIBCH | NP_055177.2 | Enzyme, misc. | K365 | KWKPADLkEVTEEDL | 168 |
| 170 | HIBCH | NP_932164.1 | Enzyme, misc. | K87 | ETFLIIIkGAGGKAF | 169 |
| 171 | IDH2 | NP_002159.2 | Enzyme, misc. | K180 | HAHGDQYkATDFVAD | 170 |
| 172 | IDH2 | NP_002159.2 | Enzyme, misc. | K166 | LVPGWTkPITIGR | 171 |
| 173 | IDH2 | NP_002159.2 | Enzyme, misc. | K133 | MWkSPNGTIR | 172 |
| 174 | IDH2 | NP_002159.2 | Enzyme, misc. | K155 | NILGGTVFREPIICkNIPR | 173 |
| 175 | IDH2 | NP_002159.2 | Enzyme, misc. | K199 | FKMVFTPkDGSGVKE | 174 |
| 176 | IDH2 | NP_002159.2 | Enzyme, misc. | K360 | EHQkGRPTSTNPIASIFAWTR | 175 |
| 177 | IDH2 | NP_002159.2 | Enzyme, misc. | K400 | RFAQMLEkVCVETVE | 176 |
| 178 | IDH3A | NP_005521.1 | Enzyme, misc. | K177 | LITEGASkRIAEFAF | 177 |
| 179 | IDH3A | NP_005521.1 | Enzyme, misc. | K58 | IFDAAkAPIQWEER | 178 |
| 180 | IDH3A | NP_005521.1 | Enzyme, misc. | K96 | MGLkGPLK | 179 |
| 181 | IDH3A | NP_005521.1 | Enzyme, misc. | K100 | GPLkTPIAAGHPSMNLLLR | 180 |
| 182 | IDH3A | NP_005521.1 | Enzyme, misc. | K336 | IEAACFATIkDGK | 181 |
| 183 | IDH3A | NP_005521.1 | Enzyme, misc. | K339 | IEAACFATIKDGk | 182 |
| 184 | IDH3A | NP_005521.1 | Enzyme, misc. | K343 | SLTkDLGGNAK | 183 |
| 185 | IDH3A | NP_005521.1 | Enzyme, misc. | K363 | RVkDLD | 184 |
| 186 | IDH3B | NP_008830.2 | Enzyme, misc. | K354 | DAVKKVIkVGKVRTR | 185 |
| 187 | IDH3B | NP_777280.1 | Enzyme, misc. | K122 | IHTPMEYkGELASYD | 186 |
| 188 | IDH3B | NP_777280.1 | Enzyme, misc. | K146 | FANVVHVkSLPGYMT | 187 |
| 189 | IDH3B | NP_008830.2 | Enzyme, misc. | K350 | HLNLEYHSSMIADAVkK | 188 |
| 190 | IDH3B | NP_008830.2 | Enzyme, misc. | K351 | MIADAVKkVIKVGKV | 189 |
| 191 | IDH3G | NP_004126.1 | Enzyme, misc. | K159 | HkDIDILIVR | 190 |
| 192 | IDH3G | NP_777358.1 | Enzyme, misc. | K206 | IAEYAFkLAQESGR | 191 |
| 193 | IDH3G | NP_004126.1 | Enzyme, misc. | K226 | ANIMkLGDGLFLQCCR | 192 |
| 194 | KMO | NP_003670.1 | Enzyme, misc. | K138 | HFNHRLLkCNPEEGM | 193 |
| 195 | KMO | NP_003670.1 | Enzyme, misc. | K179 | TVRSHLMkKPRFDYS | 194 |
| 196 | MCCC1 | NP_064551.3 | Mitochondrial protein | K721 | FEEEESDkRESE | 195 |
| 197 | MCCC2 | NP_071415.1 | Enzyme, misc. | K70 | IKLGGGEkARALHIS | 196 |
| 198 | MCCC2 | NP_071415.1 | Enzyme, misc. | K141 | IANDATVkGGAYYPV | 197 |
| 199 | MCEE | NP_115990.2 | Enzyme, misc. | K114 | PIAGFLQkNKAGGMH | 198 |
| 200 | MCEE | NP_115990.2 | Enzyme, misc. | K150 | RSLSEEVkIGAHGKP | 199 |
| 201 | MDH2 | NP_005909.2 | Enzyme, misc. | K78 | IETKAAVkGYLGPEQ | 200 |
| 202 | MDH2 | NP_005909.2 | Enzyme, misc. | K328 | ASIkKGEDFVK | 201 |
| 203 | MDH2 | NP_005909.2 | Enzyme, misc. | K335 | KKGEDFVkTLK | 202 |
| 204 | MDH2 | NP_005909.2 | Enzyme, misc. | K203 | VNVPVIGGHAGkTIIPLISQCTPK | 203 |
| 205 | MDH2 | NP_005909.2 | Enzyme, misc. | K307 | EKNLGIGkVSSFEEK | 204 |
| 206 | MECR | NP_057095.2 | Enzyme, misc. | K248 | ELRRPEMkNFFKDMP | 205 |
| 207 | MECR | NP_057095.2 | Enzyme, misc. | K252 | PEMKNFFkDMPQPRL | 206 |
| 208 | MECR | NP_001019903.1 | Enzyme, misc. | K191 | LALNCVGGkSSTELLR | 207 |
| 209 | MECR | NP_057095.2 | Enzyme, misc. | K362 | SALEASMkPFISSKQ | 208 |
| 210 | MECR | NP_001019903.1 | Enzyme, misc. | K241 | FWLSQWKkDHSPDQF | 209 |
| 211 | NDUFA12 | NP_061326.1 | Enzyme, misc. | K145 | IPPSTPYk | 210 |
| 212 | NDUFA2 | NP_002479.1 | Enzyme, misc. | K98 | LENVLSGkA | 211 |
| 213 | NDUFA7 | NP_004992.2 | Enzyme, misc. | K102 | VTPAPPIkRWELSSD | 212 |
| 214 | NDUFA7 | NP_004992.2 | Enzyme, misc. | K40 | TQPPPkLPVGPSHK | 213 |
| 215 | NDUFA7 | NP_004992.2 | Enzyme, misc. | K92 | SAVAATEkKAVTPAP | 214 |
| 216 | NDUFA7 | NP_004992.2 | Enzyme, misc. | K33 | LRYQEISkRTQPPPK | 215 |
| 217 | NDUFB6 | NP_002484.1 | Enzyme, misc. | K24 | WLkDQELSPR | 216 |
| 218 | NDUFS1 | NP_004997.4 | Enzyme, misc. | K98 | VVAACAMPVMkGWNILTNSEK | 217 |
| 219 | NDUFS1 | NP_004997.4 | Enzyme, misc. | K298 | FAYDGLkR | 218 |
| 220 | NDUFS1 | NP_004997.4 | Enzyme, misc. | K311 | NEkGLLTYTSWEDALSR | 219 |
| 221 | Ndufs3 | NP_004542.1 | Enzyme, misc. | K264 | GDKKPDAk | 220 |
| 222 | Ndufs3 | NP_004542.1 | Enzyme, misc. | K260 | KLEAGDKkPDAK | 221 |
| 223 | NDUFS4 | NP_002486.1 | Enzyme, misc. | K168 | SYGANFSWNkR | 222 |
| 224 | Ndufs6 | NP_004544.1 | Enzyme, misc. | K49 | TGQVYDDkDYRRIRF | 223 |
| 225 | NDUFV3 | NP_066553.3 | Enzyme, misc. | K187 | KGRGGLRkPEASHSF | 224 |
| 226 | NDUFV3 | NP_066553.3 | Enzyme, misc. | K127 | TLVEFPQkVLSPFRK | 225 |
| 227 | OGDH | NP_002532.2 | Enzyme, misc. | K276 | WSSEkR | 226 |
| 228 | OGDH | NP_002532.2 | Enzyme, misc. | K1020 | AFDLDVFkNFS | 227 |
| 229 | OGDH | NP_002532.2 | Enzyme, misc. | K999 | AAPATGNkKTH | 228 |
| 230 | OGDH | NP_002532.2 | Enzyme, misc. | K74 | SVHkSWDIFFR | 229 |
| 231 | OGDH | NP_002532.2 | Enzyme, misc. | K402 | TKAEQFYCGDTEGKk | 230 |
| 232 | OGDH | NP_002532.2 | Enzyme, misc. | K697 | HHVLHDQNVDkR | 231 |
| 233 | OGDH | NP_002532.2 | Enzyme, misc. | K1000 | AKPVWYAGRDPAAAPATGNKk | 232 |
| 234 | OGDH | NP_002532.2 | Enzyme, misc. | K899 | AQNPENVkRLLFCTG | 233 |
| 235 | OXCT1 | NP_000427.1 | Enzyme, misc. | K293 | KEGDGEAkSAKPGDD | 234 |
| 236 | OXCT1 | NP_000427.1 | Enzyme, misc. | K296 | DGEAKSAkPGDDVRE | 235 |
| 237 | OXCT1 | NP_000427.1 | Enzyme, misc. | K421 | MIPGKMVkGMGGAMD | 236 |
| 238 | OXCT1 | NP_000427.1 | Enzyme, misc. | K480 | KAVFDVDkKKGLTLI | 237 |
| 239 | PCCA | NP_000273.2 | Enzyme, misc. | K227 | ASAGGGGkGMR | 238 |
| 240 | PCCA | NP_000273.2 | Enzyme, misc. | K464 | SDRTEALkRMADALD | 239 |
| 241 | PCCA | NP_000273.2 | Enzyme, misc. | K513 | DVYPDGFkGHMLTKS | 240 |
| 242 | PCCA | NP_000273.2 | Enzyme, misc. | K496 | FVkGDISTK | 241 |
| 243 | PCCA | NP_000273.2 | Enzyme, misc. | K132 | SYLNMDAIMEAIkK | 242 |
| 244 | PCCA | NP_000273.2 | Enzyme, misc. | K407 | VYAEDPYkSFGLPSIGR | 243 |
| 245 | PCCB | NP_000523.2 | Enzyme, misc. | K474 | GAVEIIFkGHENVEA | 244 |
| 246 | PDHA1 | NP_000275.1 | Enzyme, misc. | K77 | MELkADQLYK | 245 |
| 247 | PDHA1 | NP_000275.1 | Enzyme, misc. | K277 | SGkGPILMELQTYR | 246 |
| 248 | PDHA1 | NP_000275.1 | Enzyme, misc. | K385 | GANQWIkFK | 247 |
| 249 | PDHA1 | NP_000275.1 | Enzyme, misc. | K39 | NFANDATFEIkK | 248 |
| 250 | PDHA1 | NP_000275.1 | Enzyme, misc. | K63 | EDGLkYYR | 249 |
| 251 | PDHA1 | NP_000275.1 | Enzyme, misc. | K244 | AAASTDYYkR | 250 |
| 252 | PDHA1 | NP_000275.1 | Enzyme, misc. | K85 | RMELKADQLYKQk | 251 |
| 253 | PDHA2 | NP_005381.1 | Enzyme, misc. | K75 | RMELkADQLYK | 252 |
| 254 | PDHA2 | NP_005381.1 | Enzyme, misc. | K83 | RMELKADQLYKQk | 253 |
| 255 | PDHB | NP_000916.2 | Enzyme, misc. | K68 | VFLLGEEVAQYDGAYkVSR | 254 |
| 256 | PECI | NP_006108.2 | Enzyme, misc. | K62 | AkWDAWNALGSLPK | 255 |
| 257 | PECI | NP_006108.2 | Enzyme, misc. | K24 | MNQVKLLkKDPGNEV | 256 |
| 258 | PECI | NP_006108.2 | Enzyme, misc. | K130 | IMFNRPKkKNAINTE | 257 |
| 259 | PECI | NP_006108.2 | Enzyme, misc. | K25 | NQVKLLKkDPGNEVK | 258 |
| 260 | PECI | NP_006108.2 | Enzyme, misc. | K131 | MFNRPKKkNAINTEM | 259 |
| 261 | PPA2 | NP_008834.3 | Enzyme, misc. | K230 | FAFNGEFkNK | 260 |
| 262 | PPID | NP_005029.1 | Enzyme, misc. | K257 | LRYVDSSkAVIETAD | 261 |
| 263 | PPIF | NP_005720.1 | Enzyme, misc. | K73 | ADVVPkTAENFR | 262 |
| 264 | PPIF | NP_005720.1 | Enzyme, misc. | K182 | EGMDVVkK | 263 |
| 265 | PPIF | NP_005720.1 | Enzyme, misc. | K190 | KIESFGSkSGRTSKK | 264 |
| 266 | PPIF | NP_005720.1 | Enzyme, misc. | K91 | ALCTGEKGFGYkGSTFHR | 265 |
| 267 | PPIG | NP_004783.2 | Enzyme, misc. | K180 | ILSCGELIPkSKVK | 266 |
| 268 | PPIG | NP_004783.2 | Enzyme, misc. | K182 | ILSCGELIPKSkVK | 267 |
| 269 | PPIG | NP_004783.2 | Enzyme, misc. | K184 | ILSCGELIPKSKVk | 268 |
| 270 | PRDX5 | NP_036226.1 | Enzyme, misc. | K83 | VNLAELFkGK | 269 |
| 271 | RARS2 | NP_064716.1 | Enzyme, misc. | K568 | SVLANGMkLLGITPVCR | 270 |
| 272 | SCS-beta | NP_003841.1 | Enzyme, misc. | K368 | LITSDkK | 271 |
| 273 | SCS-beta | NP_003841.1 | Enzyme, misc. | K78 | VPKGYVAkSPDEAYA | 272 |
| 274 | SCS-beta | NP_003841.1 | Enzyme, misc. | K89 | kLGSKDVVIK | 273 |
| 275 | SCS-beta | NP_003841.1 | Enzyme, misc. | K93 | LGSkDVVIK | 274 |
| 276 | SCS-beta | NP_003841.1 | Enzyme, misc. | K108 | VLAGGRGkGTFESGL | 275 |
| 277 | SCS-beta | NP_003841.1 | Enzyme, misc. | K116 | GTFESGLkGGVKIVF | 276 |
| 278 | SCS-beta | NP_003841.1 | Enzyme, misc. | K139 | SSQMIGKkLFTKQTG | 277 |
| 279 | SCS-beta | NP_003841.1 | Enzyme, misc. | K143 | IGKKLFTkQTGEKGR | 278 |
| 280 | SCS-beta | NP_003841.1 | Enzyme, misc. | K148 | FTKQTGEkGRICNQV | 279 |
| 281 | SCS-beta | NP_003841.1 | Enzyme, misc. | K215 | IDIEEGIkKEQALQL | 280 |
| 282 | SCS-beta | NP_003841.1 | Enzyme, misc. | K98 | LGSKDVVIkAQVLAGGR | 281 |
| 283 | SCS-beta | NP_003841.1 | Enzyme, misc. | K286 | INFDSNSAYRQkK | 282 |
| 284 | SUCLG2 | NP_003839.2 | Enzyme, misc. | K431 | KAVASVAkK | 283 |
| 285 | SUCLG2 | NP_003839.2 | Enzyme, misc. | K73 | EALEAAkR | 284 |
| 286 | SUCLG2 | NP_003839.2 | Enzyme, misc. | K423 | IDLEDAAkKAVASVA | 285 |
| 287 | SUCLG2 | NP_003839.2 | Enzyme, misc. | K424 | DLEDAAKkAVASVAK | 286 |
| 288 | TPMT | NP_000358.1 | Enzyme, misc. | K50 | HQLLkK | 287 |
| 289 | ARHGAP4 | NP_001657.3 | G protein or regulator | K215 | kSSLKKGGR | 288 |
| 290 | SDPR | NP_004648.1 | Lipid binding protein | K140 | DRQCAQVkRLENNHA | 289 |
| 291 | ACAD8 | NP_055199.1 | Mitochondrial protein | K231 | GTPGLSFGkK | 290 |
| 292 | ACADM | NP_000007.1 | Mitochondrial protein | K420 | REHIDKYkN | 291 |
| 293 | ACADM | NP_000007.1 | Mitochondrial protein | K212 | ARSDPDPkAPANKAF | 292 |
| 294 | ACADM | NP_000007.1 | Mitochondrial protein | K178 | GIKTKAEkKGDEYII | 293 |
| 295 | ACADM | NP_000007.1 | Mitochondrial protein | K259 | FEDVKVPkENVLIGD | 294 |
| 296 | ACADM | NP_000007.1 | Mitochondrial protein | K175 | DVAGIKTkAEKKGDE | 295 |
| 297 | ACADM | NP_000007.1 | Mitochondrial protein | K271 | IGDGAGFkVAMGAFD | 296 |
| 298 | ACADM | NP_000007.1 | Mitochondrial protein | K179 | IKTKAEKkGDEYIIN | 297 |
| 299 | ACADM | NP_000007.1 | Mitochondrial protein | K236 | PGIQIGRkELNMGQR | 298 |
| 300 | ACO2 | NP_001089.1 | Mitochondrial protein | K700 | IHETNLkK | 299 |
| 301 | ACO2 | NP_001089.1 | Mitochondrial protein | K31 | SVLCQRAkVAMSHFE | 300 |
| 302 | ACO2 | NP_001089.1 | Mitochondrial protein | K138 | VAVPSTIHCDHLIEAQVGGEkD | 301 |
| | | | | | LR | |
| 303 | ACO2 | NP_001089.1 | Mitochondrial protein | K160 | DINQEVYNFLATAGAk | 302 |
| 304 | ACO2 | NP_001089.1 | Mitochondrial protein | K228 | CPkVIGVK | 303 |
| 305 | ACO2 | NP_001089.1 | Mitochondrial protein | K304 | MkKYLSK | 304 |
| 306 | ACO2 | NP_001089.1 | Mitochondrial protein | K305 | MKkYLSK | 305 |
| 307 | ACO2 | NP_001089.1 | Mitochondrial protein | K401 | SAAVAkQALAHGLKCK | 306 |
| 308 | ACO2 | NP_001089.1 | Mitochondrial protein | K409 | SAAVAKQALAHGLkCK | 307 |
| 309 | ACO2 | NP_001089.1 | Mitochondrial protein | K411 | CkSQFTITPGSEQIR | 308 |
| 310 | ACO2 | NP_001089.1 | Mitochondrial protein | K458 | CIGQWDRkDIKKGEK | 309 |
| 311 | ACO2 | NP_001089.1 | Mitochondrial protein | K462 | kGEKNTIVTSYNR | 310 |
| 312 | ACO2 | NP_001089.1 | Mitochondrial protein | K465 | KGEkNTIVTSYNR | 311 |
| 313 | ACO2 | NP_001089.1 | Mitochondrial protein | K591 | GkCTTDHISAAGPWLK | 312 |
| 314 | ACO2 | NP_001089.1 | Mitochondrial protein | K605 | CTTDHISAAGPWLkFR | 313 |
| 315 | ACO2 | NP_001089.1 | Mitochondrial protein | K628 | GHLDNISNNLLIGAINIENGkAN | 314 |
| | | | | | SVR | |
| 316 | ACO2 | NP_001089.1 | Mitochondrial protein | K743 | KPLKCIIkHPNGTQE | 315 |
| 317 | ACO2 | NP_001089.1 | Mitochondrial protein | K577 | LQLLEPFDKWDGkDLEDLQILIK | 316 |
| 318 | ACO2 | NP_001089.1 | Mitochondrial protein | K461 | DIkKGEKNTIVTSYNR | 317 |
| 319 | ACO2 | NP_001089.1 | Mitochondrial protein | K651 | YYkKHGIR | 318 |
| 320 | ACO2 | NP_001089.1 | Mitochondrial protein | K652 | YYKkHGIR | 319 |
| 321 | ACOT2 | NP_006812.3 | Mitochondrial protein | K368 | PLEGPDQkSFIPVER | 320 |
| 322 | ACOT2 | NP_006812.3 | Mitochondrial protein | K284 | LLSHPEVkGPGVGLL | 321 |
| 323 | AFG3L2 | NP_006787.1 | Mitochondrial protein | K96 | PKEVMGEkKESKPAA | 322 |
| 324 | AFG3L2 | NP_006787.1 | Mitochondrial protein | K117 | GGGGGGGkRGGKKDD | 323 |
| 325 | AK3 | NP_057366.2 | Mitochondrial protein | K189 | PVLEYYQkKGVLETF | 324 |
| 326 | AK3 | NP_057366.2 | Mitochondrial protein | K20 | AVIMGAPGSGkGTVSSR | 325 |
| 327 | AK3 | NP_057366.2 | Mitochondrial protein | K64 | KAFIDQGkLIPDDVM | 326 |
| 328 | AK3 | NP_057366.2 | Mitochondrial protein | K171 | TVGIDDLTGEPLIQREDDKPET | 327 |
| | | | | | VIkR | |
| 329 | AK3 | NP_057366.2 | Mitochondrial protein | K190 | VLEYYQKkGVLETFS | 328 |
| 330 | AK3 | NP_057366.2 | Mitochondrial protein | K165 | VYNIEFNPPKTVGIDDLTGEPLI | 329 |
| | | | | | QREDDkPETVIKR | |
| 331 | CLYBL | NP_996531.1 | Mitochondrial protein | K82 | EDGVAANkKNEARLR | 330 |
| 332 | CLYBL | NP_996531.1 | Mitochondrial protein | K83 | DGVAANKkNEARLRI | 331 |
| 333 | CLYBL | NP_996531.1 | Mitochondrial protein | K309 | KEHQQLGkGAFTFQG | 332 |
| 334 | CLYBL | NP_996531.1 | Mitochondrial protein | K57 | RAVLYVPGNDEkK | 333 |
| 335 | CLYBL | NP_996531.1 | Mitochondrial protein | K58 | VPGNDEKkIKKIPSL | 334 |
| 336 | COX7C | NP_001858.1 | Mitochondrial protein | K62 | VVRHQLLkT | 335 |
| 337 | CS | NP_004068.2 | Mitochondrial protein | K76 | GMkGLVYETSVLDPDEGIR | 336 |
| 338 | CS | NP_004068.2 | Mitochondrial protein | K382 | KLVAQLYkIVPNVLL | 337 |
| 339 | CS | NP_004068.2 | Mitochondrial protein | K43 | ILADLIPkEQARIKT | 338 |
| 340 | CS | NP_004068.2 | Mitochondrial protein | K49 | PKEQARIkTFRQQHG | 339 |
| 341 | CS | NP_004068.2 | Mitochondrial protein | K103 | FSIPECQkLLPKAKG | 340 |
| 342 | CS | NP_004068.2 | Mitochondrial protein | K327 | EVGKDVSDEkLR | 341 |
| 343 | CS | NP_004068.2 | Mitochondrial protein | K366 | CQREFALkHLPNDPM | 342 |
| 344 | CS | NP_004068.2 | Mitochondrial protein | K393 | NVLLEQGkAKNPWPN | 343 |
| 345 | CS | NP_004068.2 | Mitochondrial protein | K450 | GFPLERPkSMSTEGL | 344 |
| 346 | CS | NP_004068.2 | Mitochondrial protein | K57 | TFRQQHGkTVVGQIT | 345 |
| 347 | CS | NP_004068.2 | Mitochondrial protein | K464 | LMKFVDSkSG | 346 |
| 348 | DLD | NP_000099.2 | Mitochondrial protein | K273 | FkLNTKVTGATK | 347 |
| 349 | DLD | NP_000099.2 | Mitochondrial protein | K277 | FKLNTkVTGATK | 348 |
| 350 | DLD | NP_000099.2 | Mitochondrial protein | K505 | NLAASFGkSINF | 349 |
| 351 | DLD | NP_000099.2 | Mitochondrial protein | K146 | AHLFKQNkVVHVNGY | 350 |
| 352 | DLD | NP_000099.2 | Mitochondrial protein | K104 | ALLNNSHYYHMAHGkDFASR | 351 |
| 353 | DLD | NP_000099.2 | Mitochondrial protein | K132 | EQKSTAVkALTGGIA | 352 |
| 354 | ECH1 | NP_001389.2 | Mitochondrial protein | K327 | LKTVTFSkL | 353 |
| 355 | ETFA | NP_000117.1 | Mitochondrial protein | K232 | SGENFkLLYDLADQLHAAVGA | 354 |
| | | | | | SR | |
| 356 | ETFA | NP_000117.1 | Mitochondrial protein | K62 | VAGTKCDkVAQDLCK | 355 |
| 357 | ETFA | NP_000117.1 | Mitochondrial protein | K69 | KVAQDLCkVAGIAKV | 356 |
| 358 | ETFA | NP_000117.1 | Mitochondrial protein | K139 | ISDIIAIkSPDTFVR | 357 |
| 359 | ETFA | NP_000117.1 | Mitochondrial protein | K226 | GLkSGENFK | 358 |
| 360 | ETFA | NP_000117.1 | Mitochondrial protein | K331 | VVPEMTEILkK | 359 |
| 361 | ETFA | NP_000117.1 | Mitochondrial protein | K332 | VVPEMTEILKkK | 360 |
| 362 | ETFB | NP_001014763.1 | Mitochondrial protein | K150 | LKEKKLVkEVIAVSC | 361 |
| 363 | ETFB | NP_001976.1 | Mitochondrial protein | K23 | YAVKIRVkPDRTGVV | 362 |
| 364 | ETFB | NP_001014763.1 | Mitochondrial protein | K126 | GVVTDGVkHSMNPFC | 363 |
| 365 | ETFB | NP_001976.1 | Mitochondrial protein | K116 | AKLAEKEkVDLVLLG | 364 |
| 366 | ETFB | NP_001976.1 | Mitochondrial protein | K56 | AVRLKEKkLVKEVIA | 365 |
| 367 | HSPE1 | NP_002148.1 | Mitochondrial protein | K54 | VAVGSGSkGKGGEIQ | 366 |
| 368 | HSPE1 | NP_002148.1 | Mitochondrial protein | K56 | VGSGSKGkGGEIQPV | 367 |
| 369 | SDHA | NP_004159.2 | Mitochondrial protein | K182 | AFGGQSLKFGkGGQAHR | 368 |
| 370 | SDHA | NP_004159.2 | Mitochondrial protein | K598 | GAHAREDYkVR | 369 |
| 371 | SDHA | NP_004159.2 | Mitochondrial protein | K608 | IDEYDYSkPIQGQQK | 370 |
| 372 | SDHA | NP_004159.2 | Mitochondrial protein | K517 | ELRLSMQkSMQNHAA | 371 |
| 373 | SDHB | NP_002991.2 | Mitochondrial protein | K55 | DPDKAGDkPHMQTYE | 372 |
| 374 | SDHB | NP_002991.2 | Mitochondrial protein | K233 | FTEERLAkLQDPFSL | 373 |
| 375 | SDHB | NP_002991.2 | Mitochondrial protein | K267 | AIAEIkK | 374 |
| 376 | SUCLG1 | NP_003840.2 | Mitochondrial protein | K192 | IGIMPGHIHkK | 375 |
| 377 | SUCLG1 | NP_003840.2 | Mitochondrial protein | K308 | MGHAGAIIAGGkGGAK | 376 |
| 378 | SUCLG1 | NP_003840.2 | Mitochondrial protein | K312 | MGHAGAIIAGGKGGAk | 377 |
| 379 | SUCLG1 | NP_003840.2 | Mitochondrial protein | K342 | ISALQSAGVVVSMSPAQLGTTI | 378 |
| | | | | | YKEFEkR | |
| 380 | SUCLG1 | NP_003840.2 | Mitochondrial protein | K57 | LYVDKNTkIICQGFT | 379 |
| 381 | SUCLG1 | NP_003840.2 | Mitochondrial protein | K66 | IICQGFTGkQGTFHSQQALEY | 380 |
| | | | | | GTK | |
| 382 | SUCLG1 | NP_003840.2 | Mitochondrial protein | K90 | VGGTTPGkGGQTHLG | 381 |
| 383 | SUCLG1 | NP_003840.2 | Mitochondrial protein | K193 | IGIMPGHIHKk | 382 |
| 384 | SUCLG1 | NP_003840.2 | Mitochondrial protein | K54 | RQHLYVDkNTKIICQ | 383 |
| 385 | SUCLG1 | NP_003840.2 | Mitochondrial protein | K338 | ISALQSAGVVVSMSPAQLGTTI | 384 |
| | | | | | YkEFEKR | |
| 386 | SUCLG1 | NP_003840.2 | Mitochondrial protein | K314 | MGHAGAIIAGGKGGAKEk | 385 |
| 387 | UCP1 | NP_068605.1 | Mitochondrial protein | K56 | TSSVIRYkGVLGTIT | 386 |
| 388 | UCP1 | NP_068605.1 | Mitochondrial protein | K73 | VKTEGRMkLYSGLPA | 387 |
| 389 | UCP1 | NP_068605.1 | Mitochondrial protein | K151 | QSHLHGIkPRYTGTY | 388 |
| 390 | LOC146909 | XP_946528.1 | Motor or contractile | K62 | GGTHDGPkKKGKDLT | 389 |
| | | | protein | | | |
| 391 | LOC146909 | XP_946528.1 | Motor or contractile | K63 | GTHDGPKkKGKDLTF | 390 |
| | | | protein | | | |
| 392 | LOC146909 | XP_946528.1 | Motor or contractile | K64 | THDGPKKkGKDLTFV | 391 |
| | | | protein | | | |
| 393 | MYO5A | NP_000250.2 | Motor or contractile | K1263 | SAPEVTAPGAPAYRVLMEQLT | 392 |
| | | | protein | | SVSEELDVRk | |
| 394 | MASP2 | NP_006601.2 | Protease | K626 | AGLESGGkDSCRGDS | 393 |
| 395 | BCKDK | NP_005872.1 | Protein kinase, atypical | K163 | QLLDDHkDVVTLLAEGLR | 394 |
| 396 | NDR1 | NP_009202.1 | Protein kinase, Ser/Thr | K223 | DIKPDNLLLDSkGHVK | 395 |
| | | | (non-receptor) | | | |
| 397 | Titin | NP_003310.3 | Protein kinase, Ser/Thr | K15206 | KAGQRWIkCNKKTLT | 396 |
| | | | (non-receptor) | | | |
| 398 | Titin | NP_003310.3 | Protein kinase, Ser/Thr | K16096 | LEIkSTDFATSLSVK | 397 |
| | | | (non-receptor) | | | |
| 399 | Titin | NP_003310.3 | Protein kinase, Ser/Thr | K5402 | TKHSMVIkSAAFEDE | 398 |
| | | | (non-receptor) | | | |
| 400 | Pyk2 | NP_004094.3 | Protein kinase, Tyr (non- | K956 | DLAELINkMRLAQQN | 399 |
| | | | receptor) | | | |
| 401 | BPI | NP_001716.2 | Receptor, channel, | K121 | ANIKISGkWKAQKRF | 400 |
| | | | transporter or cell | | | |
| | | | surface protein | | | |
| 402 | BPI | NP_001716.2 | Receptor, channel, | K126 | SGKWKAQkRFLKMSG | 401 |
| | | | transporter or cell | | | |
| | | | surface protein | | | |
| 403 | C9 | NP_001728.1 | Receptor, channel, | K490 | KMKNAHLkKQNLERA | 402 |
| | | | transporter or cell | | | |
| | | | surface protein | | | |
| 404 | CHRM3 | NP_000731.1 | Receptor, channel, | K260 | IYKETEkR | 403 |
| | | | transporter or cell | | | |
| | | | surface protein | | | |
| 405 | exportin 5 | NP_065801.1 | Receptor, channel, | K320 | EKHYVFLkRLCQVLC | 404 |
| | | | transporter or cell | | | |
| | | | surface protein | | | |
| 406 | nAChRA4 | NP_000735.1 | Receptor, channel, | K178 | MkFGSWTYDKAK | 405 |
| | | | transporter or cell | | | |
| | | | surface protein | | | |
| 407 | nAChRA4 | NP_000735.1 | Receptor, channel, | K186 | MKFGSWTYDkAK | 406 |
| | | | transporter or cell | | | |
| | | | surface protein | | | |
| 408 | SLC25A1 | NP_005975.1 | Receptor, channel, | K97 | GLSSLLYGSIPkAAVR | 407 |
| | | | transporter or cell | | | |
| | | | surface protein | | | |
| 409 | SLC25A1 | NP_005975.1 | Receptor, channel, | K255 | MQGLEAHkYR | 408 |
| | | | transporter or cell | | | |
| | | | surface protein | | | |
| 410 | SLC25A1 | NP_005975.1 | Receptor, channel, | K178 | EQGLkGTYQGLTATVLK | 409 |
| | | | transporter or cell | | | |
| | | | surface protein | | | |
| 411 | SLC25A1 | NP_005975.1 | Receptor, channel, | K277 | AFYkGTVPR | 410 |
| | | | transporter or cell | | | |
| | | | surface protein | | | |
| 412 | SLC25A20 | NP_000378.1 | Receptor, channel, | K157 | TGTLDCAkKLYQEFG | 411 |
| | | | transporter or cell | | | |
| | | | surface protein | | | |
| 413 | SLC25A20 | NP_000378.1 | Receptor, channel, | K170 | FGIRGIYkGTVLTLM | 412 |
| | | | transporter or cell | | | |
| | | | surface protein | | | |
| 414 | SLC25A3 | NP_002626.1 | Receptor, channel, | K100 | MQVDPQKYk | 413 |
| | | | transporter or cell | | | |
| | | | surface protein | | | |
| 415 | SLC25A4 | NP_001142.2 | Receptor, channel, | K33 | VkLLLQVQHASK | 414 |
| | | | transporter or cell | | | |
| | | | surface protein | | | |
| 416 | SLC25A4 | NP_001142.2 | Receptor, channel, | K63 | IPkEQGFLSFWR | 415 |
| | | | transporter or cell | | | |
| | | | surface protein | | | |
| 417 | SLC25A4 | NP_001142.2 | Receptor, channel, | K147 | RLAADVGkGAAQREF | 416 |
| | | | transporter or cell | | | |
| | | | surface protein | | | |
| 418 | SLC25A4 | NP_001142.2 | Receptor, channel, | K92 | YFPTQALNFAFkDK | 417 |
| | | | transporter or cell | | | |
| | | | surface protein | | | |
| 419 | SLC25A5 | NP_001143.2 | Receptor, channel, | K33 | VkLLLQVQHASK | 418 |
| | | | transporter or cell | | | |
| | | | surface protein | | | |
| 420 | SLC25A5 | NP_001143.2 | Receptor, channel, | K63 | IPkEQGVLSFWR | 419 |
| | | | transporter or cell | | | |
| | | | surface protein | | | |
| 421 | SLC25A5 | NP_001143.2 | Receptor, channel, | K10 | TDAAVSFAkDFLAGGVAAAISK | 420 |
| | | | transporter or cell | | | |
| | | | surface protein | | | |
| 422 | SLC25A6 | NP_001627.2 | Receptor, channel, | K147 | RLAADVGkSGTEREF | 421 |
| | | | transporter or cell | | | |
| | | | surface protein | | | |
| 423 | SLC25A6 | NP_001627.2 | Receptor, channel, | K163 | GLGDCLVkITKSDGI | 422 |
| | | | transporter or cell | | | |
| | | | surface protein | | | |
| 424 | SLC25A6 | NP_001627.2 | Receptor, channel, | K33 | VkLLLQVQHASK | 423 |
| | | | transporter or cell | | | |
| | | | surface protein | | | |
| 425 | SLC25A6 | NP_001627.2 | Receptor, channel, | K63 | IPkEQGVLSFWR | 424 |
| | | | transporter or cell | | | |
| | | | surface protein | | | |
| 426 | SLC25A6 | NP_001627.2 | Receptor, channel, | K105 | IFLGGVDkHTQFWRY | 425 |
| | | | transporter or cell | | | |
| | | | surface protein | | | |
| 427 | AUH | NP_001689.1 | RNA processing | K123 | AVDALKSDKk | 426 |
| 428 | AUH | NP_001689.1 | RNA processing | K329 | DRLEGLLAFkEK | 427 |
| 429 | LRPPRC | NP_573566.2 | RNA processing | K966 | YNLLKLYkINGDWQR | 428 |
| 430 | PAPD1 | NP_060579.2 | RNA processing | K174 | NQLPRSNkQLFELLC | 429 |
| 431 | PNPT1 | NP_149100.1 | RNA processing | K616 | TVQVPLSkRAKFVGP | 430 |
| 432 | PNPT1 | NP_149100.1 | RNA processing | K357 | LNEYkR | 431 |
| 433 | PNPT1 | NP_149100.1 | RNA processing | K246 | SQIVMLEASAENILQQDFCHAI | 432 |
| | | | | | kVGVK | |
| 434 | FOXA3 | NP_004488.2 | Transcriptional regulator | K214 | LRRQKRFkLEEKVKK | 433 |
| 435 | FOXA3 | NP_004488.2 | Transcriptional regulator | K218 | KRFKLEEkVKKGGSG | 434 |
| 436 | FOXA3 | NP_004488.2 | Transcriptional regulator | K221 | KLEEKVKkGGSGAAT | 435 |
| 437 | HEY1 | NP_001035798.1 | Transcriptional regulator | K253 | PVVTSASkLSPPLLS | 436 |
| 438 | SND1 | NP_055205.2 | Transcriptional regulator | K869 | EkQFQKVITEYLNAQESAK | 437 |
| 439 | WHSC2 | NP_005654.2 | Transcriptional regulator | K285 | LDAEVVEkPAKEETV | 438 |
| 440 | MRPL12 | NP_002940.2 | Translation | K162 | GINLVQAkKLVESLP | 439 |
| 441 | MRPL12 | NP_002940.2 | Translation | K150 | VKLIKEIkNYIQGIN | 440 |
| 442 | MRPS18C | NP_057151.1 | Translation | K102 | HITGLCGkK | 441 |
| 443 | MRPS36 | NP_150597.1 | Translation | K78 | PDTAEIIkTLPQKYR | 442 |
| 444 | NGDN | NP_056329.1 | Translation | K191 | TEAEREKkRLERAKR | 443 |
| 445 | LETMD1 | NP_001019839.1 | Tumor suppressor or | K45 | QMLWADAkKARRIKT | 444 |
| | | | oncoprotein | | | |
| 446 | LETMD1 | NP_056231.3 | Tumor suppressor or | K112 | RIKTNMWkHNIKFHQ | 445 |
| | | | oncoprotein | | | |
| 447 | LETMD1 | NP_001019839.1 | Tumor suppressor or | K118 | IRHFWTPkQQTDFLD | 446 |
| | | | oncoprotein | | | |
| 448 | BIRC6 | NP_057336.3 | Ubiquitin conjugating | K4459 | RSKRENVkTGVKPDA | 447 |
| | | | system | | | |
| 449 | UBP-M | NP_006438.1 | Protease | K682 | HVYTNAKkQMLISLA | 448 |
| 450 | 1700021F05Rik | NP_057571.1 | Unknown function | K133 | PTVVKNYkDLEKAVQ | 449 |
| 451 | ACAD10 | NP_079523.3 | Mitochondrial protein | K776 | RYGTEAQkARWLIPL | 450 |
| 452 | ACAD10 | NP_079523.3 | Mitochondrial protein | K1052 | VHRATVAkLELKHRI | 451 |
| 453 | ACAD10 | NP_079523.3 | Mitochondrial protein | K1056 | TVAKLELkHRI | 452 |
| 454 | ACAD10 | NP_079523.3 | Mitochondrial protein | K606 | KEGFRVFkEMPFTNP | 453 |
| 455 | ANUBL1 | NP_777550.1 | Unknown function | K239 | KNMNLSKkPKKAVKI | 454 |
| 456 | C14orf159 | NP_079228.4 | Unknown function | K94 | MGHPQFWkYEFGACT | 455 |
| 457 | C21orf33 | NP_004640.2 | Unknown function | K233 | VEAHVDQkNKVVTTP | 456 |
| 458 | C21orf33 | NP_004640.2 | Unknown function | K223 | LGAKHCVkEVVEAHV | 457 |
| 459 | C21orf33 | NP_004640.2 | Unknown function | K203 | GVEVTVGHEQEEGGkWPYAG | 458 |
| | | | | | TAEAIK | |
| 460 | C21orf33 | NP_004640.2 | Unknown function | K151 | NLSTFAVDGkDCK | 459 |
| 461 | C21orf33 | NP_004640.2 | Unknown function | K157 | VNkEVER | 460 |
| 462 | CBR4 | NP_116172.2 | Enzyme, misc. | K195 | DLKEEHLkKNIPLGR | 461 |
| 463 | CBR4 | NP_116172.2 | Enzyme, misc. | K152 | QSVYSASkGGLVGFS | 462 |
| 464 | CBR4 | NP_116172.2 | Enzyme, misc. | K190 | TDMTKDLkEEHLKKN | 463 |
| 465 | CBR4 | AAH21973.1 | Enzyme, misc. | K72 | NTFEEMEkHLGRVNF | 464 |
| 466 | CBR4 | NP_116172.2 | Enzyme, misc. | K196 | LKEEHLKkNIPLGRF | 465 |
| 467 | CCDC90A | NP_001026883.1 | Unknown function | K228 | MSQIANVkKDMIILE | 466 |
| 468 | CCDC90A | NP_001026883.1 | Unknown function | K291 | VKELYSLNEkK | 467 |
| 469 | COQ9 | NP_064708.1 | Unknown function | K175 | LVQLGQAEkR | 468 |
| 470 | COQ9 | NP_064708.1 | Unknown function | K51 | LRSSDEQkQQPPNSF | 469 |
| 471 | DMXL1 | NP_005500.4 | Unknown function | K2741 | RGASVMIkKAINNVR | 470 |
| 472 | ECHDC1 | NP_001002030.1 | Unknown function | K295 | ANLEAIAkKGKFNK | 471 |
| 473 | ECHDC1 | NP_001002030.1 | Unknown function | K53 | GGSIDLQkEDNGIGI | 472 |
| 474 | FAHD1 | NP_112485.1 | Mitochondrial protein | K113 | RDVQDECkKKGLPWT | 473 |
| 475 | FAHD1 | NP_112485.1 | Mitochondrial protein | K114 | DVQDECKkKGLPWTL | 474 |
| 476 | FTSJ3 | NP_060117.2 | Enzyme, misc. | K746 | VAEAkAR | 475 |
| 477 | HSDL2 | NP_115679.2 | Mitochondrial protein | K32 | AAkDGANIVIAAK | 476 |
| 478 | HSDL2 | NP_115679.2 | Mitochondrial protein | K116 | FGGIDILVNNASAISLTNTLDTP | 477 |
| | | | | | TkR | |
| 479 | KIAA0564 | NP_055873.1 | Unknown function | K360 | MAVEGVLkR | 478 |
| 480 | KIAA0564 | NP_055873.1 | Unknown function | K1612 | DAVPEEVkRAAREMG | 479 |
| 481 | KIAA0564 | NP_055873.1 | Unknown function | K1887 | SFVAMDTkDIPQILQ | 480 |
| 482 | KLRG2 | NP_940910.1 | Unknown function | K228 | CKELGLEkEDAALLP | 481 |
| 483 | LHX5 | NP_071758.1 | Unknown function | K188 | GPRTTIkAKQLETLK | 482 |
| 484 | LHX5 | NP_071758.1 | Unknown function | K190 | GPRTTIKAkQLETLK | 483 |
| 485 | PCBD2 | NP_115527.3 | Enzyme, misc. | K114 | HDCGELTkKDVKLAK | 484 |
| 486 | PCBD2 | NP_115527.3 | Enzyme, misc. | K86 | VALQAEkMNHHPEWFNVYNK | 485 |
| 487 | PRR8 | NP_444271.2 | Unknown function | K821 | QQQAGARKkELLER | 486 |
| 488 | TMEM81 | NP_976310.1 | Unknown function | K199 | QSLTEDQkLIDEGLE | 487 |
| 489 | VWA3A | NP_775886.2 | Unknown function | K224 | WLKVNGLkAKKLSLY | 488 |
| 490 | ZADH2 | NP_787103.1 | Enzyme, misc. | K35 | AIPQAMQkLVVTRLS | 489 |
| 491 | ZADH2 | NP_787103.1 | Enzyme, misc. | K376 | LPHSVNSkL | 490 |
| 492 | ACOT2 | NP_006812.3 | Mitochondrial protein | K104 | RASLRDEkGALFQAH | 491 |
| 493 | 4930429A22Rik | NP_689596.4 | RNA processing | K252 | RAATGFLkLLADKNS | 492 |
| 494 | 4930429A22Rik | NP_689596.4 | RNA processing | K257 | FLKLLADkNSELFRK | 493 |
| 495 | 4930429A22Rik | NP_689596.4 | RNA processing | K264 | KNSELFRkYALFSPS | 494 |
| 496 | AADAT | NP_057312.1 | Mitochondrial protein | K69 | QFGEEMMkRALQYSP | 495 |
| 497 | AADAT | NP_057312.1 | Mitochondrial protein | K188 | EDAKNPQkNTPKFLY | 496 |
| 498 | AASS | NP_005754.2 | Mitochondrial protein | K93 | ACLILGVkRPPEEKL | 497 |
| 499 | AASS | NP_005754.2 | Mitochondrial protein | K70 | NRRAIHDkDYVKAGG | 498 |
| 500 | ABCA5 | NP_061142.2 | Receptor, channel, | K1274 | DVKAERLkVKELMGC | 499 |
| | | | transporter or cell | | | |
| | | | surface protein | | | |
| 501 | ABCC2 | NP_000383.1 | Receptor, channel, | K493 | TIQVKNMkNKDKRLK | 500 |
| | | | transporter or cell | | | |
| | | | surface protein | | | |
| 502 | ACAA1 | NP_001598.1 | Enzyme, misc. | K395 | ITLLNELkRRGKRAY | 501 |
| 503 | ACAA1 | NP_001598.1 | Enzyme, misc. | K237 | VHDDKGTkRSITVTQ | 502 |
| 504 | ACAD9 | NP_054768.2 | Enzyme, misc. | K202 | RATLSEDkKHYILNG | 503 |
| 505 | ACADVL | NP_000009.1 | Enzyme, misc. | K51 | AAQLALDkSDSHPSD | 504 |
| 506 | ACADVL | NP_000009.1 | Enzyme, misc. | K195 | GILLFGTkAQKEKYL | 505 |
| 507 | ACADVL | NP_000009.1 | Enzyme, misc. | K492 | SGLGSALkNPFGNAG | 506 |
| 508 | ACO2 | NP_001089.1 | Mitochondrial protein | K730 | KLTIQGLkDFTPGKP | 507 |
| 509 | ACOT1 | NP_001032238.1 | Enzyme, misc. | K42 | RASLRDEkGALFQAH | 508 |
| 510 | ACOT4 | NP_689544.3 | Enzyme, misc. | K42 | RASLRDEkGALFRAH | 509 |
| 511 | ACOT8 | NP_005460.2 | Enzyme, misc. | K318 | KPQVSESkL | 510 |
| 512 | ACOX1 | NP_004026.2 | Enzyme, misc. | K349 | QFVGAYMkETYHRIN | 511 |
| 513 | ACOX1 | NP_004026.2 | Enzyme, misc. | K637 | ENLFEWAkNSPLNKA | 512 |
| 514 | ACOX1 | NP_004026.2 | Enzyme, misc. | K659 | HLKSLQSkL | 513 |
| 515 | ACOX1 | NP_004026.2 | Enzyme, misc. | K260 | LMKYAQVkPDGTYVK | 514 |
| 516 | ACOX1 | NP_004026.2 | Enzyme, misc. | K216 | IREIGTHkPLPGITV | 515 |
| 517 | ACOX1 | NP_004026.2 | Enzyme, misc. | K654 | HESYKHLkSLQSKL | 516 |
| 518 | ACOX1 | NP_004026.2 | Enzyme, misc. | K542 | VVKLFSEkLLKIQDK | 517 |
| 519 | ACOX1 | NP_004026.2 | Enzyme, misc. | K72 | RKSAIMVkKMREFGI | 518 |
| 520 | ACOX2 | NP_003491.1 | Enzyme, misc. | K419 | CGGHGYSkLSGLPSL | 519 |
| 521 | ACSF2 | NP_079425.3 | Enzyme, misc. | K182 | LVFPKQFkTQQYYNV | 520 |
| 522 | ACSF2 | NP_079425.3 | Enzyme, misc. | K510 | CKIVGRSkDMIIRGG | 521 |
| 523 | ACSM1 | NP_443188.2 | Mitochondrial protein | K253 | PSFPGSRkLRSLKTS | 522 |
| 524 | ACSM1 | NP_443188.2 | Mitochondrial protein | K204 | LDFRSLVkSASPEHT | 523 |
| 525 | ACSM1 | NP_443188.2 | Mitochondrial protein | K356 | PKDQEEWkRRTGLLL | 524 |
| 526 | ACSM1 | NP_443188.2 | Mitochondrial protein | K538 | KELQQHVkSVTAPYK | 525 |
| 527 | ACSM1 | NP_443188.2 | Mitochondrial protein | K67 | AQKEKEGkRGPNPAF | 526 |
| 528 | ACSM3 | NP_005613.2 | Mitochondrial protein | K547 | KEIQEHVkKTTAPYK | 527 |
| 529 | ADH1C | NP_000660.1 | Mitochondrial protein | K355 | TNILPFEkINEGFDL | 528 |
| 530 | ADH1C | NP_000660.1 | Mitochondrial protein | K9 | STAGKVIkCKAAVLW | 529 |
| 531 | ADH1C | NP_000660.1 | Mitochondrial protein | K340 | VADFMAKkFSLDALI | 530 |
| 532 | ADH4 | NP_000661.2 | Enzyme, misc. | K115 | PLTNLCGkISNLKSP | 531 |
| 533 | AEBP1 | NP_001120.3 | Transcriptional regulator | K98 | KDKGKKGkKDKGPKV | 532 |
| 534 | AEBP1 | NP_001120.3 | Transcriptional regulator | K99 | DKGKKGKkDKGPKVP | 533 |
| 535 | AEBP1 | NP_001120.3 | Transcriptional regulator | K101 | GKKGKKDkGPKVPKE | 534 |
| 536 | AFF3 | NP_002276.2 | Transcriptional regulator | K1085 | LKRDHAVkYSKALID | 535 |
| 537 | AGXT | NP_000021.1 | Mitochondrial protein | K312 | LGLQLFVkDPALRLP | 536 |
| 538 | AGXT2 | NP_114106.1 | Enzyme, misc. | K71 | NRVLEIHkEHLSPVV | 537 |
| 539 | AK2 | NP_001616.1 | Kinase (non-protein) | K62 | ASGSELGkKLKATMD | 538 |
| 540 | ALDH1B1 | NP_000683.3 | Enzyme, misc. | K51 | EWQDAVSkKTFPTVN | 539 |
| 541 | ALDH1B1 | NP_000683.3 | Enzyme, misc. | K399 | GERGFFIkPTVFGGV | 540 |
| 542 | ALDH3A2 | NP_000373.1 | Enzyme, misc. | K260 | LQNQIVWkIKETVKE | 541 |
| 543 | ALDH4A1 | NP_003739.2 | Enzyme, misc. | K531 | RASGTNDkPGGPHYI | 542 |
| 544 | ALDH4A1 | NP_003739.2 | Enzyme, misc. | K552 | QVIKETHkPLGDWSY | 543 |
| 545 | ALDH4A1 | NP_003739.2 | Enzyme, misc. | K365 | HSLWPQIkGRLLEEH | 544 |
| 546 | ALDH4A1 | NP_003739.2 | Enzyme, misc. | K119 | ARKEWDLkPIADRAQ | 545 |
| 547 | ALDH4A1 | NP_003739.2 | Enzyme, misc. | K93 | NHGHKVAkFCYADKS | 546 |
| 548 | ALDH5A1 | NP_001071.1 | Enzyme, misc. | K365 | KAFAEAMkKNLRVGN | 547 |
| 549 | ALDH6A1 | NP_005580.1 | Enzyme, misc. | K52 | GGKFVESkSDKWIDI | 548 |
| 550 | ALDH6A1 | NP_005580.1 | Enzyme, misc. | K87 | DAAIASCkRAFPAWA | 549 |
| 551 | ALDH6A1 | NP_005580.1 | Enzyme, misc. | K330 | AVLVGEAkKWLPELV | 550 |
| 552 | ALDH9A1 | NP_000687.3 | Enzyme, misc. | K485 | ELPFGGYkKSGFGRE | 551 |
| 553 | Alix | NP_037506.2 | Adaptor/scaffold | K268 | SILAKQQkKFGEEIA | 552 |
| 554 | Alix | NP_037506.2 | Adaptor/scaffold | K269 | ILAKQQKkFGEEIAR | 553 |
| 555 | AMACR | NP_055139.4 | Mitochondrial protein | K58 | RSLVLDLkQPRGAAV | 554 |
| 556 | AMACR | AAF22610.1 | Mitochondrial protein | K277 | KFADVFAkKTKAEWC | 555 |
| 557 | APOA1BP | NP_658985.2 | Secreted protein | K148 | YYPKRPNkPLFTALV | 556 |
| 558 | APOL6 | NP_085144.1 | Apoptosis | K231 | GTTLAMTkNARVLGG | 557 |
| 559 | ARFGEF3 | NP_065073.3 | Receptor, channel, | K2002 | EKKDPSRkKEWWENA | 558 |
| | | | transporter or cell | | | |
| | | | surface protein | | | |
| 560 | ARL6IP | NP_055976.1 | Unknown function | K188 | LKYIGMAkREINKLL | 559 |
| 561 | ARPP-21 | NP_057384.2 | Inhibitor protein | K62 | KSKSGAGkGKLTRSL | 560 |
| 562 | ARPP-21 | NP_057384.2 | Inhibitor protein | K64 | KSGAGKGkLTRSLAV | 561 |
| 563 | ARPP-21 | NP_057384.2 | Inhibitor protein | K57 | NQERRKSkSGAGKGK | 562 |
| 564 | ASS1 | NP_000041.2 | Endoplasmic reticulum or | K228 | DILEIEFkKGVPVKV | 563 |
| | | | golgi | | | |
| 565 | ATAD3A | NP_060658.2 | Mitochondrial protein | K562 | AQFDYGRkCSEVARL | 564 |
| 566 | ATOX1 | NP_004036.1 | Receptor, channel, | K60 | ATLKKTGkTVSYLGL | 565 |
| | | | transporter or cell | | | |
| | | | surface protein | | | |
| 567 | ATP13A1 | NP_065143.2 | Unknown function | K845 | ARVAPKQkEFVITSL | 566 |
| 568 | ATP13A1 | NP_065143.2 | Unknown function | K843 | VFARVAPkQKEFVIT | 567 |
| 569 | ATP1A3 | NP_689509.1 | Receptor, channel, | K458 | ELSSGSVkLMRERNK | 568 |
| | | | transporter or cell | | | |
| | | | surface protein | | | |
| 570 | ATP5A1 | NP_004037.1 | Enzyme, misc. | K161 | LGNAIDGkGPIGSKT | 569 |
| 571 | ATP5A1 | NP_004037.1 | Enzyme, misc. | K241 | DGSDEKKkLYCIYVA | 570 |
| 572 | ATP5B | NP_001677.2 | Enzyme, misc. | K124 | EGLVRGQkVLDSGAP | 571 |
| 573 | ATP5C | NP_005165.1 | Enzyme, misc. | K89 | IKGPEDKkKHLLIGV | 572 |
| 574 | ATP5F1 | NP_001679.2 | Mitochondrial protein | K248 | ADLKLLAkKAQAQPV | 573 |
| 575 | ATP5H | NP_006347.1 | Receptor, channel, | K148 | PETKLDKkKYPYWPH | 574 |
| | | | transporter or cell | | | |
| | | | surface protein | | | |
| 576 | ATP5J | NP_001676.2 | Mitochondrial protein | K46 | IQKLFVDkIREYKSK | 575 |
| 577 | ATP5J | NP_001676.2 | Mitochondrial protein | K79 | ERELFKLkQMFGNAD | 576 |
| 578 | ATP5J2 | NP_004880.1 | Mitochondrial protein | K22 | DKKLLEVkLGELPSW | 577 |
| 579 | ATP5J2 | NP_004880.1 | Mitochondrial protein | K17 | PVPVKDKkLLEVKLG | 578 |
| 580 | ATP5O | NP_001688.1 | Mitochondrial protein | K84 | SVLNPYVkRSIKVKS | 579 |
| 581 | ATP5O | NP_001688.1 | Mitochondrial protein | K90 | VKRSIKVkSLNDITA | 580 |
| 582 | ATPIF1 | NP_057395.1 | Inhibitor protein | K49 | EAGGAFGkREQAEEE | 581 |
| 583 | BAHD1 | NP_055767.3 | Unknown function | K214 | LNAAAFLkLSQEREL | 582 |
| 584 | BAT2 | NP_542417.2 | RNA processing | K1685 | GAAEGPPkRPGGSSP | 583 |
| 585 | BC024868 | XP_001723021.1 | Unassigned | K443 | DLDREPKkEAVKSFI | 584 |
| 586 | BCKDH E1- | NP_000700.1 | Mitochondrial protein | K356 | DEVNYWDkQDHPISR | 585 |
| | alpha | | | | | |
| 587 | BCKDH E1- | NP_000700.1 | Mitochondrial protein | K445 | YPLDHFDk | 586 |
| | alpha | | | | | |
| 588 | BCKDH E1- | NP_000700.1 | Mitochondrial protein | K224 | VGAAYAAkRANANRV | 587 |
| | alpha | | | | | |
| 589 | BPHL | NP_004323.1 | Enzyme, misc. | K109 | KDAVDLMkALKFKKV | 588 |
| 590 | BPHL | NP_004323.1 | Enzyme, misc. | K69 | PQLKNLNkKLFTVVA | 589 |
| 591 | BPHL | NP_004323.1 | Enzyme, misc. | K243 | DFIHKHVkGSRLHLM | 590 |
| 592 | BPHL | NP_004323.1 | Enzyme, misc. | K167 | EGIRDVSkWSERTRK | 591 |
| 593 | BPNT1 | NP_006076.4 | Enzyme, misc. | K244 | VFASPGCkKWDTCAP | 592 |
| 594 | BRP44L | NP_057182.1 | Unknown function | K46 | AAINDMKkSPEIISG | 593 |
| 595 | BRP44L | NP_057182.1 | Unknown function | K45 | IAAINDMkKSPEIIS | 594 |
| 596 | C10orf33 | NP_116098.1 | Enzyme, misc. | K105 | IYTDLELkKHGLRLH | 595 |
| 597 | C15orf48 | NP_115789.1 | Unassigned | K61 | VDPTVPQkLITINQQ | 596 |
| 598 | C15orf48 | NP_115789.1 | Unassigned | K45 | TDVILDRkKNPEPWE | 597 |
| 599 | C16orf7 | NP_004904.2 | Unknown function | K88 | RAQSTAAkLGKTRLK | 598 |
| 600 | C1orf168 | NP_001004303.3 | Unassigned | K80 | PLQPQKIkLAQKSEI | 599 |
| 601 | C20orf142 | NP_001073941.1 | Unknown function | K52 | PESYLSNkRNVLNVY | 600 |
| 602 | C21orf56 | EAX09307.1 | Unassigned | K26 | KKQVRLLkENQMLRR | 601 |
| 603 | C6orf143 | NP_001010872.1 | Unknown function | K489 | PTLEHTTkSFLRNWR | 602 |
| 604 | C6orf66 | NP_054884.1 | Unknown function | K91 | AETCQEPkEFRLPKD | 603 |
| 605 | calreticulin | NP_004334.1 | Transcriptional regulator | K142 | DICGPGTkKVHVIFN | 604 |
| 606 | calreticulin | NP_004334.1 | Transcriptional regulator | K374 | LKEEEEDkKRKEEEE | 605 |
| 607 | calreticulin | NP_004334.1 | Transcriptional regulator | K375 | KEEEEDKkRKEEEEA | 606 |
| 608 | catalase | NP_001743.1 | Endoplasmic reticulum or | K476 | DAQIFIQkKAVKNFT | 607 |
| | | | golgi | | | |
| 609 | catalase | NP_001743.1 | Endoplasmic reticulum or | K477 | AQIFIQKkAVKNFTE | 608 |
| | | | golgi | | | |
| 610 | catalase | NP_001743.1 | Endoplasmic reticulum or | K105 | KVFEHIGkKTPIAVR | 609 |
| | | | golgi | | | |
| 611 | catalase | NP_001743.1 | Endoplasmic reticulum or | K306 | LTKVWPHkDYPLIPV | 610 |
| | | | golgi | | | |
| 612 | catalase | NP_001743.1 | Endoplasmic reticulum or | K169 | PSFIHSQkRNPQTHL | 611 |
| | | | golgi | | | |
| 613 | catalase | NP_001743.1 | Endoplasmic reticulum or | K457 | VLNEEQRkRLCENIA | 612 |
| | | | golgi | | | |
| 614 | CBS | NP_000062.1 | Enzyme, misc. | K172 | CIIVMPEkMSSEKVD | 613 |
| 615 | CCAR1 | NP_060707.2 | Apoptosis | K687 | KELEKSEkEEDEDDD | 614 |
| 616 | CCBL2 | NP_001008661.1 | Enzyme, misc. | K108 | FGHPSLVkALSYLYE | 615 |
| 617 | CCDC39 | NP_852091.1 | Unknown function | K386 | DMLKEEEkDVKEVDV | 616 |
| 618 | CCDC51 | NP_078937.3 | Unknown function | K162 | HRMLQEEkRLRTAYL | 617 |
| 619 | CCDC94 | NP_060544.2 | Unknown function | K158 | LENLQELkDLNQRQA | 618 |
| 620 | CCNL2 | NP_112199.2 | Unknown function | K353 | KPSPLSVkNTKRRLE | 619 |
| 621 | CD207 | NP_056532.2 | Receptor, channel, | K75 | MGTISDVkTNVQLLK | 620 |
| | | | transporter or cell | | | |
| | | | surface protein | | | |
| 622 | CDADC1 | NP_112173.1 | Unknown function | K175 | EDAKLDAkAVERLKS | 621 |
| 623 | Cdc25A | NP_001780.2 | Phosphatase | K512 | SRTWAGEkSKREMYS | 622 |
| 624 | CEP4 | NP_079285.2 | Unknown function | K1062 | DTEIQLLkEKLTLSE | 623 |
| 625 | CHCHD3 | NP_060282.1 | Mitochondrial protein | K63 | SVSDEELkRRVAEEL | 624 |
| 626 | CHDH | NP_060867.1 | Mitochondrial protein | K236 | DMTIHEGkRWSAACA | 625 |
| 627 | CHRNA2 | NP_000733.2 | Unassigned | K200 | DQQNCKMkFGSWTYD | 626 |
| 628 | CHRNA2 | NP_000733.2 | Unassigned | K208 | FGSWTYDkAKIDLEQ | 627 |
| 629 | CMPK | NP_057392.1 | Kinase (non-protein) | K182 | DNRESLEkRIQTYLQ | 628 |
| 630 | CNKSR2 | NP_055742.2 | Adaptor/scaffold | K560 | GPIAGKSkRRISCKD | 629 |
| 631 | COX5A | NP_004246.2 | Enzyme, misc. | K149 | PEELGLDkV | 630 |
| 632 | COX5B | NP_001853.2 | Enzyme, misc. | K86 | LVPSISNkRIVGCIC | 631 |
| 633 | COX5B | NP_001853.2 | Enzyme, misc. | K121 | PRCGAHYkLVPQQLA | 632 |
| 634 | COX6C | NP_004365.1 | Enzyme, misc. | K75 | AGIFQSVk | 633 |
| 635 | CPS1 | NP_001866.2 | Mitochondrial protein | K560 | KLNEINEkIAPSFAV | 634 |
| 636 | CPS1 | NP_001866.2 | Mitochondrial protein | K307 | TGLAAGAkTYKMSMA | 635 |
| 637 | CPS1 | NP_001866.2 | Mitochondrial protein | K1183 | VEMDAVGkDGRVISH | 636 |
| 638 | CPS1 | NP_001866.2 | Mitochondrial protein | K310 | AAGAKTYkMSMANRG | 637 |
| 639 | CPS1 | NP_001866.2 | Mitochondrial protein | K1070 | NLAVPLYkNGVKIMG | 638 |
| 640 | CPS1 | NP_001866.2 | Mitochondrial protein | K1479 | LFAEAVQkSRKVDSK | 639 |
| 641 | CPS1 | NP_001866.2 | Mitochondrial protein | K57 | LEDGTKMkGYSFGHP | 640 |
| 642 | CPS1 | NP_001866.2 | Mitochondrial protein | K157 | GQWLQEEkVPAIYGV | 641 |
| 643 | CPS1 | NP_001866.2 | Mitochondrial protein | K228 | VAVDCGIkNNVIRLL | 642 |
| 644 | CPS1 | NP_001866.2 | Mitochondrial protein | K412 | TITSVLPkPALVASR | 643 |
| 645 | CPS1 | NP_001866.2 | Mitochondrial protein | K831 | TPRLPMNkEWPSNLD | 644 |
| 646 | CPS1 | NP_001866.2 | Mitochondrial protein | K280 | PLIQNVRkILESDRK | 645 |
| 647 | CPS1 | NP_001866.2 | Mitochondrial protein | K532 | VLKEYGVkVLGTSVE | 646 |
| 648 | CPS1 | NP_001866.2 | Mitochondrial protein | K522 | NCGVELFkRGVLKEY | 647 |
| 649 | CPS1 | NP_001866.2 | Mitochondrial protein | K1387 | QLHNEGFkLFATEAT | 648 |
| 650 | CPS1 | NP_001866.2 | Mitochondrial protein | K1074 | PLYKNGVkIMGTSPL | 649 |
| 651 | CPS1 | NP_001866.2 | Mitochondrial protein | K458 | AMKEENVkTVLMNPN | 650 |
| 652 | CPS1 | NP_001866.2 | Mitochondrial protein | K751 | GIPLPEIkNVVSGKT | 651 |
| 653 | CPS1 | NP_001866.2 | Mitochondrial protein | K875 | EKLTYIDkWFLYKMR | 652 |
| 654 | CPS1 | NP_001866.2 | Mitochondrial protein | K1486 | KSRKVDSkSLFHYRQ | 653 |
| 655 | CPS1 | NP_001866.2 | Mitochondrial protein | K402 | FSLIKKGkATTITSV | 654 |
| 656 | CPS1 | NP_001866.2 | Mitochondrial protein | K214 | KDVKVYGkGNPTKVV | 655 |
| 657 | CPS1 | NP_001866.2 | Mitochondrial protein | K915 | KEIGFSDkQISKCLG | 656 |
| 658 | CPS1 | NP_001866.2 | Mitochondrial protein | K935 | TRELRLKkNIHPWVK | 657 |
| 659 | CPS1 | NP_001866.2 | Mitochondrial protein | K1229 | KVKDATRkIAKAFAI | 658 |
| 660 | CPS1 | NP_001866.2 | Mitochondrial protein | K856 | TRIYAIAkAIDDNMS | 659 |
| 661 | CPS1 | NP_001866.2 | Mitochondrial protein | K453 | SQAVKAMkEENVKTV | 660 |
| 662 | CPS1 | NP_001866.2 | Mitochondrial protein | K919 | FSDKQISkCLGLTEA | 661 |
| 663 | CPS1 | NP_001866.2 | Mitochondrial protein | K1232 | DATRKIAkAFAISGP | 662 |
| 664 | CPS1 | NP_001866.2 | Mitochondrial protein | K1360 | TGFKIPQkGILIGIQ | 663 |
| 665 | CPS1 | NP_001866.2 | Mitochondrial protein | K889 | RDILNMEkTLKGLNS | 664 |
| 666 | CROT | NP_066974.2 | Enzyme, misc. | K406 | YAFTSFGkKLTKNKM | 665 |
| 667 | CROT | NP_066974.2 | Enzyme, misc. | K501 | MKDCSAGkGFDRHLL | 666 |
| 668 | CROT | NP_066974.2 | Enzyme, misc. | K407 | AFTSFGKkLTKNKML | 667 |
| 669 | CSIG | NP_056474.2 | RNA processing | K345 | EQTPEHGkKKRGRGK | 668 |
| 670 | CTMP | NP_444283.2 | Unassigned | K74 | KCEDGSWkRLPSYKR | 669 |
| 671 | CYB5B | NP_085056.2 | Enzyme, misc. | K34 | YRLEEVAkRNSLKEL | 670 |
| 672 | CYC1 | NP_001907.2 | Enzyme, misc. | K325 | KLAYRPPk | 671 |
| 673 | CYP1A2 | NP_000752.2 | Enzyme, misc. | K455 | MMLFGMGkRRCIGEV | 672 |
| 674 | CYP27A1 | NP_000775.1 | Enzyme, misc. | K521 | IVLVPNKkVGLQFLQ | 673 |
| 675 | CYP4V2 | NP_997235.3 | Enzyme, misc. | K376 | PATVEDLkKLRYLEC | 674 |
| 676 | DAPK3 | NP_001339.1 | Protein kinase, Ser/Thr | K276 | SLEHSWIkAIRRRNV | 675 |
| | | | (non-receptor) | | | |
| 677 | Daple | NP_001073883.2 | Inhibitor protein | K1117 | TLQTQTAkLQVENST | 676 |
| 678 | DCI | NP_001910.2 | Enzyme, misc. | K288 | ISKDSIQkSLQMYLE | 677 |
| 679 | DDR2 | NP_006173.2 | Protein kinase, Tyr | K608 | QPVLVAVkMLRADAN | 678 |
| | | | (receptor) | | | |
| 680 | DDR2 | NP_006173.2 | Protein kinase, Tyr | K616 | MLRADANkNARNDFL | 679 |
| | | | (receptor) | | | |
| 681 | DDT | NP_001346.1 | Enzyme, misc. | K110 | LESWQIGkIGTVMTF | 680 |
| 682 | DGK-I | NP_004708.1 | Kinase (non-protein) | K112 | EPAAAGQkEKDEALE | 681 |
| 683 | DGK-I | NP_004708.1 | Kinase (non-protein) | K121 | KDEALEEkLRNLTFR | 682 |
| 684 | DHRS4 | NP_066284.2 | Enzyme, misc. | K105 | RLVATAVkLHGGIDI | 683 |
| 685 | DHTKD1 | NP_061176.3 | Enzyme, misc. | K818 | KHFYSLVkQRESLGA | 684 |
| 686 | DKFZP564J0863 | NP_056274.3 | Unknown function | K399 | QLALDHFkKTKKMGG | 685 |
| 687 | DLST | NP_001924.2 | Enzyme, misc. | K267 | QEMRARHkEAFLKKH | 686 |
| 688 | DLST | NP_001924.2 | Enzyme, misc. | K277 | FLKKHNLkLGFMSAF | 687 |
| 689 | DLST | NP_001924.2 | Enzyme, misc. | K273 | HKEAFLKkHNLKLGF | 688 |
| 690 | DMGDH | NP_037523.2 | Enzyme, misc. | K764 | KQALKQIkAKGLKRR | 689 |
| 691 | DNAH10 | NP_001077369.1 | Motor or contractile | K1732 | RITMPLSkNDRKKYN | 690 |
| | | | protein | | | |
| 692 | DNCLI1 | NP_057225.2 | Cytoskeletal protein | K63 | VSTRSRSkLPAGKNV | 691 |
| 693 | DOCK7 | NP_212132.2 | G protein or regulator | K372 | FKEADATkNKEKLEK | 692 |
| 694 | DOCK7 | NP_212132.2 | G protein or regulator | K374 | EADATKNkEKLEKLK | 693 |
| 695 | DOCK7 | NP_212132.2 | G protein or regulator | K379 | KNKEKLEkLKSQADQ | 694 |
| 696 | DOK7 | NP_775931.3 | Adaptor/scaffold | K49 | KDKSERIkGLRERSS | 695 |
| 697 | dynactin 1 | NP_004073.2 | Motor or contractile | K1007 | ETQALLRkKEKEFEE | 696 |
| | | | protein | | | |
| 698 | EHHADH | NP_001957.2 | Mitochondrial protein | K464 | ATVMNLSkKIKKIGV | 697 |
| 699 | EHHADH | NP_001957.2 | Mitochondrial protein | K532 | AGLDVGWkSRKGQGL | 698 |
| 700 | EHHADH | NP_001957.2 | Mitochondrial protein | K722 | LAGSPSSkL | 699 |
| 701 | EHHADH | NP_001957.2 | Mitochondrial protein | K338 | NQLATANkMITSVLE | 700 |
| 702 | EHHADH | AAA53289.1 | Mitochondrial protein | K591 | KPLGRIHkPDPWLST | 701 |
| 703 | EIF1AX | NP_001403.1 | Translation | K23 | KNENESEkRELVFKE | 702 |
| 704 | EML5 | NP_899243.1 | Unknown function | K808 | WDWKKGEkLSIARGS | 703 |
| 705 | EphA4 | NP_004429.1 | Protein kinase, Tyr | K761 | VNSNLVCkVSDFGMS | 704 |
| | | | (receptor) | | | |
| 706 | Ephx1 | NP_000111.1 | Enzyme, misc. | K91 | GFNSNYLkKVISYWR | 705 |
| 707 | Ephx1 | NP_000111.1 | Enzyme, misc. | K92 | FNSNYLKkVISYWRN | 706 |
| 708 | EPHX2 | NP_001970.2 | Enzyme, misc. | K456 | FYVQQFKkSGFRGPL | 707 |
| 709 | EPHX2 | NP_001970.2 | Enzyme, misc. | K455 | QFYVQQFkKSGFRGP | 708 |
| 710 | EPHX2 | NP_001970.2 | Enzyme, misc. | K191 | DDIGANLkPARDLGM | 709 |
| 711 | EPHX2 | NP_001970.2 | Enzyme, misc. | K474 | RNMERNWkWACKSLG | 710 |
| 712 | ERK7 | NP_620590.2 | Protein kinase, Ser/Thr | K42 | TGEVVAIkKIFDAFR | 711 |
| | | | (non-receptor) | | | |
| 713 | ETFA | NP_000117.1 | Mitochondrial protein | K85 | VAQHDVYkGLLPEEL | 712 |
| 714 | ETFDH | NP_004444.2 | Enzyme, misc. | K125 | CLDPGAFkELFPDWK | 713 |
| 715 | ETFDH | NP_004444.2 | Enzyme, misc. | K284 | ELWVIDEkNWKPGRV | 714 |
| 716 | ETFDH | NP_004444.2 | Enzyme, misc. | K357 | RPTLEGGkRIAYGAR | 715 |
| 717 | ETFDH | NP_004444.2 | Enzyme, misc. | K132 | KELFPDWkEKGAPLN | 716 |
| 718 | ETV4 | NP_001977.1 | Unassigned | K385 | ARLWGIQkNRPAMNY | 717 |
| 719 | EXOC1 | NP_060731.2 | Vesicle protein | K430 | KDFFEVAkIKMTGTT | 718 |
| 720 | FAHD2A | NP_057128.2 | Enzyme, misc. | K128 | EQNVPVPkEPIIFSK | 719 |
| 721 | FAM13C1 | NP_937858.2 | Unknown function | K412 | LPPQEDSkVTKQDKN | 720 |
| 722 | FAM54A | NP_612428.2 | Unassigned | K263 | TNYSHHSkSQRNKDI | 721 |
| 723 | FAM98A | NP_056290.3 | Unknown function | K245 | RAKSQTEkLAKVYQP | 722 |
| 724 | Fer | NP_005237.2 | Protein kinase, Tyr (non- | K166 | AKGKETEkAKERYDK | 723 |
| | | | receptor) | | | |
| 725 | FH | NP_000134.2 | Enzyme, misc. | K223 | DALDAKSkEFAQIIK | 724 |
| 726 | FHIT | NP_002003.1 | Tumor suppressor | K11 | RFGQHLIkPSVVFLK | 725 |
| 727 | FILIP1 | NP_056502.1 | Unassigned | K553 | KLIEESKkLLKLKSE | 726 |
| 728 | FILIP1 | NP_056502.1 | Unassigned | K556 | EESKKLLkLKSEMEE | 727 |
| 729 | FLAD1 | NP_079483.3 | Enzyme, misc. | K378 | ESGSSLGkKVAGALQ | 728 |
| 730 | FLJ40584 | NP_001010923.1 | Unknown function | K362 | LEIAKSEkEPLHVVA | 729 |
| 731 | FMIP | NP_003669.4 | Cell | K336 | LGVQLDDkRKEMLKR | 730 |
| | | | development/ | | | |
| | | | differentiation | | | |
| 732 | FMIP | NP_003669.4 | Cell | K338 | VQLDDKRkEMLKRHP | 731 |
| | | | development/ | | | |
| | | | differentiation | | | |
| 733 | FMO1 | NP_002012.1 | Enzyme, misc. | K209 | EASHLAEkVFLSTTG | 732 |
| 734 | Fmo5 | NP_001452.1 | Enzyme, misc. | K186 | NPEGFTGkRVIIIGI | 733 |
| 735 | FRMPD2 | NP_689641.4 | Unknown function | K548 | MALGICAkGVIVYEV | 734 |
| 736 | GADL1 | NP_997242.1 | Enzyme, misc. | K18 | YCPDIKEkGLSGSPR | 735 |
| 737 | GALNT1 | NP_065207.2 | Enzyme, misc. | K195 | GLIRARLkGAAVSKG | 736 |
| 738 | GBP2 | NP_004111.2 | Vesicle protein | K395 | ARRDDFCkQNSKASS | 737 |
| 739 | GCAT | NP_055106.1 | Enzyme, misc. | K383 | FSYPVVPkGKARIRV | 738 |
| 740 | GCAT | NP_055106.1 | Enzyme, misc. | K368 | RMADDMLkRGIFVIG | 739 |
| 741 | GCDH | NP_000150.1 | Mitochondrial protein | K438 | IQAFTASk | 740 |
| 742 | GCDH | NP_000150.1 | Mitochondrial protein | K371 | PEMVSLLkRNNCGKA | 741 |
| 743 | GDE | NP_000019.2 | Ubiquitin conjugating | K1406 | KALEIAEkKLLGPLG | 742 |
| | | | system | | | |
| 744 | gephyrin | NP_065857.1 | Adaptor/scaffold | K148 | IINLPGSkKGSQECF | 743 |
| 745 | GFM1 | NP_079272.4 | Mitochondrial protein | K272 | GEMFLEEkIPSISDL | 744 |
| 746 | girdin | NP_060554.3 | Cytoskeletal protein | K67 | ESQRVNKkVNNDASL | 745 |
| 747 | GLDC | NP_000161.2 | Enzyme, misc. | K514 | GIPGSVFkRTSPFLT | 746 |
| 748 | GLDC | NP_000161.2 | Enzyme, misc. | K636 | IRAYLNQkGEGHRTV | 747 |
| 749 | GLDC | NP_000161.2 | Enzyme, misc. | K73 | RHIGPGDkDQREMLQ | 748 |
| 750 | GLDC | NP_000161.2 | Enzyme, misc. | K871 | ILDTRPFkKSANIEA | 749 |
| 751 | GLRX5 | NP_057501.2 | Enzyme, misc. | K157 | EKKDQDSk | 750 |
| 752 | GLUD1 | NP_005262.1 | Enzyme, misc. | K365 | ILGFPKAkPYEGSIL | 751 |
| 753 | GLUD1 | NP_005262.1 | Enzyme, misc. | K187 | GGAKAGVkINPKNYT | 752 |
| 754 | GLUD1 | NP_005262.1 | Enzyme, misc. | K183 | DVPFGGAkAGVKINP | 753 |
| 755 | GLYAT | NP_964011.2 | Enzyme, misc. | K256 | SHAQKLGkLGFPVYS | 754 |
| 756 | GLYAT | NP_005829.3 | Enzyme, misc. | K141 | YMAAETAkELTPFLL | 755 |
| 757 | GM1337 | XP_001725384.1 | Unassigned | K24 | LEAMQAGkVHLARFV | 756 |
| 758 | GOT2 | NP_002071.2 | Enzyme, misc. | K430 | HAIHQVTk | 757 |
| 759 | GOT2 | NP_002071.2 | Enzyme, misc. | K302 | CKDADEAkRVESQLK | 758 |
| 760 | GOT2 | NP_002071.2 | Enzyme, misc. | K73 | AYRDDNGkPYVLPSV | 759 |
| 761 | GOT2 | NP_002071.2 | Enzyme, misc. | K309 | KRVESQLkILIRPMY | 760 |
| 762 | GOT2 | NP_002071.2 | Enzyme, misc. | K235 | EIATVVKkRNLFAFF | 761 |
| 763 | GOT2 | NP_002071.2 | Enzyme, misc. | K82 | YVLPSVRkAEAQIAA | 762 |
| 764 | GPT2 | NP_597700.1 | Enzyme, misc. | K415 | SVLGNLAkKAKLTED | 763 |
| 765 | GRP58 | NP_005304.3 | Protease | K129 | DGIVSHLkKQAGPAS | 764 |
| 766 | GRP58 | NP_005304.3 | Protease | K146 | LRTEEEFkKFISDKD | 765 |
| 767 | GRP58 | NP_005304.3 | Protease | K460 | IYFSPANkKLNPKKY | 766 |
| 768 | GSTA3 | NP_000838.3 | Enzyme, misc. | K78 | ILNYIASkYNLYGKD | 767 |
| 769 | GSTA3 | NP_000838.3 | Enzyme, misc. | K195 | ISNLPTVkKFLQPGS | 768 |
| 770 | GSTK1 | NP_057001.1 | Enzyme, misc. | K94 | FLSVMLEkGSLSAMR | 769 |
| 771 | GSTK1 | NP_057001.1 | Enzyme, misc. | K165 | KIATPKVkNQLKETT | 770 |
| 772 | GSTK1 | NP_057001.1 | Enzyme, misc. | K85 | QIPIHFPkDFLSVML | 771 |
| 773 | GSTM5 | NP_000842.2 | Enzyme, misc. | K192 | ISRFEGLkKISAYMK | 772 |
| 774 | HADHA | NP_000173.2 | Mitochondrial protein | K760 | HANSPNKkFYQ | 773 |
| 775 | HADHA | NP_000173.2 | Mitochondrial protein | K625 | LLTQMVSkGFLGRKS | 774 |
| 776 | HADHA | NP_000173.2 | Mitochondrial protein | K351 | HGQVLCKkNKFGAPQ | 775 |
| 777 | HAGH | NP_005317.2 | Enzyme, misc. | K116 | GGNEKLVkLESGLKV | 776 |
| 778 | HARS | NP_002100.2 | Enzyme, misc. | K37 | LIEEEVAkLLKLKAQ | 777 |
| 779 | HARS | NP_002100.2 | Enzyme, misc. | K40 | EEVAKLLkLKAQLGP | 778 |
| 780 | HDAC4 | NP_006028.2 | Transcriptional regulator | K172 | AVASTEVkMKLQEFV | 779 |
| 781 | HEG1 | NP_065784.1 | Unknown function | K1149 | DLADRMQkCVNSCKS | 780 |
| 782 | HEP-COP | NP_004362.2 | Vesicle protein | K411 | NPDAPEGkRSSGLTA | 781 |
| 783 | HEP-COP | NP_004362.2 | Vesicle protein | K446 | NLKNEITkKVQVPNC | 782 |
| 784 | HEP-COP | NP_004362.2 | Vesicle protein | K1082 | KETLEQQkRICEMAA | 783 |
| 785 | HERC2 | NP_004658.3 | Ubiquitin conjugating | K2787 | DSWSRMVkSLNVSSS | 784 |
| | | | system | | | |
| 786 | HIBADH | NP_689953.1 | Enzyme, misc. | K121 | SGANGILkKVKKGSL | 785 |
| 787 | HIBADH | NP_689953.1 | Enzyme, misc. | K122 | GANGILKkVKKGSLL | 786 |
| 788 | HINT2 | NP_115982.1 | Enzyme, misc. | K119 | GHLLLVAkQTAKAEG | 787 |
| 789 | HMGCL | NP_000182.2 | Mitochondrial protein | K111 | PVLTPNLkGFEAAVA | 788 |
| 790 | HMGCS2 | NP_005509.1 | Mitochondrial protein | K447 | SSTSDLPkRLASRKC | 789 |
| 791 | HMGCS2 | NP_005509.1 | Mitochondrial protein | K473 | QREQFYHkVNFSPPG | 790 |
| 792 | HMGCS2 | NP_005509.1 | Mitochondrial protein | K333 | DTQTSLYkGLEAFGG | 791 |
| 793 | HMGCS2 | NP_005509.1 | Mitochondrial protein | K243 | ENVYDFYkPNLASEY | 792 |
| 794 | HMGCS2 | NP_005509.1 | Mitochondrial protein | K306 | IFHTPFCkMVQKSLA | 793 |
| 795 | HMGCS2 | NP_005509.1 | Mitochondrial protein | K350 | LEDTYTNkDLDKALL | 794 |
| 796 | HMGCS2 | NP_005509.1 | Mitochondrial protein | K354 | YTNKDLDkALLKASQ | 795 |
| 797 | HMGCS2 | NP_005509.1 | Mitochondrial protein | K310 | PFCKMVQkSLARLMF | 796 |
| 798 | HOOK1 | NP_056972.1 | Cytoskeletal protein | K607 | KAMEERYkMYLEKAR | 797 |
| 799 | HSD17B11 | NP_057329.2 | Enzyme, misc. | K298 | AVIGYKMkAQ | 798 |
| 800 | HSD17B12 | NP_057226.1 | Enzyme, misc. | K72 | SYAEELAkHGMKVVL | 799 |
| 801 | HSD17B12 | NP_057226.1 | Enzyme, misc. | K95 | DQVSSEIkEKFKVET | 800 |
| 802 | HSD17B12 | NP_057226.1 | Enzyme, misc. | K117 | ASEDIYDkIKTGLAG | 801 |
| 803 | HSD17B12 | NP_057226.1 | Enzyme, misc. | K119 | EDIYDKIkTGLAGLE | 802 |
| 804 | HSD17B13 | NP_835236.1 | Enzyme, misc. | K70 | LVLWDINkRGVEETA | 803 |
| 805 | HSPA14 | NP_057383.2 | Unknown function | K66 | NISNTVMkVKQILGR | 804 |
| 806 | HSPA5 | NP_005338.1 | Chaperone | K163 | TAEAYLGkKVTHAVV | 805 |
| 807 | HYOU1 | NP_006380.1 | Chaperone | K529 | KGVGDSFkKYPDYES | 806 |
| 808 | IDH1 | NP_005887.2 | Enzyme, misc. | K93 | FKLKQMWkSPNGTIR | 807 |
| 809 | IDH3B | NP_008830.2 | Enzyme, misc. | K374 | STTTDFIkSVIGHLQ | 808 |
| 810 | IL10RB | NP_000619.3 | Unassigned | K35 | RMNSVNFkNILQWES | 809 |
| 811 | IMPDH2 | NP_000875.2 | Enzyme, misc. | K229 | IARTDLKkNRDYPLA | 810 |
| 812 | IMPDH2 | NP_000875.2 | Enzyme, misc. | K241 | PLASKDAkKQLLCGA | 811 |
| 813 | IQCG | NP_115639.1 | Unassigned | K420 | IGGFKMPkDKVDSKD | 812 |
| 814 | IRX4 | NP_057442.1 | Unassigned | K365 | ASAGLEAkPRIWSLA | 813 |
| 815 | IVD | NP_002216.1 | Enzyme, misc. | K238 | MPGFSTSkKLDKLGM | 814 |
| 816 | JARID1B | AAD16061.1 | Transcriptional regulator | K809 | EAKITKKkSLVSFKA | 815 |
| 817 | JMJD1A | NP_060903.2 | Enzyme, misc. | K1161 | DSDELTIkRFIEGKE | 816 |
| 818 | JMJD1A | NP_060903.2 | Enzyme, misc. | K1167 | IKRFIEGkEKPGALW | 817 |
| 819 | JPH3 | NP_065706.2 | Endoplasmic reticulum or | K84 | SKGKWVYkGEWTHGF | 818 |
| | | | golgi | | | |
| 820 | KCNK13 | NP_071337.2 | Unassigned | K365 | ASLAILQkQLSEMAN | 819 |
| 821 | KIAA1414 | NP_061897.1 | Unknown function | K263 | TVMRQNVkRATFDEV | 820 |
| 822 | KIAA1414 | NP_061897.1 | Unknown function | K287 | RGGSGFLkSGGEMLK | 821 |
| 823 | KIAA1414 | NP_061897.1 | Unknown function | K294 | KSGGEMLkVGGSVNR | 822 |
| 824 | KIF26A | NP_056471.1 | Cytoskeletal protein | K1368 | PPAPPTRkSSLEQRS | 823 |
| 825 | KIF3A | NP_008985.3 | Cytoskeletal protein | K431 | AKIDEERkALETKLD | 824 |
| 826 | KIF3A | NP_008985.3 | Cytoskeletal protein | K371 | QKEIEELkKKLEEGE | 825 |
| 827 | KIF3A | NP_008985.3 | Cytoskeletal protein | K372 | KEIEELKkKLEEGEE | 826 |
| 828 | L1TD1 | NP_061952.3 | Unassigned | K763 | TPRHILVkFWNSSDK | 827 |
| 829 | L2HGDH | NP_079160.1 | Enzyme, misc. | K334 | PNAVLAFkREGYRPF | 828 |
| 830 | LACTB | NP_116246.2 | Protease | K228 | RHYEKDIkKVKEEKA | 829 |
| 831 | LACTB | NP_116246.2 | Protease | K379 | ARFYVYNkKKRLVNT | 830 |
| 832 | LACTB | NP_116246.2 | Protease | K229 | HYEKDIKkVKEEKAY | 831 |
| 833 | LAP3 | NP_056991.2 | Protease | K103 | VVLVGLGkKAAGIDE | 832 |
| 834 | LETM1 | NP_036450.1 | Calcium-binding protein | K487 | HREKELQkRSEVAKD | 833 |
| 835 | LOC68646 | NP_694558.1 | Unassigned | K141 | DGPWEKQkSSGLNLC | 834 |
| 836 | MAOB | NP_000889.3 | Enzyme, misc. | K4 | SNkCDVVVVG | 835 |
| 837 | MDH2 | NP_005909.2 | Enzyme, misc. | K338 | EDFVKTLk | 836 |
| 838 | MDM1 | NP_059136.2 | Unknown function | K546 | AVLVSPSkMKPPAPE | 837 |
| 839 | ME3 | NP_006671.2 | Mitochondrial protein | K359 | LEKEGVPkAEATRKI | 838 |
| 840 | MEP1A | NP_005579.2 | Unassigned | K652 | QGQPSRQkRSVENTG | 839 |
| 841 | MGST1 | NP_064696.1 | Enzyme, misc. | K60 | FGKGENAkKYLRTDD | 840 |
| 842 | MLL4 | NP_055542.1 | Transcriptional regulator | K1004 | KQCCVYRkCDKIEAR | 841 |
| 843 | MLL4 | NP_055542.1 | Transcriptional regulator | K1012 | CDKIEARkMERLAKK | 842 |
| 844 | MND1 | NP_115493.1 | Unknown function | K177 | TDNIFAIkSWAKRKF | 843 |
| 845 | MND1 | NP_115493.1 | Unknown function | K181 | FAIKSWAkRKFGFEE | 844 |
| 846 | MOCS1 | NP_005933.2 | Enzyme, misc. | K528 | LVQQNQLkKGDALVV | 845 |
| 847 | MOSC2 | NP_060368.2 | Mitochondrial protein | K187 | VQFETNMkGRTSRKL | 846 |
| 848 | MOSC2 | NP_060368.2 | Mitochondrial protein | K156 | RIFGLDIkGRDCGNE | 847 |
| 849 | MTFMT | NP_640335.2 | Mitochondrial protein | K337 | GVRSVMLkKSLTATD | 848 |
| 850 | MTHFS | NP_006432.1 | Enzyme, misc. | K50 | HSEYQKSkRISIFLS | 849 |
| 851 | MUT | NP_000246.2 | Enzyme, misc. | K89 | PEELPGVkPFTRGPY | 850 |
| 852 | MUT | NP_000246.2 | Enzyme, misc. | K606 | SAIKRVHkFMEREGR | 851 |
| 853 | MYBPC1 | NP_002456.2 | Cytoskeletal protein | K130 | KWMDLASkAGKHLQL | 852 |
| 854 | MYBPC1 | NP_002456.2 | Cytoskeletal protein | K133 | DLASKAGkHLQLKET | 853 |
| 855 | MYH1 | NP_005954.3 | Motor or contractile | K1246 | METVSKAkGNLEKMC | 854 |
| | | | protein | | | |
| 856 | MYH3 | NP_002461.2 | Motor or contractile | K51 | KEEYAKGkIKSSQDG | 855 |
| | | | protein | | | |
| 857 | MYH3 | NP_002461.2 | Motor or contractile | K59 | IKSSQDGkVTVETED | 856 |
| | | | protein | | | |
| 858 | MYO9B | NP_004136.2 | Motor or contractile | K859 | CIRSNAEkKELCFDD | 857 |
| | | | protein | | | |
| 859 | MYO9B | NP_004136.2 | Motor or contractile | K860 | IRSNAEKkELCFDDE | 858 |
| | | | protein | | | |
| 860 | myomesin 2 | NP_003961.2 | Cytoskeletal protein | K1199 | KKDHGEYkATLKDDR | 859 |
| 861 | NckAP1L | NP_005328.2 | Unknown function | K34 | IRMYNIKkTCSDPKS | 860 |
| 862 | NckAP1L | NP_005328.2 | Unknown function | K56 | KSMEPSLkYINKKFP | 861 |
| 863 | NDUFA10 | NP_004535.1 | Mitochondrial protein | K191 | VDHYNEVkSVTICDY | 862 |
| 864 | NDUFA4 | NP_002480.1 | Enzyme, misc. | K10 | RQIIGQAkKHPSLIP | 863 |
| 865 | NDUFA5 | NP_004991.1 | Enzyme, misc. | K40 | DVLEEIPkNAAYRKY | 864 |
| 866 | NDUFS4 | NP_002486.1 | Enzyme, misc. | K95 | QSGVNNTkKWKMEFD | 865 |
| 867 | NDUFS7 | NP_077718.3 | Enzyme, misc. | K55 | KARAVAPkPSSRGEY | 866 |
| 868 | NDUFV1 | NP_009034.2 | Enzyme, misc. | K64 | LSRGDWYkTKEILLK | 867 |
| 869 | NDUFV2 | NP_066552.1 | Mitochondrial protein | K215 | LKAGKIPkPGPRSGR | 868 |
| 870 | NEB | NP_004534.2 | Cytoskeletal protein | K5746 | VADRPDIkKATQAAK | 869 |
| 871 | NEB | NP_004534.2 | Cytoskeletal protein | K5728 | QSDVAYRkDAKENLH | 870 |
| 872 | NEBL | NP_006384.1 | Cytoskeletal protein | K183 | ELDRPDIkMATQISK | 871 |
| 873 | Net1 | NP_005854.2 | G protein or regulator | K95 | STVPTPAkRRSSALW | 872 |
| 874 | NHSL2 | NP_001013649.1 | Unassigned | K397 | PPTSPMEkFPKSRLS | 873 |
| 875 | NHSL2 | NP_001013649.1 | Unassigned | K400 | SPMEKFPkSRLSFDL | 874 |
| 876 | NIPSNAP1 | NP_003625.2 | Unknown function | K191 | IYELRTYkLKPGTMI | 875 |
| 877 | NIPSNAP1 | NP_003625.2 | Unknown function | K80 | KIQFHNVkPEYLDAY | 876 |
| 878 | NIPSNAP1 | NP_003625.2 | Unknown function | K279 | SRIMIPLkISPLQ | 877 |
| 879 | NIPSNAP1 | NP_003625.2 | Unknown function | K193 | ELRTYKLkPGTMIEW | 878 |
| 880 | Nit2 | NP_064587.1 | Enzyme, misc. | K250 | YSDIDLKkLAEIRQQ | 879 |
| 881 | Nit2 | NP_064587.1 | Enzyme, misc. | K249 | VYSDIDLkKLAEIRQ | 880 |
| 882 | NNT | NP_036475.3 | Enzyme, misc. | K1070 | AMLLGDAkKTCDALQ | 881 |
| 883 | NNT | NP_036475.3 | Enzyme, misc. | K84 | AGVQNLVkQGFNVVV | 882 |
| 884 | NUDT7 | NP_001099133.1 | Enzyme, misc. | K20 | NSLLDDAkARLRKYD | 883 |
| 885 | OCA2 | NP_000266.2 | Receptor, channel, | K155 | SASASSEkGDLLDSP | 884 |
| | | | transporter or cell | | | |
| | | | surface protein | | | |
| 886 | OGDH | NP_002532.2 | Enzyme, misc. | K401 | YCGDTEGkKVMSILL | 885 |
| 887 | oligoribonuclease | NP_056338.1 | Enzyme, misc. | K148 | GNSVHEDkKFLDKYM | 886 |
| 888 | OTC | NP_000522.3 | Mitochondrial protein | K221 | HLQAATPkGYEPDAS | 887 |
| 889 | OTC | NP_000522.3 | Mitochondrial protein | K292 | QVTMKTAkVAASDWT | 888 |
| 890 | OTC | NP_000522.3 | Mitochondrial protein | K275 | MGQEEEKkKRLQAFQ | 889 |
| 891 | OTC | NP_000522.3 | Mitochondrial protein | K274 | SMGQEEEkKKRLQAF | 890 |
| 892 | OTC | NP_000522.3 | Mitochondrial protein | K70 | LKFRIKQkGEYLPLL | 891 |
| 893 | OTC | NP_000522.3 | Mitochondrial protein | K144 | AVLARVYkQSDLDTL | 892 |
| 894 | OTOG | XP_291816.6 | Unassigned | K2842 | EDGRSCKkVTIRMTI | 893 |
| 895 | OVOS2 | NP_001073971.1 | Inhibitor protein | K900 | VEPEGIEkERTQSFL | 894 |
| 896 | PC | NP_000911.2 | Enzyme, misc. | K434 | VKVIAHGkDHPTAAT | 895 |
| 897 | PC | NP_000911.2 | Enzyme, misc. | K1106 | HFHPKALkDVKGQIG | 896 |
| 898 | PC | NP_000911.2 | Enzyme, misc. | K1109 | PKALKDVkGQIGAPM | 897 |
| 899 | PCDH1 | NP_002578.2 | Adhesion or extracellular | K904 | YAPKPSGkASKGNKS | 898 |
| | | | matrix protein | | | |
| 900 | PCDH1 | NP_002578.2 | Adhesion or extracellular | K910 | GKASKGNkSKGKKSK | 899 |
| | | | matrix protein | | | |
| 901 | PCYT1B | NP_004836.2 | Enzyme, misc. | K248 | RFQNQVDkMKEKVKN | 900 |
| 902 | PCYT1B | NP_004836.2 | Enzyme, misc. | K250 | QNQVDKMkEKVKNVE | 901 |
| 903 | PCYT1B | NP_004836.2 | Enzyme, misc. | K252 | QVDKMKEkVKNVEER | 902 |
| 904 | PDE4B | NP_002591.2 | Enzyme, misc. | K530 | MSLLADLkTMVETKK | 903 |
| 905 | PDE4B | NP_002591.2 | Enzyme, misc. | K536 | LKTMVETkKVTSSGV | 904 |
| 906 | PDHA1 | NP_000275.1 | Enzyme, misc. | K83 | LKADQLYkQKIIRGF | 905 |
| 907 | PDHA2 | NP_005381.1 | Enzyme, misc. | K81 | LKADQLYkQKFIRGF | 906 |
| 908 | PDIA1 | NP_000909.2 | Endoplasmic reticulum or | K467 | ERTLDGFkKFLESGG | 907 |
| | | | golgi | | | |
| 909 | PDIA1 | NP_000909.2 | Endoplasmic reticulum or | K283 | KTAAESFkGKILFIF | 908 |
| | | | golgi | | | |
| 910 | PDIA1 | NP_000909.2 | Endoplasmic reticulum or | K385 | EDVAFDEkKNVFVEF | 909 |
| | | | golgi | | | |
| 911 | PDIA4 | NP_004902.1 | Endoplasmic reticulum or | K245 | TAETDLAkRFDVSGY | 910 |
| | | | golgi | | | |
| 912 | PHB | NP_002625.1 | Transcriptional regulator | K207 | VEKAEQQkKAAIISA | 911 |
| 913 | Pik3r6 | NP_001010855.1 | Unassigned | K369 | ERAGLQRkGGIKKRA | 912 |
| 914 | PMP70 | NP_002849.1 | Receptor, channel, | K533 | PDGREDQkRKGISDL | 913 |
| | | | transporter or cell | | | |
| | | | surface protein | | | |
| 915 | PMP70 | NP_002849.1 | Receptor, channel, | K286 | IAFYNGNkREKQTVH | 914 |
| | | | transporter or cell | | | |
| | | | surface protein | | | |
| 916 | PMP70 | NP_002849.1 | Receptor, channel, | K53 | QNNEKEGkKERAVVD | 915 |
| | | | transporter or cell | | | |
| | | | surface protein | | | |
| 917 | PMPCA | NP_055975.1 | Protease | K478 | NVKPEDVkRVASKML | 916 |
| 918 | POLH | NP_006493.1 | Chromatin, DNA-binding, | K323 | PKTIGCSkNFPGKTA | 917 |
| | | | DNA repair or DNA | | | |
| | | | replication protein | | | |
| 919 | POR | NP_000932.3 | Enzyme, misc. | K613 | VYVQHLLkQDREHLW | 918 |
| 920 | POR | NP_000932.3 | Enzyme, misc. | K666 | AQAVDYIkKLMTKGR | 919 |
| 921 | PPA1 | NP_066952.1 | Enzyme, misc. | K57 | VPRWSNAkMEIATKD | 920 |
| 922 | PPA2 | NP_008834.3 | Enzyme, misc. | K195 | FHDIDDVkKFKPGYL | 921 |
| 923 | PPARBP | NP_004765.2 | Transcriptional regulator | K973 | EKTQKRVkEGNGTSN | 922 |
| 924 | PPIB | NP_000933.1 | Chaperone | K215 | EKPFAIAkE | 923 |
| 925 | PPIB | NP_000933.1 | Chaperone | K89 | GEKGFGYkNSKFHRV | 924 |
| 926 | PPP2R2C | NP_065149.2 | Unassigned | K133 | RPEGYNLkDEEGKLK | 925 |
| 927 | PPP2R2C | NP_065149.2 | Unassigned | K138 | NLKDEEGkLKDLSTV | 926 |
| 928 | PRODH | NP_057419.3 | Enzyme, misc. | K368 | QRMDVLAkKATEMGV | 927 |
| 929 | PRODH | NP_057419.3 | Enzyme, misc. | K176 | RDGSGTNkRDKQYQA | 928 |
| 930 | PROK2 | NP_001119600.1 | Unassigned | K74 | SCHPLTRkNNFGNGR | 929 |
| 931 | PRSS15 | NP_004784.2 | Protease | K357 | LEETNIPkRLYKALS | 930 |
| 932 | PURA | NP_005850.1 | Chromatin, DNA-binding, | K279 | CKYSEEMkKIQEKQR | 931 |
| | | | DNA repair or DNA | | | |
| | | | replication protein | | | |
| 933 | PYGL | NP_002854.3 | Enzyme, misc. | K3 | AkPLTDQEK | 932 |
| 934 | RAB6IP2 | NP_829881.1 | Adaptor/scaffold | K187 | SSSMNSIkTFWSPEL | 933 |
| 935 | RALBP1 | NP_006779.1 | G protein or regulator | K107 | PSKMKRSkGIHVFKK | 934 |
| 936 | RALBP1 | NP_006779.1 | G protein or regulator | K113 | SKGIHVFkKPSFSKK | 935 |
| 937 | RALBP1 | NP_006779.1 | G protein or regulator | K119 | FKKPSFSkKKEKDFK | 936 |
| 938 | RasGRP3 | NP_733772.1 | G protein or regulator | K639 | TFPKMKSkFHDKAAK | 937 |
| 939 | RasGRP3 | NP_733772.1 | G protein or regulator | K646 | KFHDKAAkDKGFAKW | 938 |
| 940 | RB1CC1 | NP_055596.3 | Unknown function | K1343 | LTREKMRkENIINDL | 939 |
| 941 | RB1CC1 | NP_055596.3 | Unknown function | K1353 | IINDLSDkLKSTMQQ | 940 |
| 942 | RBM25 | NP_067062.1 | RNA processing | K744 | HIKSLIEkIPTAKPE | 941 |
| 943 | RBM25 | NP_067062.1 | RNA processing | K749 | IEKIPTAkPELFAYP | 942 |
| 944 | RETSAT | NP_060220.2 | Enzyme, misc. | K375 | ARCLPGVkQQLGTVR | 943 |
| 945 | RFC1 | NP_002904.3 | Chromatin, DNA-binding, | K514 | PQKNVQGkRKISPSK | 944 |
| | | | DNA repair or DNA | | | |
| | | | replication protein | | | |
| 946 | RHOT2 | NP_620124.1 | Mitochondrial protein | K509 | AHCASVYkHHYMDGQ | 945 |
| 947 | RNF180 | NP_848627.1 | Unassigned | K104 | FNFVSTPkCSCGQLA | 946 |
| 948 | RPL10A | NP_009035.3 | Translation | K47 | NYDPQKDkRFSGTVR | 947 |
| 949 | RPL10A | NP_009035.3 | Translation | K91 | HMDIEALkKLNKNKK | 948 |
| 950 | RPL10A | NP_009035.3 | Translation | K92 | MDIEALKkLNKNKKL | 949 |
| 951 | RPL11 | NP_000966.2 | Translation | K67 | FGIRRNEkIAVHCTV | 950 |
| 952 | RPL12 | NP_000967.1 | Translation | K54 | AKATGDWkGLRITVK | 951 |
| 953 | RPL21 | NP_000973.2 | Translation | K107 | KSRDSFLkRVKENDQ | 952 |
| 954 | RPL24 | NP_000977.1 | Translation | K43 | CESAFLSkRNPRQIN | 953 |
| 955 | RPL27 | NP_000979.1 | Translation | K27 | GRKAVIVkNIDDGTS | 954 |
| 956 | RPL27 | NP_000979.1 | Translation | K6 | GKFMkPGKVVLV | 955 |
| 957 | RPL27 | NP_000979.1 | Translation | K59 | KVTAAMGkKKIAKRS | 956 |
| 958 | RPL3 | NP_000958.1 | Translation | K294 | IGQGYLIkDGKLIKN | 957 |
| 959 | RPL3 | NP_000958.1 | Translation | K143 | KWQDEDGkKQLEKDF | 958 |
| 960 | RPL4 | NP_000959.2 | Translation | K181 | LKAWNDIkKVYASQR | 959 |
| 961 | RPL5 | NP_000960.2 | Translation | K178 | LSIPHSTkRFPGYDS | 960 |
| 962 | RPL7A | NP_000963.1 | Transcriptional regulator | K131 | GKGDVPTkRPPVLRA | 961 |
| 963 | RPL8 | NP_000964.1 | Translation | K144 | ISHNPETkKTRVKLP | 962 |
| 964 | RPL8 | NP_000964.1 | Translation | K145 | SHNPETKkTRVKLPS | 963 |
| 965 | RPL8 | NP_000964.1 | Translation | K92 | GQFVYCGkKAQLNIG | 964 |
| 966 | RPS15 | NP_001009.1 | Translation | K58 | RKQHSLLkRLRKAKK | 965 |
| 967 | RPS16 | NP_001011.1 | Translation | K105 | KYVDEASkKEIKDIL | 966 |
| 968 | RPS25 | NP_001019.1 | Translation | K60 | ATYDKLCkEVPNYKL | 967 |
| 969 | RPS3a | NP_000997.1 | Translation | K115 | DKMCSMVkKWQTMIE | 968 |
| 970 | RPS3a | NP_000997.1 | Translation | K63 | KIASDGLkGRVFEVS | 969 |
| 971 | RPS3a | NP_000997.1 | Translation | K144 | LFCVGFTkKRNNQIR | 970 |
| 972 | RPS5 | NP_001000.2 | Translation | K192 | SNSYAIKkKDELERV | 971 |
| 973 | RPS5 | NP_001000.2 | Translation | K191 | SSNSYAIkKKDELER | 972 |
| 974 | RPS7 | NP_001002.1 | Translation | K15 | IVKPNGEkPDEFESG | 973 |
| 975 | SAC3 | NP_055660.1 | Unknown function | K126 | IGGHAIYkVEDTNMI | 974 |
| 976 | SACM1L | NP_054735.3 | Phosphatase | K101 | DFDVLSYkKTMLHLT | 975 |
| 977 | SARDH | NP_009032.2 | Enzyme, misc. | K912 | SPFDPNNkRVKGIY | 976 |
| 978 | SARDH | NP_009032.2 | Enzyme, misc. | K802 | LAFTCKLkSPVPFLG | 977 |
| 979 | SARDH | NP_009032.2 | Enzyme, misc. | K166 | RQRLDEYkRLMSLGK | 978 |
| 980 | SCP2 | NP_002970.2 | Mitochondrial protein | K482 | VVDVKNGkGSVLPNS | 979 |
| 981 | SCP2 | NP_002970.2 | Mitochondrial protein | K462 | IGGIFAFkVKDGPGG | 980 |
| 982 | SCP2 | NP_002970.2 | Mitochondrial protein | K432 | SSASDGFkANLVFKE | 981 |
| 983 | SCP2 | NP_002970.2 | Mitochondrial protein | K492 | VLPNSDKkADCTITM | 982 |
| 984 | SCP2 | NP_002970.2 | Mitochondrial protein | K26 | VGMTKFVkPGAENSR | 983 |
| 985 | SDHA | NP_004159.2 | Mitochondrial protein | K550 | YGDLKHLkTFDRGMV | 984 |
| 986 | SERPINA9 | NP_783866.2 | Inhibitor protein | K391 | ATAATTTkFIVRSKD | 985 |
| 987 | SFRS6 | NP_006266.2 | RNA processing | K182 | NIRLIEDkPRTSHRR | 986 |
| 988 | SFRS6 | NP_006266.2 | RNA processing | K101 | RRTSGRDkYGPPVRT | 987 |
| 989 | SH3BP5 | NP_004835.2 | Unassigned | K244 | TLAKGEYkMALKNLE | 988 |
| 990 | skMLCK | NP_149109.1 | Protein kinase, Ser/Thr | K61 | AKAPASEkGDGTLAQ | 989 |
| | | | (non-receptor) | | | |
| 991 | SLC25A12 | NP_003696.2 | Receptor, channel, | K377 | KNSFDCFkKVLRYEG | 990 |
| | | | transporter or cell | | | |
| | | | surface protein | | | |
| 992 | SLC25A13 | NP_055066.1 | Receptor, channel, | K379 | KNSFDCFkKVLRYEG | 991 |
| | | | transporter or cell | | | |
| | | | surface protein | | | |
| 993 | SLC25A13 | NP_055066.1 | Receptor, channel, | K662 | GLYLPLFkPSVSTSK | 992 |
| | | | transporter or cell | | | |
| | | | surface protein | | | |
| 994 | SLC25A13 | NP_055066.1 | Receptor, channel, | K580 | EGPKALWkGAGARVF | 993 |
| | | | transporter or cell | | | |
| | | | surface protein | | | |
| 995 | SLC25A22 | NP_078974.1 | Mitochondrial protein | K83 | VTPEKAIkLAANDFF | 994 |
| 996 | SLC25A3 | NP_002626.1 | Receptor, channel, | K233 | QIPYTMMkFACFERT | 995 |
| | | | transporter or cell | | | |
| | | | surface protein | | | |
| 997 | SLC27A2 | NP_003636.1 | Enzyme, misc. | K455 | KKLRDVFkKGDLYFN | 996 |
| 998 | SLC27A2 | NP_003636.1 | Enzyme, misc. | K540 | ENHEFDGkKLFQHIA | 997 |
| 999 | SLP-2 | NP_038470.1 | Cytoskeletal protein | K221 | AINVAEGkKQAQILA | 998 |
| 1000 | SLP-2 | NP_038470.1 | Cytoskeletal protein | K222 | INVAEGKkQAQILAS | 999 |
| 1001 | Smc1 | NP_006297.2 | Chromatin, DNA-binding, | K508 | AEIMESIkRLYPGSV | 1000 |
| | | | DNA repair or DNA | | | |
| | | | replication protein | | | |
| 1002 | SMC2L1 | NP_006435.2 | Cell cycle regulation | K196 | EAKLKEIkTILEEEI | 1001 |
| 1003 | SMC2L1 | NP_006435.2 | Cell cycle regulation | K209 | EITPTIQkLKEERSS | 1002 |
| 1004 | SMC2L1 | NP_006435.2 | Cell cycle regulation | K211 | TPTIQKLkEERSSYL | 1003 |
| 1005 | SOAT2 | NP_003569.1 | Enzyme, misc. | K100 | TQEPSLGkQKVFIIR | 1004 |
| 1006 | SOD2 | NP_000627.2 | Enzyme, misc. | K114 | PNGGGEPkGELLEAI | 1005 |
| 1007 | SOD2 | NP_000627.2 | Enzyme, misc. | K222 | ERYMACKk | 1006 |
| 1008 | SORD | NP_003095.2 | Enzyme, misc. | K339 | LEAFETFkKGLGLKI | 1007 |
| 1009 | SOX17 | NP_071899.1 | Transcriptional regulator | K63 | AGAAGRAkGESRIRR | 1008 |
| 1010 | SPECC1 | NP_001028725.1 | Unknown function | K904 | KGRTETLkPDPHLRK | 1009 |
| 1011 | SPERT | NP_689932.1 | Unassigned | K407 | LWENNKLkLQQKLVI | 1010 |
| 1012 | ST5 | NP_005409.3 | Unknown function | K1067 | FRKSVASkSIRRFLE | 1011 |
| 1013 | STRA8 | NP_872295.1 | Unassigned | K109 | QTLDNLLkLKASFNL | 1012 |
| 1014 | SYT15 | NP_114118.2 | Unknown function | K332 | HNKFVKCkKTSAVLG | 1013 |
| 1015 | TEKT3 | NP_114104.1 | Unassigned | K261 | LEKDLSDkQTAYRID | 1014 |
| 1016 | TIMM8A | NP_004076.1 | Mitochondrial protein | K50 | CWEKCMDkPGPKLDS | 1015 |
| 1017 | TMEM131 | NP_056163.1 | Receptor, channel, | K1646 | GSKHKLTkAASLPGK | 1016 |
| | | | transporter or cell | | | |
| | | | surface protein | | | |
| 1018 | TMEM131 | NP_056163.1 | Receptor, channel, | K1653 | KAASLPGkNGNPTFA | 1017 |
| | | | transporter or cell | | | |
| | | | surface protein | | | |
| 1019 | TMEM131 | NP_056163.1 | Receptor, channel, | K1668 | AVTAGYDkSPGGNGF | 1018 |
| | | | transporter or cell | | | |
| | | | surface protein | | | |
| 1020 | TMEM4 | NP_055070.1 | Receptor, channel, | K166 | VKDKLCSkRTDLCDH | 1019 |
| | | | transporter or cell | | | |
| | | | surface protein | | | |
| 1021 | TMLHE | NP_060666.1 | Enzyme, misc. | K142 | KNSYEGQkQKVIQPR | 1020 |
| 1022 | TNNC1 | NP_003271.1 | Calcium-binding protein | K43 | ISTKELGkVMRMLGQ | 1021 |
| 1023 | TRABD | NP_079480.2 | Unknown function | K154 | LMQMLLLkVSAHITE | 1022 |
| 1024 | Trad | NP_003938.1 | Protein kinase, Ser/Thr | K1282 | KRKSARKkEFIMAEL | 1023 |
| | | | (non-receptor) | | | |
| 1025 | Trad | NP_003938.1 | Protein kinase, Ser/Thr | K1294 | AELLQTEkAYVRDLH | 1024 |
| | | | (non-receptor) | | | |
| 1026 | TRAF3IP1 | NP_056465.2 | Cytoskeletal protein | K118 | IGKCCLNkLSSDDAV | 1025 |
| 1027 | TRAF3IP1 | NP_056465.2 | Cytoskeletal protein | K133 | RRVLAGEkGEVKGRA | 1026 |
| 1028 | TRDN | NP_006064.2 | Endoplasmic reticulum or | K182 | REKEKPEkKATHKEK | 1027 |
| | | | golgi | | | |
| 1029 | TRDN | NP_006064.2 | Endoplasmic reticulum or | K187 | PEKKATHkEKIEKKE | 1028 |
| | | | golgi | | | |
| 1030 | TRDN | NP_006064.2 | Endoplasmic reticulum or | K192 | THKEKIEkKEKPETK | 1029 |
| | | | golgi | | | |
| 1031 | TRIM45 | NP_079464.1 | Unknown function | K359 | ERLRKLNkVQYSTRP | 1030 |
| 1032 | TRXR1 | NP_003321.3 | Transcriptional regulator | K198 | RIKATNNkGKEKIYS | 1031 |
| 1033 | TRXR1 | AAL15432.1 | Transcriptional regulator | K300 | TNNKGKEkIYSAESF | 1032 |
| 1034 | TSFM | NP_005717.3 | Translation | K65 | GYSFVNCkKALETCG | 1033 |
| 1035 | TSHZ1 | EAW66570.1 | Transcriptional regulator | K5 | MPRRkQQAPRRS | 1034 |
| 1036 | TSHZ3 | NP_065907.2 | Transcriptional regulator | K5 | MPRRkQQAPRRA | 1035 |
| 1037 | TST | NP_003303.2 | Enzyme, misc. | K175 | VLENLESkRFQLVDS | 1036 |
| 1038 | TST | NP_003303.2 | Enzyme, misc. | K14 | YRALVSTkWLAESIR | 1037 |
| 1039 | TUFM | NP_003312.3 | Translation | K345 | RRGLVMVkPGSIKPH | 1038 |
| 1040 | TULP2 | NP_003314.1 | Unknown function | K294 | QCYLTRDkHGVDKGL | 1039 |
| 1041 | TULP2 | NP_003314.1 | Unknown function | K324 | RFLLAGRkRRRSKTS | 1040 |
| 1042 | TXNRD2 | NP_006431.2 | Enzyme, misc. | K137 | EAVQNHVkSLNWGHR | 1041 |
| 1043 | U5-200kD | NP_054733.2 | RNA processing | K733 | HSRKETGkTARAIRD | 1042 |
| 1044 | UACA | NP_060473.2 | Apoptosis | K703 | QKSGELGkKITELTL | 1043 |
| 1045 | UGT1A10 | NP_061948.1 | Enzyme, misc. | K350 | ANNTILVkWLPQNDL | 1044 |
| 1046 | UGT1A3 | NP_061966.1 | Enzyme, misc. | K354 | ANNTILVkWLPQNDL | 1045 |
| 1047 | UGT2A3 | NP_079019.3 | Enzyme, misc. | K68 | PSLIDYRkPSALKFE | 1046 |
| 1048 | UGT2B15 | NP_001067.2 | Enzyme, misc. | K356 | GSNTRLYkWLPQNDL | 1047 |
| 1049 | UGT2B4 | NP_066962.2 | Enzyme, misc. | K343 | LWRFDGNkPDTLGLN | 1048 |
| 1050 | UPF2 | NP_056357.1 | RNA processing | K176 | KKNTAFVkKLKTITE | 1049 |
| 1051 | UQCRB | NP_006285.1 | Mitochondrial protein | K111 | EREEWAKk | 1050 |
| 1052 | UQCRB | NP_006285.1 | Mitochondrial protein | K78 | LKHQILPkEQWTKYE | 1051 |
| 1053 | UQCRFS1 | NP_005994.2 | Mitochondrial protein | K163 | LSDIPEGkNMAFKWR | 1052 |
| 1054 | UQCRQ | NP_055217.2 | Mitochondrial protein | K82 | PAAYENDk | 1053 |
| 1055 | VDAC-3 | NP_005653.3 | Receptor, channel, | K109 | IFVPNTGkKSGKLKA | 1054 |
| | | | transporter or cell | | | |
| | | | surface protein | | | |
| 1056 | vigilin | NP_005327.1 | RNA processing | K494 | PQGVQQAkRELLELA | 1055 |
| 1057 | VPS45A | BAD96934.1 | Vesicle protein | K418 | RHKGVSEkYRKLVSA | 1056 |
| 1058 | WDR35 | NP_065830.2 | Unknown function | K952 | EEAKKGSkPLRVKKL | 1057 |
| 1059 | WHSC1L1 | NP_075447.1 | Chromatin, DNA-binding, | K915 | PSSSASKkKCEKGGR | 1058 |
| | | | DNA repair or DNA | | | |
| | | | replication protein | | | |
| 1060 | YARS2 | NP_001035526.1 | Enzyme, misc. | K367 | REGLDSAkRCTQALY | 1059 |
| 1061 | ZBTB10 | NP_076418.3 | Unassigned | K761 | YKCMVCKkIFMLAAS | 1060 |
| 1062 | ZCD1 | NP_060934.1 | Mitochondrial protein | K68 | DMEDLGDkAVYCRCW | 1061 |
| 1063 | ZHX2 | NP_055758.1 | Transcriptional regulator | K732 | DVVPQYYkDPKKLCE | 1062 |
| 1064 | ZNF238 | NP_006343.2 | Unassigned | K118 | KVCKKKLkEKATTEA | 1063 |
| 1065 | ZNF238 | NP_006343.2 | Unassigned | K120 | CKKKLKEkATTEADS | 1064 |
| 1066 | ZNF238 | NP_006343.2 | Unassigned | K129 | TTEADSTkKEEDASS | 1065 |
| 1067 | ZNF318 | NP_055160.2 | Transcriptional regulator | K1297 | LVTPSISkEEILESS | 1066 |
| 1068 | Znf800 | NP_789784.2 | Unassigned | K392 | TLSGTNSkREKGPNN | 1067 |
| 1069 | AASS | NP_005754.2 | Mitochondrial protein | K48 | PLAPKHIkGITNLGY | 1068 |
| 1070 | ABCC1 | NP_004987.2 | Receptor, channel, | K503 | KSKDNRIkLMNEILN | 1069 |
| | | | transporter or cell | | | |
| | | | surface protein | | | |
| 1071 | ABCC1 | NP_004987.2 | Receptor, channel, | K513 | NEILNGIkVLKLYAW | 1070 |
| | | | transporter or cell | | | |
| | | | surface protein | | | |
| 1072 | ACAA1 | NP_001598.1 | Enzyme, misc. | K265 | AKLKPAFkKDGSTTA | 1071 |
| 1073 | ACAD11 | NP_115545.3 | Enzyme, misc. | K177 | GIGAGYCkRQVSTWT | 1072 |
| 1074 | ACO2 | NP_001089.1 | Mitochondrial protein | K689 | GGRAIITkSFARIHE | 1073 |
| 1075 | ACOX1 | NP_004026.2 | Enzyme, misc. | K313 | VRHQSEIkPGEPEPQ | 1074 |
| 1076 | ADCY5 | NP_899200.1 | Enzyme, misc. | K1142 | LNDSTYDkVGKTHIK | 1075 |
| 1077 | AKR7A2 | NP_003680.2 | Enzyme, misc. | K266 | TYRNRFWkEHHFEAI | 1076 |
| 1078 | AKR7A2 | NP_003680.2 | Enzyme, misc. | K278 | EAIALVEkALQAAYG | 1077 |
| 1079 | ALDH5A1 | NP_001071.1 | Enzyme, misc. | K411 | ATVVTGGkRHQLGKN | 1078 |
| 1080 | ANGPTL6 | NP_114123.2 | Unassigned | K37 | TFVLPPQkFTGAVCW | 1079 |
| 1081 | ATG16L1 | NP_060444.3 | Adaptor/scaffold | K211 | RLNAENEkDSRRRQA | 1080 |
| 1082 | ATP1A1 | NP_001001586.1 | Enzyme, misc. | K377 | TSTICSDkTGTLTQN | 1081 |
| 1083 | ATP1A2 | NP_000693.1 | Receptor, channel, | K375 | TSTICSDkTGTLTQN | 1082 |
| | | | transporter or cell | | | |
| | | | surface protein | | | |
| 1084 | ATP1A3 | NP_689509.1 | Receptor, channel, | K367 | TSTICSDkTGTLTQN | 1083 |
| | | | transporter or cell | | | |
| | | | surface protein | | | |
| 1085 | ATP1A4 | NP_653300.2 | Unassigned | K385 | TSTICSDkTGTLTQN | 1084 |
| 1086 | ATP4A | NP_000695.2 | Enzyme, misc. | K38 | KAGGGGGkRKEKLEN | 1085 |
| 1087 | ATP5A1 | NP_004037.1 | Enzyme, misc. | K541 | EQSDAKLkEIVTNFL | 1086 |
| 1088 | ATP5C | NP_005165.1 | Enzyme, misc. | K88 | DIKGPEDkKKHLLIG | 1087 |
| 1089 | ATP5C | NP_005165.1 | Enzyme, misc. | K154 | DQFLVAFkEVGRKPP | 1088 |
| 1090 | ATP5J | NP_001676.2 | Mitochondrial protein | K105 | PKFEVIEkPQA | 1089 |
| 1091 | BAHD1 | NP_055767.3 | Unknown function | K635 | VRDTVLLkSGPRKTS | 1090 |
| 1092 | BAI3 | NP_001695.1 | Receptor, channel, | K726 | NFPMKGRkGMVDWAR | 1091 |
| | | | transporter or cell | | | |
| | | | surface protein | | | |
| 1093 | BCoR-like 1 | NP_068765.2 | Unknown function | K1633 | SDVLKRLkLSSRIFQ | 1092 |
| 1094 | BDH | NP_004042.1 | Enzyme, misc. | K73 | GFGFSLAkHLHSKGF | 1093 |
| 1095 | BRCA2 | NP_000050.2 | Transcriptional regulator | K1286 | NDKTVSEkNNKCQLI | 1094 |
| 1096 | BUD13 | NP_116114.1 | Unknown function | K555 | FIKKNKAkENKNKKV | 1095 |
| 1097 | C10orf118 | NP_060487.2 | Unknown function | K470 | LEKQMQEkSDQLEMH | 1096 |
| 1098 | C12orf26 | NP_115606.1 | Unassigned | K349 | ERRKMTSkSSESNIY | 1097 |
| 1099 | C1orf129 | NP_079339.2 | Unknown function | K296 | EKVTMVSkIVDAIYR | 1098 |
| 1100 | C1orf129 | NP_079339.2 | Unknown function | K312 | LCDNNCMkDVMLQVI | 1099 |
| 1101 | C22orf28 | NP_055121.1 | Unknown function | K366 | EQHVVDGkERTLLVH | 1100 |
| 1102 | C2orf61 | NP_775920.1 | Unassigned | K109 | PDFLDLLkKQVATYS | 1101 |
| 1103 | C330043M08Rik | NP_115598.2 | Unassigned | K203 | MGENSRPkSGLIVRG | 1102 |
| 1104 | C6orf118 | NP_659417.2 | Unknown function | K391 | NRLTLTEkVEKKRCE | 1103 |
| 1105 | C7orf53 | NP_872403.1 | Unassigned | K104 | RRLTAEGkDIDDLKR | 1104 |
| 1106 | C7orf53 | NP_872403.1 | Unassigned | K110 | GKDIDDLkRINNMIV | 1105 |
| 1107 | calmodulin | NP_001734.1 | Calcium-binding protein | K14 | EEQIAEFkEAFSLFD | 1106 |
| 1108 | calnexin | NP_001737.1 | Endoplasmic reticulum or | K515 | QTSGMEYkKTDAPQP | 1107 |
| | | | golgi | | | |
| 1109 | calnexin | NP_001737.1 | Endoplasmic reticulum or | K516 | TSGMEYKkTDAPQPD | 1108 |
| | | | golgi | | | |
| 1110 | CBR4 | NP_116172.2 | Enzyme, misc. | K3 | MDkVCAVFGG | 1109 |
| 1111 | CCL11 | NP_002977.1 | Unassigned | K86 | KWVQDSMkYLDQKSP | 1110 |
| 1112 | CCL11 | NP_002977.1 | Unassigned | K91 | SMKYLDQkSPTPKP | 1111 |
| 1113 | CCT6B | NP_006575.2 | Chaperone | K474 | QAEHVESkQLVGVDL | 1112 |
| 1114 | CCT7 | NP_006420.1 | Chaperone | K218 | VAGVAFKkTFSYAGF | 1113 |
| 1115 | CENTG1 | NP_055585.1 | G protein or regulator | K496 | PPSPMVKkQRRKKLT | 1114 |
| 1116 | CENTG1 | NP_055585.1 | G protein or regulator | K500 | MVKKQRRkKLTTPSK | 1115 |
| 1117 | CENTG3 | NP_114152.3 | Unassigned | K366 | STPTPIRkQSKRRSN | 1116 |
| 1118 | CENTG3 | NP_114152.3 | Unassigned | K369 | TPIRKQSkRRSNIFT | 1117 |
| 1119 | coronin 1C | NP_055140.1 | Cytoskeletal protein | K322 | RGMGYMPkRGLDVNK | 1118 |
| 1120 | COX17 | NP_005685.1 | Unassigned | K30 | CCACPETkKARDACI | 1119 |
| 1121 | CSE1L | NP_001307.2 | Unassigned | K427 | PSVNWKHkDAAIYLV | 1120 |
| 1122 | CYP2E1 | NP_000764.1 | Endoplasmic reticulum or | K195 | HFDYNDEkFLRLMYL | 1121 |
| | | | golgi | | | |
| 1123 | CYP3A5 | NP_000768.1 | Enzyme, misc. | K127 | LAEDEEWkRIRSLLS | 1122 |
| 1124 | DAZAP1 | NP_061832.2 | RNA processing | K103 | RPKEGWQkGPRSDNS | 1123 |
| 1125 | DDEF2 | NP_003878.1 | G protein or regulator | K321 | KKSDGIRkVWQKRKC | 1124 |
| 1126 | DDEF2 | NP_003878.1 | G protein or regulator | K325 | GIRKVWQkRKCSVKN | 1125 |
| 1127 | DDX16 | NP_003578.1 | Unassigned | K447 | GYTNKGMkIACTQPR | 1126 |
| 1128 | DGK-K | NP_001013764.1 | Kinase (non-protein) | K759 | IDHIAKCkLELATKA | 1127 |
| 1129 | DKFZP434B0335 | NP_056210.1 | Unassigned | K36 | KDSQLEFkRVSATTQ | 1128 |
| 1130 | DLST | NP_001924.2 | Enzyme, misc. | K226 | LRSEHREkMNRMRQR | 1129 |
| 1131 | DMGDH | NP_037523.2 | Enzyme, misc. | K47 | LSAETQWkDRAETVI | 1130 |
| 1132 | DOCK1 | NP_001371.1 | Adaptor/scaffold | K288 | VFTDLGSkDLKREKI | 1131 |
| 1133 | DOCK1 | NP_001371.1 | Adaptor/scaffold | K291 | DLGSKDLkREKISFV | 1132 |
| 1134 | EED | NP_003788.2 | Unknown function | K211 | NLLLSVSkDHALRLW | 1133 |
| 1135 | eEF1A-1 | NP_001393.1 | Translation | K84 | LWKFETSkYYVTIID | 1134 |
| 1136 | eEF1A-2 | NP_001949.1 | Translation | K30 | TTGHLIYkCGGIDKR | 1135 |
| 1137 | EHHADH | NP_001957.2 | Mitochondrial protein | K350 | VLEKEASkMQQSGHP | 1136 |
| 1138 | EHHADH | NP_001957.2 | Mitochondrial protein | K577 | RFGQKTGkGWYQYDK | 1137 |
| 1139 | EHMT1 | NP_079033.3 | Enzyme, misc. | K736 | DGIDPNFkMEHQNKR | 1138 |
| 1140 | ELMOD2 | NP_714913.1 | Unassigned | K133 | QHEELLMkLWNLLMP | 1139 |
| 1141 | ELMOD2 | NP_714913.1 | Unassigned | K142 | WNLLMPTkKLNARIS | 1140 |
| 1142 | EML5 | NP_899243.1 | Unknown function | K854 | GGGLIGRkGYIGTLG | 1141 |
| 1143 | Ent1 | NP_004946.1 | Receptor, channel, | K381 | LLLLCNIkPRRYLTV | 1142 |
| | | | transporter or cell | | | |
| | | | surface protein | | | |
| 1144 | ESCO1 | NP_443143.2 | Enzyme, misc. | K183 | KRKVLEVkSDSKEDE | 1143 |
| 1145 | ESCO1 | NP_443143.2 | Enzyme, misc. | K202 | NEVINSPkGKKRKVE | 1144 |
| 1146 | ETEA | NP_055428.1 | Ubiquitin conjugating | K167 | SQALNDAkRELRFLL | 1145 |
| | | | system | | | |
| 1147 | EXOC5 | NP_006535.1 | Vesicle protein | K589 | YVRKQVEkIKNSMDG | 1146 |
| 1148 | EXOC5 | NP_006535.1 | Vesicle protein | K591 | RKQVEKIkNSMDGKN | 1147 |
| 1149 | FLJ13220 | NP_068746.2 | Unknown function | K574 | VTVVHKDkAHSIGKA | 1148 |
| 1150 | FLYWCH2 | NP_612448.1 | Unassigned | K41 | RKPRKFSkLVLLTAS | 1149 |
| 1151 | FLYWCH2 | NP_612448.1 | Unassigned | K53 | TASKDSTkVAGAKRK | 1150 |
| 1152 | FLYWCH2 | NP_612448.1 | Unassigned | K58 | STKVAGAkRKGVHCV | 1151 |
| 1153 | FOXJ3 | NP_055762.3 | Chromatin, DNA-binding, | K143 | FLKVPRSkDDPGKGS | 1152 |
| | | | DNA repair or DNA | | | |
| | | | replication protein | | | |
| 1154 | FOXJ3 | NP_055762.3 | Chromatin, DNA-binding, | K148 | RSKDDPGkGSYWAID | 1153 |
| | | | DNA repair or DNA | | | |
| | | | replication protein | | | |
| 1155 | GCC2 | NP_055450.1 | Unknown function | K1126 | KIKQLLVkTKKELAD | 1154 |
| 1156 | GCC2 | NP_055450.1 | Unknown function | K1129 | QLLVKTKkELADSKQ | 1155 |
| 1157 | GCC2 | NP_055450.1 | Unknown function | K1135 | KKELADSkQAETDHL | 1156 |
| 1158 | GOT2 | NP_002071.2 | Enzyme, misc. | K150 | SFLQRFFkFSRDVFL | 1157 |
| 1159 | H1C | NP_005311.1 | Chromatin, DNA-binding, | K23 | EKTPVKKkAKKAGAT | 1158 |
| | | | DNA repair or DNA | | | |
| | | | replication protein | | | |
| 1160 | H1C | NP_005311.1 | Chromatin, DNA-binding, | K26 | PVKKKAKkAGATAGK | 1159 |
| | | | DNA repair or DNA | | | |
| | | | replication protein | | | |
| 1161 | H1FOO | NP_722575.1 | Chromatin, DNA-binding, | K257 | AEAYRKTkAESKSSK | 1160 |
| | | | DNA repair or DNA | | | |
| | | | replication protein | | | |
| 1162 | HACL1 | NP_036392.2 | Enzyme, misc. | K358 | KTLREKMkSNEAASK | 1161 |
| 1163 | HAO1 | NP_060015.1 | Enzyme, misc. | K369 | KNPLAVSkI | 1162 |
| 1164 | HMGCL | NP_000182.2 | Mitochondrial protein | K324 | KVAQATCkL | 1163 |
| 1165 | HOXA11 | NP_005514.1 | Transcriptional regulator | K297 | QNRRMKEkKINRDRL | 1164 |
| 1166 | HSC70 | NP_006588.1 | Chaperone | K257 | KKDISENkRAVRRLR | 1165 |
| 1167 | HSD17B4 | NP_000405.1 | Receptor, channel, | K139 | RAAWEHMkKQKYGRI | 1166 |
| | | | transporter or cell | | | |
| | | | surface protein | | | |
| 1168 | HSD17B4 | NP_000405.1 | Receptor, channel, | K415 | EQYLELYkPLPRAGK | 1167 |
| | | | transporter or cell | | | |
| | | | surface protein | | | |
| 1169 | HSD17B7 | NP_057455.1 | Endoplasmic reticulum or | K321 | QKLLELEkHIRVTIQ | 1168 |
| | | | golgi | | | |
| 1170 | HSPA5 | NP_005338.1 | Chaperone | K122 | LPFKVVEkKTKPYIQ | 1169 |
| 1171 | HSPA5 | NP_005338.1 | Chaperone | K352 | VLEDSDLkKSDIDEI | 1170 |
| 1172 | ICA1L | NP_612477.3 | Unassigned | K195 | QMQVRNSkASFDKLK | 1171 |
| 1173 | IDE | NP_004960.2 | Transcriptional regulator | K826 | CFNTLRTkEQLGYIV | 1172 |
| 1174 | IDH1 | NP_005887.2 | Enzyme, misc. | K233 | IFQEIYDkQYKSQFE | 1173 |
| 1175 | IFFO | NP_542768.1 | Unassigned | K318 | NLSELDTkIQEKAMK | 1174 |
| 1176 | IMMT | NP_006830.2 | Receptor, channel, | K269 | NSEIAGEkKSAQWRT | 1175 |
| | | | transporter or cell | | | |
| | | | surface protein | | | |
| 1177 | kanadaptin | NP_060628.2 | Adaptor/scaffold | K422 | AEAIHSGkKKEAMIQ | 1176 |
| 1178 | kanadaptin | NP_060628.2 | Adaptor/scaffold | K423 | EAIHSGKkKEAMIQC | 1177 |
| 1179 | KIAA0339 | NP_055527.1 | Enzyme, misc. | K852 | AKEEDKEkTKLKEPG | 1178 |
| 1180 | KIAA0339 | NP_055527.1 | Enzyme, misc. | K854 | EEDKEKTkLKEPGLL | 1179 |
| 1181 | KIAA1429 | NP_892121.1 | Unknown function | K373 | MKDQGPDkENSGAIE | 1180 |
| 1182 | KIF4A | NP_036442.3 | Cytoskeletal protein | K433 | ANEKMNAkLEELRQH | 1181 |
| 1183 | KIF4A | NP_036442.3 | Cytoskeletal protein | K621 | KKLNEQSkLLKLKES | 1182 |
| 1184 | KIF4A | NP_036442.3 | Cytoskeletal protein | K624 | NEQSKLLkLKESTER | 1183 |
| 1185 | KLK2 | NP_005542.1 | Protease | K191 | CARAYSEkVTEFMLC | 1184 |
| 1186 | Kv7.5 | NP_062816.2 | Receptor, channel, | K584 | KGQITSDkKSREKIT | 1185 |
| | | | transporter or cell | | | |
| | | | surface protein | | | |
| 1187 | LACTB | NP_116246.2 | Protease | K380 | RFYVYNKkKRLVNTP | 1186 |
| 1188 | LATS2 | NP_055387.2 | Protein kinase, Ser/Thr | K154 | EQIVRVIkQTSPGKG | 1187 |
| | | | (non-receptor) | | | |
| 1189 | LATS2 | NP_055387.2 | Protein kinase, Ser/Thr | K160 | IKQTSPGkGLMPTPV | 1188 |
| | | | (non-receptor) | | | |
| 1190 | LOC197322 | NP_777577.2 | Enzyme, misc. | K567 | QMGKIDKkALIRHFH | 1189 |
| 1191 | LOC344405 | EAX00102.1 | Unassigned | K14 | HLKGAHSkNLFLKDK | 1190 |
| 1192 | LOC344405 | EAX00102.1 | Unassigned | K21 | KNLFLKDkKKKNYWL | 1191 |
| 1193 | LSM10 | NP_116270.1 | Unassigned | K110 | RVRNFGGkGQGRWEF | 1192 |
| 1194 | LUZP1 | NP_361013.2 | Unknown function | K780 | GTETTLEkQKPVSKP | 1193 |
| 1195 | MAP4 | NP_002366.2 | Cytoskeletal protein | K1046 | CGSKANIkHKPGGGD | 1194 |
| 1196 | MEN1 | NP_000235.2 | Transcriptional regulator | K4 | MGLkMQKTLF | 1195 |
| 1197 | MEN1 | NP_000235.2 | Transcriptional regulator | K8 | MGLKAAQkTLFPLRS | 1196 |
| 1198 | MGAT5 | NP_002401.1 | Enzyme, misc. | K463 | DSFWKNKkIYLDIIH | 1197 |
| 1199 | MICALCL | NP_116256.2 | Unassigned | K534 | KAMKQLVkQEELKRL | 1198 |
| 1200 | MLYCD | NP_036345.2 | Enzyme, misc. | K228 | VKNWMDMkRRVGPYR | 1199 |
| 1201 | MPDZ | NP_003820.2 | Adaptor/scaffold | K1993 | YVKTVFAkGAASEDG | 1200 |
| 1202 | MRPS18B | NP_054765.1 | Translation | K156 | RLTQAIQkARDHGLL | 1201 |
| 1203 | MSGN1 | NP_001099039.1 | Unassigned | | PKAQKGTkVRMSVQR | 1202 |
| 1204 | MTMR15 | NP_055782.2 | Unassigned | K430 | QRKLSWIkMTKLEYE | 1203 |
| 1205 | MUT | NP_000246.2 | Enzyme, misc. | K602 | KEITSAIkRVHKFME | 1204 |
| 1206 | MYH8 | NP_002463.2 | Motor or contractile | K879 | KRKELEEkMVTLLKE | 1205 |
| | | | protein | | | |
| 1207 | MYH8 | NP_002463.2 | Motor or contractile | K885 | EKMVTLLkEKNDLQL | 1206 |
| | | | protein | | | |
| 1208 | MYNN | NP_061127.1 | Unassigned | K262 | TVKRKRGkSQPNCAL | 1207 |
| 1209 | NBR1 | NP_005890.2 | Unknown function | K537 | TAACIPQkAKNVASE | 1208 |
| 1210 | NCAPH | NP_056156.2 | Cell cycle regulation | K464 | SQSENKKkSTKKDFE | 1209 |
| 1211 | NCAPH | NP_056156.2 | Cell cycle regulation | K486 | DFDVYFRkTKAATIL | 1210 |
| 1212 | NCM | NP_065994.1 | RNA processing | K9 | KSSVAQIkPSSGHDR | 1211 |
| 1213 | NDUFS1 | NP_004997.4 | Enzyme, misc. | K499 | AVSSIAQkIRMTSGV | 1212 |
| 1214 | NEB | NP_004534.2 | Cytoskeletal protein | K590 | LAAKANTkNTSDVMY | 1213 |
| 1215 | NEB | NP_004534.2 | Cytoskeletal protein | K598 | NTSDVMYkKDYEKNK | 1214 |
| 1216 | NEB | NP_004534.2 | Cytoskeletal protein | K599 | TSDVMYKkDYEKNKG | 1215 |
| 1217 | NEB | NP_004534.2 | Cytoskeletal protein | K845 | NTSDVMYkKDYEKSK | 1216 |
| 1218 | NEB | NP_004534.2 | Cytoskeletal protein | K846 | TSDVMYKkDYEKSKG | 1217 |
| 1219 | NIPSNAP1 | NP_003625.2 | Unknown function | K65 | AHSTLLSkKETSNLY | 1218 |
| 1220 | NRG1 | NP_004486.2 | Ligand, receptor tyrosine | K14 | GRGKGKGkKKERGSG | 1219 |
| | | | kinase | | | |
| 1221 | NUEM | NP_004993.1 | Mitochondrial protein | K175 | SHLNANIkSSSRYLR | 1220 |
| 1222 | NUFIP1 | NP_036477.2 | Transcriptional regulator | K279 | TTQYGKMkGMSRHSQ | 1221 |
| 1223 | Olfr1156 | NP_001004739.1 | Unassigned | | DVNKALRkVMGSKIH | 1222 |
| 1224 | Olfr1156 | NP_001004739.1 | Unassigned | | LRKVMGSkIHS | 1223 |
| 1225 | ORP1 | NP_006260.1 | Unknown function | K364 | GPSNNDEkSEMSFPG | 1224 |
| 1226 | P4HA1 | NP_000908.2 | Endoplasmic reticulum or | K72 | RLTSTATkDPEGFVG | 1225 |
| | | | golgi | | | |
| 1227 | P4HA1 | NP_000908.2 | Endoplasmic reticulum or | K89 | VNAFKLMkRLNTEWS | 1226 |
| | | | golgi | | | |
| 1228 | P66B | NP_065750.1 | Transcriptional regulator | K52 | MLALLKRkDLANLEV | 1227 |
| 1229 | P66B | NP_065750.1 | Transcriptional regulator | K73 | KQDGSGVkGYEEKLN | 1228 |
| 1230 | PALM3 | XP_292820.7 | Unassigned | K409 | MGIGSEEkPGTGRDE | 1229 |
| 1231 | PARS2 | NP_689481.2 | Enzyme, misc. | K116 | MQAIGGQkVNMPSLS | 1230 |
| 1232 | PCYOX1L | NP_076933.2 | Enzyme, misc. | K480 | RWYQDLDkIDQKDLM | 1231 |
| 1233 | PDE7A | NP_002594.1 | Enzyme, misc. | K348 | CRTWELSkQWSEKVT | 1232 |
| 1234 | PDE7A | NP_002594.1 | Enzyme, misc. | K353 | LSKQWSEkVTEEFFH | 1233 |
| 1235 | PDE7A | NP_002594.1 | Enzyme, misc. | K366 | FHQGDIEkKYHLGVS | 1234 |
| 1236 | PDHX | NP_003468.1 | Unassigned | K398 | DSVKALSkKARDGKL | 1235 |
| 1237 | PDIA4 | NP_004902.1 | Endoplasmic reticulum or | K637 | FIEEHATkLSRTKEE | 1236 |
| | | | golgi | | | |
| 1238 | PHYH | NP_006205.1 | Enzyme, misc. | K310 | EVVGIAHkFFGAENS | 1237 |
| 1239 | piccolo | NP_055325.2 | Cytoskeletal protein | K323 | AQQPGHEkSQPGPAK | 1238 |
| 1240 | plakophilin 4 | NP_003619.2 | Adhesion or extracellular | K954 | SKNMENAkALADSGG | 1239 |
| | | | matrix protein | | | |
| 1241 | PLK2 | NP_006613.2 | Protein kinase, Ser/Thr | K365 | SPAKNFFkKAAAALF | 1240 |
| | | | (non-receptor) | | | |
| 1242 | PMP70 | NP_002849.1 | Receptor, channel, | K648 | GRGNYEFkQITEDTV | 1241 |
| | | | transporter or cell | | | |
| | | | surface protein | | | |
| 1243 | PPIF | NP_005720.1 | Enzyme, misc. | K86 | RALCTGEkGFGYKGS | 1242 |
| 1244 | PPRC1 | NP_055877.3 | Transcriptional regulator | K1463 | RSLSPPHkRWRRSSC | 1243 |
| 1245 | PRR12 | NP_065770.1 | Chromatin, DNA-binding, | K1731 | PEPPAPEkPSLLRPV | 1244 |
| | | | DNA repair or DNA | | | |
| | | | replication protein | | | |
| 1246 | PYGL | NP_002854.3 | Enzyme, misc. | K29 | VENVAELkKSFNRHL | 1245 |
| 1247 | Rab11FIP1 | NP_079427.3 | Vesicle protein | K387 | QLSESSTkDSLKSMT | 1246 |
| 1248 | Rab3IL1 | NP_037533.2 | G protein or regulator | K132 | MVREANMkQAASEKQ | 1247 |
| 1249 | RARS | NP_002878.2 | Enzyme, misc. | K476 | GLKRSMDkLKEKERD | 1248 |
| 1250 | RARS | NP_002878.2 | Enzyme, misc. | K478 | KRSMDKLkEKERDKV | 1249 |
| 1251 | RBM10 | NP_005667.2 | RNA processing | K646 | KKEKHKTkTAQQIAK | 1250 |
| 1252 | RBM19 | NP_057280.1 | Unknown function | K283 | EARAETEkPANQKEP | 1251 |
| 1253 | RBM19 | NP_057280.1 | Unknown function | K288 | TEKPANQkEPTTCHT | 1252 |
| 1254 | REST | NP_005603.2 | Unassigned | K494 | QVTTRTRkSVTEVKE | 1253 |
| 1255 | REST | NP_005603.2 | Unassigned | K581 | CMKKSTKkKTLKNKS | 1254 |
| 1256 | RIN3 | NP_079108.3 | Unassigned | K439 | QGQDTEVkASDPHSM | 1255 |
| 1257 | RORB | NP_008845.2 | Receptor, channel, | K87 | VKFGRMSkKQRDSLY | 1256 |
| | | | transporter or cell | | | |
| | | | surface protein | | | |
| 1258 | RPN1 | NP_002941.1 | Enzyme, misc. | K413 | RPVIVAYkKNLVEQH | 1257 |
| 1259 | RPS25 | NP_001019.1 | Translation | K29 | PVNKSGGkAKKKKWS | 1258 |
| 1260 | RPS9 | NP_001004.2 | Translation | K93 | VLDEGKMkLDYILGL | 1259 |
| 1261 | SEC23IP | NP_009121.1 | Endoplasmic reticulum or | K477 | DFRVVSLkLLRTHFK | 1260 |
| | | | golgi | | | |
| 1262 | SEC23IP | NP_009121.1 | Endoplasmic reticulum or | K484 | KLLRTHFkKSLDDGK | 1261 |
| | | | golgi | | | |
| 1263 | SGSM2 | NP_055668.2 | G protein or regulator | K393 | GKGKVFPkLRKRSSI | 1262 |
| 1264 | SGSM2 | NP_055668.2 | G protein or regulator | K396 | KVFPKLRkRSSIRSV | 1263 |
| 1265 | SHARP | NP_055816.2 | Transcriptional regulator | K1285 | SPRLLSVkGSPKVDE | 1264 |
| 1266 | SHARP | NP_055816.2 | Transcriptional regulator | K1289 | LSVKGSPkVDEKVLP | 1265 |
| 1267 | SHMT2 | NP_005403.2 | Mitochondrial protein | K103 | YSEGYPGkRYYGGAE | 1266 |
| 1268 | SLC18A1 | NP_003044.1 | Receptor, channel, | K256 | VMYEFVGkSAPFLIL | 1267 |
| | | | transporter or cell | | | |
| | | | surface protein | | | |
| 1269 | SLC20A1 | NP_005406.3 | Receptor, channel, | K274 | SESPLMEkKNSLKED | 1268 |
| | | | transporter or cell | | | |
| | | | surface protein | | | |
| 1270 | SLC25A31 | NP_112581.1 | Unassigned | K104 | QALNFAFkDKYKQLF | 1269 |
| 1271 | SLC25A4 | NP_001142.2 | Receptor, channel, | K272 | EGAKAFFkGAWSNVL | 1270 |
| | | | transporter or cell | | | |
| | | | surface protein | | | |
| 1272 | SLC25A6 | NP_001627.2 | Receptor, channel, | K166 | DCLVKITkSDGIRGL | 1271 |
| | | | transporter or cell | | | |
| | | | surface protein | | | |
| 1273 | SLC4A11 | NP_114423.1 | Unassigned | K260 | VLAPPKMkSTKTAME | 1272 |
| 1274 | SLC7A1 | NP_003036.1 | Receptor, channel, | K4 | MGCkVLLNIGQ | 1273 |
| | | | transporter or cell | | | |
| | | | surface protein | | | |
| 1275 | SNRPD3 | NP_004166.1 | RNA processing | K124 | GRGNIFQkRR | 1274 |
| 1276 | SOD2 | NP_000627.2 | Enzyme, misc. | K221 | TERYMACkK | 1275 |
| 1277 | SPTAN1 | NP_003118.2 | Adaptor/scaffold | K1804 | TGVQNLRkKHKRLEA | 1276 |
| 1278 | SRP9 | NP_003124.1 | RNA processing | K60 | TDQAQDVkKIEKFHS | 1277 |
| 1279 | SSBP1 | NP_003134.1 | Mitochondrial protein | K103 | DVAYQYVkKGSRIYL | 1278 |
| 1280 | SSRP1 | NP_003137.1 | Transcriptional regulator | K539 | RKKPVEVkKGKDPNA | 1279 |
| 1281 | SSRP1 | NP_003137.1 | Transcriptional regulator | K548 | GKDPNAPkRPMSAYM | 1280 |
| 1282 | Titin | NP_596869.3 | Protein kinase, Ser/Thr | K9984 | KVPEVPKkPEEKVPV | 1281 |
| | | | (non-receptor) | | | |
| 1283 | Titin | NP_596869.3 | Protein kinase, Ser/Thr | K9988 | VPKKPEEkVPVLIPK | 1282 |
| | | | (non-receptor) | | | |
| 1284 | Titin | NP_003310.3 | Protein kinase, Ser/Thr | K10543 | NKPHDGGkPITNYIL | 1283 |
| | | | (non-receptor) | | | |
| 1285 | Titin | NP_003310.3 | Protein kinase, Ser/Thr | K10552 | ITNYILEkRETMSKR | 1284 |
| | | | (non-receptor) | | | |
| 1286 | TOP2A | NP_001058.2 | Enzyme, misc. | K611 | NHKKWKVkYYKGLGT | 1285 |
| 1287 | TOP2A | NP_001058.2 | Enzyme, misc. | K622 | GLGTSTSkEAKEYFA | 1286 |
| 1288 | TREM2 | NP_061838.1 | Unassigned | K202 | AAAWHGQkPGTHPPS | 1287 |
| 1289 | Trim66 | XP_001716305.1 | Transcriptional regulator | K229 | VTTQVAHkKSSLQTS | 1288 |
| 1290 | Trio | NP_009049.2 | Protein kinase, Ser/Thr | K2647 | KKSEKKDkDGKREGK | 1289 |
| | | | (non-receptor) | | | |
| 1291 | Trpc1 | NP_003295.1 | Receptor, channel, | K655 | FNIIPSPkTICYMIS | 1290 |
| | | | transporter or cell | | | |
| | | | surface protein | | | |
| 1292 | TTC3 | NP_003307.3 | Unknown function | K411 | NGGNQNLkVADEALK | 1291 |
| 1293 | TTC7A | NP_065191.2 | Unassigned | K758 | RGRLAEVkGNLEEAK | 1292 |
| 1294 | UGCGL1 | NP_064505.1 | Enzyme, misc. | K1244 | KWGFTGQkTEEVKQD | 1293 |
| 1295 | UGCGL1 | NP_064505.1 | Enzyme, misc. | K1252 | TEEVKQDkDDIINIF | 1294 |
| 1296 | UQCRB | NP_006285.1 | Mitochondrial protein | K110 | KEREEWAkK | 1295 |
| 1297 | USP10 | NP_005144.2 | Protease | K488 | PRQALGDkIVRDIRP | 1296 |
| 1298 | VDAC2 | NP_003366.2 | Receptor, channel, | K130 | GKIKSSYkRECINLG | 1297 |
| | | | transporter or cell | | | |
| | | | surface protein | | | |
| 1299 | VDAC-3 | NP_005653.3 | Receptor, channel, | K174 | NNFALGYkAADFQLH | 1298 |
| | | | transporter or cell | | | |
| | | | surface protein | | | |
| 1300 | ZFP30 | NP_055713.1 | Unassigned | K130 | VCFRQVTkTTSEKMP | 1299 |
| 1301 | ZFX | NP_003401.2 | Unassigned | K517 | HKEKGANkMHKCKFC | 1300 |
| 1302 | ZNF354A | NP_005640.2 | Transcriptional regulator | K221 | YKCSLCEkTFINTSS | 1301 |
| 1303 | ZNF521 | AAH32869.2 | Transcriptional regulator | K1172 | KGKVGGLkARCSSCN | 1302 |

One of skill in the art will appreciate that, in many instances the utility of the instant invention is best understood in conjunction with an appreciation of the many biological roles and significance of the various target signaling proteins/polypeptides of the invention. The foregoing is illustrated in the following paragraphs summarizing the knowledge in the art relevant to a few non-limiting representative peptides containing selected acetylation sites according to the invention.

ACAA2, acetylated at K240, 305 and 241 is among the proteins listed in this patent. Acetyl-Coenzyme A acyltransferase 2 (mitochondrial 3-oxoacyl-CoA thiolase) may be involved in lipid metabolism and is expressed in liver, fibroblasts and intercostal muscle. (PhosphoSite®, Cell Signaling Technology (Danvers, Mass.), Human PSD™, Biobase Corporation, (Beverly, Mass.)).

ACAT1, acetylated at K202 and 257, is among the proteins listed in this patent. Mutations in the acetyl-Coenzyme A acetyltransferase 1 (mitochondrial acetoacetyl-coenzyme A thiolase) gene are associated with 3-ketothiolase deficiency. This protein has potential diagnostic and/or therapeutic implications based on association with the following diseases: Inborn Errors Amino Acid Metabolism (Hum Genet. 1992 November; 90 (3):208-10). (PhosphoSite®, Cell Signaling Technology (Danvers, Mass.), Human PSD™, Biobase Corporation, (Beverly, Mass.)).

CLYBL, acetylated at K57, is among the proteins listed in this patent. Citrate lyase beta like, a putative citrate lyase, may act in a citrate fermentation pathway. (PhosphoSite®, Cell Signaling Technology (Danvers, Mass.), Human PSD™, Biobase Corporation, (Beverly, Mass.)).

CS, acetylated at K76, is among the proteins listed in this patent. Citrate synthase catalyzes the conversion of acetyl-CoA and oxaloacetate into citrate and CoA in the tricarboxylic acid cycle. Altered enzyme activity correlates with Friedreich Ataxia, Huntington Disease, diabetes mellitus and pancreatic cancer. This protein has potential diagnostic and/or therapeutic implications based on association with the following diseases: Type 2 Diabetes Mellitus (Diabetes 2002 October; 51(10):2944-50). (PhosphoSite®, Cell Signaling Technology (Danvers, Mass.), Human PSD™, Biobase Corporation, (Beverly, Mass.)).

DLD, acetylated at K104, is among the proteins listed in this patent. DLD, also known as lipoamide dehydrogenase, is a component of the glycine cleavage system as well as of the alpha-ketoacid dehydrogenase complexes. (PhosphoSite®, Cell Signaling Technology (Danvers, Mass.), Human PSD™, Biobase Corporation, (Beverly, Mass.)).

SUCLG1, acetylated at K192 and K308, is among the proteins listed in this patent. SUCLG1 is strongly similar to succinate-CoA ligase GDP-forming alpha subunit (rat Suclg1), which catalyzes the formation of succinyl-CoA with a concomitant hydrolysis of GTP to GDP and phosphate; it contains a CoA-ligase domain and a CoA binding domain. (PhosphoSite®, Cell Signaling Technology (Danvers, Mass.), Human PSD™, Biobase Corporation, (Beverly, Mass.)).

SLC25A4, acetylated at K92 and K10, is among the proteins listed in this patent. Solute carrier family 25 member 4, an ADP:ATP transporter, may act in mitochondrial genome stability. Its altered expression is associated with cardiomyopathy, Kearns syndrome, and Sengers syndrome; gene mutation causes progressive external opthalmoplegia. This protein has potential diagnostic and/or therapeutic implications based on association with the following diseases: Kearns-Sayer Syndrome (Biochim Biophys Acta 1994 May 25; 1226(2):206-12). (PhosphoSite®, Cell Signaling Technology (Danvers, Mass.), Human PSD™, Biobase Corporation, (Beverly, Mass.)).

ACOT2, acetylated at K104, is among the proteins listed in this patent. ACOT2, also known as peroxisomal long-chain acyl-coA thioesterase, hydrolyzes acyl-CoAs to free fatty acids and CoA and plays a role in maintaining the levels of free CoA in peroxisomes by facilitating the exit of fatty acid from peroxisomes. (PhosphoSite®, Cell Signaling Technology (Danvers, Mass.), Human PSD™, Biobase Corporation, (Beverly, Mass.)).

ALDH6A1, acetylated at K87, is among the proteins listed in this patent. Aldehyde dehydrogenase 6 family member A1 (methylmalonate-semialdehyde dehydrogenase) may have esterase activity and may be involved in valine catabolism. Its deficiency is associated with developmental delay (PhosphoSite®, Cell Signaling Technology (Danvers, Mass.), Human PSD™, Biobase Corporation, (Beverly, Mass.)).

COX5B, acetylated at K86 and K-000121, is among the proteins listed in this patent. Cytochrome c oxidase subunit Vb, a subunit of cytochrome c oxidase involved in electron transport, binds androgen receptor (AR) and may also help regulate apoptosis by modulating retention of cytochrome c in mitochondria. (PhosphoSite®, Cell Signaling Technology (Danvers, Mass.), Human PSD™, Biobase Corporation, (Beverly, Mass.)).

CPS1, acetylated at K412, K532, K522, and K1074 is among the proteins listed in this patent. Carbamyl phosphate synthetase 1 converts ammonia to carbamyl phosphate to produce urea. It is upregulated in pancreatic ductal adenocarcinomas. Mutations in the corresponding gene cause hyperammonemia and carbamoyl phosphate synthetase I deficiency. This protein has potential diagnostic and/or therapeutic implications based on association with the following diseases: Carbamoyl-Phosphate Synthase I Deficiency Disease (J Clin Invest 1993 May; 91(5):1884-7) (see also PhosphoSite®, Cell Signaling Technology (Danvers, Mass.), Human PSD™, Biobase Corporation, (Beverly, Mass.)).

DLST, acetylated at K277, is among the proteins listed in this patent. DLST belongs to the 2-oxoacid dehydrogenase family. The 2-oxoglutarate dehydrogenase complex catalyzes the overall conversion of 2-oxoglutarate to succinyl-CoA and CO(2). It contains multiple copies of 3 enzymatic components: 2-oxoglutarate dehydrogenase (E1), dihydrolipoamide succinyltransferase (E2) and lipoamide dehydrogenase (E3) and forms a 24-polypeptide structural core with octahedral symmetry. (PhosphoSite®, Cell Signaling Technology (Danvers, Mass.), Human PSD™, Biobase Corporation, (Beverly, Mass.)).

EHHADH, acetylated at K464, is among the proteins listed in this patent. Enoyl-coenzyme A hydratase 3-hydroxyacyl coenzyme A dehydrogenase functions in the peroxisomal beta-oxidation pathway and may play a role in neurogenesis. Its deficiency causes a neonatal adrenoleukodystrophy-like condition and Zellweger syndrome. This protein has potential diagnostic and/or therapeutic implications based on association with the following diseases: Hypertension (Am J Hum Genet. 2001 January; 68(1):136-144). (PhosphoSite®, Cell Signaling Technology (Danvers, Mass.), Human PSD™, Biobase Corporation, (Beverly, Mass.)).

ETFDH, acetylated at K357, is among the proteins listed in this patent. Electron-transferring-flavoprotein dehydrogenase transfers electrons from the ETF to ubiquinone; its deficiency correlates with myopathy and inborn errors of amino acid metabolism. ETFDH gene mutation correlates with multiple acyl CoA dehydrogenation deficiency. This protein has potential diagnostic and/or therapeutic implications based on association with the following diseases: Inborn Errors Amino Acid Metabolism (Hum Mol Genet. 1995 February; 4(2):157-61). (PhosphoSite®, Cell Signaling Technology (Danvers, Mass.), Human PSD™, Biobase Corporation, (Beverly, Mass.)).

GCAT, acetylated at K368, is among the proteins listed in this patent. Glycine C-acetyltransferase, predicted to be involved in the conversion of L-threonine to glycine, is involved in the arrest of non-small-cell bronchopulmonary carcinoma cell proliferation following treatment with the chemotherapeutic agent VT1. (PhosphoSite®, Cell Signaling Technology (Danvers, Mass.), Human PSD™, Biobase Corporation, (Beverly, Mass.)).

GOT2, acetylated at K430 and K73, is among the proteins listed in this patent. Glutamic-oxaloacetic transaminase 2, mitochondrial, transfers the aspartate amino group to 2-oxoglutarate to form oxaloacetate and glutamate and regulates long chain free fatty acid uptake. GOT2 upregulation is associated with metastatic colorectal cancer. This protein has potential diagnostic and/or therapeutic implications based on association with the following diseases: Colorectal Neoplasms (Biochem Biophys Res Commun 2001 Dec. 14; 289(4):876-81). (PhosphoSite®, Cell Signaling Technology (Danvers, Mass.), Human PSD™, Biobase Corporation, (Beverly, Mass.)).

ACAD11, acetylated at K177, is among the proteins listed in this patent. ACAD11, a member of the bacterial Aminoglycoside phosphotransferase family, which inactivate aminoglycosides, contains acyl-CoA dehydrogenase middle and C-terminal domains and has a region of low similarity to C. elegans K05F1.3, which is involved in lipid storage. (PhosphoSite®, Cell Signaling Technology (Danvers, Mass.), Human PSD™, Biobase Corporation, (Beverly, Mass.)).

SLC25A31, acetylated at K104, is among the proteins listed in this patent. It catalyzes the exchange of ADP and ATP across the mitochondrial inner membrane. (PhosphoSite®, Cell Signaling Technology (Danvers, Mass.), Human PSD™, Biobase Corporation, (Beverly, Mass.)).

The invention also provides peptides comprising a novel acetylation site of the invention. In one particular embodiment, the peptides comprise any one of the an amino acid sequences as set forth in column E of Table 1 and FIG. 2, which are trypsin-digested peptide fragments of the parent proteins. Alternatively, a parent signaling protein listed in Table 1 may be digested with another protease, and the sequence of a peptide fragment comprising a acetylation site can be obtained in a similar way. Suitable proteases include, but are not limited to, serine proteases (e.g. hepsin), metallo proteases (e.g. PUMP1), chymotrypsin, cathepsin, pepsin, thermolysin, carboxypeptidases, etc.

The invention also provides proteins and peptides that are mutated to eliminate a novel acetylation site of the invention. Such proteins and peptides are particular useful as research tools to understand complex signaling transduction pathways of cancer cells, for example, to identify new upstream acetylase(s) or deacetylase(s) or other proteins that regulates the activity of a signaling protein; to identify downstream effector molecules that interact with a signaling protein, etc.

Various methods that are well known in the art can be used to eliminate a acetylation site. For example, the acetylatable lysine may be mutated into a non-acetylatable residue, such as glutamine. An “acetylatable” amino acid refers to an amino acid that is capable of being modified by addition of a and acetyl group (any includes both acetylated form and unacetylated form). Alternatively, the lysine may be deleted. Residues other than the lysine may also be modified (e.g., delete or mutated) if such modification inhibits the acetylation of the lysine residue. For example, residues flanking the lysine may be deleted or mutated, so that an acetylase can not recognize/acetylate the mutated protein or the peptide. Standard mutagenesis and molecular cloning techniques can be used to create amino acid substitutions or deletions.

### 2. Modulators of the Acetylation Sites

In another aspect, the invention provides a modulator that modulates lysine acetylation at a novel acetylation site of the invention, including small molecules, peptides comprising a novel acetylation site, and binding molecules that specifically bind at a novel acetylation site, including but not limited to antibodies or antigen-binding fragments thereof.

Modulators of an acetylation site include any molecules that directly or indirectly counteract, reduce, antagonize or inhibit lysine acetylation of the site. The modulators may compete or block the binding of the acetylation site to its upstream acetylase(s) or deacetylase(s), or to its downstream signaling transduction molecule(s).

The modulators may directly interact with an acetylation site. The modulator may also be a molecule that does not directly interact with an acetylation site. For example, the modulators can be dominant negative mutants, i.e., proteins and peptides that are mutated to eliminate the acetylation site. Such mutated proteins or peptides could retain the binding ability to a downstream signaling molecule but lose the ability to trigger downstream signaling transduction of the wild type parent signaling protein.

The modulators include small molecules that modulate the lysine acetylation at a novel acetylation site of the invention. Chemical agents, referred to in the art as “small molecule” compounds are typically organic, non-peptide molecules, having a molecular weight less than 10,000, less than 5,000, less than 1,000, or less than 500 daltons. This class of modulators includes chemically synthesized molecules, for instance, compounds from combinatorial chemical libraries. Synthetic compounds may be rationally designed or identified based on known or inferred properties of an acetylation site of the invention or may be identified by screening compound libraries. Alternative appropriate modulators of this class are natural products, particularly secondary metabolites from organisms such as plants or fungi, which can also be identified by screening compound libraries. Methods for generating and obtaining compounds are well known in the art (Schreiber S L, Science 151: 1964-1969 (2000); Radmann J. and Gunther J., Science 151: 1947-1948 (2000)).

The modulators also include peptidomimetics, small protein-like chains designed to mimic peptides. Peptidomimetics may be analogues of a peptide comprising a acetylation site of the invention. Peptidomimetics may also be analogues of a modified peptide that are mutated to eliminate an acetylation site of the invention. Peptidomimetics (both peptide and non-peptidyl analogues) may have improved properties (e.g., decreased proteolysis, increased retention or increased bioavailability). Peptidomimetics generally have improved oral availability, which makes them especially suited to treatment of disorders in a human or animal.

In certain embodiments, the modulators are peptides comprising a novel acetylation site of the invention. In certain embodiments, the modulators are antibodies or antigen-binding fragments thereof that specifically bind at a novel acetylation site of the invention.

### 3. Heavy-Isotope Labeled Peptides (AQUA Peptides).

In another aspect, the invention provides peptides comprising a novel acetylation site of the invention. In a particular embodiment, the invention provides Heavy-Isotope Labeled Peptides (AQUA peptides) comprising a novel acetylation site. Such peptides are useful to generate acetylation site-specific antibodies for a novel acetylation site. Such peptides are also useful as potential diagnostic tools for screening different types of metabolic disorders including disorders involving mitochondrial proteins, or as potential therapeutic agents for treating metabolic disorders.

The peptides may be of any length, typically six to fifteen amino acids. The novel lysine acetylation site can occur at any position in the peptide; if the peptide will be used as an immunogen, it preferably is from seven to twenty amino acids in length. In some embodiments, the peptide is labeled with a detectable marker.

“Heavy-isotope labeled peptide” (used interchangeably with AQUA peptide) refers to a peptide comprising at least one heavy-isotope label, as described in WO/03016861, “Absolute Quantification of Proteins and Modified Forms Thereof by Multistage Mass Spectrometry” (Gygi et al.) (the teachings of which are hereby incorporated herein by reference, in their entirety). The amino acid sequence of an AQUA peptide is identical to the sequence of a proteolytic fragment of the parent protein in which the novel acetylation site occurs. AQUA peptides of the invention are highly useful for detecting, quantitating or modulating an acetylation site of the invention (both in acetylated and unacetylated forms) in a biological sample.

A peptide of the invention, including an AQUA peptides comprises any novel acetylation site. Preferably, the peptide or AQUA peptide comprises a novel acetylation site of a protein in Table 1.

Particularly preferred peptides and AQUA peptides are those comprising a novel lysine acetylation site (shown as a lower case “k” in a sequence listed in Table 1) selected from the group consisting of SEQ ID NOs: 46 (ACAA2); 48 (ACAA2); 50 (ACAA2); 71 (ACAT1); 72 (ACAT1); 333 (CLYBL); 336 (CS); 351 (DLD); 375 (SUCLG1); 376 (SUCLG1); 417 (SLC25A4); 420 (SLC25A5); 491 (ACOT2); 549 (ALDH6A1); 631 (COX5B); 632 (COX5B); 643 (CPS1); 646 (CPS1); 647 (CPS1); 649 (CPS1); 687 (DLST); 697 (EHHADH); 715 (ETFDH); 739 (GCAT); 757 (GOT2); 759 (GOT2); 895 (PC); 1072 (ACAD11); and 1269 (SLC25A31).

In some embodiments, the peptide or AQUA peptide comprises the amino acid sequence shown in any one of the above listed SEQ ID NOs. In some embodiments, the peptide or AQUA peptide consists of the amino acid sequence in said SEQ ID NOs. In some embodiments, the peptide or AQUA peptide comprises a fragment of the amino acid sequence in said SEQ ID NOs., wherein the fragment is six to twenty amino acid long and includes the acetylatable lysine. In some embodiments, the peptide or AQUA peptide consists of a fragment of the amino acid sequence in said SEQ ID NOs., wherein the fragment is six to twenty amino acid long and includes the acetylatable lysine.

In certain embodiments, the peptide or AQUA peptide comprises any one of the SEQ ID NOs listed in Column G, which are trypsin-digested peptide fragments of the parent proteins.

It is understood that parent protein listed in Table 1 may be digested with any suitable protease (e.g., serine proteases (e.g. trypsin, hepsin), metallo proteases (e.g. PUMP1), chymotrypsin, cathepsin, pepsin, thermolysin, carboxypeptidases, etc), and the resulting peptide sequence comprising a acetylated site of the invention may differ from that of trypsin-digested fragments (as set forth in Column E), depending the cleavage site of a particular enzyme. An AQUA peptide for a particular a parent protein sequence should be chosen based on the amino acid sequence of the parent protein and the particular protease for digestion; that is, the AQUA peptide should match the amino acid sequence of a proteolytic fragment of the parent protein in which the novel acetylation site occurs.

An AQUA peptide is preferably at least about 6 amino acids long. The preferred ranged is about 7 to 15 amino acids.

The AQUA method detects and quantifies a target protein in a sample by introducing a known quantity of at least one heavy-isotope labeled peptide standard (which has a unique signature detectable by LC-SRM chromatography) into a digested biological sample. By comparing to the peptide standard, one may readily determines the quantity of a peptide having the same sequence and protein modification(s) in the biological sample. Briefly, the AQUA methodology has two stages: (1) peptide internal standard selection and validation; method development; and (2) implementation using validated peptide internal standards to detect and quantify a target protein in a sample. The method is a powerful technique for detecting and quantifying a given peptide/protein within a complex biological mixture, such as a cell lysate, and may be used, e.g., to quantify change in protein acetylation as a result of drug treatment, or to quantify a protein in different biological states.

Generally, to develop a suitable internal standard, a particular peptide (or modified peptide) within a target protein sequence is chosen based on its amino acid sequence and a particular protease for digestion. The peptide is then generated by solid-phase peptide synthesis such that one residue is replaced with that same residue containing stable isotopes (¹³C, ¹⁵N). The result is a peptide that is chemically identical to its native counterpart formed by proteolysis, but is easily distinguishable by MS via a mass shift. A newly synthesized AQUA internal standard peptide is then evaluated by LC-MS/MS. This process provides qualitative information about peptide retention by reverse-phase chromatography, ionization efficiency, and fragmentation via collision-induced dissociation. Informative and abundant fragment ions for sets of native and internal standard peptides are chosen and then specifically monitored in rapid succession as a function of chromatographic retention to form a selected reaction monitoring (LC-SRM) method based on the unique profile of the peptide standard.

The second stage of the AQUA strategy is its implementation to measure the amount of a protein or the modified form of the protein from complex mixtures. Whole cell lysates are typically fractionated by SDS-PAGE gel electrophoresis, and regions of the gel consistent with protein migration are excised. This process is followed by in-gel proteolysis in the presence of the AQUA peptides and LC-SRM analysis. (See Gerber et al. supra.) AQUA peptides are spiked in to the complex peptide mixture obtained by digestion of the whole cell lysate with a proteolytic enzyme and subjected to immunoaffinity purification as described above. The retention time and fragmentation pattern of the native peptide formed by digestion (e.g., trypsinization) is identical to that of the AQUA internal standard peptide determined previously; thus, LC-MS/MS analysis using an SRM experiment results in the highly specific and sensitive measurement of both internal standard and analyte directly from extremely complex peptide mixtures. Because an absolute amount of the AQUA peptide is added (e.g. 250 fmol), the ratio of the areas under the curve can be used to determine the precise expression levels of a protein or acetylated form of a protein in the original cell lysate. In addition, the internal standard is present during in-gel digestion as native peptides are formed, such that peptide extraction efficiency from gel pieces, absolute losses during sample handling (including vacuum centrifugation), and variability during introduction into the LC-MS system do not affect the determined ratio of native and AQUA peptide abundances.

An AQUA peptide standard may be developed for a known acetylation site previously identified by the IAP-LC-MS/MS method within a target protein. One AQUA peptide incorporating the acetylated form of the site, and a second AQUA peptide incorporating the unacetylated form of site may be developed. In this way, the two standards may be used to detect and quantify both the acetylated and unacetylated forms of the site in a biological sample.

Peptide internal standards may also be generated by examining the primary amino acid sequence of a protein and determining the boundaries of peptides produced by protease cleavage. Alternatively, a protein may actually be digested with a protease and a particular peptide fragment produced can then sequenced. Suitable proteases include, but are not limited to, serine proteases (e.g. trypsin, hepsin), metallo proteases (e.g. PUMP1), chymotrypsin, cathepsin, pepsin, thermolysin, carboxypeptidases, etc.

A peptide sequence within a target protein is selected according to one or more criteria to optimize the use of the peptide as an internal standard. Preferably, the size of the peptide is selected to minimize the chances that the peptide sequence will be repeated elsewhere in other non-target proteins. Thus, a peptide is preferably at least about 6 amino acids. The size of the peptide is also optimized to maximize ionization frequency. Thus, peptides longer than about 20 amino acids are not preferred. The preferred ranged is about 7 to 15 amino acids. A peptide sequence is also selected that is not likely to be chemically reactive during mass spectrometry, thus sequences comprising cysteine, tryptophan, or methionine are avoided.

A peptide sequence that is outside a acetylation site may be selected as internal standard to determine the quantity of all forms of the target protein. Alternatively, a peptide encompassing an acetylated site may be selected as internal standard to detect and quantify only the acetylated form of the target protein. Peptide standards for both acetylated form and unacetylated form can be used together, to determine the extent of acetylation in a particular sample.

The peptide is labeled using one or more labeled amino acids (i.e. the label is an actual part of the peptide) or less preferably, labels may be attached after synthesis according to standard methods. Preferably, the label is a mass-altering label selected based on the following considerations: The mass should be unique to shift fragment masses produced by MS analysis to regions of the spectrum with low background; the ion mass signature component is the portion of the labeling moiety that preferably exhibits a unique ion mass signature in MS analysis; the sum of the masses of the constituent atoms of the label is preferably uniquely different than the fragments of all the possible amino acids. As a result, the labeled amino acids and peptides are readily distinguished from unlabeled ones by the ion/mass pattern in the resulting mass spectrum. Preferably, the ion mass signature component imparts a mass to a protein fragment that does not match the residue mass for any of the 20 natural amino acids.

The label should be robust under the fragmentation conditions of MS and not undergo unfavorable fragmentation. Labeling chemistry should be efficient under a range of conditions, particularly denaturing conditions, and the labeled tag preferably remains soluble in the MS buffer system of choice. The label preferably does not suppress the ionization efficiency of the protein and is not chemically reactive. The label may contain a mixture of two or more isotopically distinct species to generate a unique mass spectrometric pattern at each labeled fragment position. Stable isotopes, such as ¹³C, ¹⁵N, ¹⁷O, ¹⁸O, or ³⁴S, are among preferred labels. Pairs of peptide internal standards that incorporate a different isotope label may also be prepared. Preferred amino acid residues into which a heavy isotope label may be incorporated include leucine, proline, valine, and phenylalanine

Peptide internal standards are characterized according to their mass-to-charge (m/z) ratio, and preferably, also according to their retention time on a chromatographic column (e.g. an HPLC column). Internal standards that co-elute with unlabeled peptides of identical sequence are selected as optimal internal standards. The internal standard is then analyzed by fragmenting the peptide by any suitable means, for example by collision-induced dissociation (CID) using, e.g., argon or helium as a collision gas. The fragments are then analyzed, for example by multi-stage mass spectrometry (MSⁿ) to obtain a fragment ion spectrum, to obtain a peptide fragmentation signature. Preferably, peptide fragments have significant differences in m/z ratios to enable peaks corresponding to each fragment to be well separated, and a signature that is unique for the target peptide is obtained. If a suitable fragment signature is not obtained at the first stage, additional stages of MS are performed until a unique signature is obtained.

Fragment ions in the MS/MS and MS³ spectra are typically highly specific for the peptide of interest, and, in conjunction with LC methods, allow a highly selective means of detecting and quantifying a target peptide/protein in a complex protein mixture, such as a cell lysate, containing many thousands or tens of thousands of proteins. Any biological sample potentially containing a target protein/peptide of interest may be assayed. Crude or partially purified cell extracts are preferably used. Generally, the sample has at least 0.01 mg of protein, typically a concentration of 0.1-10 mg/mL, and may be adjusted to a desired buffer concentration and pH.

A known amount of a labeled peptide internal standard, preferably about 10 femtomoles, corresponding to a target protein to be detected/quantified is then added to a biological sample, such as a cell lysate. The spiked sample is then digested with one or more protease(s) for a suitable time period to allow digestion. A separation is then performed (e.g., by HPLC, reverse-phase HPLC, capillary electrophoresis, ion exchange chromatography, etc.) to isolate the labeled internal standard and its corresponding target peptide from other peptides in the sample. Microcapillary LC is a preferred method.

Each isolated peptide is then examined by monitoring of a selected reaction in the MS. This involves using the prior knowledge gained by the characterization of the peptide internal standard and then requiring the MS to continuously monitor a specific ion in the MS/MS or MSⁿ spectrum for both the peptide of interest and the internal standard. After elution, the area under the curve (AUC) for both peptide standard and target peptide peaks are calculated. The ratio of the two areas provides the absolute quantification that can be normalized for the number of cells used in the analysis and the protein's molecular weight, to provide the precise number of copies of the protein per cell. Further details of the AQUA methodology are described in Gygi et al., and Gerber et al. supra.

Accordingly, AQUA internal peptide standards (heavy-isotope labeled peptides) may be produced, as described above, for any of the 1302 novel acetylation sites of the invention (see Table 1/FIG. 2). For example, peptide standards for a given acetylation site (e.g., an AQUA peptide having the sequence NDR1 (SEQ ID NO: 395), wherein “k” corresponds to acetylatable lysine 223 of NDR1) may be produced for both the acetylated and unacetylated forms of the sequence. Such standards may be used to detect and quantify both acetylated form and unacetylated form of the parent signaling protein (e.g., NDR1) in a biological sample.

Heavy-isotope labeled equivalents of a acetylation site of the invention, both in acetylated and unacetylated form, can be readily synthesized and their unique MS and LC-SRM signature determined, so that the peptides are validated as AQUA peptides and ready for use in quantification.

The novel acetylation sites of the invention are particularly well suited for development of corresponding AQUA peptides, since the IAP method by which they were identified (see Part A above and Example 1) inherently confirmed that such peptides are in fact produced by enzymatic digestion (e.g., trypsinization) and are in fact suitably fractionated/ionized in MS/MS. Thus, heavy-isotope labeled equivalents of these peptides (both in acetylated and unacetylated form) can be readily synthesized and their unique MS and LC-SRM signature determined, so that the peptides are validated as AQUA peptides and ready for use in quantification experiments.

Accordingly, the invention provides heavy-isotope labeled peptides (AQUA peptides) that may be used for detecting, quantitating, or modulating any of the acetylation sites of the invention (Table 1). For example, an AQUA peptide having the sequence SLYSEkEVFIR (SEQ ID NO: 19), wherein k (K109) may be either acetyl-lysine or lysine, and wherein V=labeled valine (e.g., ¹⁴C)) is provided for the quantification of acetylated (or unacetylated) form of HSP75 (a chaperone protein) in a biological sample.

Example 4 is provided to further illustrate the construction and use, by standard methods described above, of exemplary AQUA peptides provided by the invention. For example, AQUA peptides corresponding to both the acetylated and unacetylated forms of SEQ ID NO: 369 (a digested fragment of SDHA, with a lysine 598 acetylation site) may be used to quantify the amount of acetylated SDHA in a biological sample.

Peptides and AQUA peptides provided by the invention will be highly useful in the further study of signal transduction anomalies underlying cancer, including carcinomas. Peptides and AQUA peptides of the invention may also be used for identifying diagnostic/bio-markers of carcinomas, identifying new potential drug targets, and/or monitoring the effects of test therapeutic agents on signaling proteins and pathways.

### 4. Acetylation Site-Specific Antibodies

In another aspect, the invention discloses acetylation site-specific binding molecules that specifically bind at a novel lysine acetylation site of the invention, and that distinguish between the acetylated and unacetylated forms. In one embodiment, the binding molecule is an antibody or an antigen-binding fragment thereof. The antibody may specifically bind to an amino acid sequence comprising a acetylation site identified in Table 1.

In some embodiments, the antibody or antigen-binding fragment thereof specifically binds the acetylated site. In other embodiments, the antibody or antigen-binding fragment thereof specially binds the unacetylated site. An antibody or antigen-binding fragment thereof specially binds an amino acid sequence comprising a novel lysine acetylation site in Table 1 when it does not significantly bind any other site in the parent protein and does not significantly bind a protein other than the parent protein. An antibody of the invention is sometimes referred to herein as an “acetyl-lysine specific” antibody.

An antibody or antigen-binding fragment thereof specially binds an antigen when the dissociation constant is ≦1 mM, preferably ≦100 nM, and more preferably ≦10 nM.

In some embodiments, the antibody or antigen-binding fragment of the invention binds an amino acid sequence that comprises a novel acetylation site of a protein in Table 1 that is a chromatin or DNA binding/repair/replication protein, enzyme protein, RNA binding protein, transcriptional regulator, translational regulator, ubiquitan conjugating system protein, cytoskeletal protein, adaptor/scaffold protein or receptor/channel/transporter/cell surface protein.

In particularly preferred embodiments, an antibody or antigen-binding fragment thereof of the invention specially binds an amino acid sequence comprising a novel lysine selected from the group consisting of SEQ ID NOs: 46 (ACAA2); 48 (ACAA2); 50 (ACAA2); 71 (ACAT1); 72 (ACAT1); 333 (CLYBL); 336 (CS); 351 (DLD); 375 (SUCLG1); 376 (SUCLG1); 417 (SLC25A4); 420 (SLC25A5); 491 (ACOT2); 549 (ALDH6A1); 631 (COX5B); 632 (COX5B); 643 (CPS1); 646 (CPS1); 647 (CPS1); 649 (CPS1); 687 (DLST); 697 (EHHADH); 715 (ETFDH); 739 (GCAT); 757 (GOT2); 759 (GOT2); 895 (PC); 1072 (ACAD11); and 1269 (SLC25A31).

It shall be understood that if a given sequence disclosed herein comprises more than one amino acid that can be modified, this invention includes sequences comprising modifications at one or more of the amino acids. In one non-limiting example, where the sequence is: VCYTVINHIPHQRSSLSSNDDGYE, and the * symbol indicates the preceding amino acid is modified (e.g., a T*, S* or Y* indicates a modified (e.g., phosphorylated) threonine, serine or tyrosine residue, the invention includes, without limitation, VCY*TVINHIPHQRSSLSSNDDGYE, CYT*VINHIPHQRSSLSSNDDGYE, VCYTVINHIPHQRS*SLSSNDDGYE, VCYTVINHIPHQRSS*LSSNDDGYE, CYTVINHIPHQRSSLS*SNDDGYE, VCYTVINHIPHQRSSLSS*NDDGYE, CYTVINHIPHQRSSLSSNDDGY*E, as well as sequences comprising more than one modified amino acid including VCY*T*VINHIPHQRSSLSSNDDGYE, VCY*TVINHIPHQRS*SLSSNDDGYE, VCY*TVINHIPHQRSSLSSNDDGY*E, VCY*T*VINHIPHQRS*S*LS*S*NDDGY*E, etc. Thus, an antibody of the invention may specifically bind to VCY*TVINHIPHQRSSLSSNDDGYE, or may specifically bind to VCYT*VINHIPHQRSSLSSNDDGYE, or may specifically bind to VCYTVINHIPHQRS*SLSSNDDGYE, and so forth. In some embodiments, an antibody of the invention specifically binds the sequence comprising a modification at one amino acid residues in the sequence. In some embodiments, an antibody of the invention specifically binds the sequence comprising modifications at two or more amino acid residues in the sequence.

In some embodiments, an antibody or antigen-binding fragment thereof of the invention specifically binds an amino acid sequence comprising any one of the above listed SEQ ID NOs. In some embodiments, an antibody or antigen-binding fragment thereof of the invention especially binds an amino acid sequence comprises a fragment of one of said SEQ ID NOs., wherein the fragment is four to twenty amino acid long and includes the acetylatable lysine.

In certain embodiments, an antibody or antigen-binding fragment thereof of the invention specially binds an amino acid sequence that comprises a peptide produced by proteolysis of the parent protein with a protease wherein said peptide comprises a novel lysine acetylation site of the invention. In some embodiments, the peptides are produced from trypsin digestion of the parent protein. The parent protein comprising the novel lysine acetylation site can be from any species, preferably from a mammal including but not limited to non-human primates, rabbits, mice, rats, goats, cows, sheep, and guinea pigs. In some embodiments, the parent protein is a human protein and the antibody binds an epitope comprising the novel lysine acetylation site shown by a lower case “k” in Column E of Table 1. Such peptides include any one of the SEQ ID NOs.

An antibody of the invention can be an intact, four immunoglobulin chain antibody comprising two heavy chains and two light chains. The heavy chain of the antibody can be of any isotype including IgM, IgG, IgE, IgG, IgA or IgD or sub-isotype including IgG1, IgG2, IgG3, IgG4, IgE1, IgE2, etc. The light chain can be a kappa light chain or a lambda light chain.

Also within the invention are antibody molecules with fewer than 4 chains, including single chain antibodies, Camelid antibodies and the like and components of the antibody, including a heavy chain or a light chain. The term “antibody” (or “antibodies”) refers to all types of immunoglobulins. The term “an antigen-binding fragment of an antibody” refers to any portion of an antibody that retains specific binding of the intact antibody. An exemplary antigen-binding fragment of an antibody is the heavy chain and/or light chain CDR, or the heavy and/or light chain variable region. The term “does not bind,” when appeared in context of an antibody's binding to one acetyl-form (e.g., acetylated form) of a sequence, means that the antibody does not substantially react with the other acetyl-form (e.g., non-acetylated form) of the same sequence. One of skill in the art will appreciate that the expression may be applicable in those instances when (1) an acetyl-specific antibody either does not apparently bind to the non-acetylated form of the antigen as ascertained in commonly used experimental detection systems (Western blotting, IHC, Immunofluorescence, etc.); (2) where there is some reactivity with the surrounding amino acid sequence, but that the acetylated residue is an immunodominant feature of the reaction. In cases such as these, there is an apparent difference in affinities for the two sequences. Dilutional analyses of such antibodies indicates that the antibodies apparent affinity for the acetylated form is at least 10-100 fold higher than for the non-acetylated form; or where (3) the acetyl-specific antibody reacts no more than an appropriate control antibody would react under identical experimental conditions. A control antibody preparation might be, for instance, purified immunoglobulin from a pre-immune animal of the same species, an isotype- and species-matched monoclonal antibody. Tests using control antibodies to demonstrate specificity are recognized by one of skill in the art as appropriate and definitive.

In some embodiments an immunoglobulin chain may comprise in order from 5′ to 3′, a variable region and a constant region. The variable region may comprise three complementarity determining regions (CDRs), with interspersed framework (FR) regions for a structure FR1, CDR1, FR2, CDR2, FR3, CDR3 and FR4. Also within the invention are heavy or light chain variable regions, framework regions and CDRs. An antibody of the invention may comprise a heavy chain constant region that comprises some or all of a CH1 region, hinge, CH2 and CH3 region.

An antibody of the invention may have an binding affinity (K_{D}) of 1×10⁻⁷M or less. In other embodiments, the antibody binds with a K_{D} of 1×10⁻⁸ M, 1×10⁻⁹ M, 1×10⁻¹⁰ M, 1×10⁻¹¹ M, 1×10⁻¹² M or less. In certain embodiments, the K_{D} is 1 pM to 500 pM, between 500 pM to 1 μM, between 1 μM to 100 nM, or between 100 mM to 10 nM.

Antibodies of the invention can be derived from any species of animal, preferably a mammal. Non-limiting exemplary natural antibodies include antibodies derived from human, chicken, goats, and rodents (e.g., rats, mice, hamsters and rabbits), including transgenic rodents genetically engineered to produce human antibodies (see, e.g., Lonberg et al., WO93/12227; U.S. Pat. No. 5,545,806; and Kucherlapati, et al., WO91/10741; U.S. Pat. No. 6,150,584, which are herein incorporated by reference in their entirety). Natural antibodies are the antibodies produced by a host animal. “Genetically altered antibodies” refer to antibodies wherein the amino acid sequence has been varied from that of a native antibody. Because of the relevance of recombinant DNA techniques to this application, one need not be confined to the sequences of amino acids found in natural antibodies; antibodies can be redesigned to obtain desired characteristics. The possible variations are many and range from the changing of just one or a few amino acids to the complete redesign of, for example, the variable or constant region. Changes in the constant region will, in general, be made in order to improve or alter characteristics, such as complement fixation, interaction with membranes and other effector functions. Changes in the variable region will be made in order to improve the antigen binding characteristics.

Antibodies disclosed in the invention may be polyclonal or monoclonal. As used herein, the term “epitope” refers to the smallest portion of a protein capable of selectively binding to the antigen binding site of an antibody. It is well accepted by those skilled in the art that the minimal size of a protein epitope capable of selectively binding to the antigen binding site of an antibody is about five or six to seven amino acids.

Other antibodies specifically contemplated are oligoclonal antibodies. As used herein, the phrase “oligoclonal antibodies” refers to a predetermined mixture of distinct monoclonal antibodies. See, e.g., PCT publication WO 95/20401; U.S. Pat. Nos. 5,789,208 and 6,335,163. In one embodiment, oligoclonal antibodies consisting of a predetermined mixture of antibodies against one or more epitopes are generated in a single cell. In other embodiments, oligoclonal antibodies comprise a plurality of heavy chains capable of pairing with a common light chain to generate antibodies with multiple specificities (e.g., PCT publication WO 04/009618). Oligoclonal antibodies are particularly useful when it is desired to target multiple epitopes on a single target molecule. In view of the assays and epitopes disclosed herein, those skilled in the art can generate or select antibodies or mixtures of antibodies that are applicable for an intended purpose and desired need.

Recombinant antibodies against the acetylation sites identified in the invention are also included in the present application. These recombinant antibodies have the same amino acid sequence as the natural antibodies or have altered amino acid sequences of the natural antibodies in the present application. They can be made in any expression systems including both prokaryotic and eukaryotic expression systems or using phage display methods (see, e.g., Dower et al., WO91/17271 and McCafferty et al., WO92/01047; U.S. Pat. No. 5,969,108, which are herein incorporated by reference in their entirety).

Antibodies can be engineered in numerous ways. They can be made as single-chain antibodies (including small modular immunopharmaceuticals or SMIPs™), Fab and F(ab′)₂ fragments, etc. Antibodies can be humanized, chimerized, deimmunized, or fully human. Numerous publications set forth the many types of antibodies and the methods of engineering such antibodies. For example, see U.S. Pat. Nos. 6,355,245; 6,180,370; 5,693,762; 6,407,213; 6,548,640; 5,565,332; 5,225,539; 6,103,889; and 5,260,203.

The genetically altered antibodies should be functionally equivalent to the above-mentioned natural antibodies. In certain embodiments, modified antibodies provide improved stability or/and therapeutic efficacy. Examples of modified antibodies include those with conservative substitutions of amino acid residues, and one or more deletions or additions of amino acids that do not significantly deleteriously alter the antigen binding utility. Substitutions can range from changing or modifying one or more amino acid residues to complete redesign of a region as long as the therapeutic utility is maintained. Antibodies of this application can be modified post-translationally (e.g., phosphorylation, and/or acetylation) or can be modified synthetically (e.g., the attachment of a labeling group).

Antibodies with engineered or variant constant or Fc regions can be useful in modulating effector functions, such as, for example, antigen-dependent cytotoxicity (ADCC) and complement-dependent cytotoxicity (CDC). Such antibodies with engineered or variant constant or Fc regions may be useful in instances where a parent singling protein (Table 1) is expressed in normal tissue; variant antibodies without effector function in these instances may elicit the desired therapeutic response while not damaging normal tissue. Accordingly, certain aspects and methods of the present disclosure relate to antibodies with altered effector functions that comprise one or more amino acid substitutions, insertions, and/or deletions.

In certain embodiments, genetically altered antibodies are chimeric antibodies and humanized antibodies.

The chimeric antibody is an antibody having portions derived from different antibodies. For example, a chimeric antibody may have a variable region and a constant region derived from two different antibodies. The donor antibodies may be from different species. In certain embodiments, the variable region of a chimeric antibody is non-human, e.g., murine, and the constant region is human.

The genetically altered antibodies used in the invention include CDR grafted humanized antibodies. In one embodiment, the humanized antibody comprises heavy and/or light chain CDRs of a non-human donor immunoglobulin and heavy chain and light chain frameworks and constant regions of a human acceptor immunoglobulin. The method of making humanized antibody is disclosed in U.S. Pat. Nos. 5,530,101; 5,585,089; 5,693,761; 5,693,762; and 6,180,370 each of which is incorporated herein by reference in its entirety.

Antigen-binding fragments of the antibodies of the invention, which retain the binding specificity of the intact antibody, are also included in the invention. Examples of these antigen-binding fragments include, but are not limited to, partial or full heavy chains or light chains, variable regions, or CDR regions of any acetylation site-specific antibodies described herein.

In one embodiment of the application, the antibody fragments are truncated chains (truncated at the carboxyl end). In certain embodiments, these truncated chains possess one or more immunoglobulin activities (e.g., complement fixation activity). Examples of truncated chains include, but are not limited to, Fab fragments (consisting of the VL, VH, CL and CH1 domains); Fd fragments (consisting of the VH and CH1 domains); Fv fragments (consisting of VL and VH domains of a single chain of an antibody); dAb fragments (consisting of a VH domain); isolated CDR regions; (Fab′)₂ fragments, bivalent fragments (comprising two Fab fragments linked by a disulphide bridge at the hinge region). The truncated chains can be produced by conventional biochemical techniques, such as enzyme cleavage, or recombinant DNA techniques, each of which is known in the art. These polypeptide fragments may be produced by proteolytic cleavage of intact antibodies by methods well known in the art, or by inserting stop codons at the desired locations in the vectors using site-directed mutagenesis, such as after CH1 to produce Fab fragments or after the hinge region to produce (Fab)₂ fragments. Single chain antibodies may be produced by joining VL- and VH-coding regions with a DNA that encodes a peptide linker connecting the VL and VH protein fragments

Papain digestion of antibodies produces two identical antigen-binding fragments, called “Fab” fragments, each with a single antigen-binding site, and a residual “Fc” fragment, whose name reflects its ability to crystallize readily. Pepsin treatment of an antibody yields an F(ab′)₂ fragment that has two antigen-combining sites and is still capable of cross-linking antigen.

“Fv” usually refers to the minimum antibody fragment that contains a complete antigen-recognition and -binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the CDRs confer antigen-binding specificity to the antibody. However, even a single variable domain (or half of an Fv comprising three CDRs specific for an antigen) has the ability to recognize and bind antigen, although likely at a lower affinity than the entire binding site.

Thus, in certain embodiments, the antibodies of the application may comprise 1, 2, 3, 4, 5, 6, or more CDRs that recognize the acetylation sites identified in Column E of Table 1.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments that have hinge cysteines between them. Other chemical couplings of antibody fragments are also known.

“Single-chain Fv” or “scFv” antibody fragments comprise the V_{H} and V_{L} domains of an antibody, wherein these domains are present in a single polypeptide chain. In certain embodiments, the Fv polypeptide further comprises a polypeptide linker between the V_{H} and V_{L} domains that enables the scFv to form the desired structure for antigen binding. For a review of scFv see Pluckthun in The Pharmacology of Monoclonal Antibodies, vol. 113, Rosenburg and Moore, eds. (Springer-Verlag: New York, 1994), pp. 269-315.

SMIPs are a class of single-chain peptides engineered to include a target binding region and effector domain (CH2 and CH3 domains). See, e.g., U.S. Patent Application Publication No. 20050238646. The target binding region may be derived from the variable region or CDRs of an antibody, e.g., a acetylation site-specific antibody of the application. Alternatively, the target binding region is derived from a protein that binds a acetylation site.

Bispecific antibodies may be monoclonal, human or humanized antibodies that have binding specificities for at least two different antigens. In the present case, one of the binding specificities is for the acetylation site, the other one is for any other antigen, such as for example, a cell-surface protein or receptor or receptor subunit. Alternatively, a therapeutic agent may be placed on one arm. The therapeutic agent can be a drug, toxin, enzyme, DNA, radionuclide, etc.

In some embodiments, the antigen-binding fragment can be a diabody. The term “diabody” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90: 6444-6448 (1993).

Camelid antibodies refer to a unique type of antibodies that are devoid of light chain, initially discovered from animals of the camelid family. The heavy chains of these so-called heavy-chain antibodies bind their antigen by one single domain, the variable domain of the heavy immunoglobulin chain, referred to as VHH. VHHs show homology with the variable domain of heavy chains of the human VHIII family. The VHHs obtained from an immunized camel, dromedary, or llama have a number of advantages, such as effective production in microorganisms such as Saccharomyces cerevisiae.

In certain embodiments, single chain antibodies, and chimeric, humanized or primatized (CDR-grafted) antibodies, as well as chimeric or CDR-grafted single chain antibodies, comprising portions derived from different species, are also encompassed by the present disclosure as antigen-binding fragments of an antibody. The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., U.S. Pat. Nos. 4,816,567 and 6,331,415; U.S. Pat. No. 4,816,397; European Patent No. 0,120,694; WO 86/01533; European Patent No. 0,194,276 B1; U.S. Pat. No. 5,225,539; and European Patent No. 0,239,400 B1. See also, Newman et al., BioTechnology, 10: 1455-1460 (1992), regarding primatized antibody. See, e.g., Ladner et al., U.S. Pat. No. 4,946,778; and Bird et al., Science, 242: 423-426 (1988)), regarding single chain antibodies.

In addition, functional fragments of antibodies, including fragments of chimeric, humanized, primatized or single chain antibodies, can also be produced. Functional fragments of the subject antibodies retain at least one binding function and/or modulation function of the full-length antibody from which they are derived.

Since the immunoglobulin-related genes contain separate functional regions, each having one or more distinct biological activities, the genes of the antibody fragments may be fused to functional regions from other genes (e.g., enzymes, U.S. Pat. No. 5,004,692, which is incorporated by reference in its entirety) to produce fusion proteins or conjugates having novel properties.

Non-immunoglobulin binding polypeptides are also contemplated. For example, CDRs from an antibody disclosed herein may be inserted into a suitable non-immunoglobulin scaffold to create a non-immunoglobulin binding polypeptide. Suitable candidate scaffold structures may be derived from, for example, members of fibronectin type III and cadherin superfamilies.

Also contemplated are other equivalent non-antibody molecules, such as protein binding domains or aptamers, which bind, in an acetyl-specific manner, to an amino acid sequence comprising a novel acetylation site of the invention. See, e.g., Neuberger et al., Nature 312: 604 (1984). Aptamers are oligonucleic acid or peptide molecules that bind a specific target molecule. DNA or RNA aptamers are typically short oligonucleotides, engineered through repeated rounds of selection to bind to a molecular target. Peptide aptamers typically consist of a variable peptide loop attached at both ends to a protein scaffold. This double structural constraint generally increases the binding affinity of the peptide aptamer to levels comparable to an antibody (nanomolar range).

The invention also discloses the use of the acetylation site-specific antibodies with immunotoxins. Conjugates that are immunotoxins including antibodies have been widely described in the art. The toxins may be coupled to the antibodies by conventional coupling techniques or immunotoxins containing protein toxin portions can be produced as fusion proteins. In certain embodiments, antibody conjugates may comprise stable linkers and may release cytotoxic agents inside cells (see U.S. Pat. Nos. 6,867,007 and 6,884,869). The conjugates of the present application can be used in a corresponding way to obtain such immunotoxins. Illustrative of such immunotoxins are those described by Byers et al., Seminars Cell Biol 2:59-70 (1991) and by Fanger et al., Immunol Today 12:51-54 (1991). Exemplary immunotoxins include radiotherapeutic agents, ribosome-inactivating proteins (RIPs), chemotherapeutic agents, toxic peptides, or toxic proteins.

The acetylation site-specific antibodies disclosed in the invention may be used singly or in combination. The antibodies may also be used in an array format for high throughput uses. An antibody microarray is a collection of immobilized antibodies, typically spotted and fixed on a solid surface (such as glass, plastic and silicon chip).

In another aspect, the antibodies of the invention modulate at least one, or all, biological activities of a parent protein identified in Column A of Table 1. The biological activities of a parent protein identified in Column A of Table 1 include: 1) ligand binding activities (for instance, these neutralizing antibodies may be capable of competing with or completely blocking the binding of a parent signaling protein to at least one, or all, of its ligands; 2) signaling transduction activities, such as receptor dimerization, or lysine acetylation; and 3) cellular responses induced by a parent signaling protein, such as oncogenic activities (e.g., cancer cell proliferation mediated by a parent signaling protein), and/or angiogenic activities.

In certain embodiments, the antibodies of the invention may have at least one activity selected from the group consisting of: 1) inhibiting cancer cell growth or proliferation; 2) inhibiting cancer cell survival; 3) inhibiting angiogenesis; 4) inhibiting cancer cell metastasis, adhesion, migration or invasion; 5) inducing apoptosis of cancer cells; 6) incorporating a toxic conjugate; and 7) acting as a diagnostic marker.

In certain embodiments, the acetylation site specific antibodies disclosed in the invention are especially indicated for diagnostic and therapeutic applications as described herein. Accordingly, the antibodies may be used in therapies, including combination therapies, in the diagnosis and prognosis of disease, as well as in the monitoring of disease progression. The invention, thus, further includes compositions comprising one or more embodiments of an antibody or an antigen binding portion of the invention as described herein. The composition may further comprise a pharmaceutically acceptable carrier. The composition may comprise two or more antibodies or antigen-binding portions, each with specificity for a different novel lysine acetylation site of the invention or two or more different antibodies or antigen-binding portions all of which are specific for the same novel lysine acetylation site of the invention. A composition of the invention may comprise one or more antibodies or antigen-binding portions of the invention and one or more additional reagents, diagnostic agents or therapeutic agents.

The present application provides for the polynucleotide molecules encoding the antibodies and antibody fragments and their analogs described herein. Because of the degeneracy of the genetic code, a variety of nucleic acid sequences encode each antibody amino acid sequence. The desired nucleic acid sequences can be produced by de novo solid-phase DNA synthesis or by PCR mutagenesis of an earlier prepared variant of the desired polynucleotide. In one embodiment, the codons that are used comprise those that are typical for human or mouse (see, e.g., Nakamura, Y., Nucleic Acids Res. 28: 292 (2000)).

The invention also provides immortalized cell lines that produce an antibody of the invention. For example, hybridoma clones, constructed as described above, that produce monoclonal antibodies to the targeted signaling protein acetylation sties disclosed herein are also provided. Similarly, the invention includes recombinant cells producing an antibody of the invention, which cells may be constructed by well known techniques; for example the antigen combining site of the monoclonal antibody can be cloned by PCR and single-chain antibodies produced as phage-displayed recombinant antibodies or soluble antibodies in E. coli (see, e.g., ANTIBODY ENGINEERING PROTOCOLS, 1995, Humana Press, Sudhir Paul editor.)

### 5. Methods of Making Acetylation Site-Specific Antibodies

In another aspect, the invention provides a method for making acetylation site-specific antibodies.

Polyclonal antibodies of the invention may be produced according to standard techniques by immunizing a suitable animal (e.g., rabbit, goat, etc.) with an antigen comprising a novel lysine acetylation site of the invention. (i.e. a acetylation site shown in Table 1) in either the acetylated or unacetylated state, depending upon the desired specificity of the antibody, collecting immune serum from the animal, and separating the polyclonal antibodies from the immune serum, in accordance with known procedures and screening and isolating a polyclonal antibody specific for the novel lysine acetylation site of interest as further described below. Methods for immunizing non-human animals such as mice, rats, sheep, goats, pigs, cattle and horses are well known in the art. See, e.g., Harlow and Lane, Antibodies: A Laboratory Manual, New York: Cold Spring Harbor Press, 1990.

The immunogen may be the full length protein or a peptide comprising the novel lysine acetylation site of interest. In some embodiments the immunogen is a peptide of from 7 to 20 amino acids in length, preferably about 8 to 17 amino acids in length. In some embodiments, the peptide antigen desirably will comprise about 3 to 8 amino acids on each side of the acetylatable lysine. In yet other embodiments, the peptide antigen desirably will comprise four or more amino acids flanking each side of the acetylatable amino acid and encompassing it. Peptide antigens suitable for producing antibodies of the invention may be designed, constructed and employed in accordance with well-known techniques. See, e.g., Antibodies: A Laboratory Manual, Chapter 5, p. 75-76, Harlow & Lane Eds., Cold Spring Harbor Laboratory (1988); Czernik, Methods In Enzymology, 201: 264-283 (1991); Merrifield, J. Am. Chem. Soc. 85: 21-49 (1962)).

Suitable peptide antigens may comprise all or partial sequence of a trypsin-digested fragment as set forth in Column E of Table 1/FIG. 2. Suitable peptide antigens may also comprise all or partial sequence of a peptide fragment produced by another protease digestion.

Preferred immunogens are those that comprise a novel acetylation site of a protein in Table 1 that is a chromatin or DNA binding/repair/replication protein, enzyme protein, RNA binding protein, transcriptional regulator, translational regulator, ubiquitan conjugating system protein, cytoskeletal protein, adaptor/scaffold protein or receptor/channel/transporter/cell surface protein. In some embodiments, the peptide immunogen is an AQUA peptide, for example, any one of the sequences listed in column E of Table one and FIG. 2.

Particularly preferred immunogens are peptides comprising any one of the novel lysine acetylation site shown as a lower case “k” in a sequence listed in Table 1 selected from the group consisting of SEQ ID NOs: 46 (ACAA2); 48 (ACAA2); 50 (ACAA2); 71 (ACAT1); 72 (ACAT1); 333 (CLYBL); 336 (CS); 351 (DLD); 375 (SUCLG1); 376 (SUCLG1); 417 (SLC25A4); 420 (SLC25A5); 491 (ACOT2); 549 (ALDH6A1); 631 (COX5B); 632 (COX5B); 643 (CPS1); 646 (CPS1); 647 (CPS1); 649 (CPS1); 687 (DLST); 697 (EHHADH); 715 (ETFDH); 739 (GCAT); 757 (GOT2); 759 (GOT2); 895 (PC); 1072 (ACAD11); and 1269 (SLC25A31).

In some embodiments the immunogen is administered with an adjuvant. Suitable adjuvants will be well known to those of skill in the art. Exemplary adjuvants include complete or incomplete Freund's adjuvant, RIBI (muramyl dipeptides) or ISCOM (immunostimulating complexes).

For example, a peptide antigen comprising the novel receptor lysine kinase acetylation site in SEQ ID NO: 4 shown by the lower case “k” in Table 1 may be used to produce antibodies that specifically bind the novel lysine acetylation site.

When the above-described methods are used for producing polyclonal antibodies, following immunization, the polyclonal antibodies which secreted into the bloodstream can be recovered using known techniques. Purified forms of these antibodies can, of course, be readily prepared by standard purification techniques, such as for example, affinity chromatography with Protein A, anti-immunoglobulin, or the antigen itself. In any case, in order to monitor the success of immunization, the antibody levels with respect to the antigen in serum will be monitored using standard techniques such as ELISA, RIA and the like.

Monoclonal antibodies of the invention may be produced by any of a number of means that are well-known in the art. In some embodiments, antibody-producing B cells are isolated from an animal immunized with a peptide antigen as described above. The B cells may be from the spleen, lymph nodes or peripheral blood. Individual B cells are isolated and screened as described below to identify cells producing an antibody specific for the novel lysine acetylation site of interest. Identified cells are then cultured to produce a monoclonal antibody of the invention.

Alternatively, a monoclonal acetylation site-specific antibody of the invention may be produced using standard hybridoma technology, in a hybridoma cell line according to the well-known technique of Kohler and Milstein. See Nature 265: 495-97 (1975); Kohler and Milstein, Eur. J. Immunol. 6: 511 (1976); see also, Current Protocols in Molecular Biology, Ausubel et al. Eds. (1989). Monoclonal antibodies so produced are highly specific, and improve the selectivity and specificity of diagnostic assay methods provided by the invention. For example, a solution containing the appropriate antigen may be injected into a mouse or other species and, after a sufficient time (in keeping with conventional techniques), the animal is sacrificed and spleen cells obtained. The spleen cells are then immortalized by any of a number of standard means. Methods of immortalizing cells include, but are not limited to, transfecting them with oncogenes, infecting them with an oncogenic virus and cultivating them under conditions that select for immortalized cells, subjecting them to carcinogenic or mutating compounds, fusing them with an immortalized cell, e.g., a myeloma cell, and inactivating a tumor suppressor gene. See, e.g., Harlow and Lane, supra. If fusion with myeloma cells is used, the myeloma cells preferably do not secrete immunoglobulin polypeptides (a non-secretory cell line). Typically the antibody producing cell and the immortalized cell (such as but not limited to myeloma cells) with which it is fused are from the same species. Rabbit fusion hybridomas, for example, may be produced as described in U.S. Pat. No. 5,675,063, C. Knight, Issued Oct. 7, 1997. The immortalized antibody producing cells, such as hybridoma cells, are then grown in a suitable selection media, such as hypoxanthine-aminopterin-thymidine (HAT), and the supernatant screened for monoclonal antibodies having the desired specificity, as described below. The secreted antibody may be recovered from tissue culture supernatant by conventional methods such as precipitation, ion exchange or affinity chromatography, or the like.

The invention also encompasses antibody-producing cells and cell lines, such as hybridomas, as described above.

Polyclonal or monoclonal antibodies may also be obtained through in vitro immunization. For example, phage display techniques can be used to provide libraries containing a repertoire of antibodies with varying affinities for a particular antigen. Techniques for the identification of high affinity human antibodies from such libraries are described by Griffiths et al., (1994) EMBO J., 13:3245-3260; Nissim et al., ibid, pp. 692-698 and by Griffiths et al., ibid, 12:725-734, which are incorporated by reference.

The antibodies may be produced recombinantly using methods well known in the art for example, according to the methods disclosed in U.S. Pat. No. 4,349,893 (Reading) or U.S. Pat. No. 4,816,567 (Cabilly et al.) The antibodies may also be chemically constructed by specific antibodies made according to the method disclosed in U.S. Pat. No. 4,676,980 (Segel et al.)

Once a desired acetylation site-specific antibody is identified, polynucleotides encoding the antibody, such as heavy, light chains or both (or single chains in the case of a single chain antibody) or portions thereof such as those encoding the variable region, may be cloned and isolated from antibody-producing cells using means that are well known in the art. For example, the antigen combining site of the monoclonal antibody can be cloned by PCR and single-chain antibodies produced as phage-displayed recombinant antibodies or soluble antibodies in E. coli (see, e.g., Antibody Engineering Protocols, 1995, Humana Press, Sudhir Paul editor.)

Accordingly, in a further aspect, the invention provides such nucleic acids encoding the heavy chain, the light chain, a variable region, a framework region or a CDR of an antibody of the invention. In some embodiments, the nucleic acids are operably linked to expression control sequences. The invention, thus, also provides vectors and expression control sequences useful for the recombinant expression of an antibody or antigen-binding portion thereof of the invention. Those of skill in the art will be able to choose vectors and expression systems that are suitable for the host cell in which the antibody or antigen-binding portion is to be expressed.

Monoclonal antibodies of the invention may be produced recombinantly by expressing the encoding nucleic acids in a suitable host cell under suitable conditions. Accordingly, the invention further provides host cells comprising the nucleic acids and vectors described above.

Monoclonal Fab fragments may also be produced in Escherichia coli by recombinant techniques known to those skilled in the art. See, e.g., W. Huse, Science 246: 1275-81 (1989); Mullinax et al., Proc. Nat'l Acad. Sci. 87: 8095 (1990).

If monoclonal antibodies of a single desired isotype are preferred for a particular application, particular isotypes can be prepared directly, by selecting from the initial fusion, or prepared secondarily, from a parental hybridoma secreting a monoclonal antibody of different isotype by using the sib selection technique to isolate class-switch variants (Steplewski, et al., Proc. Nat'l. Acad. Sci., 82: 8653 (1985); Spira et al., J. Immunol. Methods, 74: 307 (1984)). Alternatively, the isotype of a monoclonal antibody with desirable propertied can be changed using antibody engineering techniques that are well-known in the art.

Acetylation site-specific antibodies of the invention, whether polyclonal or monoclonal, may be screened for epitope and acetyl-specificity according to standard techniques. See, e.g., Czernik et al., Methods in Enzymology, 201: 264-283 (1991). For example, the antibodies may be screened against the acetylated and/or unacetylated peptide library by ELISA to ensure specificity for both the desired antigen (i.e. that epitope including an acetylation site of the invention and for reactivity only with the acetylated (or unacetylated) form of the antigen. Peptide competition assays may be carried out to confirm lack of reactivity with other acetylatable epitopes on the parent protein. The antibodies may also be tested by Western blotting against cell preparations containing the parent signaling protein, e.g., cell lines over-expressing the parent protein, to confirm reactivity with the desired acetylated epitope/target.

Specificity against the desired acetylated epitope may also be examined by constructing mutants lacking acetylatable residues at positions outside the desired epitope that are known to be acetylated, or by mutating the desired acetylatable epitope and confirming lack of reactivity. Acetylation site-specific antibodies of the invention may exhibit some limited cross-reactivity to related epitopes in non-target proteins. This is not unexpected as most antibodies exhibit some degree of cross-reactivity, and anti-peptide antibodies will often cross-react with epitopes having high homology to the immunizing peptide. See, e.g., Czernik, supra. Cross-reactivity with non-target proteins is readily characterized by Western blotting alongside markers of known molecular weight. Amino acid sequences of cross-reacting proteins may be examined to identify acetylation sites with flanking sequences that are highly homologous to that of a acetylation site of the invention.

In certain cases, polyclonal antisera may exhibit some undesirable general cross-reactivity to acetyl-lysine itself, which may be removed by further purification of antisera, e.g., over an acetyl-lysine column. Antibodies of the invention specifically bind their target protein (i.e. a protein listed in Column A of Table 1) only when acetylated (or only when not acetylated, as the case may be) at the site disclosed in corresponding Columns D/E, and do not (substantially) bind to the other form (as compared to the form for which the antibody is specific).

Antibodies may be further characterized via immunohistochemical (IHC) staining using normal and diseased tissues to examine acetylation and activation state and level of a acetylation site in diseased tissue. IHC may be carried out according to well-known techniques. See, e.g., Antibodies: A Laboratory Manual, Chapter 10, Harlow & Lane Eds., Cold Spring Harbor Laboratory (1988). Briefly, paraffin-embedded tissue (e.g., tumor tissue) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary antibody and secondary antibody; and finally detecting using ABC avidin/biotin method according to manufacturer's instructions.

Antibodies may be further characterized by flow cytometry carried out according to standard methods. See Chow et al., Cytometry (Communications in Clinical Cytometry) 46: 72-78 (2001). Briefly and by way of example, the following protocol for cytometric analysis may be employed: samples may be centrifuged on Ficoll gradients to remove lysed erythrocytes and cell debris. Adhering cells may be scrapped off plates and washed with PBS. Cells may then be fixed with 2% paraformaldehyde for 10 minutes at 37° C. followed by permeabilization in 90% methanol for 30 minutes on ice. Cells may then be stained with the primary acetylation site-specific antibody of the invention (which detects a parent signaling protein enumerated in Table 1), washed and labeled with a fluorescent-labeled secondary antibody. Additional fluorochrome-conjugated marker antibodies (e.g., CD45, CD34) may also be added at this time to aid in the subsequent identification of specific hematopoietic cell types. The cells would then be analyzed on a flow cytometer (e.g. a Beckman Coulter FC500) according to the specific protocols of the instrument used.

Antibodies of the invention may also be advantageously conjugated to fluorescent dyes (e.g. Alexa488, PE) for use in multi-parametric analyses along with other signal transduction (phospho-CrkL, phospho-Erk 1/2) and/or cell marker (CD34) antibodies.

Acetylation site-specific antibodies of the invention may specifically bind to a signaling protein or polypeptide listed in Table 1 only when acetylated at the specified lysine residue, but are not limited only to binding to the listed signaling proteins of human species, per se. The invention includes antibodies that also bind conserved and highly homologous or identical acetylation sites in respective signaling proteins from other species (e.g., mouse, rat, monkey, yeast), in addition to binding the acetylation site of the human homologue. The term “homologous” refers to two or more sequences or subsequences that have at least about 85%, at least 90%, at least 95%, or higher nucleotide or amino acid residue identity, when compared and aligned for maximum correspondence, as measured using sequence comparison method (e.g., BLAST) and/or by visual inspection. Highly homologous or identical sites conserved in other species can readily be identified by standard sequence comparisons (such as BLAST).

Methods for making bispecific antibodies are within the purview of those skilled in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Antibody variable domains with the desired binding specificities (antibody-antigen combining sites) can be fused to immunoglobulin constant domain sequences. In certain embodiments, the fusion is with an immunoglobulin heavy-chain constant domain, including at least part of the hinge, CH2, and CH3 regions. DNAs encoding the immunoglobulin heavy-chain fusions and, if desired, the immunoglobulin light chain, are inserted into separate expression vectors, and are co-transfected into a suitable host organism. For further details of illustrative currently known methods for generating bispecific antibodies see, for example, Suresh et al., Methods in Enzymology, 121:210 (1986); WO 96/27011; Brennan et al., Science 229:81 (1985); Shalaby et al., J. Exp. Med. 175:217-225 (1992); Kostelny et al., J. Immunol. 148(5):1547-1553 (1992); Hollinger et al., Proc. Natl. Acad. Sci. USA 90:6444-6448 (1993); Gruber et al., J. Immunol. 152:5368 (1994); and Tutt et al., J. Immunol. 147:60 (1991). Bispecific antibodies also include cross-linked or heteroconjugate antibodies. Heteroconjugate antibodies may be made using any convenient cross-linking methods. Suitable cross-linking agents are well known in the art, and are disclosed in U.S. Pat. No. 4,676,980, along with a number of cross-linking techniques.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins may be linked to the Fab′ portions of two different antibodies by gene fusion. The antibody homodimers may be reduced at the hinge region to form monomers and then re-oxidized to form the antibody heterodimers. This method can also be utilized for the production of antibody homodimers. A strategy for making bispecific antibody fragments by the use of single-chain Fv (scFv) dimers has also been reported. See Gruber et al., J. Immunol., 152:5368 (1994). Alternatively, the antibodies can be “linear antibodies” as described in Zapata et al. Protein Eng. 8(10):1057-1062 (1995). Briefly, these antibodies comprise a pair of tandem Fd segments (V_{H}-C_{H}1-V_{H}-C_{H}1) which form a pair of antigen binding regions. Linear antibodies can be bispecific or monospecific.

To produce the chimeric antibodies, the portions derived from two different species (e.g., human constant region and murine variable or binding region) can be joined together chemically by conventional techniques or can be prepared as single contiguous proteins using genetic engineering techniques. The DNA molecules encoding the proteins of both the light chain and heavy chain portions of the chimeric antibody can be expressed as contiguous proteins. The method of making chimeric antibodies is disclosed in U.S. Pat. No. 5,677,427; U.S. Pat. No. 6,120,767; and U.S. Pat. No. 6,329,508, each of which is incorporated by reference in its entirety.

Fully human antibodies may be produced by a variety of techniques. One example is trioma methodology. The basic approach and an exemplary cell fusion partner, SPAZ-4, for use in this approach have been described by Oestberg et al., Hybridoma 2:361-367 (1983); Oestberg, U.S. Pat. No. 4,634,664; and Engleman et al., U.S. Pat. No. 4,634,666 (each of which is incorporated by reference in its entirety).

Human antibodies can also be produced from non-human transgenic animals having transgenes encoding at least a segment of the human immunoglobulin locus. The production and properties of animals having these properties are described in detail by, see, e.g., Lonberg et al., WO93/12227; U.S. Pat. No. 5,545,806; and Kucherlapati, et al., WO91/10741; U.S. Pat. No. 6,150,584, which are herein incorporated by reference in their entirety.

Various recombinant antibody library technologies may also be utilized to produce fully human antibodies. For example, one approach is to screen a DNA library from human B cells according to the general protocol outlined by Huse et al., Science 246:1275-1281 (1989). The protocol described by Huse is rendered more efficient in combination with phage-display technology. See, e.g., Dower et al., WO 91/17271 and McCafferty et al., WO 92/01047; U.S. Pat. No. 5,969,108, (each of which is incorporated by reference in its entirety).

Eukaryotic ribosome can also be used as means to display a library of antibodies and isolate the binding human antibodies by screening against the target antigen, as described in Coia G, et al., J. Immunol. Methods 1: 254 (1-2):191-7 (2001); Hanes J. et al., Nat. Biotechnol. 18(12):1287-92 (2000); Proc. Natl. Acad. Sci. U.S.A. 95(24):14130-5 (1998); Proc. Natl. Acad. Sci. U.S.A. 94(10):4937-42 (1997), each which is incorporated by reference in its entirety.

The yeast system is also suitable for screening mammalian cell-surface or secreted proteins, such as antibodies. Antibody libraries may be displayed on the surface of yeast cells for the purpose of obtaining the human antibodies against a target antigen. This approach is described by Yeung, et al., Biotechnol. Prog. 18(2):212-20 (2002); Boeder, E. T., et al., Nat. Biotechnol. 15(6):553-7 (1997), each of which is herein incorporated by reference in its entirety. Alternatively, human antibody libraries may be expressed intracellularly and screened via the yeast two-hybrid system (WO0200729A2, which is incorporated by reference in its entirety).

Recombinant DNA techniques can be used to produce the recombinant acetylation site-specific antibodies described herein, as well as the chimeric or humanized acetylation site-specific antibodies, or any other genetically-altered antibodies and the fragments or conjugate thereof in any expression systems including both prokaryotic and eukaryotic expression systems, such as bacteria, yeast, insect cells, plant cells, mammalian cells (for example, NS0 cells).

Once produced, the whole antibodies, their dimers, individual light and heavy chains, or other immunoglobulin forms of the present application can be purified according to standard procedures of the art, including ammonium sulfate precipitation, affinity columns, column chromatography, gel electrophoresis and the like (see, generally, Scopes, R., Protein Purification (Springer-Verlag, N.Y., 1982)). Once purified, partially or to homogeneity as desired, the polypeptides may then be used therapeutically (including extracorporeally) or in developing and performing assay procedures, immunofluorescent staining, and the like. (See, generally, Immunological Methods, Vols. I and II (Lefkovits and Pernis, eds., Academic Press, NY, 1979 and 1981).

### 6. Therapeutic Uses

In a further aspect, the invention provides methods and compositions for therapeutic uses of the peptides or proteins comprising a acetylation site of the invention, and acetylation site-specific antibodies of the invention.

In one embodiment, the invention provides for a method of treating or preventing an undesired condition, wherein the condition is associated with the acetylation state of a novel acetylation site in Table 1, whether acetylated or deacetylated, comprising: administering to a subject in need thereof a therapeutically effective amount of a peptide comprising a novel acetylation site (Table 1) and/or an antibody or antigen-binding fragment thereof that specifically bind a novel acetylation site of the invention (Table 1). The antibodies maybe full-length antibodies, genetically engineered antibodies, antibody fragments, and antibody conjugates of the invention.

The term “subject” refers to a vertebrate, such as for example, a mammal, or a human. Although present application are primarily concerned with the treatment of human subjects, the disclosed methods may also be used for the treatment of other mammalian subjects such as dogs and cats for veterinary purposes.

In one aspect, the disclosure provides a method of treating an undesired condition in which a peptide or an antibody that reduces at least one biological activity of a targeted signaling protein is administered to a subject. For example, the peptide or the antibody administered may disrupt or modulate the interaction of the target signaling protein with its ligand. Alternatively, the peptide or the antibody may interfere with, thereby reducing, the down-stream signal transduction of the parent signaling protein. An antibody that specifically binds the novel lysine acetylation site only when the lysine is acetylated, and that does not substantially bind to the same sequence when the lysine is not acetylated, thereby prevents downstream signal transduction triggered by an acetyl-lysine. Alternatively, an antibody that specifically binds the unacetylated target acetylation site reduces the acetylation at that site and thus reduces activation of the protein mediated by acetylation of that site. Similarly, an unacetylated peptide may compete with an endogenous acetylation site for same kinases, thereby preventing or reducing the acetylation of the endogenous target protein. Alternatively, a peptide comprising an acetylated novel lysine site of the invention but lacking the ability to trigger signal transduction may competitively inhibit interaction of the endogenous protein with the same down-stream ligand(s).

The antibodies of the invention may also be used to target cancer cells for effector-mediated cell death. The antibody disclosed herein may be administered as a fusion molecule that includes a acetylation site-targeting portion joined to a cytotoxic moiety to directly kill cancer cells. Alternatively, the antibody may directly kill the cancer cells through complement-mediated or antibody-dependent cellular cytotoxicity.

Accordingly in one embodiment, the antibodies of the present disclosure may be used to deliver a variety of cytotoxic compounds. Any cytotoxic compound can be fused to the present antibodies. The fusion can be achieved chemically or genetically (e.g., via expression as a single, fused molecule). The cytotoxic compound can be a biological, such as a polypeptide, or a small molecule. As those skilled in the art will appreciate, for small molecules, chemical fusion is used, while for biological compounds, either chemical or genetic fusion can be used.

Non-limiting examples of cytotoxic compounds include therapeutic drugs, radiotherapeutic agents, ribosome-inactivating proteins (RIPs), chemotherapeutic agents, toxic peptides, toxic proteins, and mixtures thereof. The cytotoxic drugs can be intracellularly acting cytotoxic drugs, such as short-range radiation emitters, including, for example, short-range, high-energy α-emitters. Enzymatically active toxins and fragments thereof, including ribosome-inactivating proteins, are exemplified by saporin, luffin, momordins, ricin, trichosanthin, gelonin, abrin, etc. Procedures for preparing enzymatically active polypeptides of the immunotoxins are described in WO84/03508 and WO85/03508, which are hereby incorporated by reference. Certain cytotoxic moieties are derived from adriamycin, chlorambucil, daunomycin, methotrexate, neocarzinostatin, and platinum, for example.

Exemplary chemotherapeutic agents that may be attached to an antibody or antigen-binding fragment thereof include taxol, doxorubicin, verapamil, podophyllotoxin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide (VP16), tamoxifen, transplatinum, 5-fluorouracil, vincristin, vinblastin, or methotrexate.

Procedures for conjugating the antibodies with the cytotoxic agents have been previously described and are within the purview of one skilled in the art.

Alternatively, the antibody can be coupled to high energy radiation emitters, for example, a radioisotope, such as ¹³¹I, a γ-emitter, which, when localized at the tumor site, results in a killing of several cell diameters. See, e.g., S. E. Order, “Analysis, Results, and Future Prospective of the Therapeutic Use of Radiolabeled Antibody in Cancer Therapy”, Monoclonal Antibodies for Cancer Detection and Therapy, Baldwin et al. (eds.), pp. 303-316 (Academic Press 1985), which is hereby incorporated by reference. Other suitable radioisotopes include α-emitters, such as ²¹²Bi, ²¹³Bi, and ²¹¹At, and β-emitters, such as ¹⁸⁶Re and ⁹⁰Y.

Because many of the signaling proteins in which novel lysine acetylation sites of the invention occur also are expressed in normal cells and tissues, it may also be advantageous to administer an acetylation site-specific antibody with a constant region modified to reduce or eliminate ADCC or CDC to limit damage to normal cells. For example, effector function of antibodies may be reduced or eliminated by utilizing an IgG1 constant domain instead of an IgG2/4 fusion domain. Other ways of eliminating effector function can be envisioned such as, e.g., mutation of the sites known to interact with FcR or insertion of a peptide in the hinge region, thereby eliminating critical sites required for FcR interaction. Variant antibodies with reduced or no effector function also include variants as described previously herein.

The peptides and antibodies of the invention may be used in combination with other therapies or with other agents. Other agents include but are not limited to polypeptides, small molecules, chemicals, metals, organometallic compounds, inorganic compounds, nucleic acid molecules, oligonucleotides, aptamers, spiegelmers, antisense nucleic acids, locked nucleic acid (LNA) inhibitors, peptide nucleic acid (PNA) inhibitors, immunomodulatory agents, antigen-binding fragments, prodrugs, and peptidomimetic compounds. In certain embodiments, the antibodies and peptides of the invention may be used in combination with cancer therapies known to one of skill in the art.

In certain aspects, the present disclosure relates to combination treatments comprising an acetylation site-specific antibody described herein and immunomodulatory compounds, vaccines or chemotherapy. Illustrative examples of suitable immunomodulatory agents that may be used in such combination therapies include agents that block negative regulation of T cells or antigen presenting cells (e.g., anti-CTLA4 antibodies, anti-PD-L1 antibodies, anti-PDL-2 antibodies, anti-PD-1 antibodies and the like) or agents that enhance positive co-stimulation of T cells (e.g., anti-CD40 antibodies or anti 4-1BB antibodies) or agents that increase NK cell number or T-cell activity (e.g., inhibitors such as IMiDs, thalidomide, or thalidomide analogs). Furthermore, immunomodulatory therapy could include cancer vaccines such as dendritic cells loaded with tumor cells, proteins, peptides, RNA, or DNA derived from such cells, patient derived heat-shock proteins (hsp's) or general adjuvants stimulating the immune system at various levels such as CpG, Luivac®, Biostim®, Ribomunyl®, Imudon®, Bronchovaxom® or any other compound or other adjuvant activating receptors of the innate immune system (e.g., toll like receptor agonist, anti-CTLA-4 antibodies, etc.). Also, immunomodulatory therapy could include treatment with cytokines such as IL-2, GM-CSF and IFN-gamma.

Furthermore, if the undesired condition is a cancer, combination of antibody therapy with chemotherapeutics could be particularly useful to reduce overall tumor burden, to limit angiogenesis, to enhance tumor accessibility, to enhance susceptibility to ADCC, to result in increased immune function by providing more tumor antigen, or to increase the expression of the T cell attractant LIGHT.

Pharmaceutical compounds that may be used for combinatory anti-tumor therapy include, merely to illustrate: aminoglutethimide, amsacrine, anastrozole, asparaginase, bcg, bicalutamide, bleomycin, buserelin, busulfan, camptothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic anti-tumor compounds may be categorized by their mechanism of action into groups, including, for example, the following classes of agents: anti-metabolites/anti-cancer agents, such as pyrimidine analogs (5-fluorouracil, floxuridine, capecitabine, gemcitabine and cytarabine) and purine analogs, folate inhibitors and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristine, vinblastine, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamineoxaliplatin, iphosphamide, melphalan, mechlorethamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); immunomodulatory agents (thalidomide and analogs thereof such as lenalidomide (Revlimid, CC-5013) and CC-4047 (Actimid)), cyclophosphamide; anti-angiogenic compounds (TNP-470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylprednisolone, prednisone, and prenisolone); growth factor signal transduction kinase inhibitors; mitochondrial dysfunction inducers and caspase activators; and chromatin disruptors.

In certain embodiments, pharmaceutical compounds that may be used for combinatory anti-angiogenesis therapy include: (1) inhibitors of release of “angiogenic molecules,” such as bFGF (basic fibroblast growth factor); (2) neutralizers of angiogenic molecules, such as anti-βbFGF antibodies; and (3) inhibitors of endothelial cell response to angiogenic stimuli, including collagenase inhibitor, basement membrane turnover inhibitors, angiostatic steroids, fungal-derived angiogenesis inhibitors, platelet factor 4, thrombospondin, arthritis drugs such as D-penicillamine and gold thiomalate, vitamin D₃ analogs, alpha-interferon, and the like. For additional proposed inhibitors of angiogenesis, see Blood et al., Biochim. Biophys. Acta, 1032:89-118 (1990), Moses et al., Science, 248:1408-1410 (1990), Ingber et al., Lab. Invest., 59:44-51 (1988), and U.S. Pat. Nos. 5,092,885, 5,112,946, 5,192,744, 5,202,352, and 6,573,256. In addition, there are a wide variety of compounds that can be used to inhibit angiogenesis, for example, peptides or agents that block the VEGF-mediated angiogenesis pathway, endostatin protein or derivatives, lysine binding fragments of angiostatin, melanin or melanin-promoting compounds, plasminogen fragments (e.g., Kringles 1-3 of plasminogen), troponin subunits, inhibitors of vitronectin α_{ν}β₃, peptides derived from Saposin B, antibiotics or analogs (e.g., tetracycline or neomycin), dienogest-containing compositions, compounds comprising a MetAP-2 inhibitory core coupled to a peptide, the compound EM-138, chalcone and its analogs, and naaladase inhibitors. See, for example, U.S. Pat. Nos. 6,395,718, 6,462,075, 6,465,431, 6,475,784, 6,482,802, 6,482,810, 6,500,431, 6,500,924, 6,518,298, 6,521,439, 6,525,019, 6,538,103, 6,544,758, 6,544,947, 6,548,477, 6,559,126, and 6,569,845.

### 7. Diagnostic Uses

In a further aspect, the invention provides methods for detecting and quantitating acetylation at a novel lysine acetylation site of the invention. For example, peptides, including AQUA peptides of the invention, and antibodies of the invention are useful in diagnostic and prognostic evaluation of cancer, wherein the particular cancer is associated with the acetylation state of a novel acetylation site in Table 1, whether acetylated or deacetylated.

Methods of diagnosis can be performed in vitro using a biological sample (e.g., blood sample, lymph node biopsy or tissue) from a subject, or in vivo. The acetylation state or level at the lysine residue identified in the corresponding row in Column D of Table 1 may be assessed. A change in the acetylation state or level at the acetylation site, as compared to a control, indicates that the subject is suffering from, or susceptible to a for of cancer; for example, carcinoma.

In one embodiment, the acetylation state or level at a novel acetylation site is determined by an AQUA peptide comprising the acetylation site. The AQUA peptide may be acetylated or unacetylated at the specified lysine position.

In another embodiment, the acetylation state or level at a acetylation site is determined by an antibody or antigen-binding fragment thereof, wherein the antibody specifically binds the acetylation site. The antibody may be one that only binds to the acetylation site when the lysine residue is acetylated, but does not bind to the same sequence when the lysine is not acetylated; or vice versa.

In particular embodiments, the antibodies of the present application are attached to labeling moieties, such as a detectable marker. One or more detectable labels can be attached to the antibodies. Exemplary labeling moieties include radiopaque dyes, radiocontrast agents, fluorescent molecules, spin-labeled molecules, enzymes, or other labeling moieties of diagnostic value, particularly in radiologic or magnetic resonance imaging techniques.

A radiolabeled antibody in accordance with this disclosure can be used for in vitro diagnostic tests. The specific activity of an antibody, binding portion thereof, probe, or ligand, depends upon the half-life, the isotopic purity of the radioactive label, and how the label is incorporated into the biological agent. In immunoassay tests, the higher the specific activity, in general, the better the sensitivity. Radioisotopes useful as labels, e.g., for use in diagnostics, include iodine (¹³¹I or ¹²⁵I), indium (¹¹¹In), technetium (⁹⁹Tc), phosphorus (³²P), carbon (¹⁴C), and tritium (³H), or one of the therapeutic isotopes listed above.

Fluorophore and chromophore labeled biological agents can be prepared from standard moieties known in the art. Since antibodies and other proteins absorb light having wavelengths up to about 310 nm, the fluorescent moieties may be selected to have substantial absorption at wavelengths above 310 nm, such as for example, above 400 nm. A variety of suitable fluorescers and chromophores are described by Stryer, Science, 162:526 (1968) and Brand et al., Annual Review of Biochemistry, 41:843-868 (1972), which are hereby incorporated by reference. The antibodies can be labeled with fluorescent chromophore groups by conventional procedures such as those disclosed in U.S. Pat. Nos. 3,940,475, 4,289,747, and 4,376,110, which are hereby incorporated by reference.

The control may be parallel samples providing a basis for comparison, for example, biological samples drawn from a healthy subject, or biological samples drawn from healthy tissues of the same subject. Alternatively, the control may be a pre-determined reference or threshold amount. If the subject is being treated with a therapeutic agent, and the progress of the treatment is monitored by detecting the lysine acetylation state level at an acetylation site of the invention, a control may be derived from biological samples drawn from the subject prior to, or during the course of the treatment.

In certain embodiments, antibody conjugates for diagnostic use in the present application are intended for use in vitro, where the antibody is linked to a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. In certain embodiments, secondary binding ligands are biotin and avidin or streptavidin compounds.

Antibodies of the invention may also be optimized for use in a flow cytometry (FC) assay to determine the activation/acetylation status of a target signaling protein in subjects before, during, and after treatment with a therapeutic agent targeted at inhibiting lysine acetylation at the acetylation site disclosed herein. For example, bone marrow cells or peripheral blood cells from patients may be analyzed by flow cytometry for target signaling protein acetylation, as well as for markers identifying various hematopoietic cell types. In this manner, activation status of the malignant cells may be specifically characterized. Flow cytometry may be carried out according to standard methods. See, e.g., Chow et al., Cytometry (Communications in Clinical Cytometry) 46: 72-78 (2001).

Alternatively, antibodies of the invention may be used in immunohistochemical (IHC) staining to detect differences in signal transduction or protein activity using normal and diseased tissues. IHC may be carried out according to well-known techniques. See, e.g., Antibodies: A Laboratory Manual, supra.

Peptides and antibodies of the invention may be also be optimized for use in other clinically-suitable applications, for example bead-based multiplex-type assays, such as IGEN, Luminex™ and/or Bioplex™ assay formats, or otherwise optimized for antibody arrays formats, such as reversed-phase array applications (see, e.g. Paweletz et al., Oncogene 20(16): 1981-89 (2001)). Accordingly, in another embodiment, the invention provides a method for the multiplex detection of the acetylation state or level at two or more acetylation sites of the invention (Table 1) in a biological sample, the method comprising utilizing two or more antibodies or AQUA peptides of the invention. In one preferred embodiment, two to five antibodies or AQUA peptides of the invention are used. In another preferred embodiment, six to ten antibodies or AQUA peptides of the invention are used, while in another preferred embodiment eleven to twenty antibodies or AQUA peptides of the invention are used.

In certain embodiments the diagnostic methods of the application may be used in combination with other cancer diagnostic tests.

The biological sample analyzed may be any sample that is suspected of having abnormal lysine acetylation at a novel acetylation site of the invention, such as a homogenized neoplastic tissue sample.

### 8. Screening Assays

In another aspect, the invention provides a method for identifying an agent that modulates lysine acetylation at a novel acetylation site of the invention, comprising: a) contacting a candidate agent with a peptide or protein comprising a novel acetylation site of the invention; and b) determining the acetylation state or level at the novel acetylation site. A change in the acetylation level of the specified lysine in the presence of the test agent, as compared to a control, indicates that the candidate agent potentially modulates lysine acetylation at a novel acetylation site of the invention.

In one embodiment, the acetylation state or level at a novel acetylation site is determined by an AQUA peptide comprising the acetylation site. The AQUA peptide may be acetylated or unacetylated at the specified lysine position.

In another embodiment, the acetylation state or level at a acetylation site is determined by an antibody or antigen-binding fragment thereof, wherein the antibody specifically binds the acetylation site. The antibody may be one that only binds to the acetylation site when the lysine residue is acetylated, but does not bind to the same sequence when the lysine is not acetylated; or vice versa.

In particular embodiments, the antibodies of the present application are attached to labeling moieties, such as a detectable marker.

The control may be parallel samples providing a basis for comparison, for example, the acetylation level of the target protein or peptide in absence of the testing agent. Alternatively, the control may be a pre-determined reference or threshold amount.

### 9. Immunoassays

In another aspect, the present application concerns immunoassays for binding, purifying, quantifying and otherwise generally detecting the acetylation state or level at a novel acetylation site of the invention.

Assays may be homogeneous assays or heterogeneous assays. In a homogeneous assay the immunological reaction usually involves a acetylation site-specific antibody of the invention, a labeled analyte, and the sample of interest. The signal arising from the label is modified, directly or indirectly, upon the binding of the antibody to the labeled analyte. Both the immunological reaction and detection of the extent thereof are carried out in a homogeneous solution. Immunochemical labels that may be used include free radicals, radioisotopes, fluorescent dyes, enzymes, bacteriophages, coenzymes, and so forth.

In a heterogeneous assay approach, the reagents are usually the specimen, a acetylation site-specific antibody of the invention, and suitable means for producing a detectable signal. Similar specimens as described above may be used. The antibody is generally immobilized on a support, such as a bead, plate or slide, and contacted with the specimen suspected of containing the antigen in a liquid phase. The support is then separated from the liquid phase and either the support phase or the liquid phase is examined for a detectable signal using means for producing such signal. The signal is related to the presence of the analyte in the specimen. Means for producing a detectable signal include the use of radioactive labels, fluorescent labels, enzyme labels, and so forth.

Acetylation site-specific antibodies disclosed herein may be conjugated to a solid support suitable for a diagnostic assay (e.g., beads, plates, slides or wells formed from materials such as latex or polystyrene) in accordance with known techniques, such as precipitation.

In certain embodiments, immunoassays are the various types of enzyme linked immunoadsorbent assays (ELISAs) and radioimmunoassays (RIA) known in the art. Immunohistochemical detection using tissue sections is also particularly useful. However, it will be readily appreciated that detection is not limited to such techniques, and Western blotting, dot and slot blotting, FACS analyses, and the like may also be used. The steps of various useful immunoassays have been described in the scientific literature, such as, e.g., Nakamura et al., in Enzyme Immunoassays: Heterogeneous and Homogeneous Systems, Chapter 27 (1987), incorporated herein by reference.

In general, the detection of immunocomplex formation is well known in the art and may be achieved through the application of numerous approaches. These methods are based upon the detection of radioactive, fluorescent, biological or enzymatic tags. Of course, one may find additional advantages through the use of a secondary binding ligand such as a second antibody or a biotin/avidin ligand binding arrangement, as is known in the art.

The antibody used in the detection may itself be conjugated to a detectable label, wherein one would then simply detect this label. The amount of the primary immune complexes in the composition would, thereby, be determined.

Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under conditions effective and for a period of time sufficient to allow the formation of secondary immune complexes. The secondary immune complexes are washed extensively to remove any non-specifically bound labeled secondary antibodies or ligands, and the remaining label in the secondary immune complex is detected.

An enzyme linked immunoadsorbent assay (ELISA) is a type of binding assay. In one type of ELISA, acetylation site-specific antibodies disclosed herein are immobilized onto a selected surface exhibiting protein affinity, such as a well in a polystyrene microtiter plate. Then, a suspected neoplastic tissue sample is added to the wells. After binding and washing to remove non-specifically bound immune complexes, the bound target signaling protein may be detected.

In another type of ELISA, the neoplastic tissue samples are immobilized onto the well surface and then contacted with the acetylation site-specific antibodies disclosed herein. After binding and washing to remove non-specifically bound immune complexes, the bound acetylation site-specific antibodies are detected.

Irrespective of the format used, ELISAs have certain features in common, such as coating, incubating or binding, washing to remove non-specifically bound species, and detecting the bound immune complexes.

The radioimmunoassay (RIA) is an analytical technique which depends on the competition (affinity) of an antigen for antigen-binding sites on antibody molecules. Standard curves are constructed from data gathered from a series of samples each containing the same known concentration of labeled antigen, and various, but known, concentrations of unlabeled antigen. Antigens are labeled with a radioactive isotope tracer. The mixture is incubated in contact with an antibody. Then the free antigen is separated from the antibody and the antigen bound thereto. Then, by use of a suitable detector, such as a gamma or beta radiation detector, the percent of either the bound or free labeled antigen or both is determined. This procedure is repeated for a number of samples containing various known concentrations of unlabeled antigens and the results are plotted as a standard graph. The percent of bound tracer antigens is plotted as a function of the antigen concentration. Typically, as the total antigen concentration increases the relative amount of the tracer antigen bound to the antibody decreases. After the standard graph is prepared, it is thereafter used to determine the concentration of antigen in samples undergoing analysis.

In an analysis, the sample in which the concentration of antigen is to be determined is mixed with a known amount of tracer antigen. Tracer antigen is the same antigen known to be in the sample but which has been labeled with a suitable radioactive isotope. The sample with tracer is then incubated in contact with the antibody. Then it can be counted in a suitable detector which counts the free antigen remaining in the sample. The antigen bound to the antibody or immunoadsorbent may also be similarly counted. Then, from the standard curve, the concentration of antigen in the original sample is determined.

### 10. Pharmaceutical Formulations and Methods of Administration

Methods of administration of therapeutic agents, particularly peptide and antibody therapeutics, are well-known to those of skill in the art.

Peptides of the invention can be administered in the same manner as conventional peptide type pharmaceuticals. Preferably, peptides are administered parenterally, for example, intravenously, intramuscularly, intraperitoneally, or subcutaneously. When administered orally, peptides may be proteolytically hydrolyzed. Therefore, oral application may not be usually effective. However, peptides can be administered orally as a formulation wherein peptides are not easily hydrolyzed in a digestive tract, such as liposome-microcapsules. Peptides may be also administered in suppositories, sublingual tablets, or intranasal spray.

If administered parenterally, a preferred pharmaceutical composition is an aqueous solution that, in addition to a peptide of the invention as an active ingredient, may contain for example, buffers such as phosphate, acetate, etc., osmotic pressure-adjusting agents such as sodium chloride, sucrose, and sorbitol, etc., antioxidative or antioxygenic agents, such as ascorbic acid or tocopherol and preservatives, such as antibiotics. The parenterally administered composition also may be a solution readily usable or in a lyophilized form which is dissolved in sterile water before administration.

The pharmaceutical formulations, dosage forms, and uses described below generally apply to antibody-based therapeutic agents, but are also useful and can be modified, where necessary, for making and using therapeutic agents of the disclosure that are not antibodies.

To achieve the desired therapeutic effect, the acetylation site-specific antibodies or antigen-binding fragments thereof can be administered in a variety of unit dosage forms. The dose will vary according to the particular antibody. For example, different antibodies may have different masses and/or affinities, and thus require different dosage levels. Antibodies prepared as Fab or other fragments will also require differing dosages than the equivalent intact immunoglobulins, as they are of considerably smaller mass than intact immunoglobulins, and thus require lower dosages to reach the same molar levels in the patient's blood. The dose will also vary depending on the manner of administration, the particular symptoms of the patient being treated, the overall health, condition, size, and age of the patient, and the judgment of the prescribing physician. Dosage levels of the antibodies for human subjects are generally between about 1 mg per kg and about 100 mg per kg per patient per treatment, such as for example, between about 5 mg per kg and about 50 mg per kg per patient per treatment. In terms of plasma concentrations, the antibody concentrations may be in the range from about 25 μg/mL to about 500 μg/mL. However, greater amounts may be required for extreme cases and smaller amounts may be sufficient for milder cases.

Administration of an antibody will generally be performed by a parenteral route, typically via injection such as intra-articular or intravascular injection (e.g., intravenous infusion) or intramuscular injection. Other routes of administration, e.g., oral (p.o.), may be used if desired and practicable for the particular antibody to be administered. An antibody can also be administered in a variety of unit dosage forms and their dosages will also vary with the size, potency, and in vivo half-life of the particular antibody being administered. Doses of a acetylation site-specific antibody will also vary depending on the manner of administration, the particular symptoms of the patient being treated, the overall health, condition, size, and age of the patient, and the judgment of the prescribing physician.

The frequency of administration may also be adjusted according to various parameters. These include the clinical response, the plasma half-life of the antibody, and the levels of the antibody in a body fluid, such as, blood, plasma, serum, or synovial fluid. To guide adjustment of the frequency of administration, levels of the antibody in the body fluid may be monitored during the course of treatment.

Formulations particularly useful for antibody-based therapeutic agents are also described in U.S. Patent App. Publication Nos. 20030202972, 20040091490 and 20050158316. In certain embodiments, the liquid formulations of the application are substantially free of surfactant and/or inorganic salts. In another specific embodiment, the liquid formulations have a pH ranging from about 5.0 to about 7.0. In yet another specific embodiment, the liquid formulations comprise histidine at a concentration ranging from about 1 mM to about 100 mM. In still another specific embodiment, the liquid formulations comprise histidine at a concentration ranging from 1 mM to 100 mM. It is also contemplated that the liquid formulations may further comprise one or more excipients such as a saccharide, an amino acid (e.g., arginine, lysine, and methionine) and a polyol. Additional descriptions and methods of preparing and analyzing liquid formulations can be found, for example, in PCT publications WO 03/106644, WO 04/066957, and WO 04/091658.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the pharmaceutical compositions of the application.

In certain embodiments, formulations of the subject antibodies are pyrogen-free formulations which are substantially free of endotoxins and/or related pyrogenic substances. Endotoxins include toxins that are confined inside microorganisms and are released when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, it is advantageous to remove even low amounts of endotoxins from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). When therapeutic proteins are administered in amounts of several hundred or thousand milligrams per kilogram body weight, as can be the case with monoclonal antibodies, it is advantageous to remove even trace amounts of endotoxin.

The amount of the formulation which will be therapeutically effective can be determined by standard clinical techniques. In addition, in vitro assays may optionally be used to help identify optimal dosage ranges. The precise dose to be used in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems. The dosage of the compositions to be administered can be determined by the skilled artisan without undue experimentation in conjunction with standard dose-response studies. Relevant circumstances to be considered in making those determinations include the condition or conditions to be treated, the choice of composition to be administered, the age, weight, and response of the individual patient, and the severity of the patient's symptoms. For example, the actual patient body weight may be used to calculate the dose of the formulations in milliliters (mL) to be administered. There may be no downward adjustment to “ideal” weight. In such a situation, an appropriate dose may be calculated by the following formula:

Dose(mL)=[patient weight(kg)×dose level(mg/kg)/drug concentration(mg/mL)]

For the purpose of treatment of disease, the appropriate dosage of the compounds (for example, antibodies) will depend on the severity and course of disease, the patient's clinical history and response, the toxicity of the antibodies, and the discretion of the attending physician. The initial candidate dosage may be administered to a patient. The proper dosage and treatment regimen can be established by monitoring the progress of therapy using conventional techniques known to those of skill in the art.

The formulations of the application can be distributed as articles of manufacture comprising packaging material and a pharmaceutical agent which comprises, e.g., the antibody and a pharmaceutically acceptable carrier as appropriate to the mode of administration.

### 11. Kits

Antibodies and peptides (including AQUA peptides) of the invention may also be used within a kit for detecting the acetylation state or level at a novel acetylation site of the invention, comprising at least one of the following: an AQUA peptide comprising the acetylation site, or an antibody or an antigen-binding fragment thereof that binds to an amino acid sequence comprising the acetylation site. Such a kit may further comprise a packaged combination of reagents in predetermined amounts with instructions for performing the diagnostic assay. Where the antibody is labeled with an enzyme, the kit will include substrates and co-factors required by the enzyme. In addition, other additives may be included such as stabilizers, buffers and the like. The relative amounts of the various reagents may be varied widely to provide for concentrations in solution of the reagents that substantially optimize the sensitivity of the assay. Particularly, the reagents may be provided as dry powders, usually lyophilized, including excipients that, on dissolution, will provide a reagent solution having the appropriate concentration.

The following Examples are provided only to further illustrate the invention, and are not intended to limit its scope, except as provided in the claims appended hereto. The invention encompasses modifications and variations of the methods taught herein which would be obvious to one of ordinary skill in the art.

### Example 1

### Isolation of Acetyl-Lysine Containing Peptides from Extracts of Tissues and Cell Lines and Identification of Novel Acetylation Sites

In order to discover novel lysine acetylation sites, IAP isolation techniques were used to identify acetyl-lysine containing peptides in extracts from selected tissues and cell lines. Tryptic acetyllysine-containing peptides were purified and analyzed from extracts of each of the cell lines mentioned above, as follows. Cells were cultured in DMEM medium or RPMI 1640 medium supplemented with 10% fetal bovine serum and penicillin/streptomycin.

Suspension cells were harvested by low speed centrifugation. After complete aspiration of medium, cells were resuspended in 1 mL lysis buffer per 1.25×10⁸ cells (20 mM HEPES pH 8.0, 9 M urea, 1 mM sodium vanadate, supplemented or not with 2.5 mM sodium pyro-phosphate, 1 mM β-glycerol-phosphate) and sonicated.

Adherent cells at about 70-80% confluency were starved in medium without serum overnight and stimulated, with ligand depending on the cell type or not stimulated. After complete aspiration of medium from the plates, cells were scraped off the plate in 10 ml lysis buffer per 2×10⁸ cells (20 mM HEPES pH 8.0, 9 M urea, 1 mM sodium vanadate, supplemented with 2.5 mM sodium pyrophosphate, 1 mM β-glycerol-phosphate) and sonicated.

Frozen tissue samples were cut to small pieces, homogenize in lysis buffer (20 mM HEPES pH 8.0, 9 M Urea, 1 mM sodium vanadate, supplemented with 2.5 mM sodium pyrophosphate, 1 mM β-glycerol-phosphate, 1 ml lysis buffer for 100 mg of frozen tissue) using a polytron for 2 times of 20 sec. each time. Homogenate is then briefly sonicated.

Sonicated cell lysates were cleared by centrifugation at 20,000×g, and proteins were reduced with DTT at a final concentration of 4.1 mM and alkylated with iodoacetamide at 8.3 mM. For digestion with trypsin, protein extracts were diluted in 20 mM HEPES pH 8.0 to a final concentration of 2 M urea and soluble TLCK-trypsin (Worthington) was added at 10-20 μg/mL. Digestion was performed for 1 day at room temperature.

Trifluoroacetic acid (TFA) was added to protein digests to a final concentration of 1%, precipitate was removed by centrifugation, and digests were loaded onto Sep-Pak C₁₈ columns (Waters) equilibrated with 0.1% TFA. A column volume of 0.7-1.0 ml was used per 2×10⁸ cells. Columns were washed with 15 volumes of 0.1% TFA, followed by 4 volumes of 5% acetonitrile (MeCN) in 0.1% TFA. Peptide fraction I was obtained by eluting columns with 2 volumes each of 8, 12, and 15% MeCN in 0.1% TFA and combining the eluates. Fractions II and III were a combination of eluates after eluting columns with 18, 22, 25% MeCN in 0.1% TFA and with 30, 35, 40% MeCN in 0.1% TFA, respectively. All peptide fractions were lyophilized.

Peptides from each fraction corresponding to 2×10⁸ cells were dissolved in 1 ml of IAP buffer (20 mM Tris/HCl or 50 mM MOPS pH 7.2, 10 mM sodium phosphate, 50 mM NaCl) and insoluble matter (mainly in peptide fractions III) was removed by centrifugation. IAP was performed on each peptide fraction separately. The acetyl-lysine (Cell Signaling Technology, Inc., catalog number 8691) was coupled at 4 mg/ml beads to protein G (Roche), respectively. Immobilized antibody (15 μl, 60 μg) was added as 1:1 slurry in IAP buffer to 1 ml of each peptide fraction, and the mixture was incubated overnight at 4° C. with gentle rotation. The immobilized antibody beads were washed three times with 1 ml IAP buffer and twice with 1 ml water, all at 4° C. Peptides were eluted from beads by incubation with 75 μl of 0.1% TFA at room temperature for 10 minutes.

Alternatively, one single peptide fraction was obtained from Sep-Pak C18 columns by elution with 2 volumes each of 10%, 15%, 20%, 25%, 30%, 35% and 40% acetonitrile in 0.1% TFA and combination of all eluates. IAP on this peptide fraction was performed as follows: After lyophilization, peptide was dissolved in 1.4 ml IAP buffer (MOPS pH 7.2, 10 mM sodium phosphate, 50 mM NaCl) and insoluble matter was removed by centrifugation. Immobilized antibody (40 μl, 160 μg) was added as 1:1 slurry in IAP buffer, and the mixture was incubated overnight at 4° C. with gentle shaking. The immobilized antibody beads were washed three times with 1 ml IAP buffer and twice with 1 ml water, all at 4° C. Peptides were eluted from beads by incubation with 55 μl of 0.15% TFA at room temperature for 10 min (eluate 1), followed by a wash of the beads (eluate 2) with 45 μl of 0.15% TFA. Both eluates were combined.

### Analysis by LC-MS/MS Mass Spectrometry.

40 μl or more of IAP eluate were purified by 0.2 μl C18 microtips (StageTips or ZipTips). Peptides were eluted from the microcolumns with 1 μl of 40% MeCN, 0.1% TFA (fractions I and II) or 1 μl of 60% MeCN, 0.1% TFA (fraction III) into 7.6-9.0 μl of 0.4% acetic acid/0.005% heptafluorobutyric acid. For single fraction analysis, 1 μl of 60% MeCN, 0.1% TFA, was used for elution from the microcolumns. This sample was loaded onto a 10 cm×75 μl PicoFrit capillary column (New Objective) packed with Magic C18 AQ reversed-phase resin (Michrom Bioresources) using a Famos autosampler with an inert sample injection valve (Dionex). The column was then developed with a 45-min linear gradient of acetonitrile delivered at 200 nl/min (Ultimate, Dionex), and tandem mass spectra were collected in a data-dependent manner with an LTQ ion trap mass spectrometer essentially as described by Gygi et al., supra.

### Database Analysis & Assignments.

MS/MS spectra were evaluated using TurboSequest in the Sequest Browser package (v. 27, rev. 12) supplied as part of BioWorks 3.0 (ThermoFinnigan). Individual MS/MS spectra were extracted from the raw data file using the Sequest Browser program CreateDta, with the following settings: bottom MW, 700; top MW, 4,500; minimum number of ions, 40; minimum TIC, 2×10³; and precursor charge state, unspecified. Spectra were extracted from the beginning of the raw data file before sample injection to the end of the eluting gradient. The IonQuest and VuDta programs were not used to further select MS/MS spectra for Sequest analysis. MS/MS spectra were evaluated with the following TurboSequest parameters: peptide mass tolerance, 2.5; fragment ion tolerance, 1.0; maximum number of differential amino acids per modification, 4; mass type parent, average; mass type fragment, average; maximum number of internal cleavage sites, 10; neutral losses of water and ammonia from b and y ions were considered in the correlation analysis. Proteolytic enzyme was specified except for spectra collected from elastase digests.

Searches were performed against the then current NCBI human protein database. Cysteine carboxamidomethylation was specified as a static modification, and acetylation was allowed as a variable modification on lysinealone.

In proteomics research, it is desirable to validate protein identifications based solely on the observation of a single peptide in one experimental result, in order to indicate that the protein is, in fact, present in a sample. This has led to the development of statistical methods for validating peptide assignments, which are not yet universally accepted, and guidelines for the publication of protein and peptide identification results (see Can et al., Mol. Cell. Proteomics 3: 531-533 (2004)), which were followed in this Example. However, because the immunoaffinity strategy separates acetylated peptides from unacetylated peptides, observing just one acetyl peptide from a protein is a common result, since many acetylated proteins have only one lysine-acetylated site. For this reason, it is appropriate to use additional criteria to validate acetyl peptide assignments. Assignments are likely to be correct if any of these additional criteria are met: (i) the same acetyl peptide sequence is assigned to co-eluting ions with different charge states, since the MS/MS spectrum changes markedly with charge state; (ii) the acetylation site is found in more than one peptide sequence context due to sequence overlaps from incomplete proteolysis or use of proteases other than trypsin; (iii) the acetylation site is found in more than one peptide sequence context due to homologous but not identical protein isoforms; (iv) the acetylation site is found in more than one peptide sequence context due to homologous but not identical proteins among species; and (v) acetylation sites validated by MS/MS analysis of synthetic acetyl peptides corresponding to assigned sequences, since the ion trap mass spectrometer produces highly reproducible MS/MS spectra. The last criterion is routinely used to confirm novel site assignments of particular interest.

All spectra and all sequence assignments made by Sequest were imported into a relational database. The following Sequest scoring thresholds were used to select acetyl peptide assignments that are likely to be correct: RSp<6, XCorr≧2.2, and DeltaCN>0.099. Further, the sequence assignments could be accepted or rejected with respect to accuracy by using the following conservative, two-step process.

In the first step, a subset of high-scoring sequence assignments should be selected by filtering for XCorr values of at least 1.5 for a charge state of +1, 2.2 for +2, and 3.3 for +3, allowing a maximum RSp value of 10. Assignments in this subset should be rejected if any of the following criteria are satisfied: (i) the spectrum contains at least one major peak (at least 10% as intense as the most intense ion in the spectrum) that can not be mapped to the assigned sequence as an a, b, or y ion, as an ion arising from neutral-loss of water or ammonia from a b or y ion, or as a multiply protonated ion; (ii) the spectrum does not contain a series of b or y ions equivalent to at least six uninterrupted residues; or (iii) the sequence is not observed at least five times in all the studies conducted (except for overlapping sequences due to incomplete proteolysis or use of proteases other than trypsin).

In the second step, assignments with below-threshold scores should be accepted if the low-scoring spectrum shows a high degree of similarity to a high-scoring spectrum collected in another study, which simulates a true reference library-searching strategy.

### Example 2

### Production of Acetylation Site-Specific Polyclonal Antibodies

Polyclonal antibodies that specifically bind a novel acetylation site of the invention (Table 1/FIG. 2) only when the lysine residue is acetylated (and does not bind to the same sequence when the lysine is not acetylated), and vice versa, are produced according to standard methods by first constructing a synthetic peptide antigen comprising the acetylation site and then immunizing an animal to raise antibodies against the antigen, as further described below. Production of exemplary polyclonal antibodies is provided below.

A. LRPPRC (lysine 996).

An 15 amino acid acetyl-peptide antigen, YNLLKLYk*INGDWQR (SEQ NO: 428; k*=acetyl-lysine), which comprises the acetylation site derived from human LRPPRC (RNA processing protein), Lys 996 being the acetylatable residue), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) acetylation site-specific polyclonal antibodies as described in Immunization/Screening below.

### B. SLC25A5 (Lysine 10).

A 22 amino acid acetyl-peptide antigen, TDAAVSFAk*DFLAGGVAAAISK (SEQ ID NO: 424; k*=acetyl-lysine, which comprises the acetylation site derived from human SLC25A5 (a receptor/channel/transporter/cell surface protein, Lys 10 being the acetylatable residue), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) acetylation site-specific polyclonal antibodies as described in Immunization/Screening below.

### C. SLC25A4 (Lysine 92).

A 14 amino acid acetyl-peptide antigen, YFPTQALNFAFk*DK (SEQ ID NO: 417; k*=acetyl-lysine, which comprises the acetylation site derived from human SLC25A4 (a receptor/channel/transporter/cell surface protein, Lys 92 being the acetylatable residue), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) acetylation site-specific polyclonal antibodies as described in Immunization/Screening below.

### Immunization/Screening.

A synthetic acetyl-peptide antigen as described in A-C above is coupled to KLH, and rabbits are injected intradermally (ID) on the back with antigen in complete Freunds adjuvant (500 μg antigen per rabbit). The rabbits are boosted with same antigen in incomplete Freund adjuvant (250 μg antigen per rabbit) every three weeks. After the fifth boost, bleeds are collected. The sera are purified by Protein A-affinity chromatography by standard methods (see ANTIBODIES: A LABORATORY MANUAL, Cold Spring Harbor, supra.). The eluted immunoglobulins are further loaded onto an unacetylated synthetic peptide antigen-resin Knotes column to pull out antibodies that bind the unacetylated form of the acetylation sites. The flow through fraction is collected and applied onto an acetyl-synthetic peptide antigen-resin column to isolate antibodies that bind the acetylated form of the acetylation sites. After washing the column extensively, the bound antibodies (i.e. antibodies that bind the acetylated peptides described in A-C above, but do not bind the unacetylated form of the peptides) are eluted and kept in antibody storage buffer.

The isolated antibody is then tested for acetyl-specificity using Western blot assay using an appropriate cell line that expresses (or overexpresses) target acetyl-protein. Cells are cultured in DMEM or RPMI supplemented with 10% FCS. Cell are collected, washed with PBS and directly lysed in cell lysis buffer. The protein concentration of cell lysates is then measured. The loading buffer is added into cell lysate and the mixture is boiled at 100° C. for 5 minutes. 20 μl (10 μg protein) of sample is then added onto 7.5% SDS-PAGE gel.

A standard Western blot may be performed according to the Immunoblotting Protocol set out in the CELL SIGNALING TECHNOLOGY, INC. 2003-04 Catalogue, p. 390. The isolated acetylation site-specific antibody is used at dilution 1:1000. Acetyl-specificity of the antibody will be shown by binding of only the acetylated form of the target amino acid sequence. Isolated acetylation site-specific polyclonal antibody does not (substantially) recognize the same target sequence when not acetylated at the specified lysine position (e.g., the antibody does not bind to SLC25A5 in the non-stimulated cells, when lysine 10 is not acetylated).

In order to confirm the specificity of the isolated antibody, different cell lysates containing various acetylated signaling proteins other than the target protein are prepared. The Western blot assay is performed again using these cell lysates. The acetylation site-specific polyclonal antibody isolated as described above is used (1:1000 dilution) to test reactivity with the different acetylated non-target proteins. The acetylation site-specific antibody does not significantly cross-react with other acetylated signaling proteins that do not have the described acetylation site, although occasionally slight binding to a highly homologous sequence on another protein may be observed. In such case the antibody may be further purified using affinity chromatography, or the specific immunoreactivity cloned by rabbit hybridoma technology.

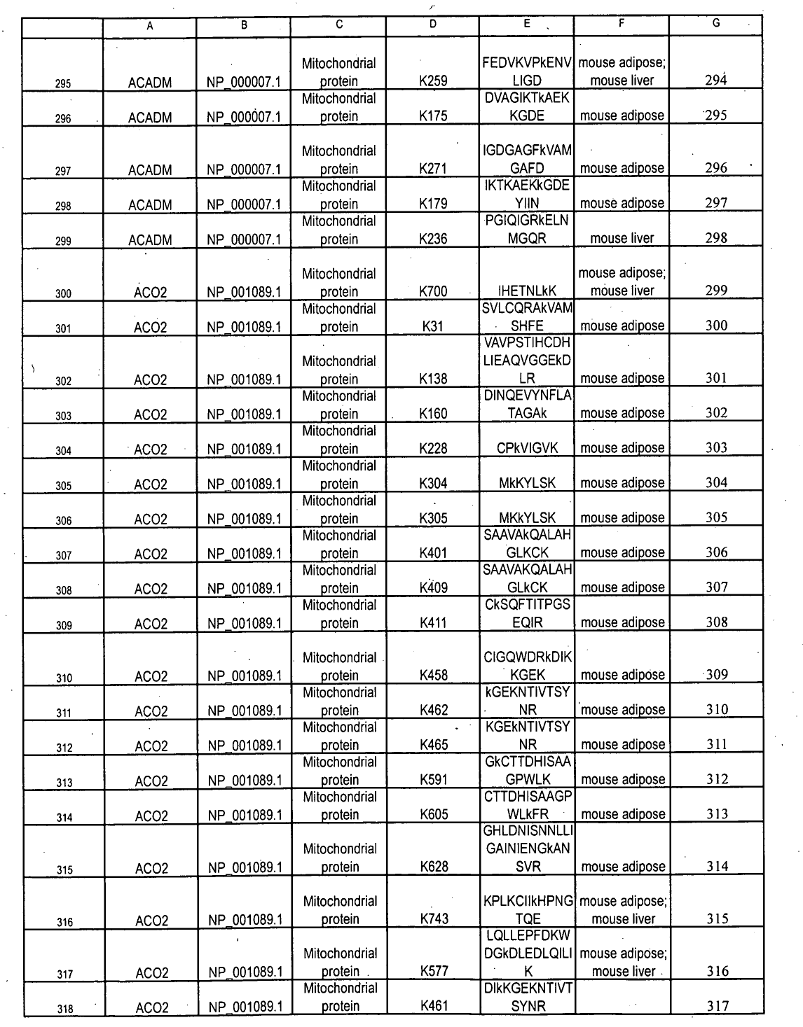

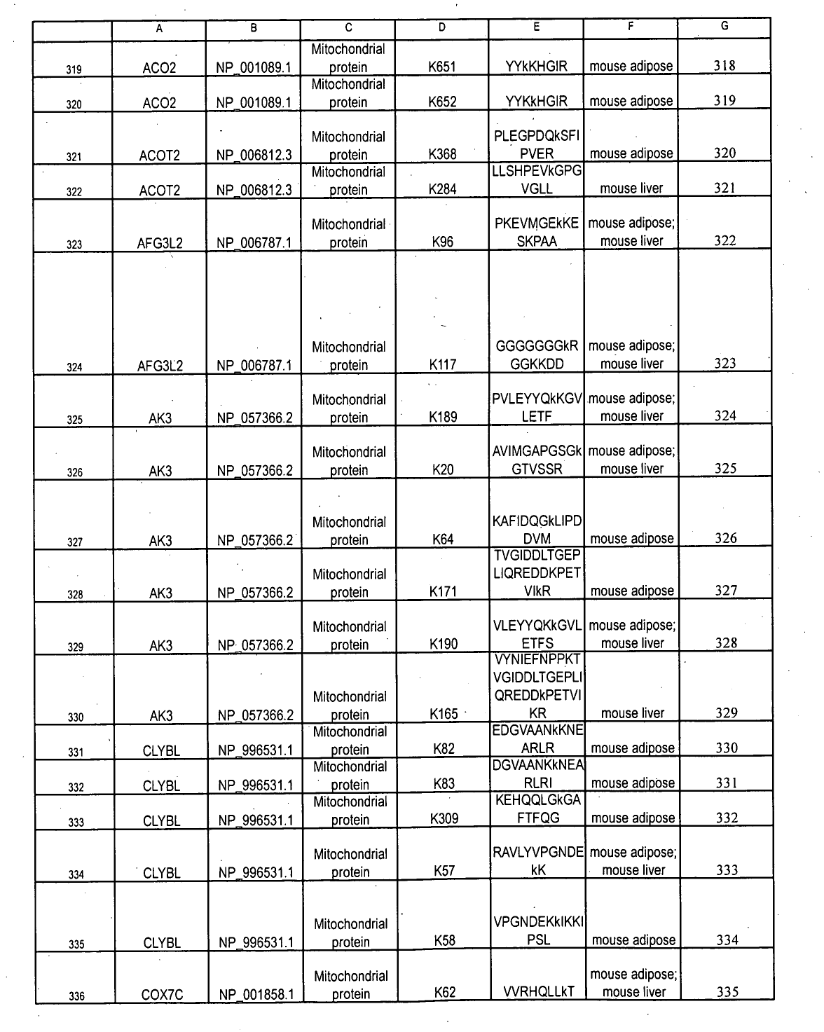

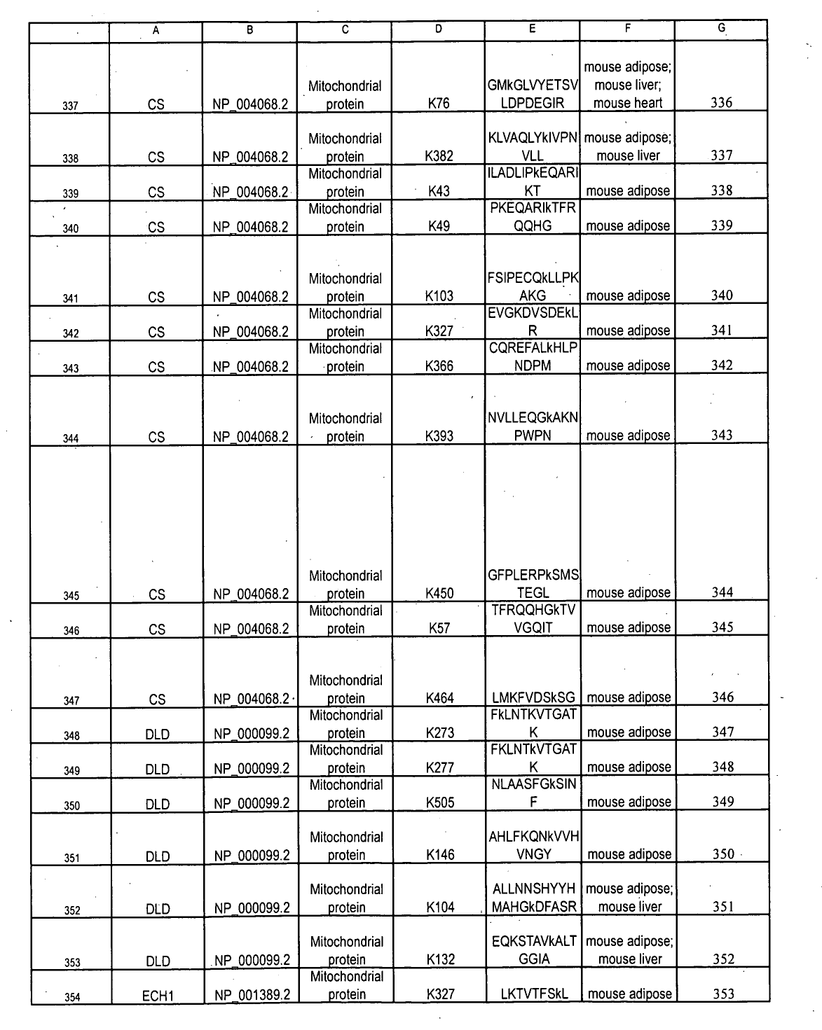

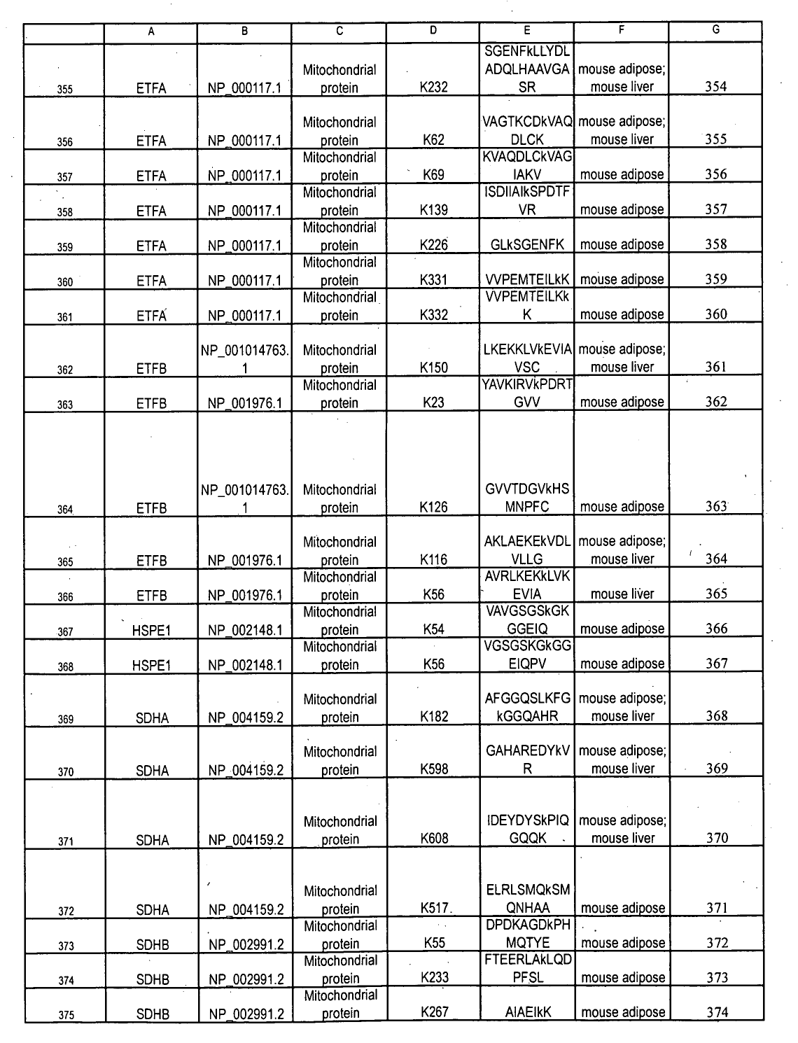

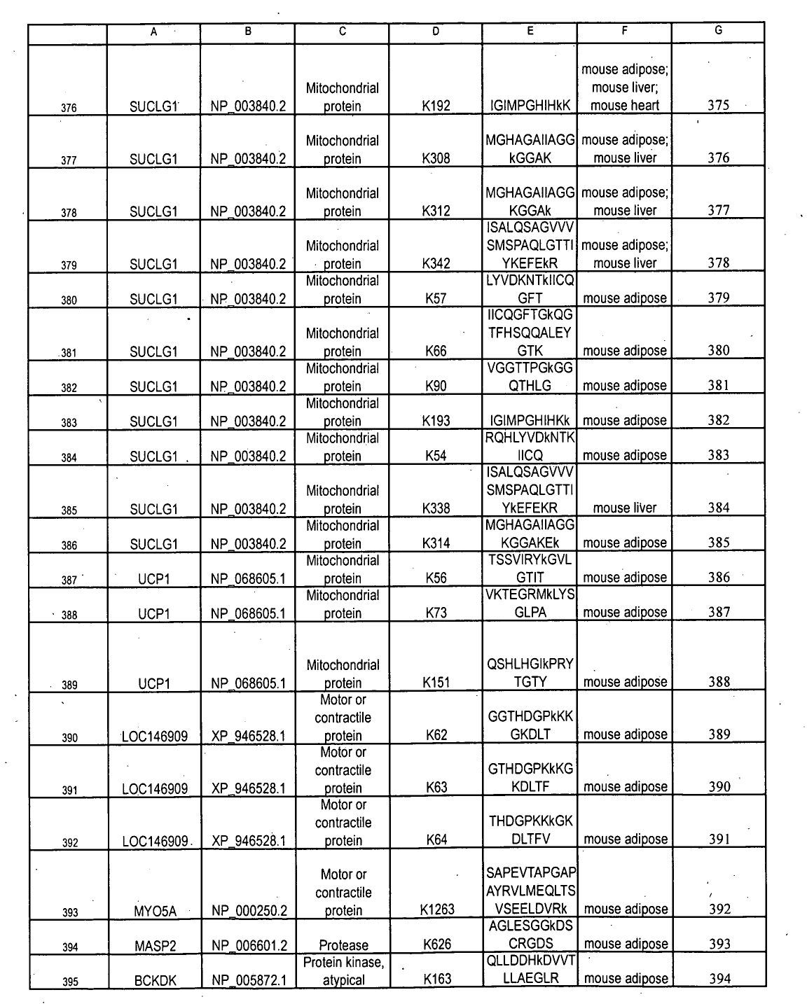

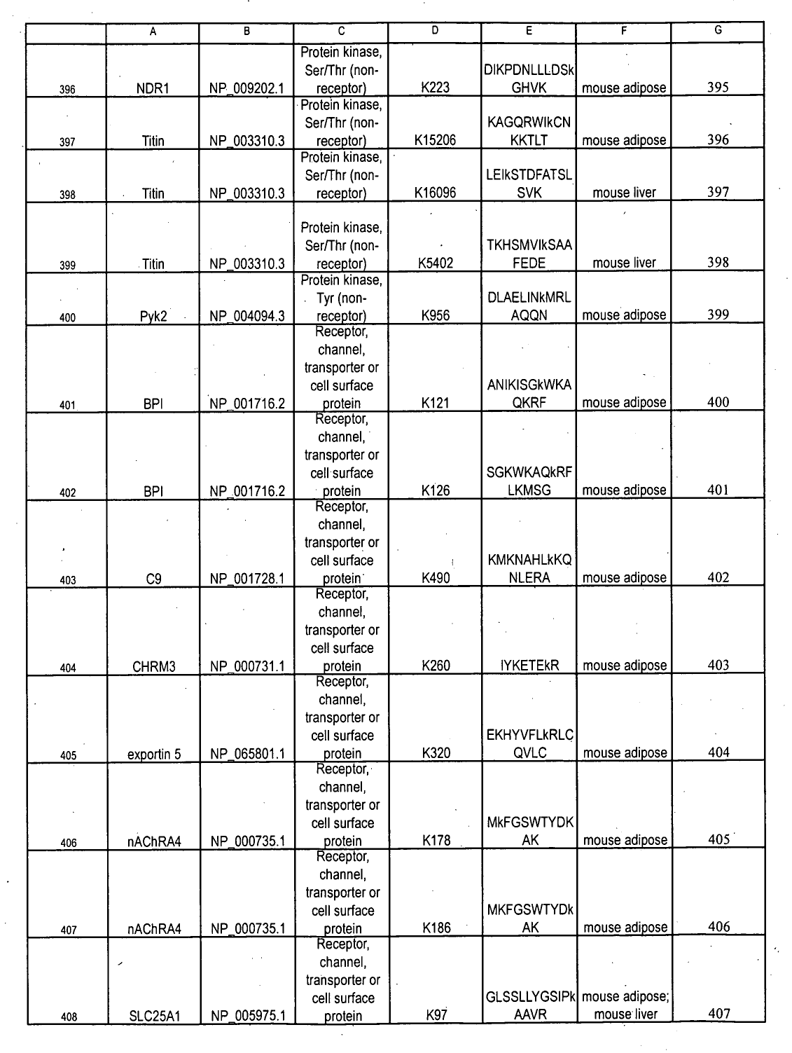

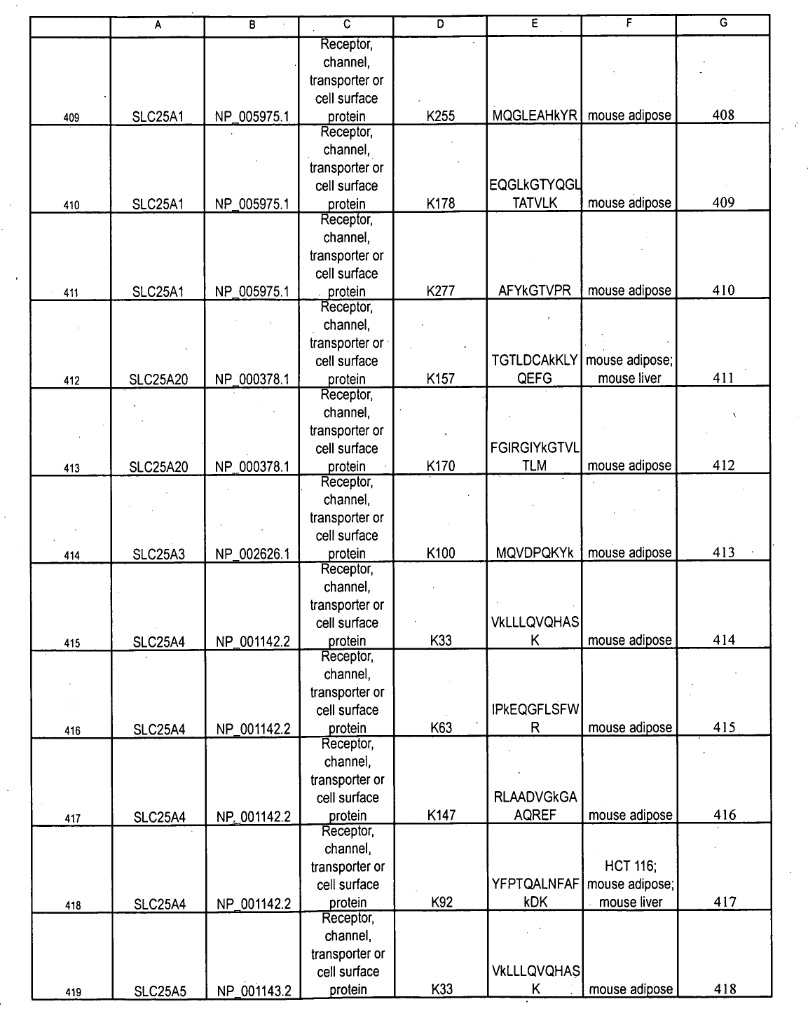

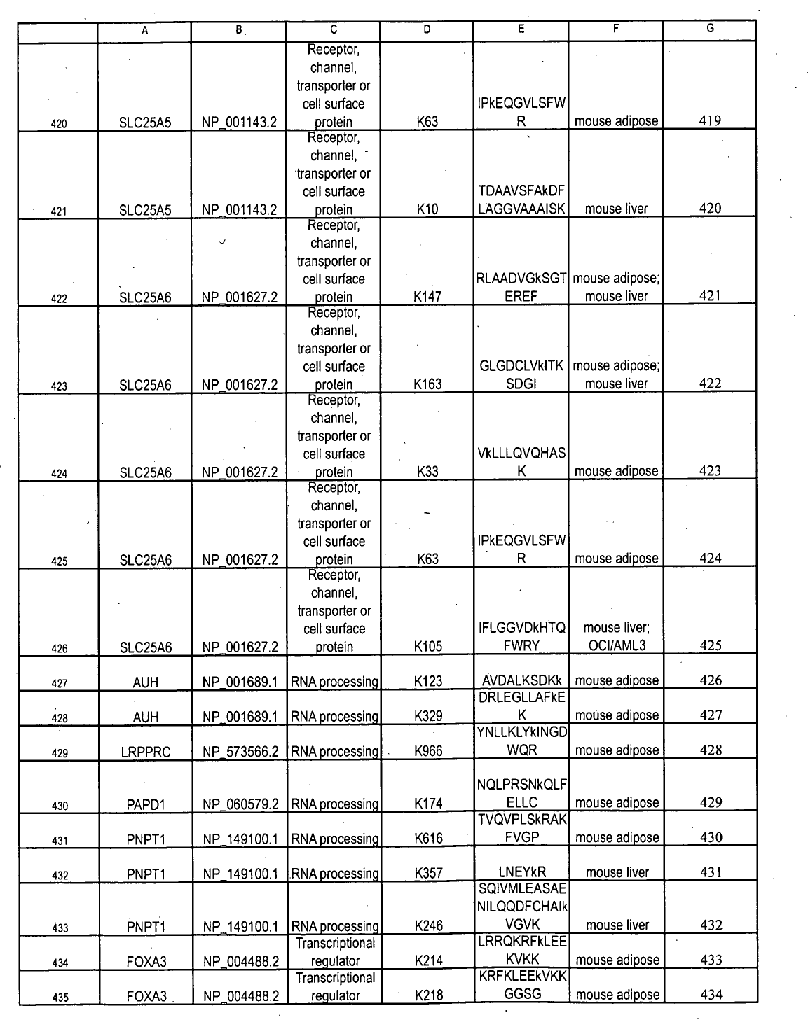

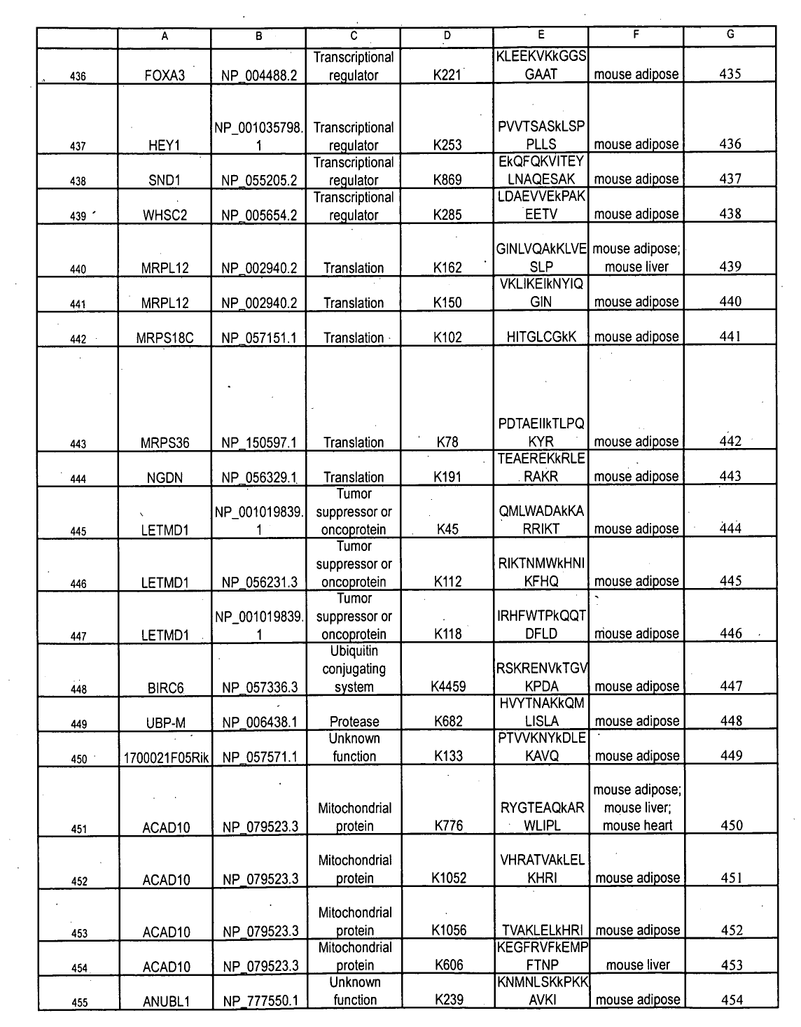

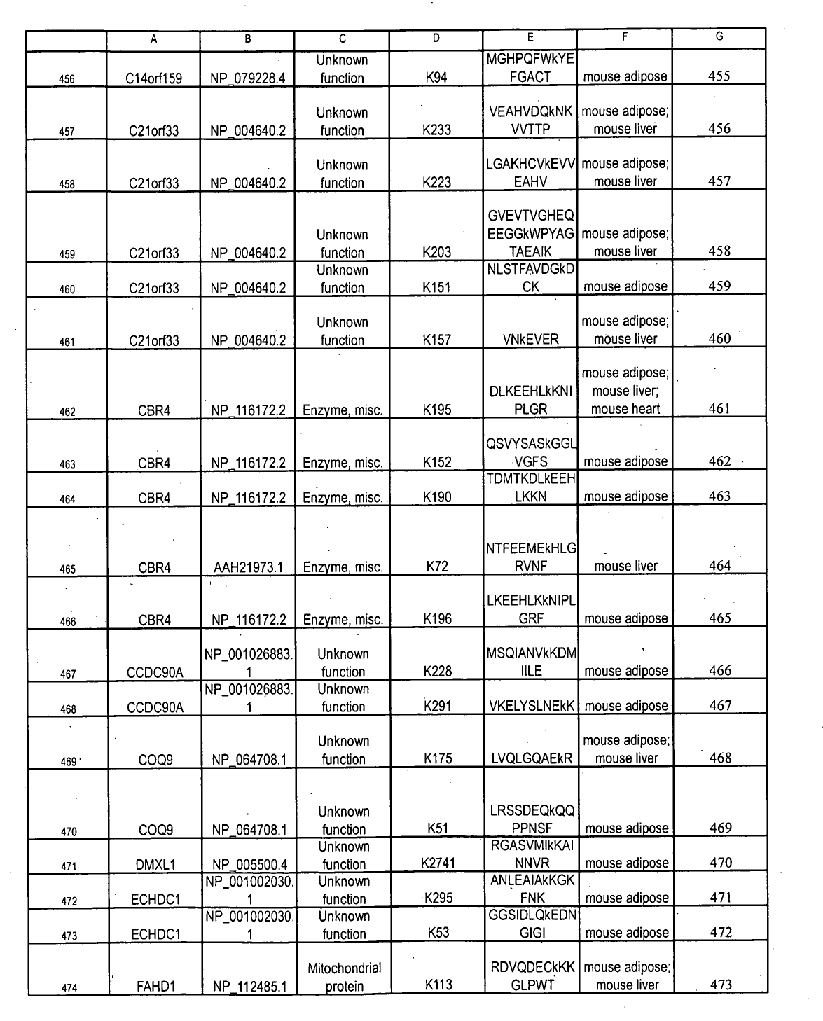

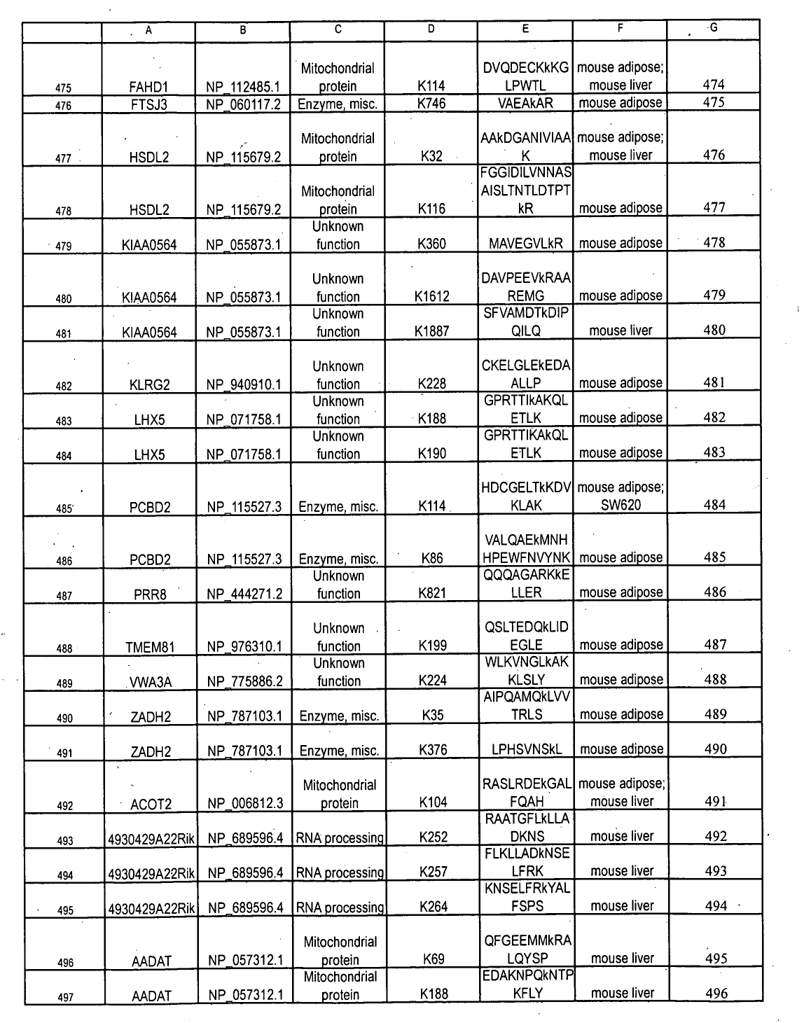

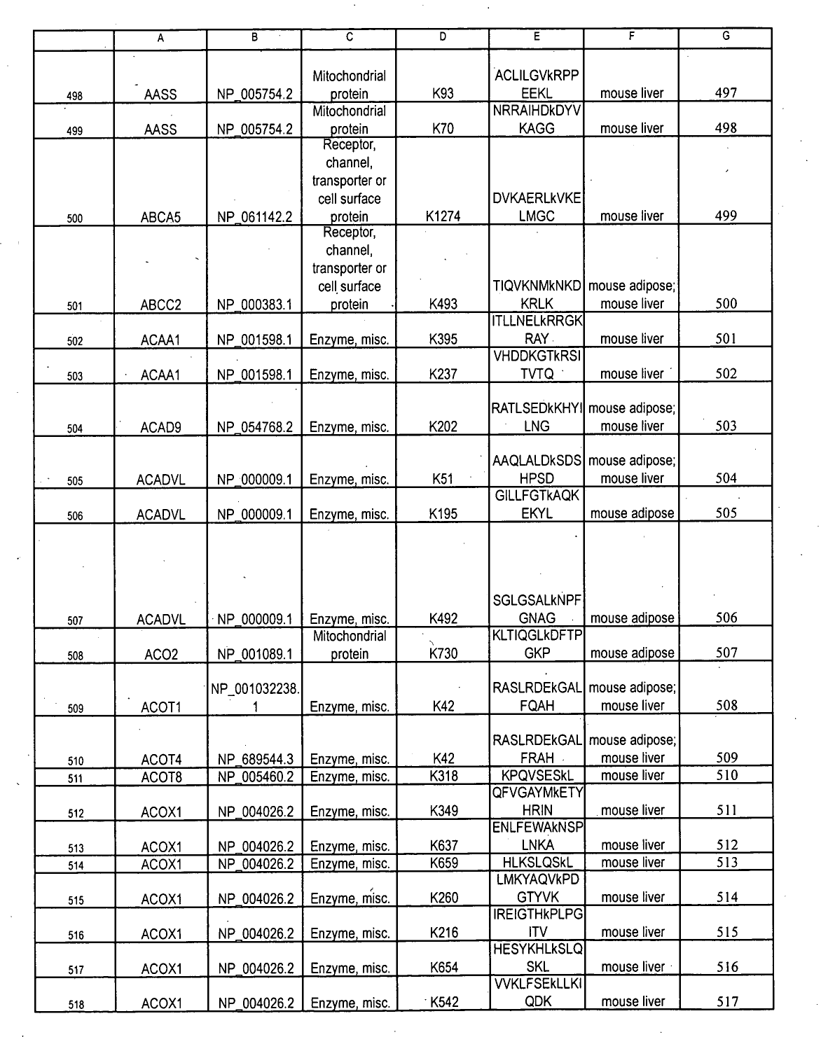

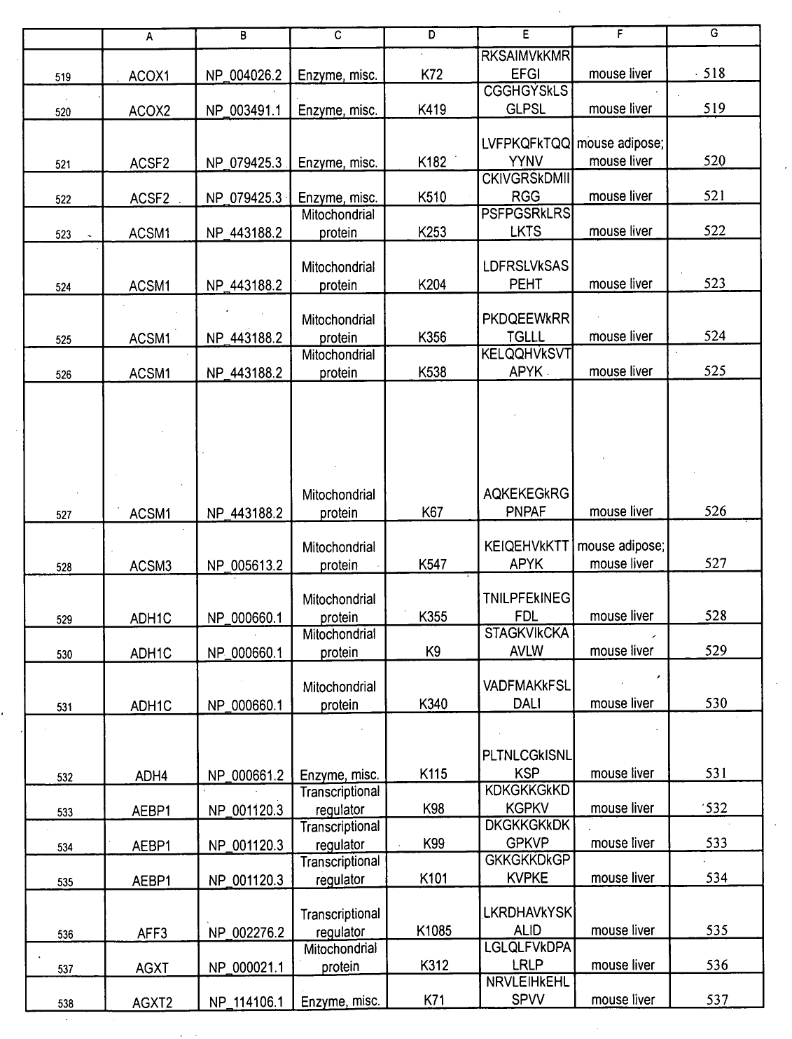

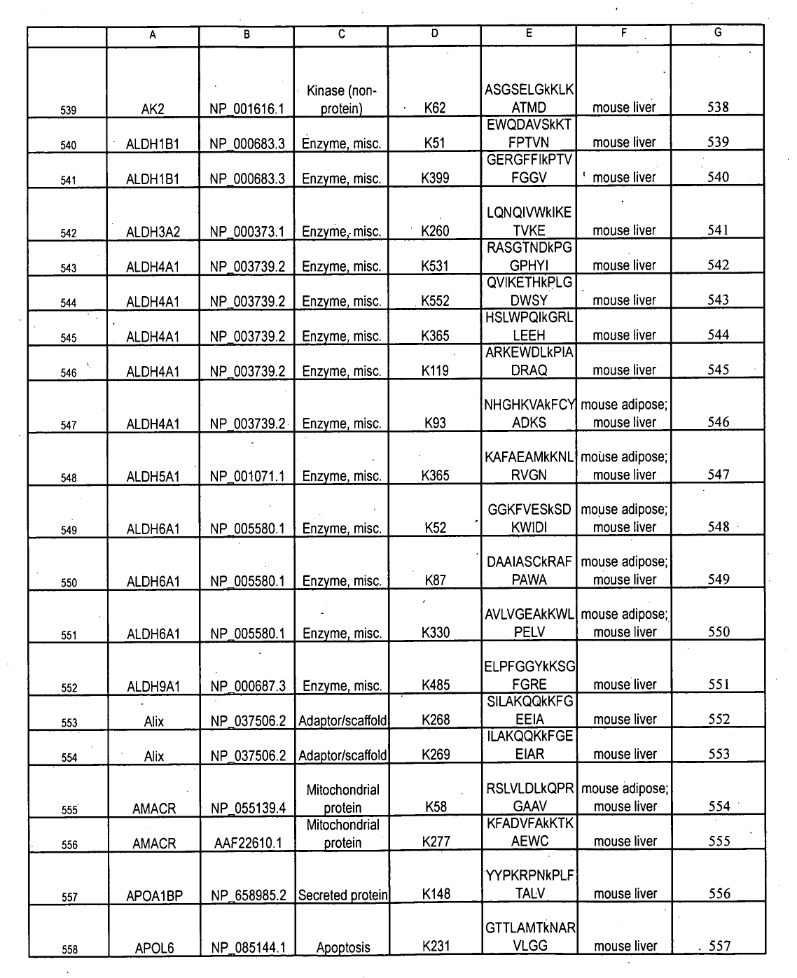

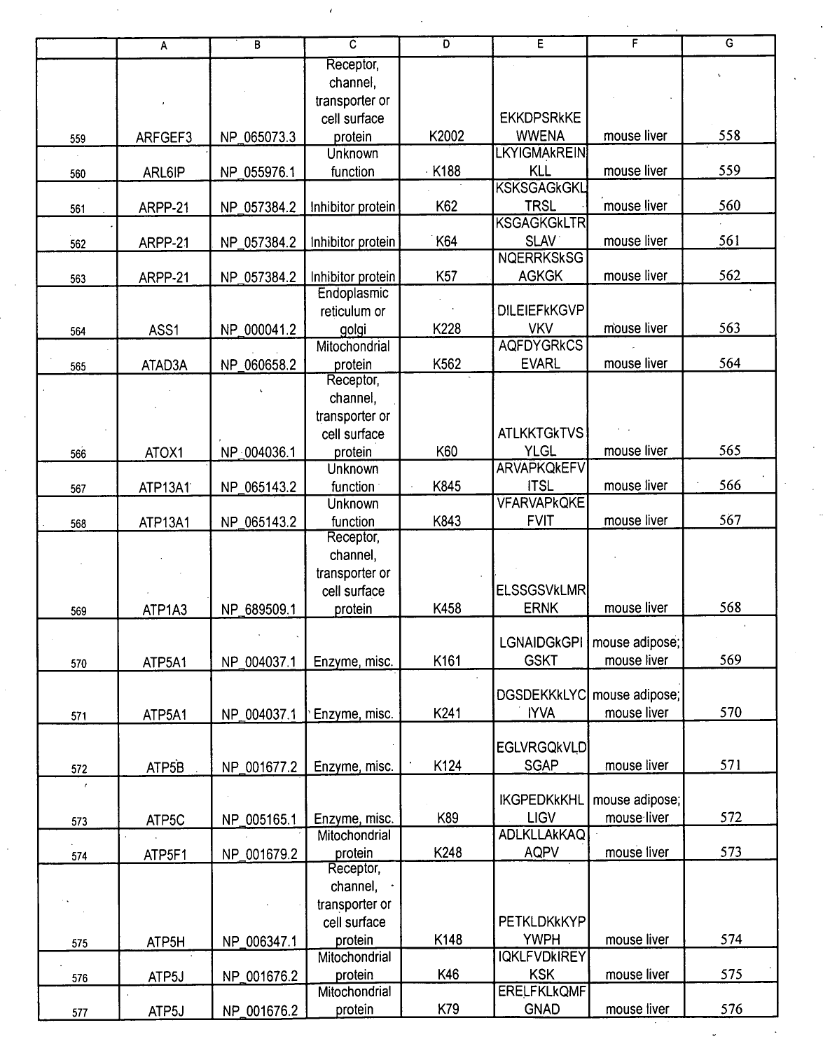

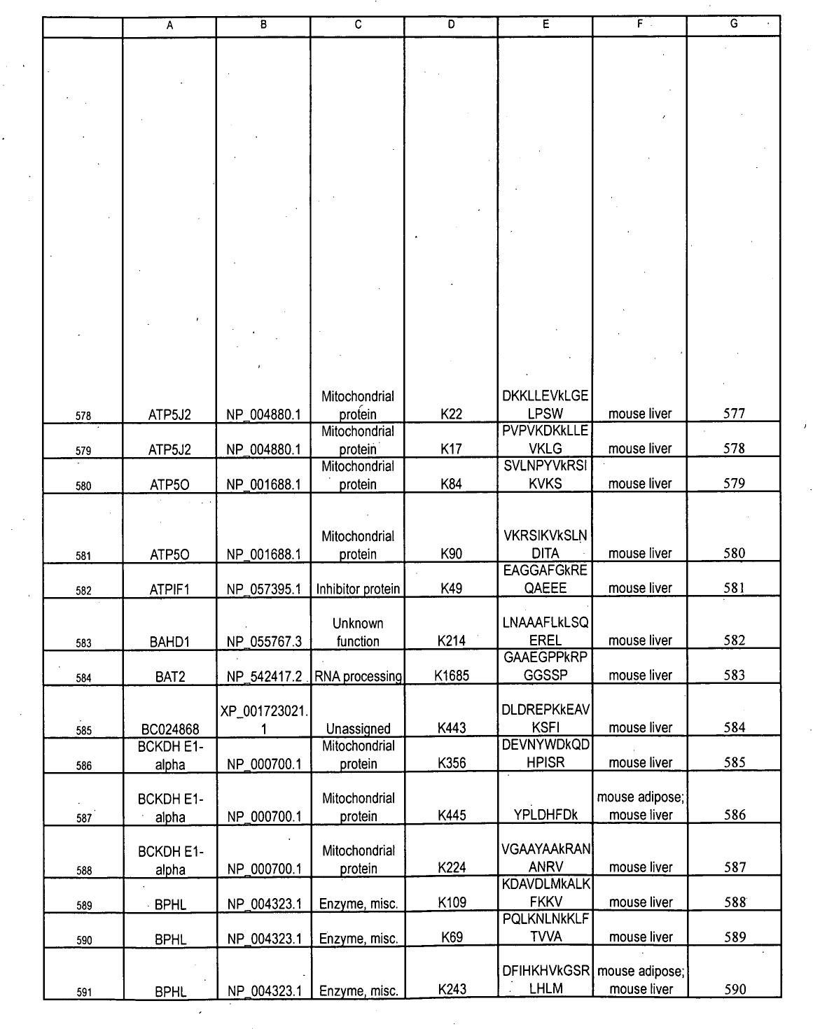

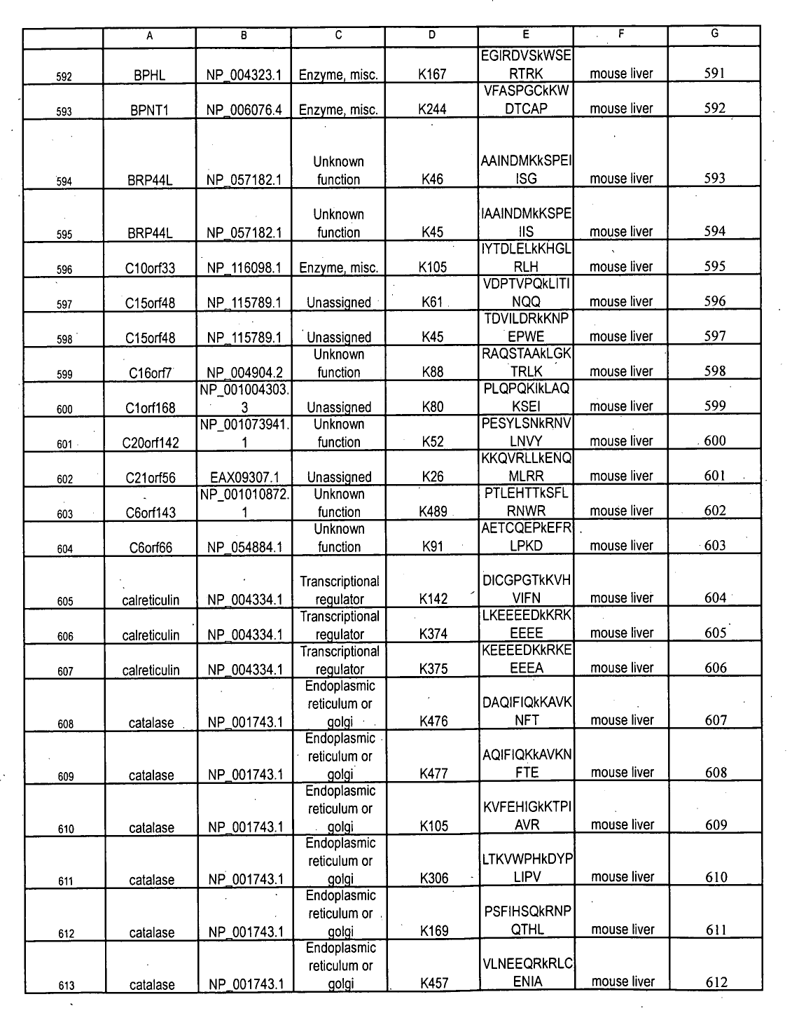

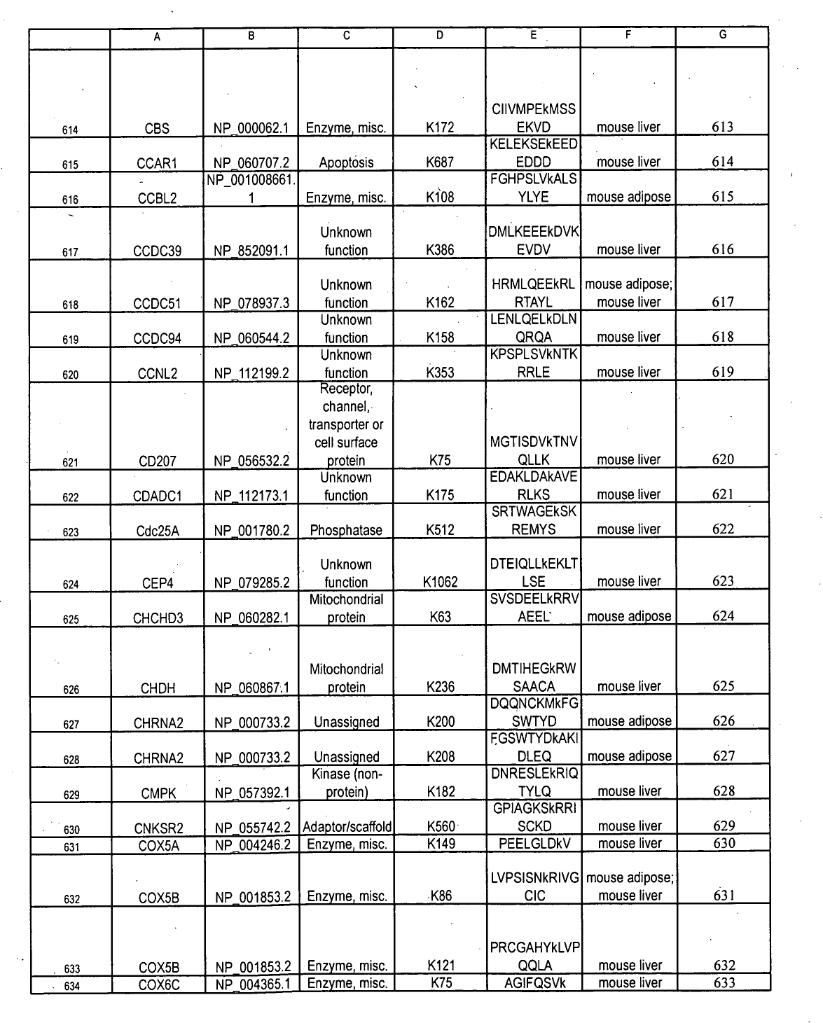

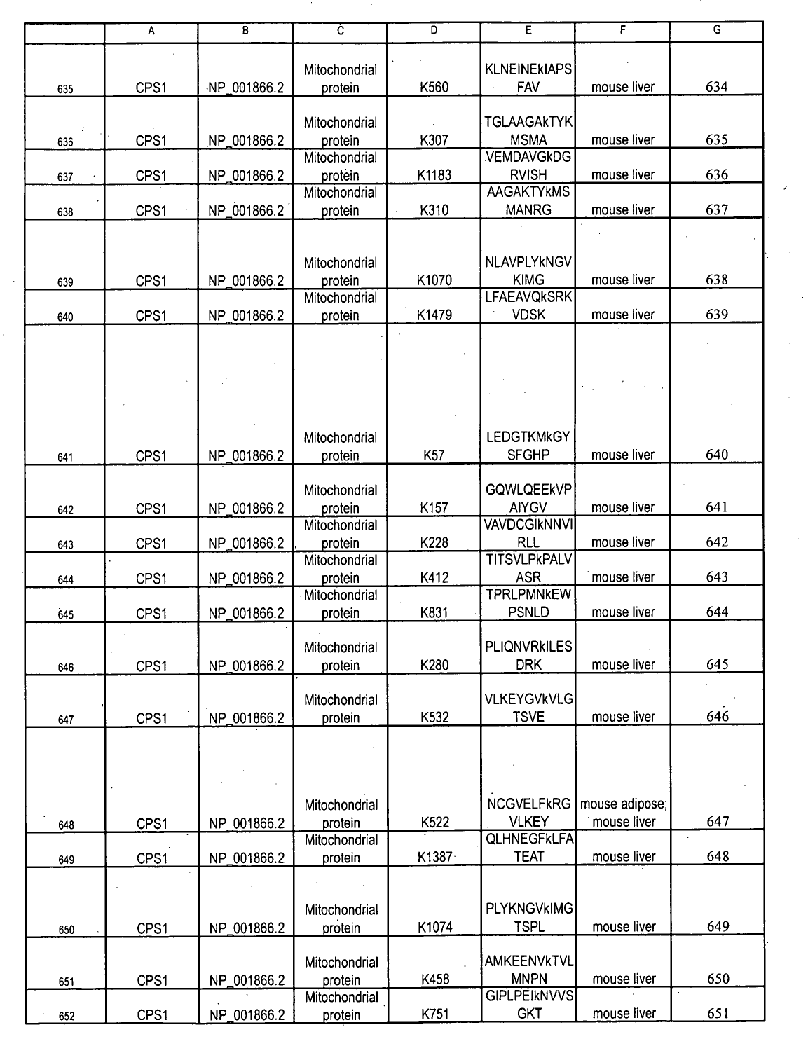

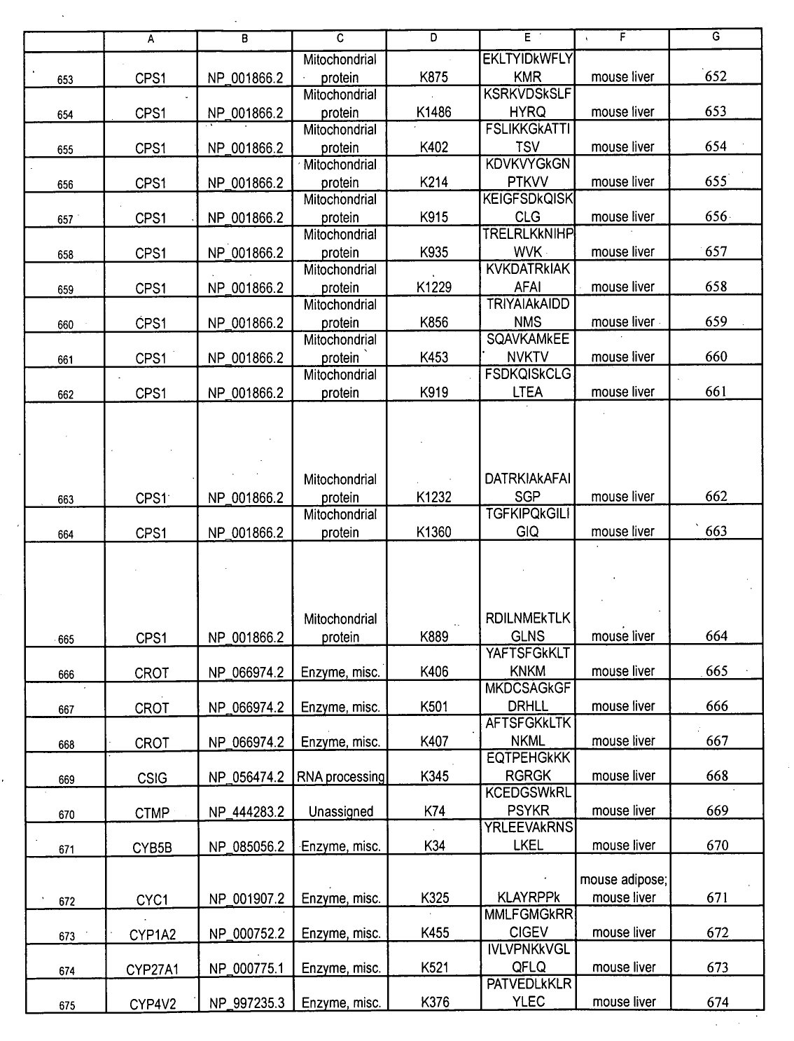

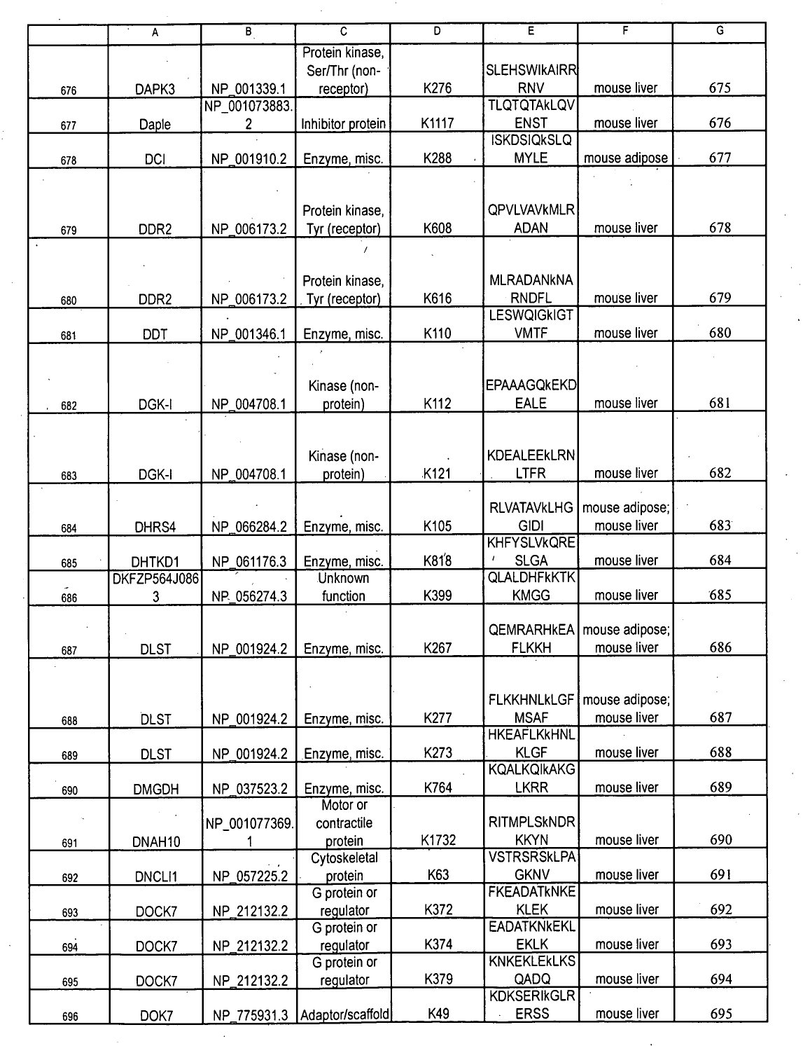

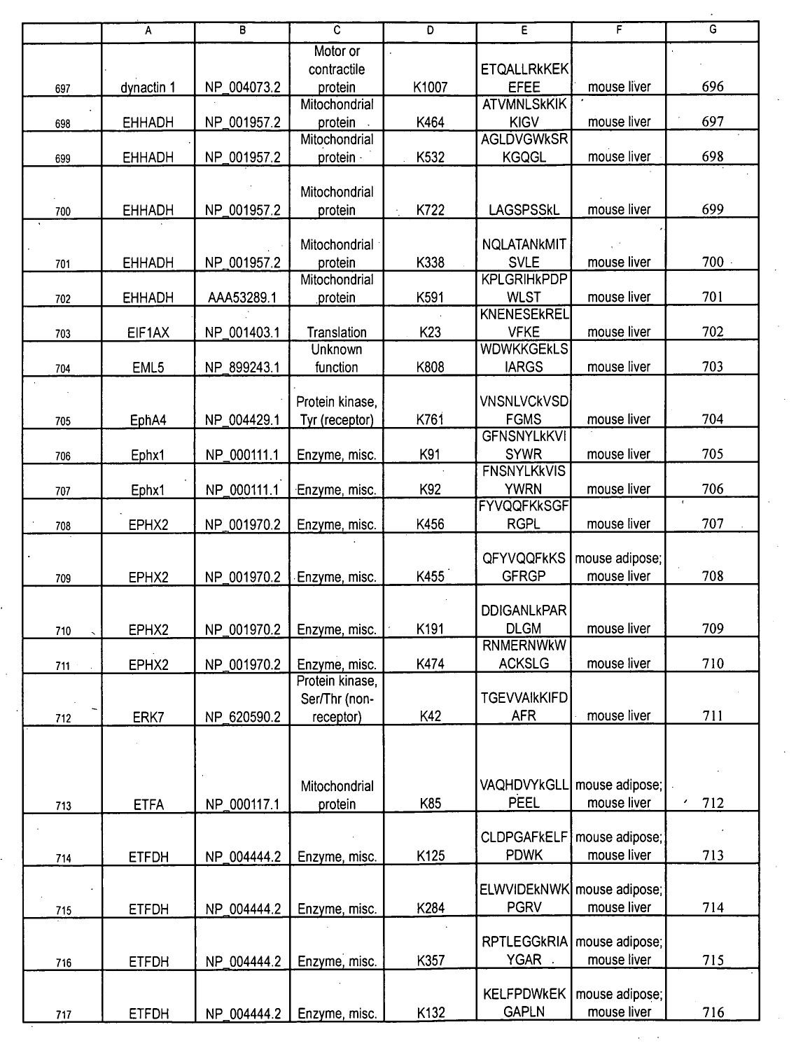

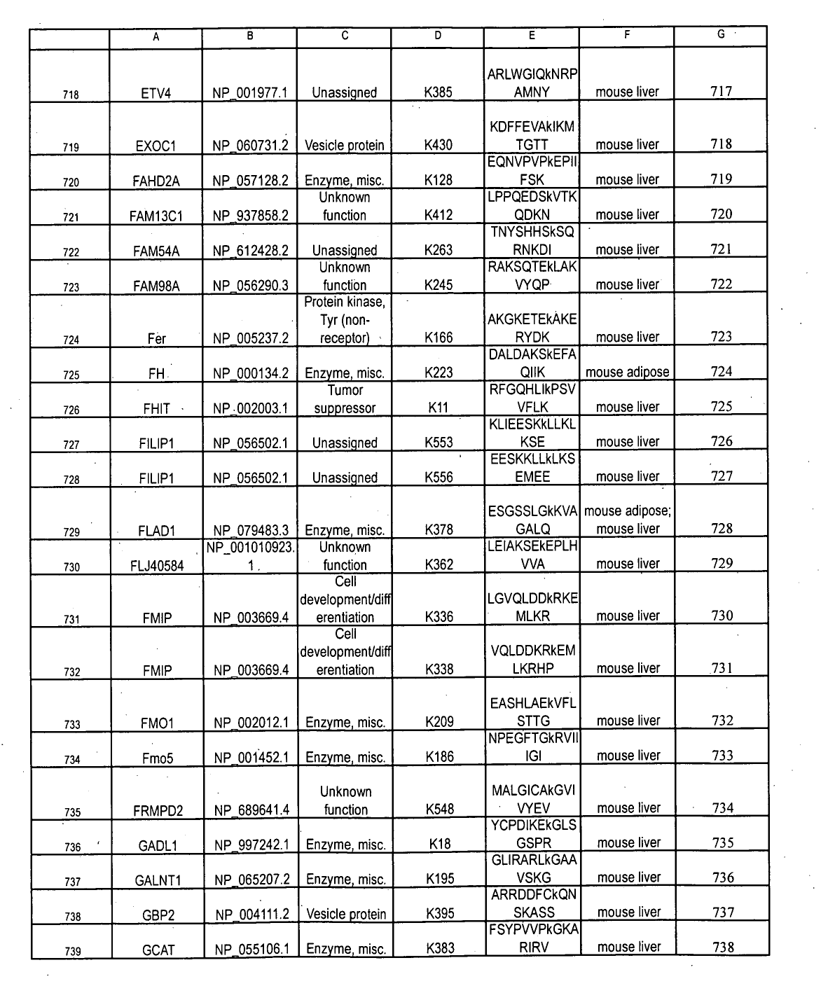

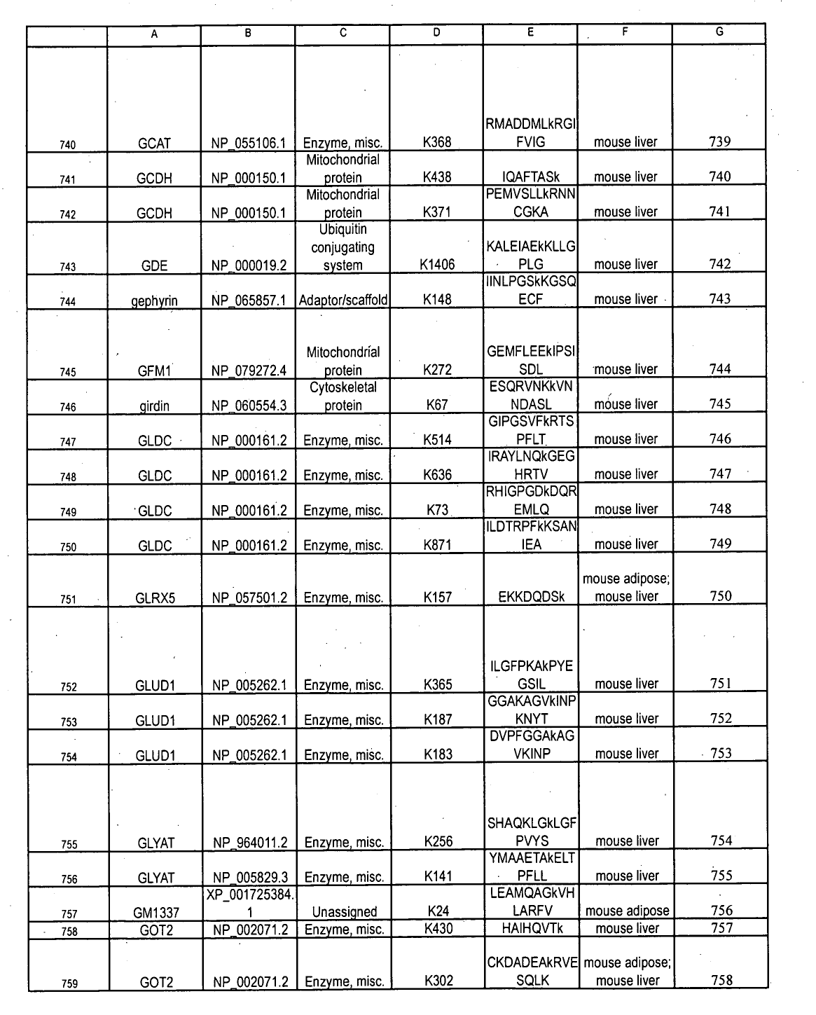

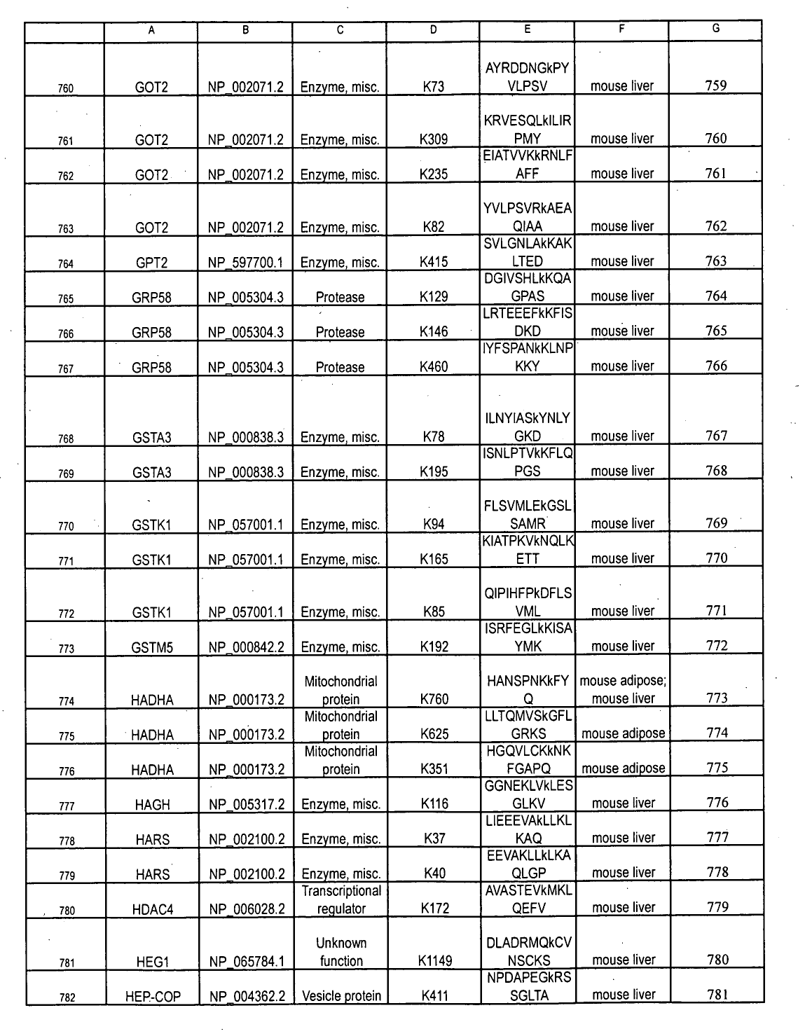

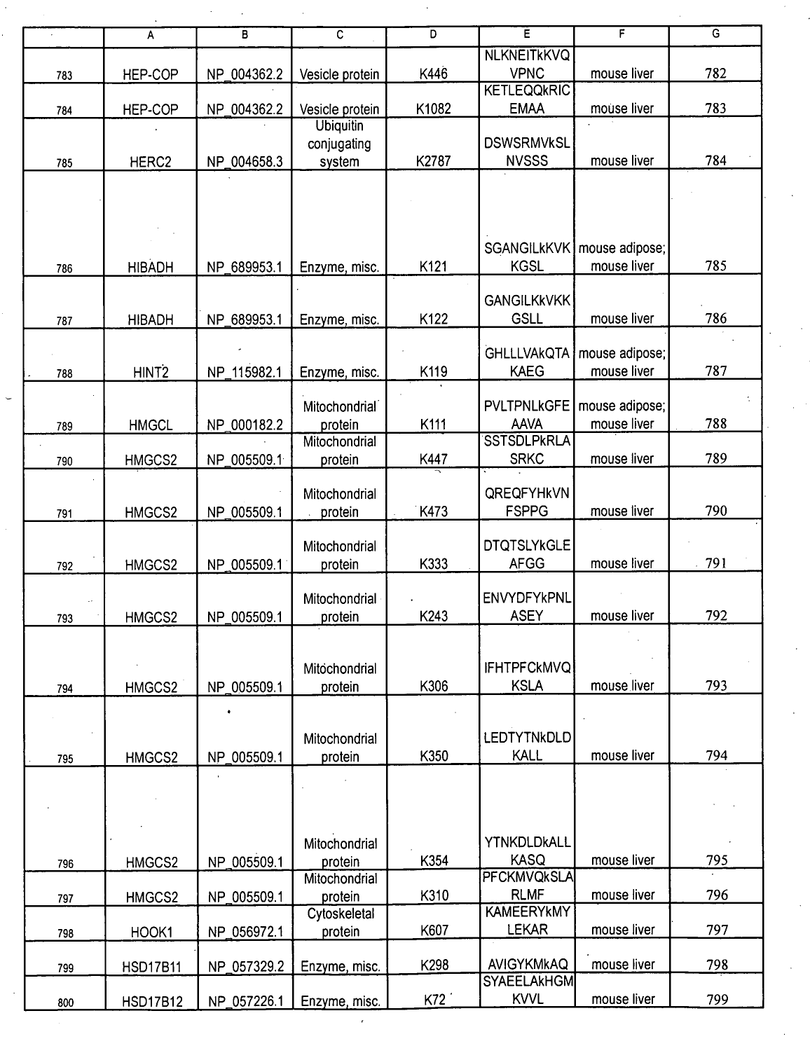

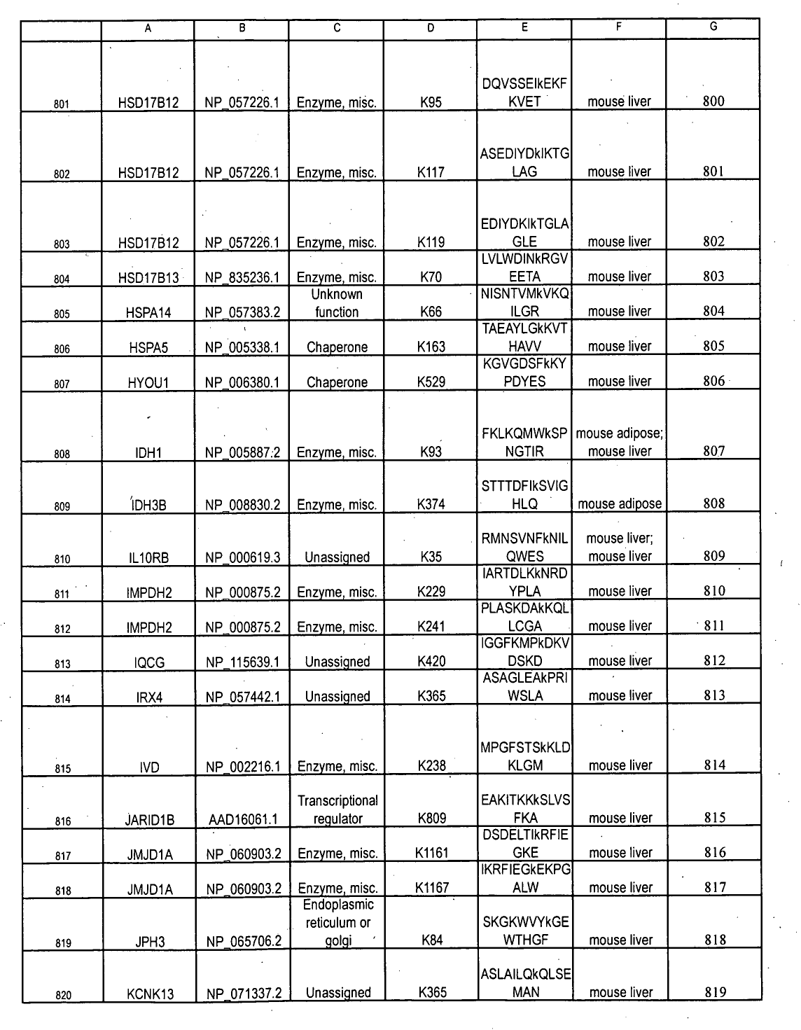

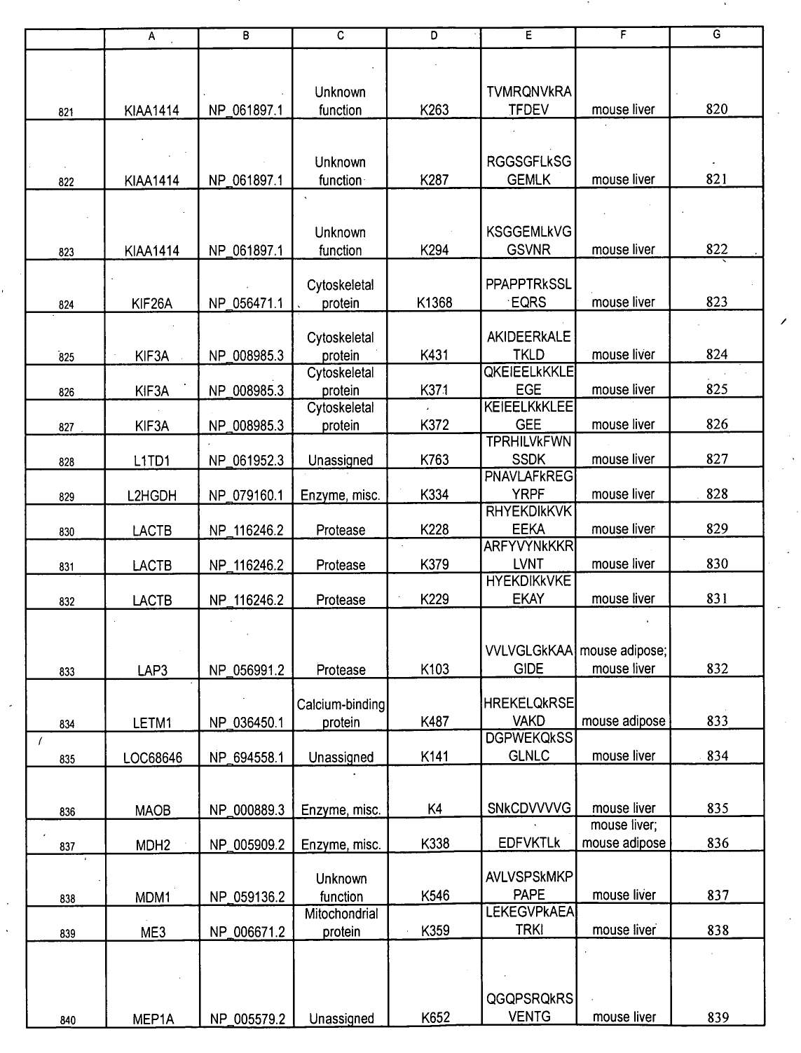

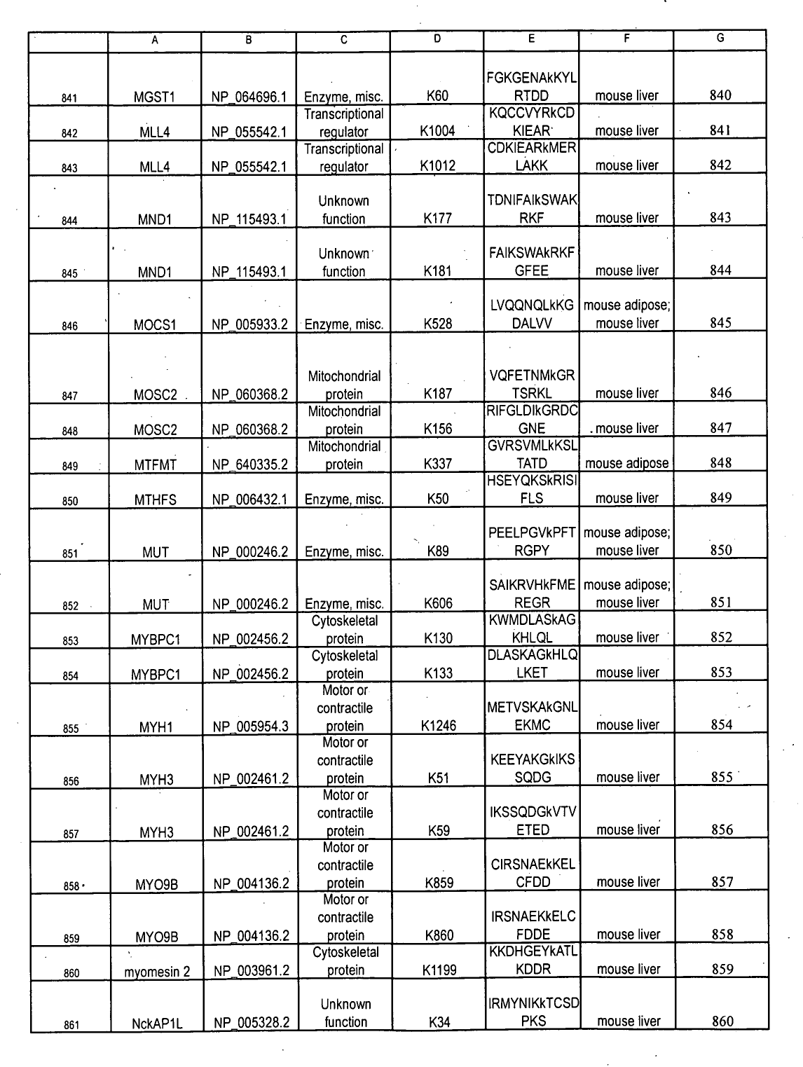

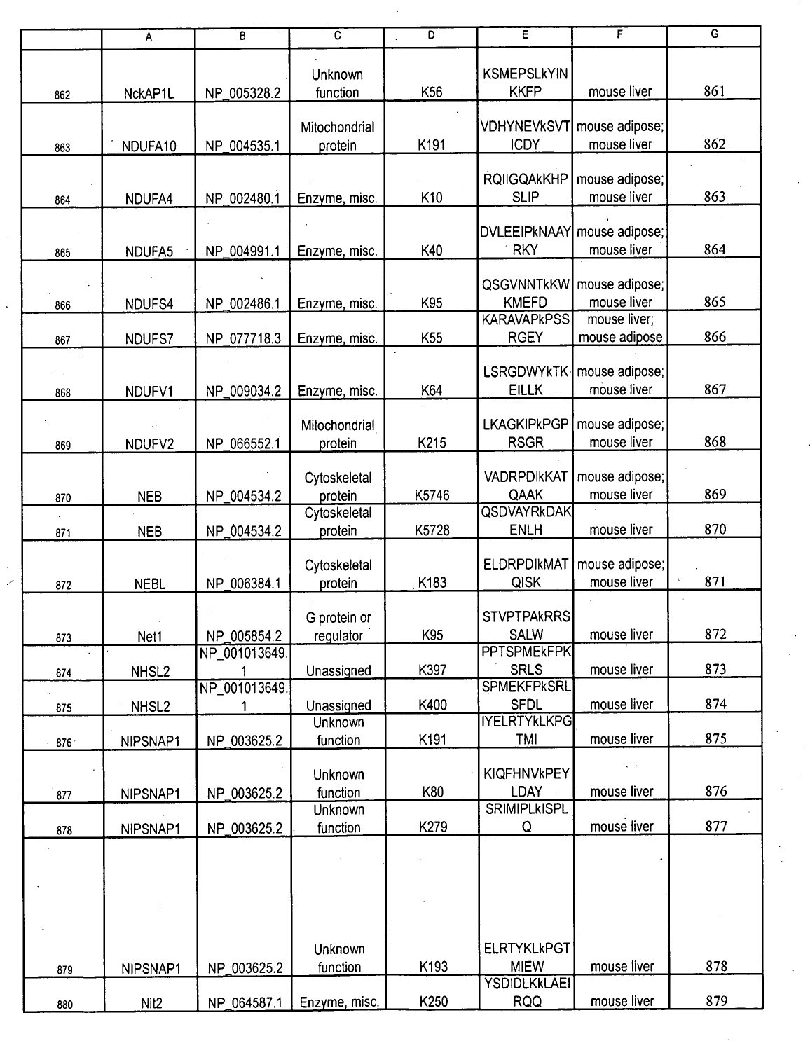

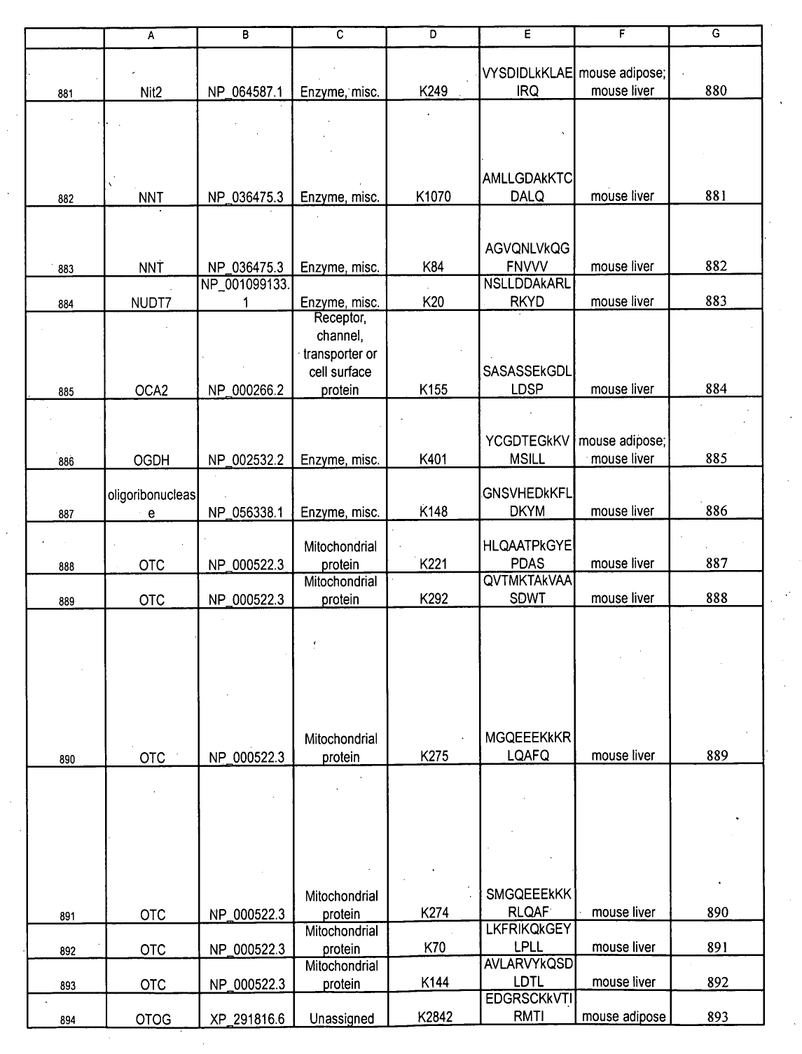

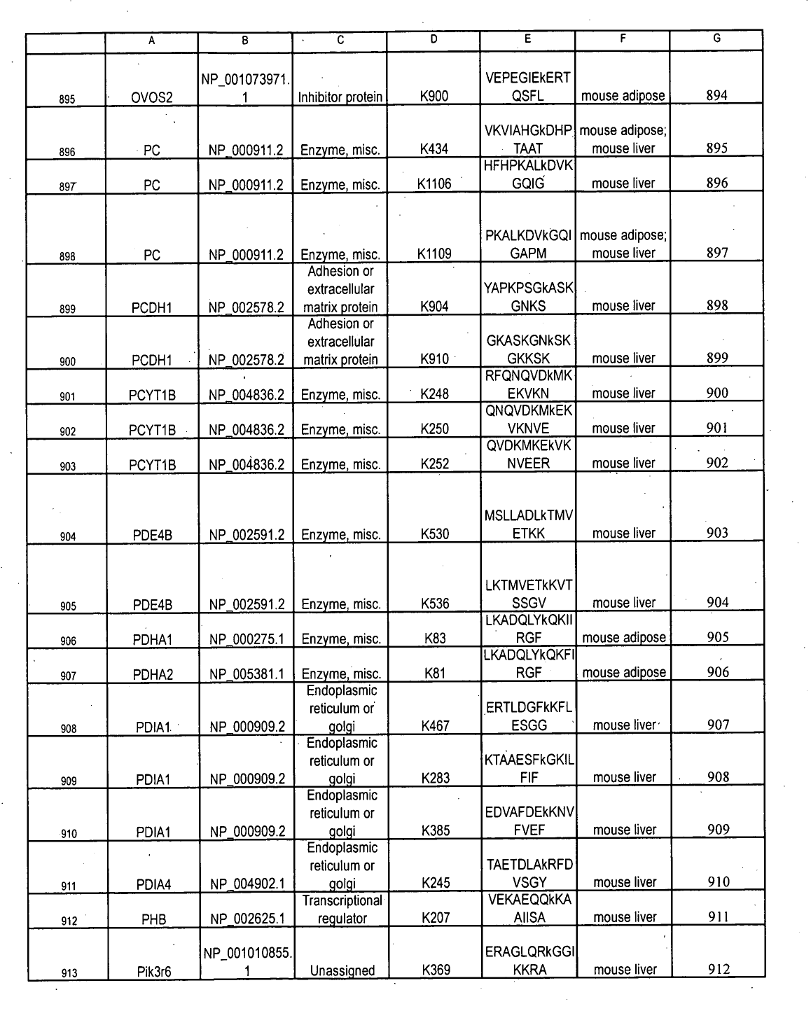

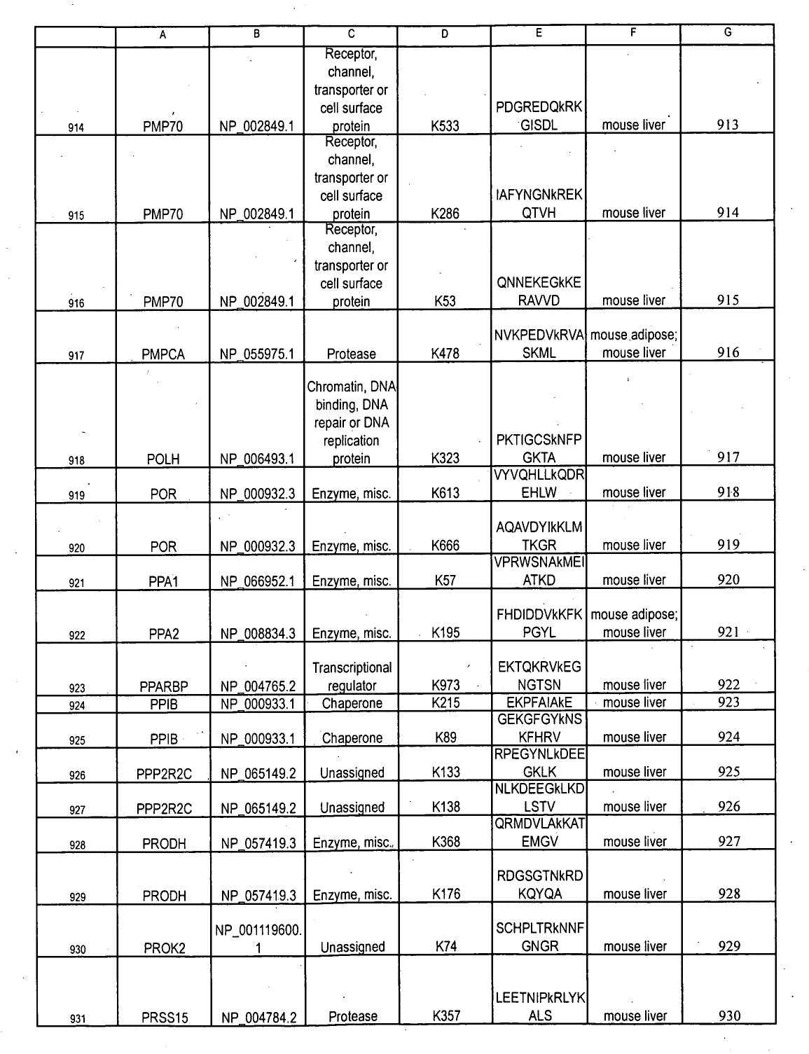

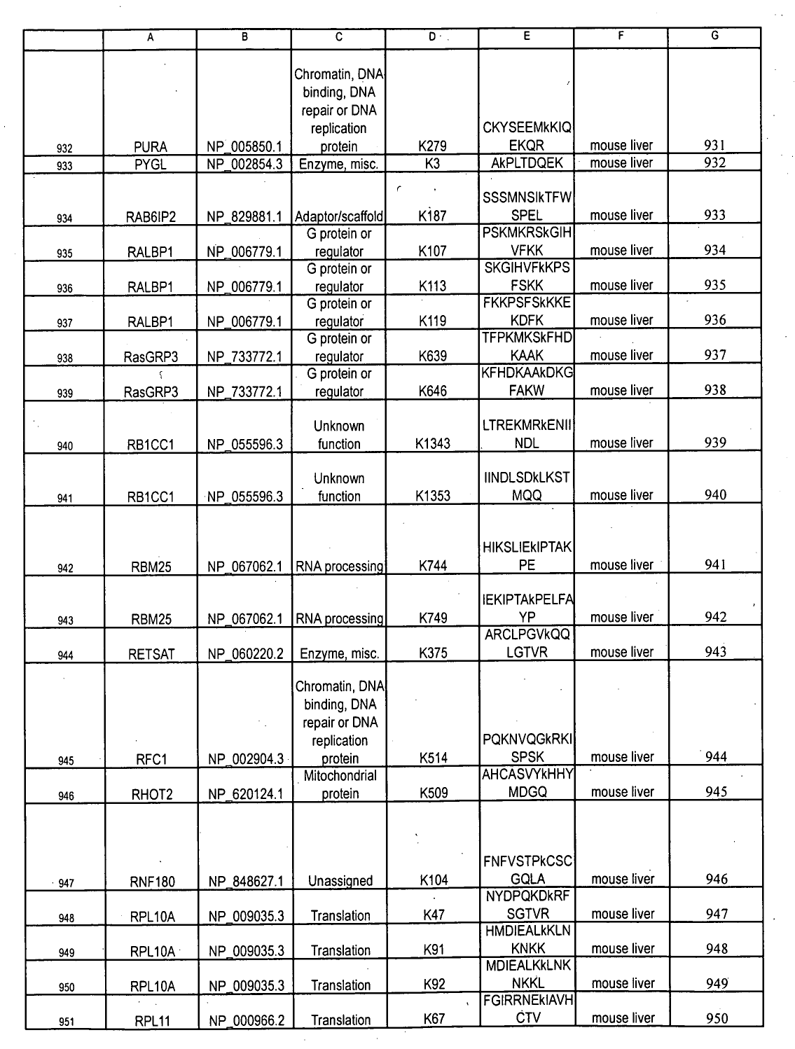

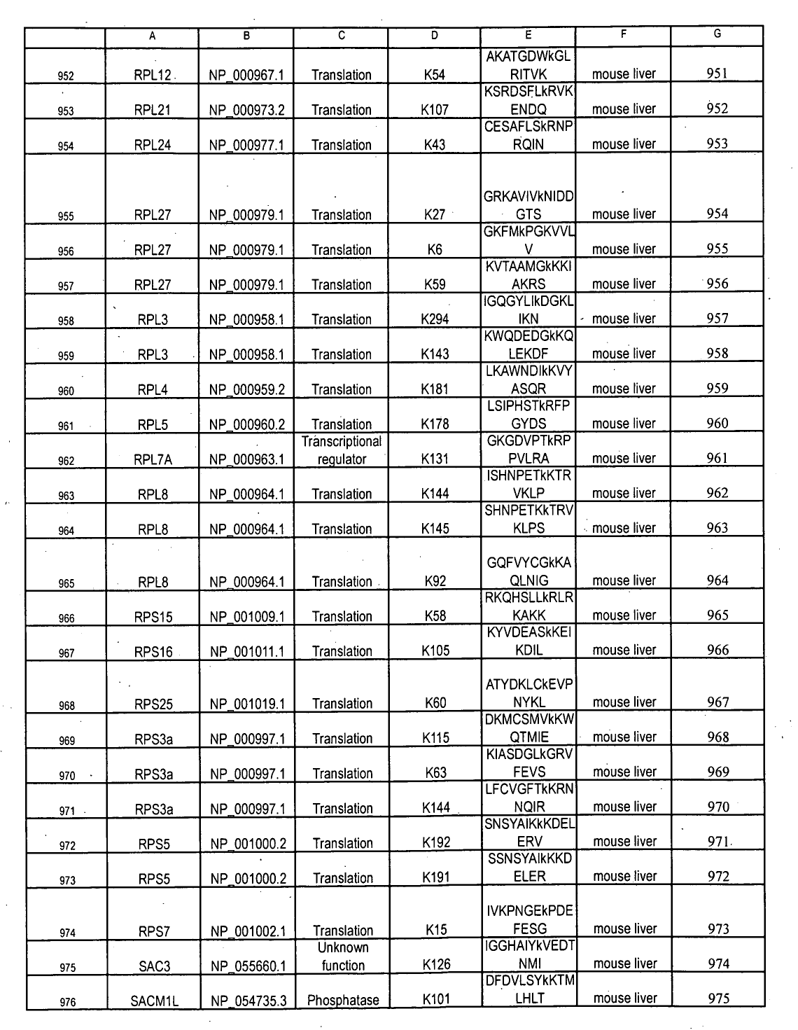

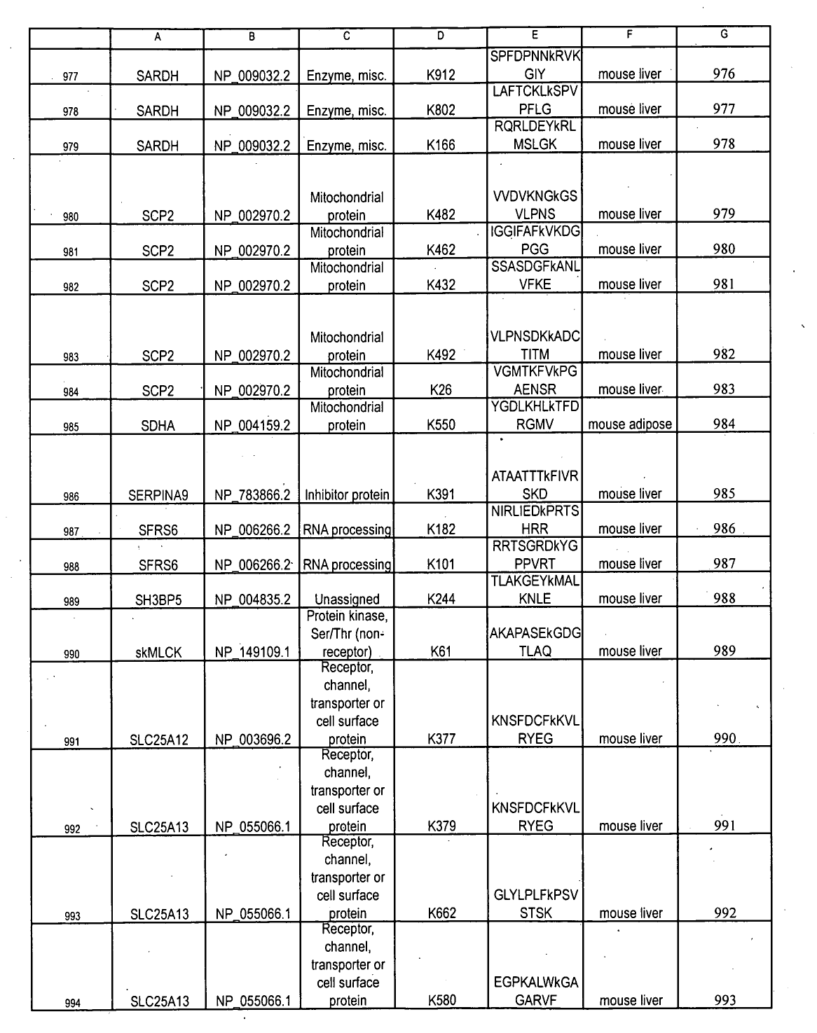

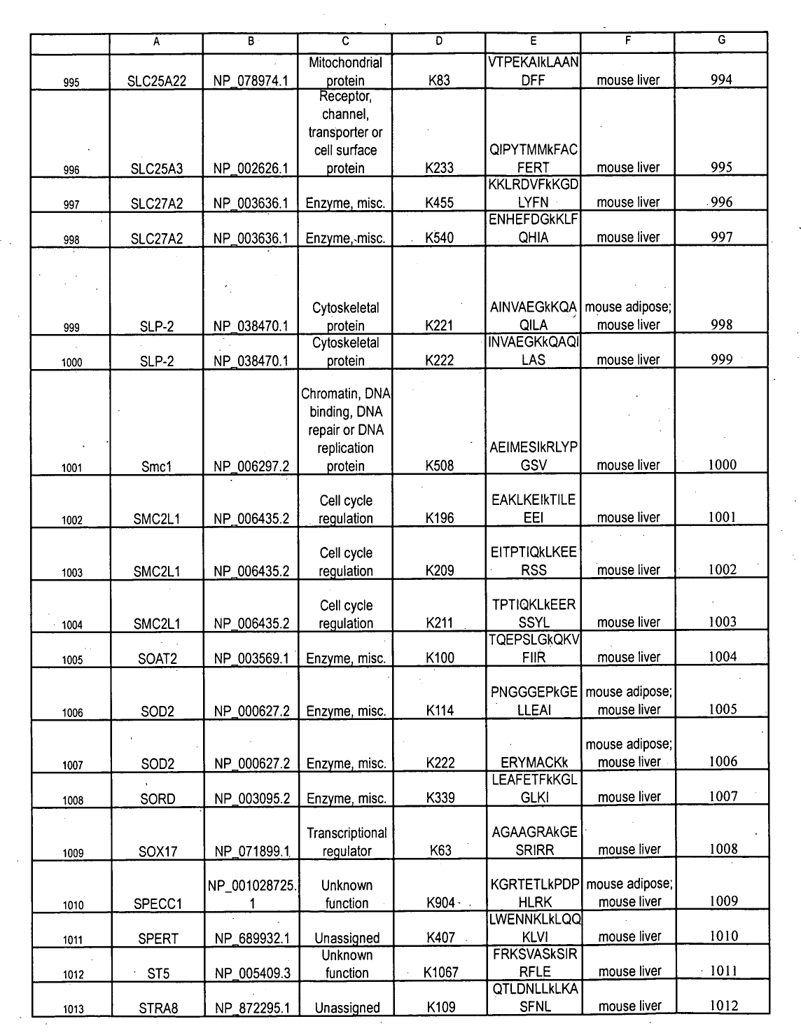

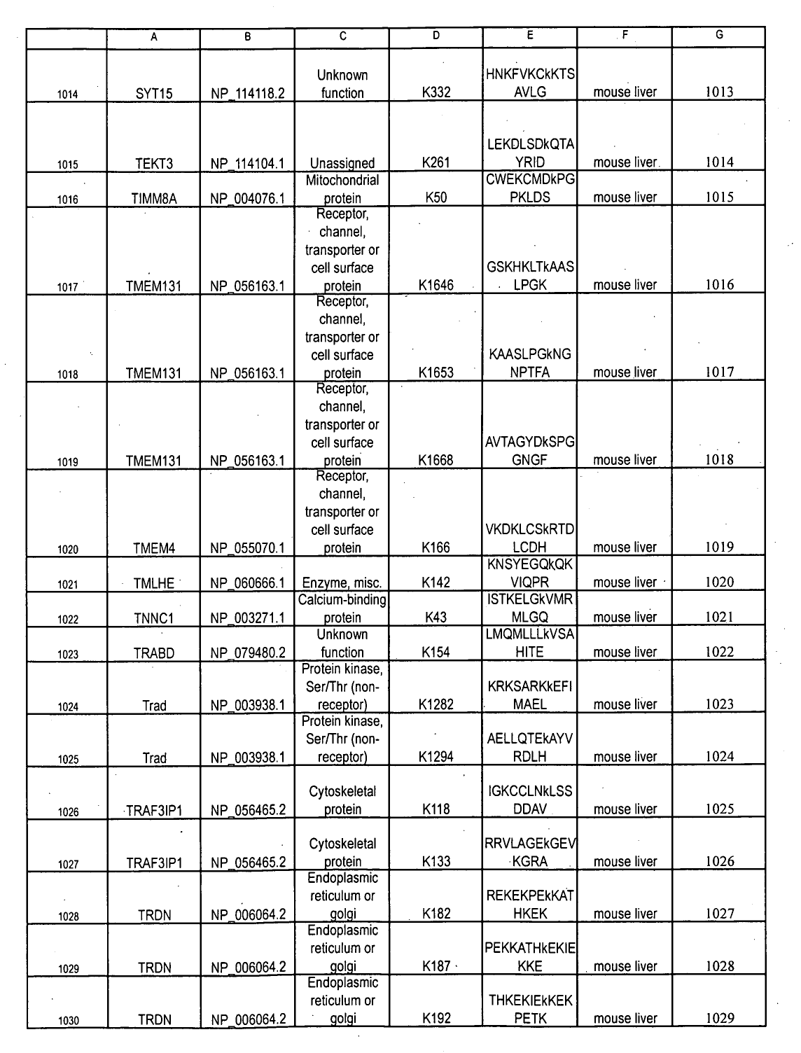

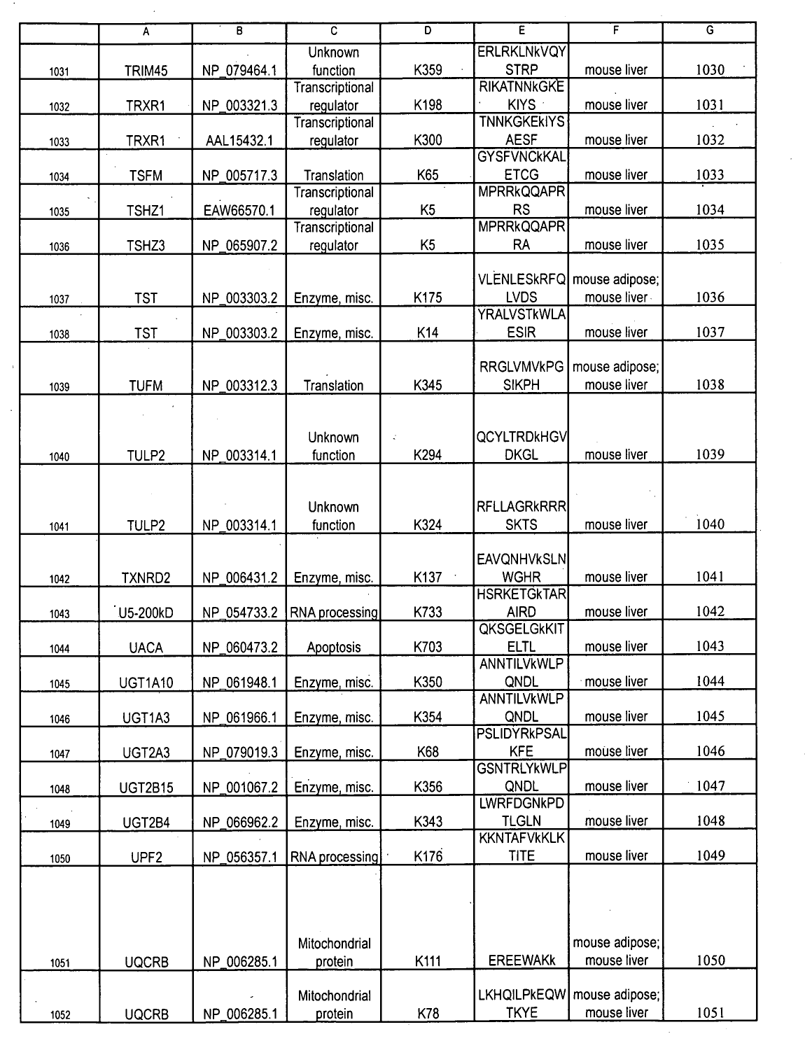

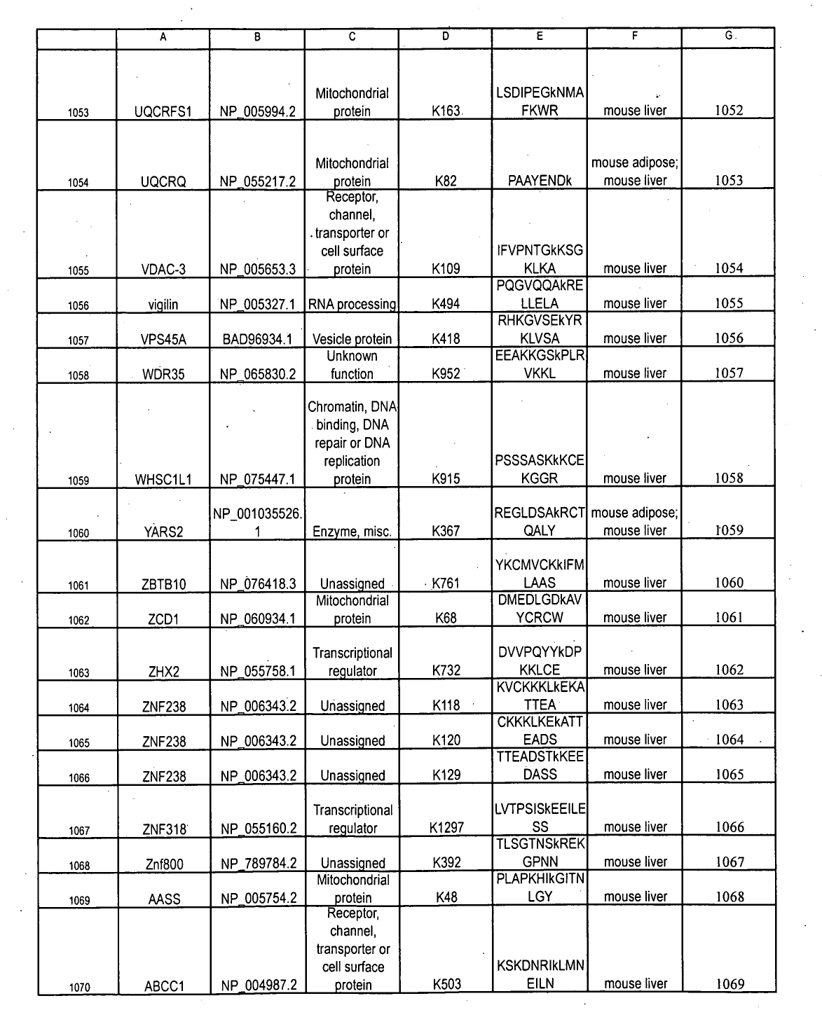

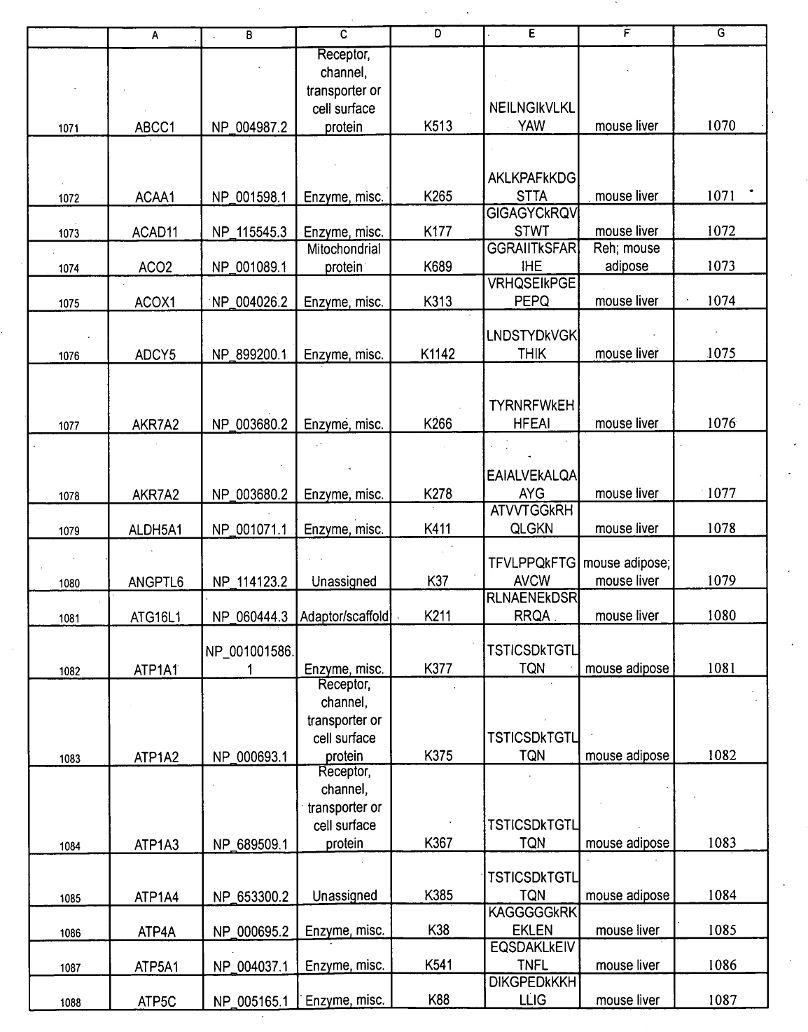

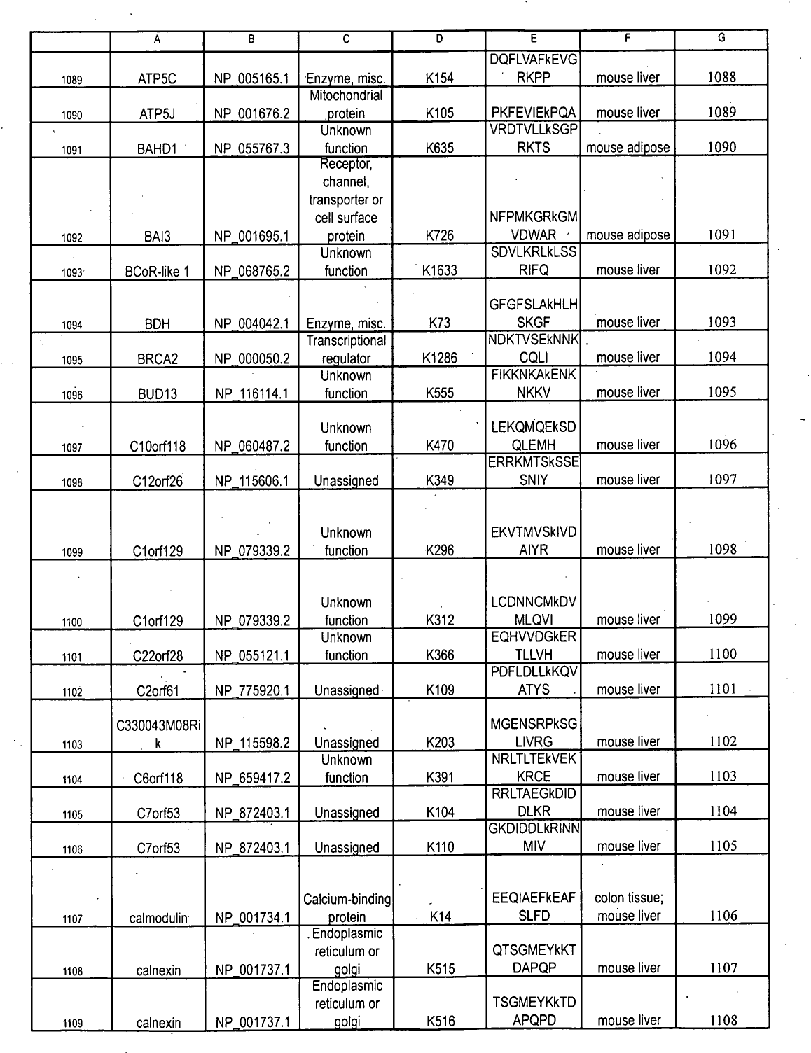

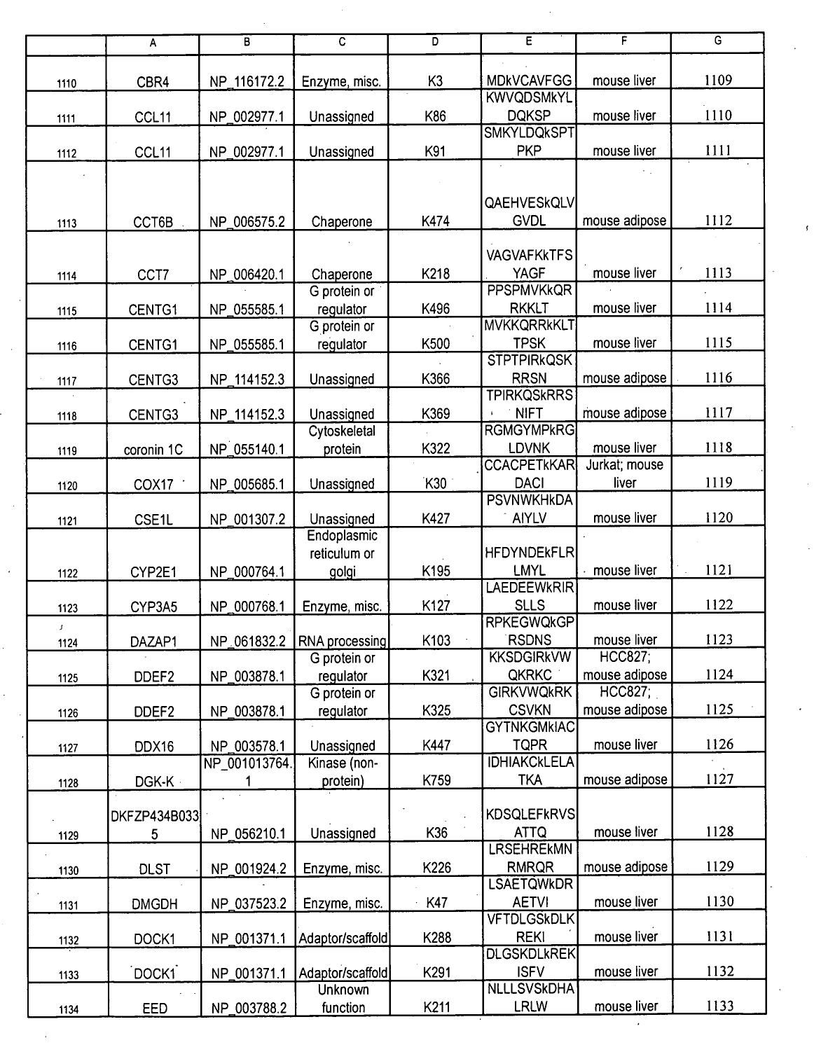

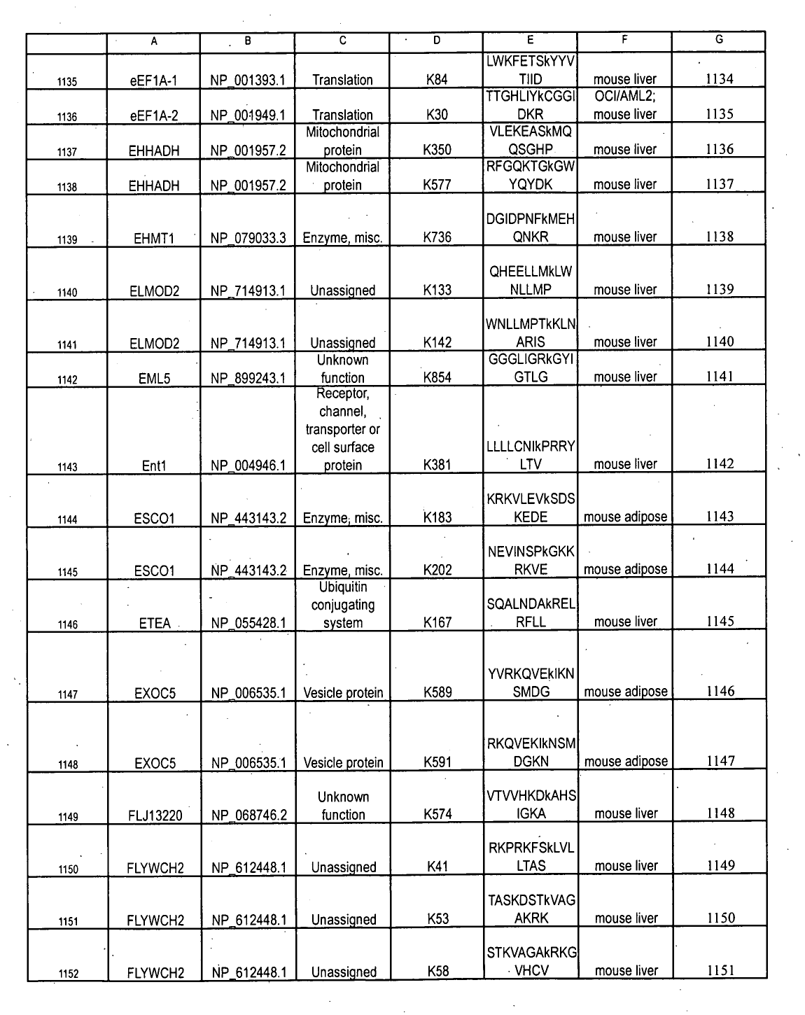

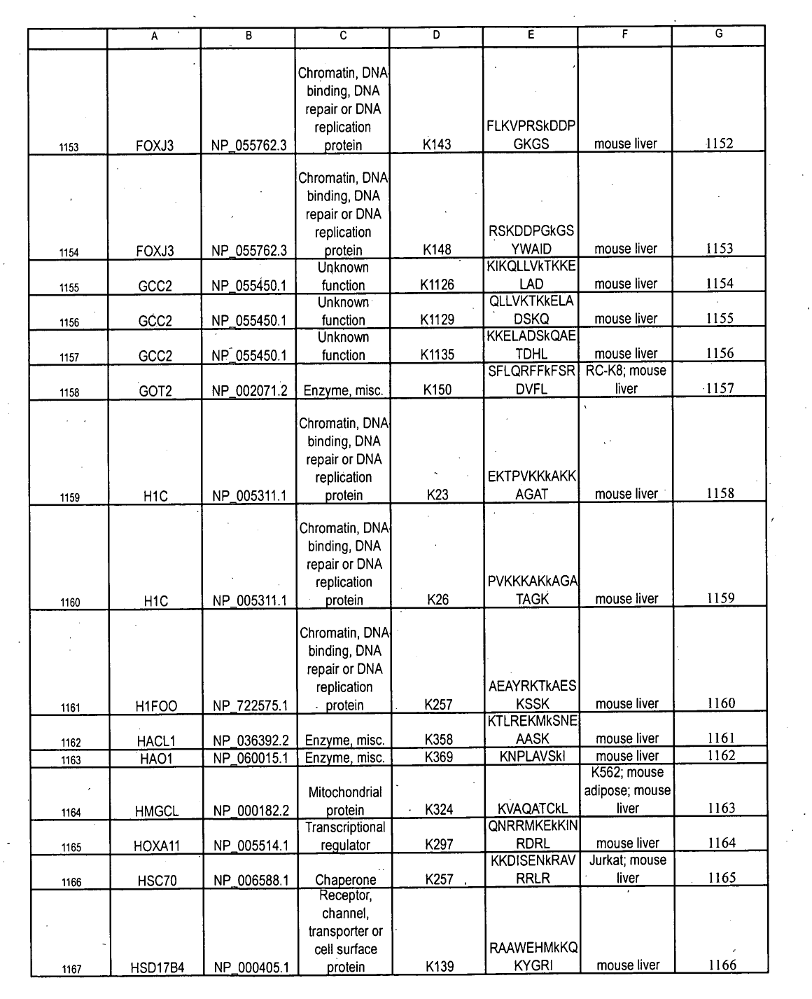

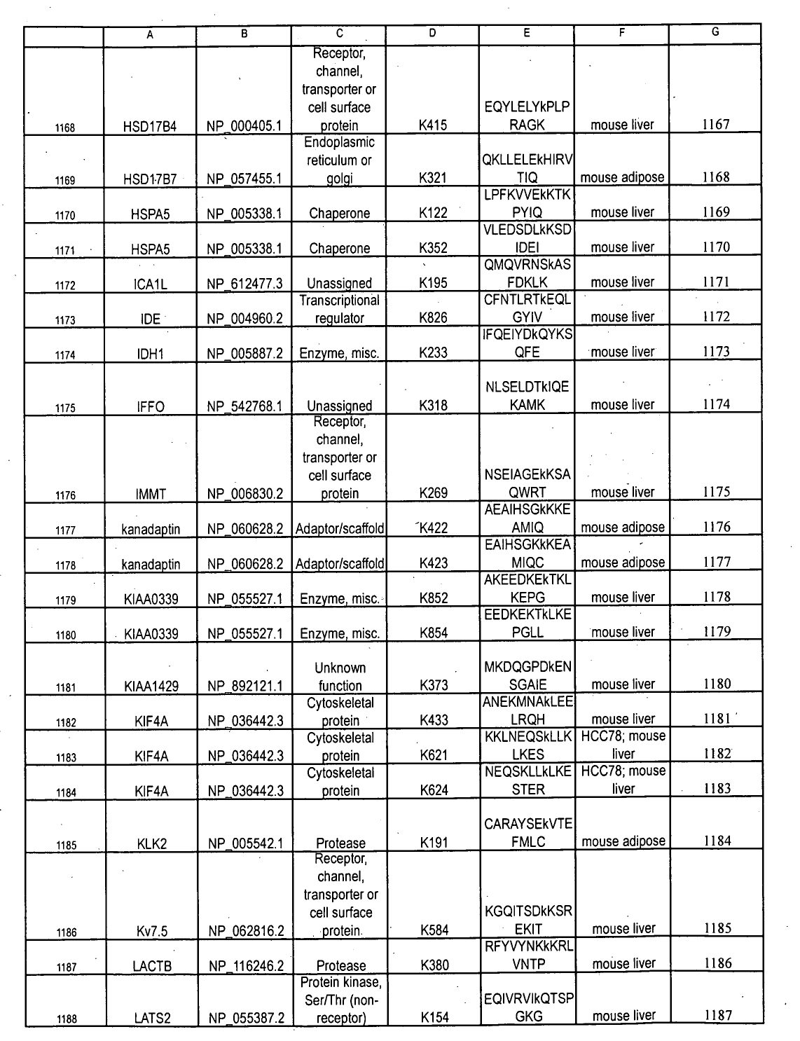

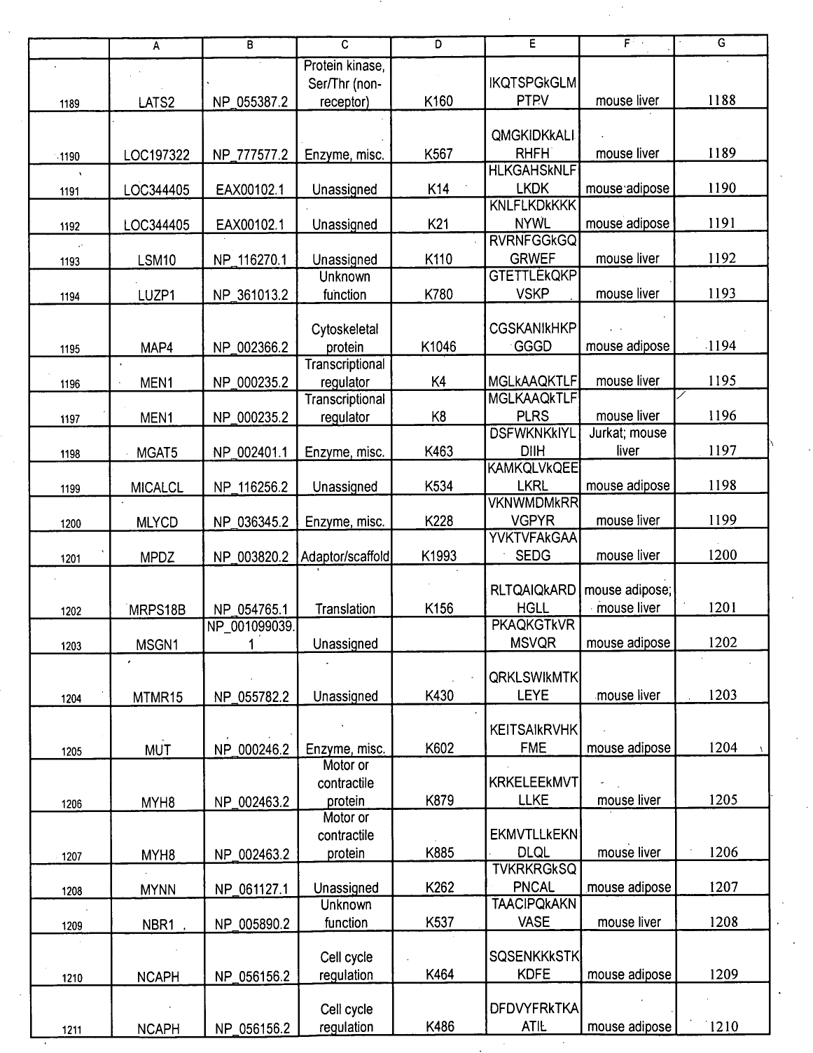

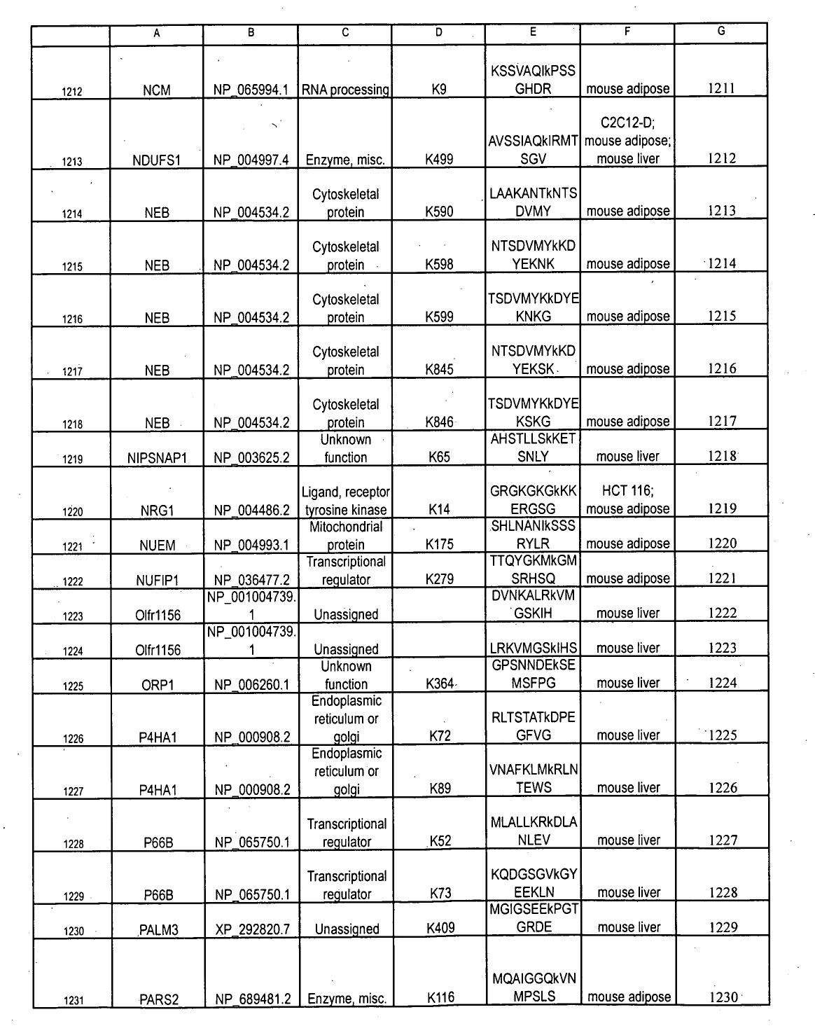

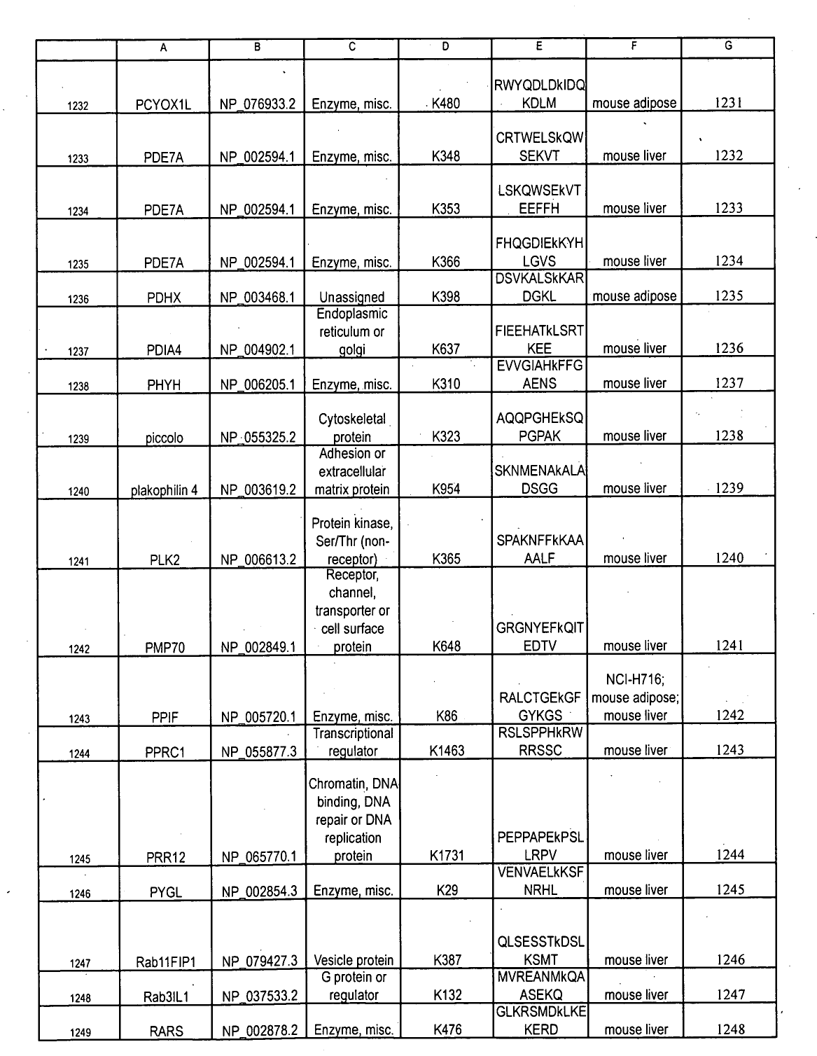

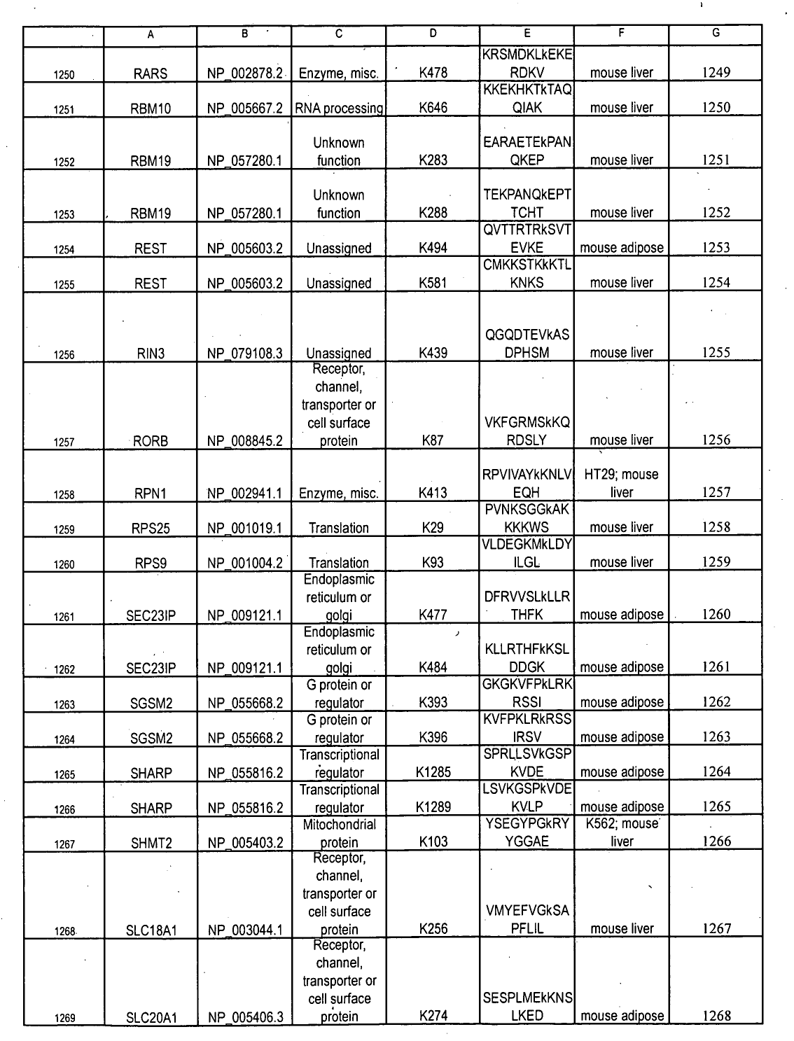

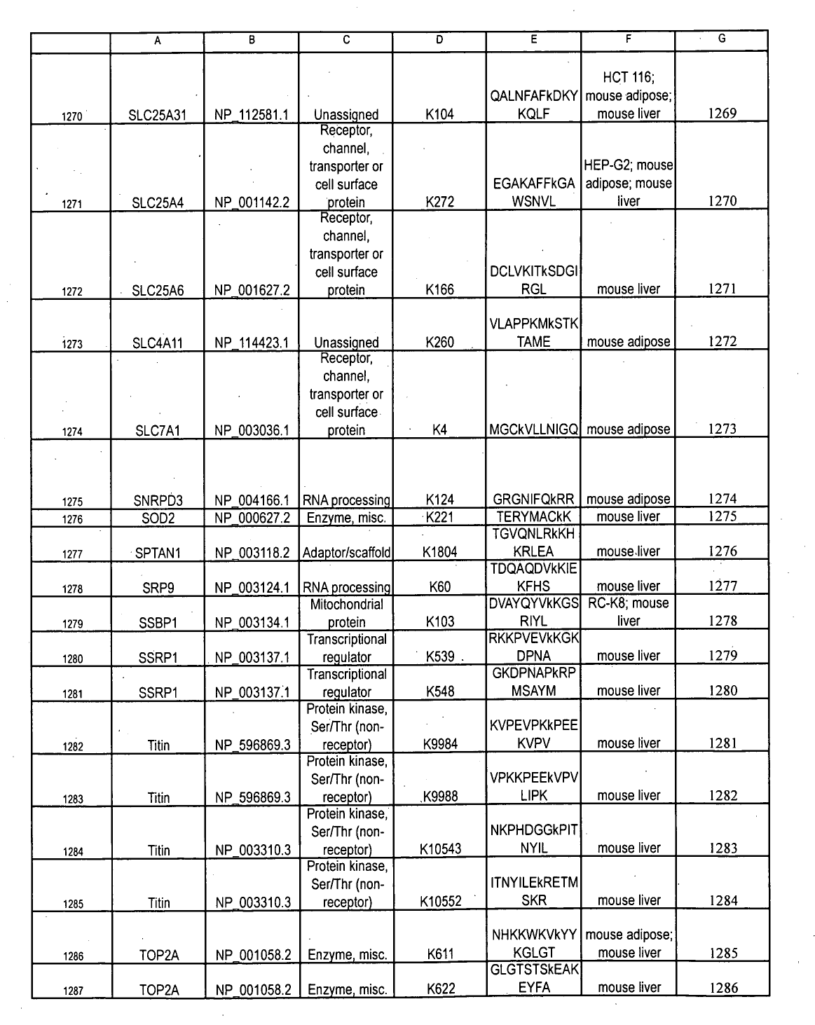

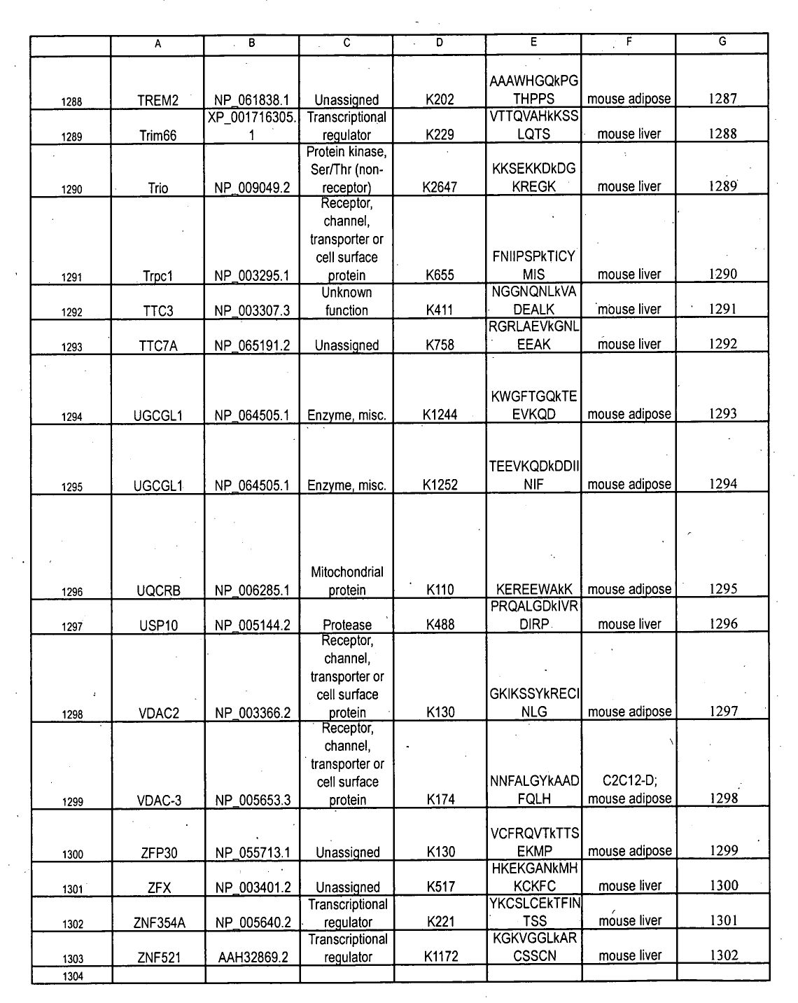

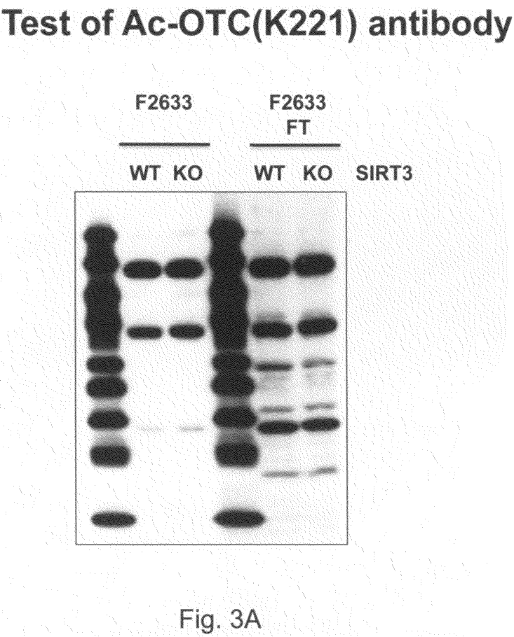

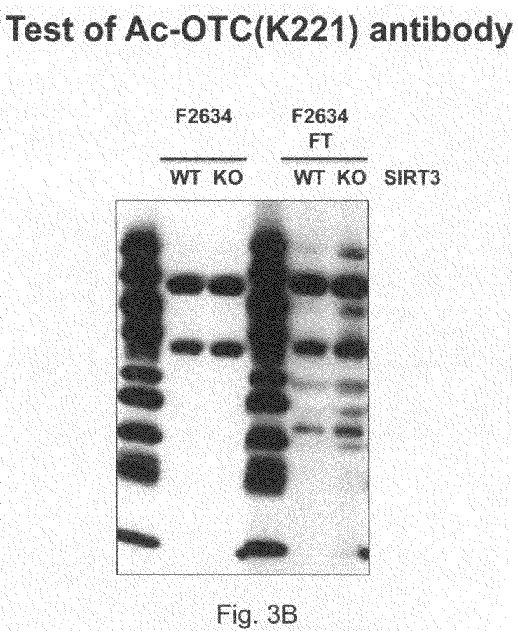

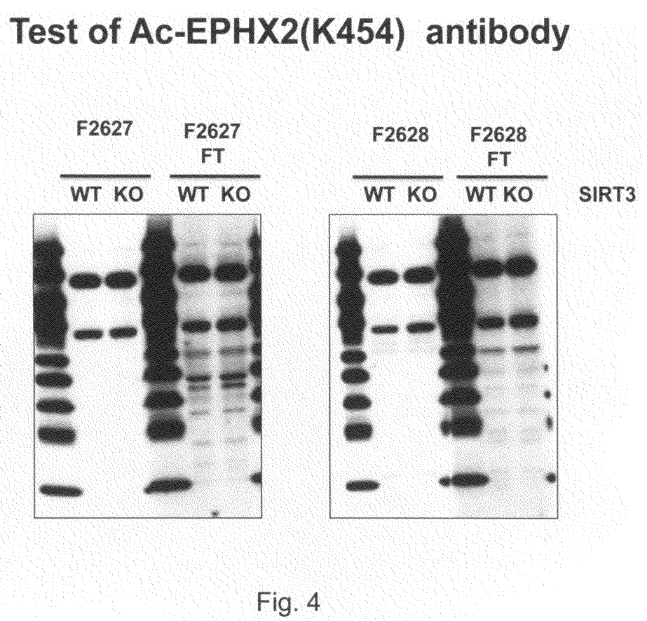

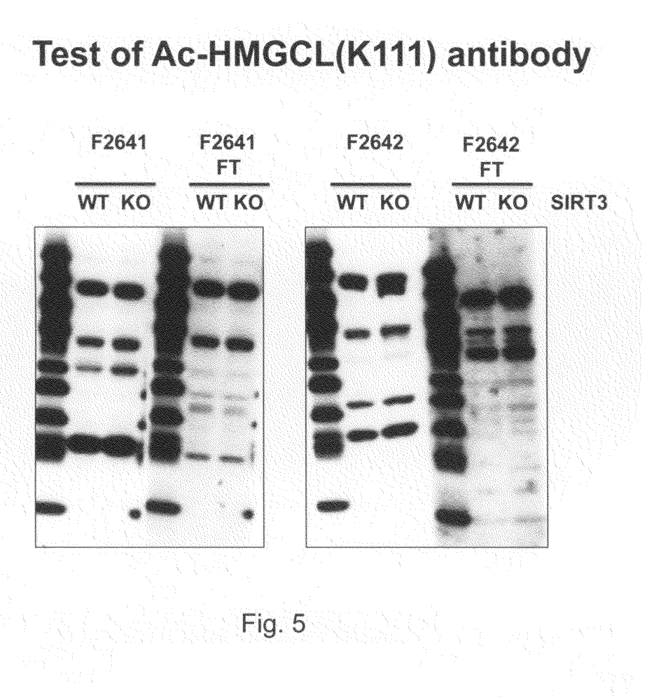

The inventors have begun to raise antibodies to at least some of the following acetylation sites: SEQ ID NO.: 525 (ACSM1); SEQ ID NO: 647 (CPS1); SEQ ID NO: 686 (DLST); SEQ ID NO: 708 (EPHX2); SEQ ID NO: 887 (OTC) and SEQ ID NO: 788 (HMGCL). FIGS. 3A and 3B show representative Western blotting analyses of mitrochondrial preparations made from wild-type (WT) or SIRT3-knock out mice using standard methods (mice and method described in, e.g., Lombard et al., Mol. Cell. Bio. 27(24):8807-14, 2007, incorporated herein by reference) using rabbit polyclonal antibodies that specifically bind to the aceylated lysine residue within SEQ ID NO: 887. FIG. 4 shows a representative Western blotting analysis of mitrochondrial preparations made from wild-type (WT) or SIRT3-knock out mice using standard methods (mice and method described in, e.g., Lombard et al., Mol. Cell. Bio. 27(24):8807-14, 2007, incorporated herein by reference) using antibodies that specifically bind to the aceylated lysine site at position 455 (also referred to as position 454) within SEQ ID NO: 708. FIG. 5 shows a representative Western blotting analysis of mitrochondrial preparations made from wild-type (WT) or SIRT3-knock out mice using standard methods (mice and method described in, e.g., Lombard et al., Mol. Cell. Bio. 27(24):8807-14, 2007, incorporated herein by reference) using antibodies that specifically bind to the aceylated lysine site at position 111 within SEQ ID NO: 788.

### Example 3

### Production of Acetylation Site-Specific Monoclonal Antibodies

Monoclonal antibodies that specifically bind a novel acetylation site of the invention (Table 1) only when the lysine residue is acetylated (and does not bind to the same sequence when the lysine is not acetylated) are produced according to standard methods by first constructing a synthetic peptide antigen comprising the acetylation site and then immunizing an animal to raise antibodies against the antigen, and harvesting spleen cells from such animals to produce fusion hybridomas, as further described below. Production of exemplary monoclonal antibodies is provided below.

### A. SUCLG1 (Lysine 192).

An 11 amino acid acetyl-peptide antigen, IGIMPGHIHk*K (SEQ ID NO: 375; k*=acetyl-lysine, which comprises the acetylation site derived from human SUCLG1 (a mitochondrial protein, Lys 192 being the acetylatable residue), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) acetylation site-specific polyclonal antibodies as described in Immunization/Screening below.

### B. SUCLG1 (Lysine 308).

A 16 amino acid acetyl-peptide antigen, MGHAGAIIAGGk*GGAK (SEQ ID NO: 376; k*=acetyl-lysine, which comprises the acetylation site derived from human SUCLG1 (a mitochondrial protein, Lys 308 being the acetylatable residue), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) acetylation site-specific polyclonal antibodies as described in Immunization/Screening below.

### C. ACOT2 (Lysine 104).

A 15 amino acid acetyl-peptide antigen, RASLRDEk*GALFQAH (SEQ ID NO: 491; k*=acetyl-lysine, which comprises the acetylation site derived from human ACOT2 (a mitochondrial protein, Lys 104 being the acetylatable residue), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) acetylation site-specific polyclonal antibodies as described in Immunization/Screening below.

### Immunization/Fusion/Screening.

A synthetic acetyl-peptide antigen as described in A-C above is coupled to KLH, and BALB/C mice are injected intradermally (ID) on the back with antigen in complete Freunds adjuvant (e.g., 50 μg antigen per mouse). The mice are boosted with same antigen in incomplete Freund adjuvant (e.g. 25 μg antigen per mouse) every three weeks. After the fifth boost, the animals are sacrificed and spleens are harvested.

Harvested spleen cells are fused to SP2/0 mouse myeloma fusion partner cells according to the standard protocol of Kohler and Milstein (1975). Colonies originating from the fusion are screened by ELISA for reactivity to the acetyl-peptide and non-acetyl-peptide forms of the antigen and by Western blot analysis (as described in Example 1 above). Colonies found to be positive by ELISA to the acetyl-peptide while negative to the non-acetyl-peptide are further characterized by Western blot analysis. Colonies found to be positive by Western blot analysis are subcloned by limited dilution. Mouse ascites are produced from a single clone obtained from subcloning, and tested for acetyl-specificity on ELISA. Clones identified as positive on Western blot analysis using cell culture supernatant as having acetyl-specificity, as indicated by a strong band in the induced lane and a weak band in the uninduced lane of the blot, are isolated and subcloned as clones producing monoclonal antibodies with the desired specificity.

Ascites fluid from isolated clones may be further tested by Western blot analysis. The ascites fluid should produce similar results on Western blot analysis as observed previously with the cell culture supernatant, indicating acetyl-specificity against the acetylated target.

### Example 4

Production and Use of AQUA Peptides for Detecting and Quantitating Acetylation at a Novel Acetylation Site

Heavy-isotope labeled peptides (AQUA peptides (internal standards)) for the detecting and quantitating a novel acetylation site of the invention (Table 1) only when the lysine residue is acetylated are produced according to the standard AQUA methodology (see Gygi et al., Gerber et al., supra.) methods by first constructing a synthetic peptide standard corresponding to the acetylation site sequence and incorporating a heavy-isotope label. Subsequently, the MSⁿ and LC-SRM signature of the peptide standard is validated, and the AQUA peptide is used to quantify native peptide in a biological sample, such as a digested cell extract. Production and use of exemplary AQUA peptides is provided below.

### A. ALDH6A1 (Lysine 87).

An AQUA peptide comprising the sequence, DAAIASCk*RAFPAWA (SEQ ID NO: 549; k*=acetyl-lysine; Proline being ¹⁴C/¹⁵N-labeled, as indicated in bold), which comprises the phosphorylation site derived from ALDH6A1 (an enzyme protein, K87 being the phosphorylatable residue), is constructed according to standard synthesis techniques using, e.g., a Rainin/Protein Technologies, Inc., Symphony peptide synthesizer (see Merrifield, supra.) as further described below in Synthesis & MS/MS Signature. The ALDH6A1 (K87) AQUA peptide is then spiked into a biological sample to quantify the amount of phosphorylated ALDH6A1 (K87) in the sample, as further described below in Analysis & Quantification.

### B. COX5B (Lysine 86).

An AQUA peptide comprising the sequence, LVPSISNk*RIVGCIC (SEQ ID NO: 631; k*=acetyl-lysine; Valine being ¹⁴C/¹⁵N-labeled, as indicated in bold), which comprises the phosphorylation site derived from COX5B (an enzyme protein, K86 being the phosphorylatable residue), is constructed according to standard synthesis techniques using, e.g., a Rainin/Protein Technologies, Inc., Symphony peptide synthesizer (see Merrifield, supra.) as further described below in Synthesis & MS/MS Signature. The COX5B (K86) AQUA peptide is then spiked into a biological sample to quantify the amount of phosphorylated COX5B (K86) in the sample, as further described below in Analysis & Quantification.

### B. COX5B (Lysine 121).

An AQUA peptide comprising the sequence, PRCGAHYk*LVPQQLA (SEQ ID NO: 631; k*=acetyl-lysine; Leucine being ¹⁴C/¹⁵N-labeled, as indicated in bold), which comprises the phosphorylation site derived from COX5B (an enzyme protein, K121 being the phosphorylatable residue), is constructed according to standard synthesis techniques using, e.g., a Rainin/Protein Technologies, Inc., Symphony peptide synthesizer (see Merrifield, supra.) as further described below in Synthesis & MS/MS Signature. The COX5B (K121) AQUA peptide is then spiked into a biological sample to quantify the amount of phosphorylated COX5B (K121) in the sample, as further described below in Analysis & Quantification.

### Synthesis & MS/MS Spectra.

Fluorenylmethoxycarbonyl (Fmoc)-derivatized amino acid monomers may be obtained from AnaSpec (San Jose, Calif.). Fmoc-derivatized stable-isotope monomers containing one ¹⁵N and five to nine ¹³C atoms may be obtained from Cambridge Isotope Laboratories (Andover, Mass.). Preloaded Wang resins may be obtained from Applied Biosystems. Synthesis scales may vary from 5 to 25 μmol. Amino acids are activated in situ with 1-H-benzotriazolium, 1-bis(dimethylamino) methylene]-hexafluorophosphate (1-),3-oxide:1-hydroxybenzotriazole hydrate and coupled at a 5-fold molar excess over peptide. Each coupling cycle is followed by capping with acetic anhydride to avoid accumulation of one-residue deletion peptide by-products. After synthesis peptide-resins are treated with a standard scavenger-containing trifluoroacetic acid (TFA)-water cleavage solution, and the peptides are precipitated by addition to cold ether. Peptides (i.e. a desired AQUA peptide described in A-D above) are purified by reversed-phase C18 HPLC using standard TFA/acetonitrile gradients and characterized by matrix-assisted laser desorption ionization-time of flight (Biflex III, Bruker Daltonics, Billerica, Mass.) and ion-trap (ThermoFinnigan, LCQ DecaXP or LTQ) MS.

MS/MS spectra for each AQUA peptide should exhibit a strong y-type ion peak as the most intense fragment ion that is suitable for use in an SRM monitoring/analysis. Reverse-phase microcapillary columns (0.1 Å˜150-220 mm) are prepared according to standard methods. An Agilent 1100 liquid chromatograph may be used to develop and deliver a solvent gradient [0.4% acetic acid/0.005% heptafluorobutyric acid (HFBA)/7% methanol and 0.4% acetic acid/0.005% HFBA/65% methanol/35% acetonitrile] to the microcapillary column by means of a flow splitter. Samples are then directly loaded onto the microcapillary column by using a FAMOS inert capillary autosampler (LC Packings, San Francisco) after the flow split. Peptides are reconstituted in 6% acetic acid/0.01% TFA before injection.

### Analysis & Quantification.

Target protein (e.g. a acetylated proteins of A-D above) in a biological sample is quantified using a validated AQUA peptide (as described above). The IAP method is then applied to the complex mixture of peptides derived from proteolytic cleavage of crude cell extracts to which the AQUA peptides have been spiked in.

LC-SRM of the entire sample is then carried out. MS/MS may be performed by using a ThermoFinnigan (San Jose, Calif.) mass spectrometer (LCQ DecaXP ion trap or TSQ Quantum triple quadrupole or LTQ). On the DecaXP, parent ions are isolated at 1.6 m/z width, the ion injection time being limited to 150 ms per microscan, with two microscans per peptide averaged, and with an AGC setting of 1×10⁸; on the Quantum, Q1 is kept at 0.4 and Q3 at 0.8 m/z with a scan time of 200 ms per peptide. On both instruments, analyte and internal standard are analyzed in alternation within a previously known reverse-phase retention window; well-resolved pairs of internal standard and analyte are analyzed in separate retention segments to improve duty cycle. Data are processed by integrating the appropriate peaks in an extracted ion chromatogram (60.15 m/z from the fragment monitored) for the native and internal standard, followed by calculation of the ratio of peak areas multiplied by the absolute amount of internal standard (e.g., 500 fmol).

### Example 5

### Label Free Quantitation

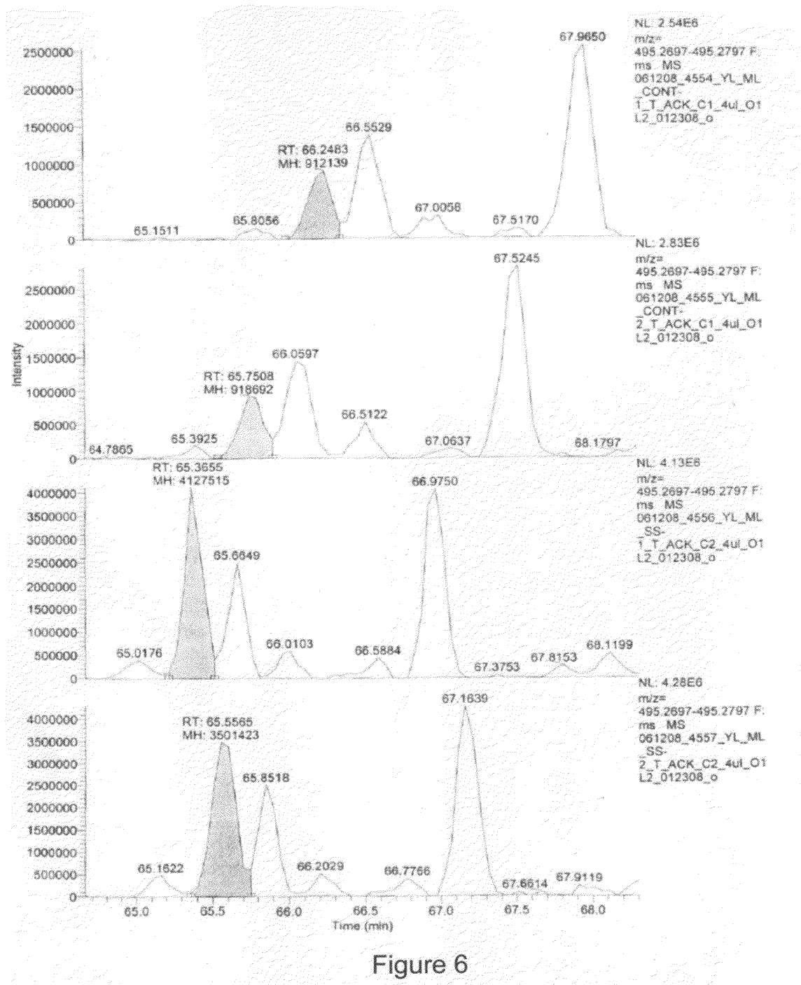

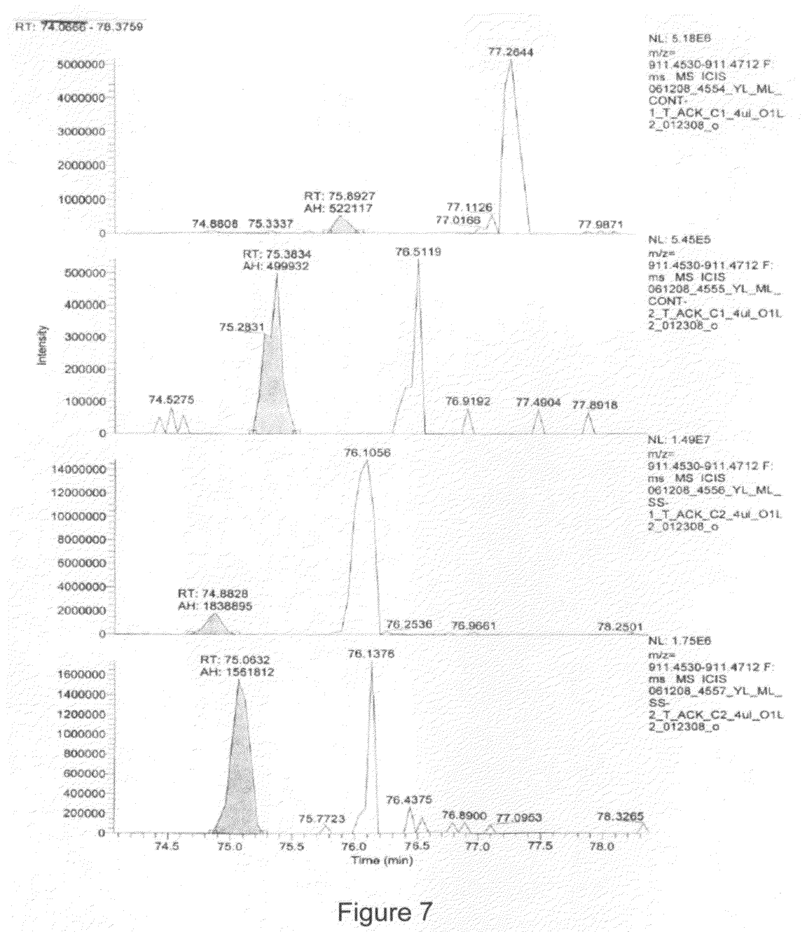

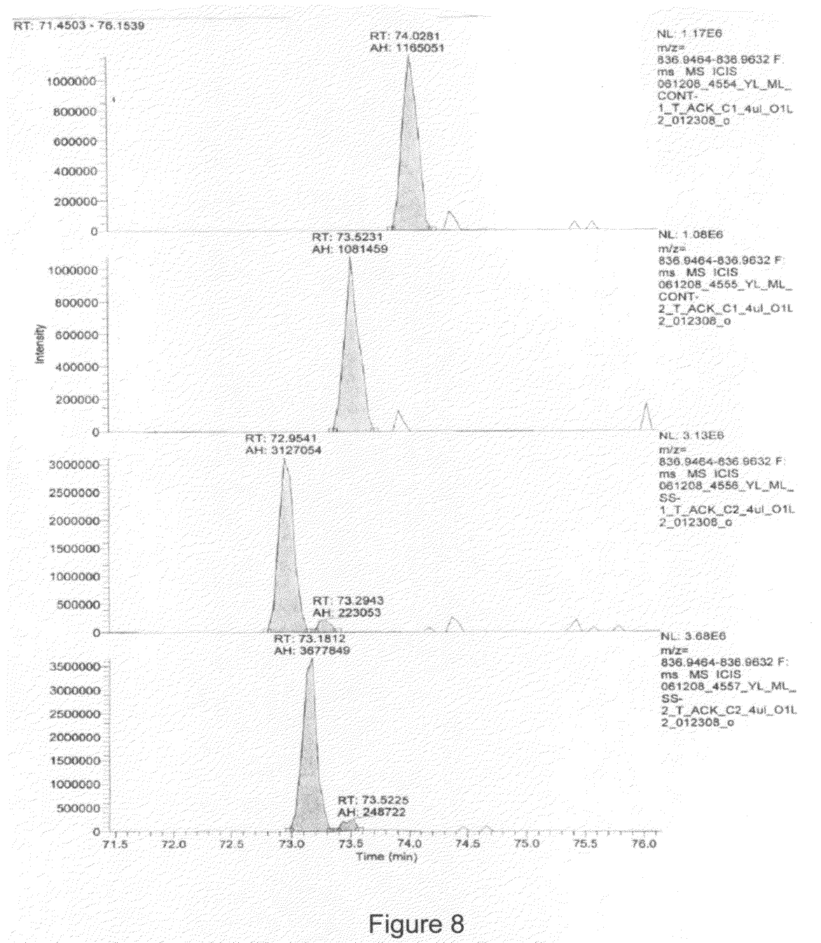

Each MS/MS spectrum arising from a parent ion observed during a survey MS scan was linked to the intensity of that parent ion at its chromatographic apex, essentially measuring the abundance of the peptide in the sample. All parent ion intensities were extracted from each sample's ion chromatogram using a signal processing algorithm (TurboSequest). Changes in acetylated peptide levels were determined from the ratio of raw intensities between treated and untreated samples. Non-limiting representative results for CSP1, GOT1, and HADHA are are represented graphically in FIGS. 6-8, respectively.

What is claimed is:

1. An antibody or antigen-binding fragment thereof, wherein the antibody specifically binds to an amino acid sequence comprising an acetylation site identified in Table 1 when the lysine in Column D is acetylated, and wherein the antibody does not bind to said amino acid sequence when the lysine is not acetylated.

2. An antibody or antigen-binding fragment thereof, wherein the antibody specifically binds to an amino acid sequence comprising an acetylation site identified in Table 1 when the lysine in Column D is not acetylated, and wherein the antibody does not bind to said amino acid sequence when the lysine is acetylated.

3. The antibody or antigen-binding fragment thereof of claim 1, wherein the acetylation site occurs in a protein in Table 1 that is selected from the group consisting of: a chromatin or DNA binding/repair/replication protein, an enzyme, an RNA binding protein, an RNA processing protein, a transcriptional regulator, a translation protein, a ubiquitin conjugating system protein, a cytoskeletal protein, mitochondrial proteins, an adaptor/scaffold protein and a receptor/channel/transporter/cell surface protein.

4. The antibody or antigen-binding fragment thereof of claim 1, wherein the antibody specifically binds to an amino acid sequence comprising a lysine acetylation site selected from the group consisting of SEQ ID NOs: 46 (ACAA2); 48 (ACAA2); 50 (ACAA2); 71 (ACAT1); 72 (ACAT1); 333 (CLYBL); 336 (CS); 351 (DLD); 375 (SUCLG1); 376 (SUCLG1); 417 (SLC25A4); 420 (SLC25A5); 491 (ACOT2); 549 (ALDH6A1); 631 (COX5B); 632 (COX5B); 643 (CPS1); 646 (CPS1); 647 (CPS1); 649 (CPS1); 687 (DLST); 697 (EHHADH); 715 (ETFDH); 739 (GCAT); 757 (GOT2); 759 (GOT2); 895 (PC); 1072 (ACAD11); and 1269 (SLC25A31).

5. The antibody or antigen-binding fragment thereof of claim 1, wherein the antibody specifically binds to an amino acid sequence comprising a lysine acetylation site selected from the group consisting of SEQ ID NOs: 708 (EPHX2), 887 (OTC), and 788 (HMGCL).

6. The antibody or antigen-binding fragment thereof of claim 1, wherein said antibody or antibody fragment is selected from the group consisting of a polyclonal antibody, a monoclonal antibody or antibody fragment, a recombinant antibody, a camelid antibody, a bispecific antibody, a diabody, a chimerized or chimeric antibody or antibody fragment, a humanized antibody or antibody fragment, a deimmunized human antibody or antibody fragment, a fully human antibody or antibody fragment, a single chain antibody, an Fv, an Fd, an Fab, an Fab′, and an F(ab′)₂.

7. The antibody or antigen-binding fragment thereof of claim 1, wherein the antibody or antibody fragment is conjugated to a cytotoxic agent.

8. A method of treating or preventing metabolic disorder in a subject, wherein the carcinoma is associated with lysine acetylation or deacetylation at a acetylation site in Table 1, comprising administering to the subject a therapeutically effective amount of an antibody or antigen-binding fragment thereof that binds to the acetylation site.

9. The method of claim 8, wherein the antibody or antigen binding fragment thereof binds to the acetylation site when the lysine identified in Column D is acetylated, and wherein the antibody does not bind to the acetylation site when the lysine identified in Column D is not acetylated.

10. The method of claim 8, wherein the antibody or antigen binding fragment thereof binds to the acetylation site when the lysine identified in Column D is not acetylated, and wherein the antibody does not bind to the acetylation site when the lysine identified in Column D is acetylated.

11. The method of claim 8, wherein the antibody or antigen-binding fragment thereof is conjugated to a cytotoxic agent.

12. A method for diagnosing a metabolic disorder in a subject, wherein the carcinoma is associated with lysine acetylation or deacetylation at a acetylation site in Table 1, comprising
a) obtaining a biological sample from the subject
b) determining the acetylation state or level at the lysine position identified in the corresponding row in Column D of Table 1.
wherein a change in the acetylation state or level at the acetylation site, as compared to a control, is indicative of the subject susceptible to, or suffering from a metabolic disorder.

13. The method of claim 12, wherein the acetylation state or level is determined by an antibody or antigen-binding fragment thereof, wherein the antibody specifically binds the acetylation site.

14. The method of claim 12, wherein the antibody binds to the acetylation site when the lysine identified in Column D is acetylated, and wherein the antibody does not bind to the same acetylation site when the lysine identified in Column D is not acetylated.

15. The method of claim 12, wherein the antibody binds to the acetylation site when the lysine identified in Column D is not acetylated, and wherein the antibody does not bind to the same acetylation site when the lysine identified in Column D is acetylated.

16. The method of claim 12, wherein the antibody is attached to a detectable marker.

17. A method for identifying an agent that modulates lysine acetylation at a acetylation site in Table 1, comprising:
a) contacting a candidate agent with an amino acid sequence comprising a acetylation site in Table 1; and
b) determining the acetylation state or level at the acetylation site,
wherein a change in the acetylation state or level of the specified lysine in the presence of the candidate agent, as compared to a control, is indicative of the candidate agent modulating lysine acetylation at the acetylation site.

18. The method of claim 17, wherein the acetylation state or level is determined by an antibody or antigen-binding fragment thereof, wherein the antibody binds the acetylation site.

19. The method of claim 18, wherein the antibody binds to the acetylation site when the lysine identified in Column D is acetylated, and wherein the antibody does not bind to the same acetylation site when the lysine identified in Column D is not acetylated.

20. The method of claim 18, wherein the antibody binds to the acetylation site when the lysine identified in Column D is not acetylated, and wherein the antibody does not bind to the same acetylation site when the lysine identified in Column D is acetylated.

21. A kit for detecting or quantitating an acetylation site identified in Table 1, comprising: an AQUA peptide comprising the acetylation site, or an antibody or an antigen-binding fragment thereof that binds to an amino acid sequence comprising the acetylation site.

22. A method for measuring changes in acetylation of proteins in signaling pathways associated with mitochondrial function in a mammal, said method comprising the steps of:
a. collecting and processing a sample from the mammal;
b. treating the processed sample from step (a) with an antibody to a site identified in Table 1; and
c. identifying and quantitating changes in acetylation patterns.

23. The method according to claim 22, wherein the changes in acetylation of proteins in signaling pathways associated with mitochondrial function occur from caloric restriction, aging or therapeutic treatment.

24. The antibody or antigen-binding fragment thereof of claim 2, wherein the acetylation site occurs in a protein in Table 1 that is selected from the group consisting of: a chromatin or DNA binding/repair/replication protein, an enzyme, an RNA binding protein, an RNA processing protein, a transcriptional regulator, a translation protein, a ubiquitin conjugating system protein, a cytoskeletal protein, mitochondrial proteins, an adaptor/scaffold protein and a receptor/channel/transporter/cell surface protein.

25. The antibody or antigen-binding fragment thereof of claim 2, wherein the antibody specifically binds to an amino acid sequence comprising a lysine acetylation site selected from the group consisting of SEQ ID NOs: 46 (ACAA2); 48 (ACAA2); 50 (ACAA2); 71 (ACAT1); 72 (ACAT1); 333 (CLYBL); 336 (CS); 351 (DLD); 375 (SUCLG1); 376 (SUCLG1); 417 (SLC25A4); 420 (SLC25A5); 491 (ACOT2); 549 (ALDH6A1); 631 (COX5B); 632 (COX5B); 643 (CPS1); 646 (CPS1); 647 (CPS1); 649 (CPS1); 687 (DLST); 697 (EHHADH); 715 (ETFDH); 739 (GCAT); 757 (GOT2); 759 (GOT2); 895 (PC); 1072 (ACAD11); and 1269 (SLC25A31).

26. The antibody or antigen-binding fragment thereof of claim 2, wherein the antibody specifically binds to an amino acid sequence comprising a lysine acetylation site selected from the group consisting of SEQ ID NOs: 708 (EPHX2), 887 (OTC), and 788 (HMGCL).

27. The antibody or antigen-binding fragment thereof of claim 2, wherein said antibody or antibody fragment is selected from the group consisting of a polyclonal antibody, a monoclonal antibody or antibody fragment, a recombinant antibody, a camelid antibody, a bispecific antibody, a diabody, a chimerized or chimeric antibody or antibody fragment, a humanized antibody or antibody fragment, a deimmunized human antibody or antibody fragment, a fully human antibody or antibody fragment, a single chain antibody, an Fv, an Fd, an Fab, an Fab′, and an F(ab′)₂.

28. The antibody or antigen-binding fragment thereof of claim 2, wherein the antibody or antibody fragment is conjugated to a cytotoxic agent.


SEQUENCE LISTING (1302 sequences)

SEQ ID NO: 1 - protein, 15 aa, Homo sapiens
LRLLDDDKDFVESRD

SEQ ID NO: 2 - protein, 15 aa, Homo sapiens
VNERDVPKATISRYS

SEQ ID NO: 3 - protein, 6 aa, Homo sapiens
KNKRRK

SEQ ID NO: 4 - protein, 6 aa, Homo sapiens
KNKRRK

SEQ ID NO: 5 - protein, 15 aa, Homo sapiens
VAVLTGKKVVQLDVR

SEQ ID NO: 6 - protein, 15 aa, Homo sapiens
DVRDNMVKLNDGSQI

SEQ ID NO: 7 - protein, 15 aa, Homo sapiens
GVAVLTGKKVVQLDV

SEQ ID NO: 8 - protein, 9 aa, Homo sapiens
EKEVAEVKS

SEQ ID NO: 9 - protein, 15 aa, Homo sapiens
VEESKASKRLTKRVQ

SEQ ID NO: 10 - protein, 15 aa, Homo sapiens
GFNPEVEKLMLQMNA

SEQ ID NO: 11 - protein, 15 aa, Homo sapiens
GDKAQIEKRIQEIIE

SEQ ID NO: 12 - protein, 10 aa, Homo sapiens
KGVITVKDGK

SEQ ID NO: 13 - protein, 15 aa, Homo sapiens
DAMLLKGKGDKAQIE

SEQ ID NO: 14 - protein, 11 aa, Homo sapiens
ISKGANPVEIR

SEQ ID NO: 15 - protein, 15 aa, Homo sapiens
VGGTSDVEVNEKKDR

SEQ ID NO: 16 - protein, 15 aa, Homo sapiens
LLKGKGDKAQIEKRI

SEQ ID NO: 17 - protein, 15 aa, Homo sapiens
IHLKSDCKEFSSEAR

SEQ ID NO: 18 - protein, 15 aa, Homo sapiens
HEFQAETKKLLDIVA

SEQ ID NO: 19 - protein, 11 aa, Homo sapiens
SLYSEKEVFIR

SEQ ID NO: 20 - protein, 20 aa, Homo sapiens
EGSGSSGTGEQKEDQKEEKQ

SEQ ID NO: 21 - protein, 8 aa, Homo sapiens
NTTIPTKK

SEQ ID NO: 22 - protein, 8 aa, Homo sapiens
HIVKEFKR

SEQ ID NO: 23 - protein, 22 aa, Homo sapiens
MEEFKDQLPADECNKLKEEISK

SEQ ID NO: 24 - protein, 8 aa, Homo sapiens
LFEMAYKK

SEQ ID NO: 25 - protein, 8 aa, Homo sapiens
LFEMAYKK

SEQ ID NO: 26 - protein, 20 aa, Homo sapiens
EGSGSSGTGEQKEDQKEEKQ

SEQ ID NO: 27 - protein, 7 aa, Homo sapiens
LKEEISK

SEQ ID NO: 28 - protein, 15 aa, Homo sapiens
TFIEYFGKMLTERGH

SEQ ID NO: 29 - protein, 15 aa, Homo sapiens
QEQSNKGKPLAFRVG

SEQ ID NO: 30 - protein, 15 aa, Homo sapiens
TCERCNGKGNEPGTK

SEQ ID NO: 31 - protein, 9 aa, Homo sapiens
KAYYQLAKK

SEQ ID NO: 32 - protein, 15 aa, Homo sapiens
PGTQTDQKIRMGGKG

SEQ ID NO: 33 - protein, 21 aa, Homo sapiens
YRAALALQITNLLTRCMFAYK

SEQ ID NO: 34 - protein, 8 aa, Homo sapiens
KGSQTNLK

SEQ ID NO: 35 - protein, 25 aa, Homo sapiens
TWIEVSGSSAKDVAKQLKEQQMVMR

SEQ ID NO: 36 - protein, 11 aa, Homo sapiens
IIEVGDTPKDR

SEQ ID NO: 37 - protein, 25 aa, Homo sapiens
TWIEVSGSSAKDVAKQLKEQQMVMR

SEQ ID NO: 38 - protein, 11 aa, Homo sapiens
IIEVGDTPKDR

SEQ ID NO: 39 - protein, 15 aa, Homo sapiens
GVFVVAAKRTPFGAY

SEQ ID NO: 40 - protein, 15 aa, Homo sapiens
LQSQQRWKAANDAGY

SEQ ID NO: 41 - protein, 15 aa, Homo sapiens
EMAPIEVKTKKGKQT

SEQ ID NO: 42 - protein, 15 aa, Homo sapiens
APIEVKTKKGKQTMQ

SEQ ID NO: 43 - protein, 23 aa, Homo sapiens
KGKQTMQVDEHARPQTTLEQLQK

SEQ ID NO: 44 - protein, 15 aa, Homo sapiens
SLDLDISKTNVNGGA

SEQ ID NO: 45 - protein, 15 aa, Homo sapiens
TTLEQLQKLPPVFKK

SEQ ID NO: 46 - protein, 15 aa, Homo sapiens
QKLPPVFKKDGTVTA

SEQ ID NO: 47 - protein, 15 aa, Homo sapiens
IASEDAVKKHNFTPL

SEQ ID NO: 48 - protein, 15 aa, Homo sapiens
PAISGALKKAGLSLK

SEQ ID NO: 49 - protein, 15 aa, Homo sapiens
AISGALKKAGLSLKD

SEQ ID NO: 50 - protein, 15 aa, Homo sapiens
KLPPVFKKDGTVTAG

SEQ ID NO: 51 - protein, 15 aa, Homo sapiens
PIEVKTKKGKQTMQV

SEQ ID NO: 52 - protein, 8 aa, Homo sapiens
LETPSAKK

SEQ ID NO: 53 - protein, 20 aa, Homo sapiens
SPAHGISLFLVENGMKGFIK

SEQ ID NO: 54 - protein, 15 aa, Homo sapiens
KLHKMGLKAQDTAEL

SEQ ID NO: 55 - protein, 15 aa, Homo sapiens
CLQLHEAKRLDSATA

SEQ ID NO: 56 - protein, 15 aa, Homo sapiens
RNYVKQRKAFGKTVA

SEQ ID NO: 57 - protein, 15 aa, Homo sapiens
KQRKAFGKTVAHLQT

SEQ ID NO: 58 - protein, 15 aa, Homo sapiens
QGIKTNAKKDGSDWI

SEQ ID NO: 59 - protein, 19 aa, Homo sapiens
GISAFLVPMPTPGLTLGKK

SEQ ID NO: 60 - protein, 15 aa, Homo sapiens
AFGAPLTKLQVIQFK

SEQ ID NO: 61 - protein, 15 aa, Homo sapiens
LFPAAQVKKMGGLGL

SEQ ID NO: 62 - protein, 15 aa, Homo sapiens
KERIQFGKRLFDFQG

SEQ ID NO: 63 - protein, 15 aa, Homo sapiens
ITHGPPEKKMGIKAS

SEQ ID NO: 64 - protein, 10 aa, Homo sapiens
EKITAFVVER

SEQ ID NO: 65 - protein, 14 aa, Homo sapiens
HKKGIVNEQFLLQR

SEQ ID NO: 66 - protein, 15 aa, Homo sapiens
INSYTRSKAAWEAGK

SEQ ID NO: 67 - protein, 15 aa, Homo sapiens
IPVTVTVKGQPDVVV

SEQ ID NO: 68 - protein, 15 aa, Homo sapiens
FSKVPKLKTVFQKEN

SEQ ID NO: 69 - protein, 10 aa, Homo sapiens
VDFSKVPKLK

SEQ ID NO: 70 - protein, 15 aa, Homo sapiens
KLKTVFQKENGTVTA

SEQ ID NO: 71 - protein, 15 aa, Homo sapiens
SCAENTAKKLNIARN

SEQ ID NO: 72 - protein, 15 aa, Homo sapiens
VKEDEEYKRVDFSKV

SEQ ID NO: 73 - protein, 15 aa, Homo sapiens
VLKDVGLKKEDIAMW

SEQ ID NO: 74 - protein, 15 aa, Homo sapiens
GQANTTLKRWLLDFA

SEQ ID NO: 75 - protein, 15 aa, Homo sapiens
RANRRKPKPPAPEDL

SEQ ID NO: 76 - protein, 9 aa, Homo sapiens
SQLETSLKR

SEQ ID NO: 77 - protein, 14 aa, Homo sapiens
YKYEDKDGKQPVGR

SEQ ID NO: 78 - protein, 10 aa, Homo sapiens
FKTIEEVVGR

SEQ ID NO: 79 - protein, 15 aa, Homo sapiens
VDETQFKKILGYINT

SEQ ID NO: 80 - protein, 19 aa, Homo sapiens
GLLLSCIEDKNPCIFFEPK

SEQ ID NO: 81 - protein, 15 aa, Homo sapiens
CSPELQQKFRSETIT

SEQ ID NO: 82 - protein, 9 aa, Homo sapiens
SFDPVLDKR

SEQ ID NO: 83 - protein, 15 aa, Homo sapiens
GKPLLGMKILDVGCG

SEQ ID NO: 84 - protein, 15 aa, Homo sapiens
KALKEKEKASWSSLS

SEQ ID NO: 85 - protein, 15 aa, Homo sapiens
RAHESVVKSEDFSLP

SEQ ID NO: 86 - protein, 15 aa, Homo sapiens
SGINSWKKRLIRIKN

SEQ ID NO: 87 - protein, 15 aa, Homo sapiens
QAFFSSGKNKAALEA

SEQ ID NO: 88 - protein, 15 aa, Homo sapiens
RRYLSAQKPLLNDGQ

SEQ ID NO: 89 - protein, 15 aa, Homo sapiens
DELFTDDKARHLLVL

SEQ ID NO: 90 - protein, 8 aa, Homo sapiens
GGKEFLKK

SEQ ID NO: 91 - protein, 15 aa, Homo sapiens
KAYNTLIKDKVNRDS

SEQ ID NO: 92 - protein, 11 aa, Homo sapiens
SPMVPLPMPKK

SEQ ID NO: 93 - protein, 15 aa, Homo sapiens
RNIIKERKGRNFSQH

SEQ ID NO: 94 - protein, 12 aa, Homo sapiens
LSEVVGSGKDGR

SEQ ID NO: 95 - protein, 12 aa, Homo sapiens
GIKLSFMPFFLK

SEQ ID NO: 96 - protein, 15 aa, Homo sapiens
AIPRFNQKGEVYKAQ

SEQ ID NO: 97 - protein, 15 aa, Homo sapiens
GKDGRILKEDILNYL

SEQ ID NO: 98 - protein, 15 aa, Homo sapiens
MPPPPKPKDMTVPIL

SEQ ID NO: 99 - protein, 15 aa, Homo sapiens
SRYDGVIKKLYYNLD

SEQ ID NO: 100 - protein, 15 aa, Homo sapiens
KPPVFTGKDKTEPIK

SEQ ID NO: 101 - protein, 14 aa, Homo sapiens
SYLENPAFMLLDLK

SEQ ID NO: 102 - protein, 15 aa, Homo sapiens
LEKLENDKSFRGVIL

SEQ ID NO: 103 - protein, 15 aa, Homo sapiens
NFVSFISKDSIQKSL

SEQ ID NO: 104 - protein, 15 aa, Homo sapiens
ELVISLEKLENDKSF

SEQ ID NO: 105 - protein, 7 aa, Homo sapiens
TKGAFSR

SEQ ID NO: 106 - protein, 7 aa, Homo sapiens
QLIKAQK

SEQ ID NO: 107 - protein, 15 aa, Homo sapiens
KFFSPLQKAMLPPNS

SEQ ID NO: 108 - protein, 15 aa, Homo sapiens
DPTGTFEKEMIGRIP

SEQ ID NO: 109 - protein, 15 aa, Homo sapiens
GEFNDLRKVTKEQWD

SEQ ID NO: 110 - protein, 15 aa, Homo sapiens
GIDLTQVKGTGPDGR

SEQ ID NO: 111 - protein, 15 aa, Homo sapiens
QWLAEFRKYLEKPIT

SEQ ID NO: 112 - protein, 10 aa, Homo sapiens
EKRKANFKDQ

SEQ ID NO: 113 - protein, 10 aa, Homo sapiens
EGMTAFVEKR

SEQ ID NO: 114 - protein, 14 aa, Homo sapiens
TAFVEKRKANFKDQ

SEQ ID NO: 115 - protein, 14 aa, Homo sapiens
ISAQDAKQAGLVSK

SEQ ID NO: 116 - protein, 15 aa, Homo sapiens
IIAEKRGKNNTVGLI

SEQ ID NO: 117 - protein, 10 aa, Homo sapiens
KNTETALDNK

SEQ ID NO: 118 - protein, 15 aa, Homo sapiens
VRSTMNFKIGGVTER

SEQ ID NO: 119 - protein, 23 aa, Homo sapiens
LMNESLMLVTALNPHIGYDKAAK

SEQ ID NO: 120 - protein, 10 aa, Homo sapiens
AAKIAKTAHK

SEQ ID NO: 121 - protein, 10 aa, Homo sapiens
AAKIAKTAHK

SEQ ID NO: 122 - protein, 7 aa, Homo sapiens
IKAAMPR

SEQ ID NO: 123 - protein, 10 aa, Homo sapiens
IGFAEKVAAK

SEQ ID NO: 124 - protein, 36 aa, Homo sapiens
VKQDMPPPGGYGPIDYKRVKQDMPPPGGYGPIDYKR

SEQ ID NO: 125 - protein, 18 aa, Homo sapiens
VKQDMPPPGGYGPIDYKR

SEQ ID NO: 126 - protein, 15 aa, Homo sapiens
KIVDRLKKYEAAYGK

SEQ ID NO: 127 - protein, 15 aa, Homo sapiens
ADKKISPKRDKGLVE

SEQ ID NO: 128 - protein, 15 aa, Homo sapiens
VYKKVEEKVRKQTKG

SEQ ID NO: 129 - protein, 8 aa, Homo sapiens
GLNDKVKK

SEQ ID NO: 130 - protein, 9 aa, Homo sapiens
GLNDKVKKK

SEQ ID NO: 131 - protein, 8 aa, Homo sapiens
KALTSFER

SEQ ID NO: 132 - protein, 15 aa, Homo sapiens
ITTEKTSKDTSASAV

SEQ ID NO: 133 - protein, 15 aa, Homo sapiens
HYFSPVDKMQLLEII

SEQ ID NO: 134 - protein, 15 aa, Homo sapiens
VIIVVKDGPGFYTTR

SEQ ID NO: 135 - protein, 15 aa, Homo sapiens
THINYGVKGDVAVVR

SEQ ID NO: 136 - protein, 15 aa, Homo sapiens
RIATKDRKTVLGTPE

SEQ ID NO: 137 - protein, 15 aa, Homo sapiens
SIRADRAKKMGLVDQ

SEQ ID NO: 138 - protein, 15 aa, Homo sapiens
IRADRAKKMGLVDQL

SEQ ID NO: 139 - protein, 15 aa, Homo sapiens
KISPKRDKGLVEKLT

SEQ ID NO: 140 - protein, 12 aa, Homo sapiens
KQTKGLYPAPLK

SEQ ID NO: 141 - protein, 11 aa, Homo sapiens
QTKGLYPAPLK

SEQ ID NO: 142 - protein, 8 aa, Homo sapiens
GLYPAPLK

SEQ ID NO: 143 - protein, 9 aa, Homo sapiens
GLNDKVKKK

SEQ ID NO: 144 - protein, 15 aa, Homo sapiens
SKGFLGRKSGKGFYI

SEQ ID NO: 145 - protein, 15 aa, Homo sapiens
FLGRKSGKGFYIYQE

SEQ ID NO: 146 - protein, 12 aa, Homo sapiens
DHANSPNKKFYQ

SEQ ID NO: 147 - protein, 15 aa, Homo sapiens
AKGLADKKISPKRDK

SEQ ID NO: 148 - protein, 15 aa, Homo sapiens
LEQMAKLKPAFIKPY

SEQ ID NO: 149 - protein, 15 aa, Homo sapiens
AAANRLRKEGGQYGL

SEQ ID NO: 150 - protein, 15 aa, Homo sapiens
KLMLDLNKAKSMGQR

SEQ ID NO: 151 - protein, 15 aa, Homo sapiens
VPFKVPGKDTVTKDN

SEQ ID NO: 152 - protein, 15 aa, Homo sapiens
KLKPAFIKPYGTVTA

SEQ ID NO: 153 - protein, 8 aa, Homo sapiens
MIVEAYPK

SEQ ID NO: 154 - protein, 15 aa, Homo sapiens
LRSHSLAKKAQDEGL

SEQ ID NO: 155 - protein, 15 aa, Homo sapiens
LRKVAKKKFAENPKA

SEQ ID NO: 156 - protein, 15 aa, Homo sapiens
DILAKSKKGIEESLR

SEQ ID NO: 157 - protein, 13 aa, Homo sapiens
LVEVIKTPMTSQK

SEQ ID NO: 158 - protein, 15 aa, Homo sapiens
ELFKRLDKFAAEHTI

SEQ ID NO: 159 - protein, 15 aa, Homo sapiens
KTPMTSQKTFESLVD

SEQ ID NO: 160 - protein, 15 aa, Homo sapiens
NKLVAENKFGKKTGE

SEQ ID NO: 161 - protein, 8 aa, Homo sapiens
KTGEGFYK

SEQ ID NO: 162 - protein, 15 aa, Homo sapiens
KKFAENLKAGDEFVE

SEQ ID NO: 163 - protein, 35 aa, Homo sapiens
GDASKEDIDTAMKLGAGYPMGPFELLDYVGLDTTK

SEQ ID NO: 164 - protein, 9 aa, Homo sapiens
TGEGFYKYK

SEQ ID NO: 165 - protein, 15 aa, Homo sapiens
KDQSPKWKPADLKEV

SEQ ID NO: 166 - protein, 15 aa, Homo sapiens
VITLNRPKFLNALTL

SEQ ID NO: 167 - protein, 15 aa, Homo sapiens
IDKDQSPKWKPADLK

SEQ ID NO: 168 - protein, 15 aa, Homo sapiens
KWKPADLKEVTEEDL

SEQ ID NO: 169 - protein, 15 aa, Homo sapiens
ETFLIIIKGAGGKAF

SEQ ID NO: 170 - protein, 15 aa, Homo sapiens
HAHGDQYKATDFVAD

SEQ ID NO: 171 - protein, 13 aa, Homo sapiens
LVPGWTKPITIGR

SEQ ID NO: 172 - protein, 10 aa, Homo sapiens
MWKSPNGTIR

SEQ ID NO: 173 - protein, 19 aa, Homo sapiens
NILGGTVFREPIICKNIPR

SEQ ID NO: 174 - protein, 15 aa, Homo sapiens
FKMVFTPKDGSGVKE

SEQ ID NO: 175 - protein, 21 aa, Homo sapiens
EHQKGRPTSTNPIASIFAWTR

SEQ ID NO: 176 - protein, 15 aa, Homo sapiens
RFAQMLEKVCVETVE

SEQ ID NO: 177 - protein, 15 aa, Homo sapiens
LITEGASKRIAEFAF

SEQ ID NO: 178 - protein, 14 aa, Homo sapiens
IFDAAKAPIQWEER

SEQ ID NO: 179 - protein, 8 aa, Homo sapiens
MGLKGPLK

SEQ ID NO: 180 - protein, 19 aa, Homo sapiens
GPLKTPIAAGHPSMNLLLR

SEQ ID NO: 181 - protein, 13 aa, Homo sapiens
IEAACFATIKDGK

SEQ ID NO: 182 - protein, 13 aa, Homo sapiens
IEAACFATIKDGK

SEQ ID NO: 183 - protein, 11 aa, Homo sapiens
SLTKDLGGNAK

SEQ ID NO: 184 - protein, 6 aa, Homo sapiens
RVKDLD

SEQ ID NO: 185 - protein, 15 aa, Homo sapiens
DAVKKVIKVGKVRTR

SEQ ID NO: 186 - protein, 15 aa, Homo sapiens
IHTPMEYKGELASYD

SEQ ID NO: 187 - protein, 15 aa, Homo sapiens
FANVVHVKSLPGYMT

SEQ ID NO: 188 - protein, 17 aa, Homo sapiens
HLNLEYHSSMIADAVKK

SEQ ID NO: 189 - protein, 15 aa, Homo sapiens
MIADAVKKVIKVGKV

SEQ ID NO: 190 - protein, 10 aa, Homo sapiens
HKDIDILIVR

SEQ ID NO: 191 - protein, 14 aa, Homo sapiens
IAEYAFKLAQESGR

SEQ ID NO: 192 - protein, 16 aa, Homo sapiens
ANIMKLGDGLFLQCCR

SEQ ID NO: 193 - protein, 15 aa, Homo sapiens
HFNHRLLKCNPEEGM

SEQ ID NO: 194 - protein, 15 aa, Homo sapiens
TVRSHLMKKPRFDYS

SEQ ID NO: 195 - protein, 12 aa, Homo sapiens
FEEEESDKRESE

SEQ ID NO: 196 - protein, 15 aa, Homo sapiens
IKLGGGEKARALHIS

SEQ ID NO: 197 - protein, 15 aa, Homo sapiens
IANDATVKGGAYYPV

SEQ ID NO: 198 - protein, 15 aa, Homo sapiens
PIAGFLQKNKAGGMH

SEQ ID NO: 199 - protein, 15 aa, Homo sapiens
RSLSEEVKIGAHGKP

SEQ ID NO: 200 - protein, 15 aa, Homo sapiens
IETKAAVKGYLGPEQ

SEQ ID NO: 201 - protein, 11 aa, Homo sapiens
ASIKKGEDFVK

SEQ ID NO: 202 - protein, 11 aa, Homo sapiens
KKGEDFVKTLK

SEQ ID NO: 203 - protein, 24 aa, Homo sapiens
VNVPVIGGHAGKTIIPLISQCTPK

SEQ ID NO: 204 - protein, 15 aa, Homo sapiens
EKNLGIGKVSSFEEK

SEQ ID NO: 205 - protein, 15 aa, Homo sapiens
ELRRPEMKNFFKDMP

SEQ ID NO: 206 - protein, 15 aa, Homo sapiens
PEMKNFFKDMPQPRL

SEQ ID NO: 207 - protein, 16 aa, Homo sapiens
LALNCVGGKSSTELLR

SEQ ID NO: 208 - protein, 15 aa, Homo sapiens
SALEASMKPFISSKQ

SEQ ID NO: 209 - protein, 15 aa, Homo sapiens
FWLSQWKKDHSPDQF

SEQ ID NO: 210 - protein, 8 aa, Homo sapiens
IPPSTPYK

SEQ ID NO: 211 - protein, 9 aa, Homo sapiens
LENVLSGKA

SEQ ID NO: 212 - protein, 15 aa, Homo sapiens
VTPAPPIKRWELSSD

SEQ ID NO: 213 - protein, 14 aa, Homo sapiens
TQPPPKLPVGPSHK

SEQ ID NO: 214 - protein, 15 aa, Homo sapiens
SAVAATEKKAVTPAP

SEQ ID NO: 215 - protein, 15 aa, Homo sapiens
LRYQEISKRTQPPPK

SEQ ID NO: 216 - protein, 10 aa, Homo sapiens
WLKDQELSPR

SEQ ID NO: 217 - protein, 21 aa, Homo sapiens
VVAACAMPVMKGWNILTNSEK

SEQ ID NO: 218 - protein, 8 aa, Homo sapiens
FAYDGLKR

SEQ ID NO: 219 - protein, 17 aa, Homo sapiens
NEKGLLTYTSWEDALSR

SEQ ID NO: 220 - protein, 8 aa, Homo sapiens
GDKKPDAK

SEQ ID NO: 221 - protein, 12 aa, Homo sapiens
KLEAGDKKPDAK

SEQ ID NO: 222 - protein, 11 aa, Homo sapiens
SYGANFSWNKR

SEQ ID NO: 223 - protein, 15 aa, Homo sapiens
TGQVYDDKDYRRIRF

SEQ ID NO: 224 - protein, 15 aa, Homo sapiens
KGRGGLRKPEASHSF

SEQ ID NO: 225 - protein, 15 aa, Homo sapiens
TLVEFPQKVLSPFRK

SEQ ID NO: 226 - protein, 6 aa, Homo sapiens
WSSEKR

SEQ ID NO: 227 - protein, 11 aa, Homo sapiens
AFDLDVFKNFS

SEQ ID NO: 228 - protein, 11 aa, Homo sapiens
AAPATGNKKTH

SEQ ID NO: 229 - protein, 11 aa, Homo sapiens
SVHKSWDIFFR

SEQ ID NO: 230 - protein, 15 aa, Homo sapiens
TKAEQFYCGDTEGKK

SEQ ID NO: 231 - protein, 12 aa, Homo sapiens
HHVLHDQNVDKR

SEQ ID NO: 232 - protein, 21 aa, Homo sapiens
AKPVWYAGRDPAAAPATGNKK

SEQ ID NO: 233 - protein, 15 aa, Homo sapiens
AQNPENVKRLLFCTG

SEQ ID NO: 234 - protein, 15 aa, Homo sapiens
KEGDGEAKSAKPGDD

SEQ ID NO: 235 - protein, 15 aa, Homo sapiens
DGEAKSAKPGDDVRE

SEQ ID NO: 236 - protein, 15 aa, Homo sapiens
MIPGKMVKGMGGAMD

SEQ ID NO: 237 - protein, 15 aa, Homo sapiens
KAVFDVDKKKGLTLI

SEQ ID NO: 238 - protein, 11 aa, Homo sapiens
ASAGGGGKGMR

SEQ ID NO: 239 - protein, 15 aa, Homo sapiens
SDRTEALKRMADALD

SEQ ID NO: 240 - protein, 15 aa, Homo sapiens
DVYPDGFKGHMLTKS

SEQ ID NO: 241 - protein, 9 aa, Homo sapiens
FVKGDISTK

SEQ ID NO: 242 - protein, 14 aa, Homo sapiens
SYLNMDAIMEAIKK

SEQ ID NO: 243 - protein, 17 aa, Homo sapiens
VYAEDPYKSFGLPSIGR

SEQ ID NO: 244 - protein, 15 aa, Homo sapiens
GAVEIIFKGHENVEA

SEQ ID NO: 245 - protein, 10 aa, Homo sapiens
MELKADQLYK

SEQ ID NO: 246 - protein, 14 aa, Homo sapiens
SGKGPILMELQTYR

SEQ ID NO: 247 - protein, 9 aa, Homo sapiens
GANQWIKFK

SEQ ID NO: 248 - protein, 12 aa, Homo sapiens
NFANDATFEIKK

SEQ ID NO: 249 - protein, 8 aa, Homo sapiens
EDGLKYYR

SEQ ID NO: 250 - protein, 10 aa, Homo sapiens
AAASTDYYKR

SEQ ID NO: 251 - protein, 13 aa, Homo sapiens
RMELKADQLYKQK

SEQ ID NO: 252 - protein, 11 aa, Homo sapiens
RMELKADQLYK

SEQ ID NO: 253 - protein, 13 aa, Homo sapiens
RMELKADQLYKQK

SEQ ID NO: 254 - protein, 19 aa, Homo sapiens
VFLLGEEVAQYDGAYKVSR

SEQ ID NO: 255 - protein, 14 aa, Homo sapiens
AKWDAWNALGSLPK

SEQ ID NO: 256 - protein, 15 aa, Homo sapiens
MNQVKLLKKDPGNEV

SEQ ID NO: 257 - protein, 15 aa, Homo sapiens
IMFNRPKKKNAINTE

SEQ ID NO: 258 - protein, 15 aa, Homo sapiens
NQVKLLKKDPGNEVK

SEQ ID NO: 259 - protein, 15 aa, Homo sapiens
MFNRPKKKNAINTEM

SEQ ID NO: 260 - protein, 10 aa, Homo sapiens
FAFNGEFKNK

SEQ ID NO: 261 - protein, 15 aa, Homo sapiens
LRYVDSSKAVIETAD

SEQ ID NO: 262 - protein, 12 aa, Homo sapiens
ADVVPKTAENFR

SEQ ID NO: 263 - protein, 8 aa, Homo sapiens
EGMDVVKK

SEQ ID NO: 264 - protein, 15 aa, Homo sapiens
KIESFGSKSGRTSKK

SEQ ID NO: 265 - protein, 18 aa, Homo sapiens
ALCTGEKGFGYKGSTFHR

SEQ ID NO: 266 - protein, 14 aa, Homo sapiens
ILSCGELIPKSKVK

SEQ ID NO: 267 - protein, 14 aa, Homo sapiens
ILSCGELIPKSKVK

SEQ ID NO: 268 - protein, 14 aa, Homo sapiens
ILSCGELIPKSKVK

SEQ ID NO: 269 - protein, 10 aa, Homo sapiens
VNLAELFKGK

SEQ ID NO: 270 - protein, 17 aa, Homo sapiens
SVLANGMKLLGITPVCR

SEQ ID NO: 271 - protein, 7 aa, Homo sapiens
LITSDKK

SEQ ID NO: 272 - protein, 15 aa, Homo sapiens
VPKGYVAKSPDEAYA

SEQ ID NO: 273 - protein, 10 aa, Homo sapiens
KLGSKDVVIK

SEQ ID NO: 274 - protein, 9 aa, Homo sapiens
LGSKDVVIK

SEQ ID NO: 275 - protein, 15 aa, Homo sapiens
VLAGGRGKGTFESGL

SEQ ID NO: 276 - protein, 15 aa, Homo sapiens
GTFESGLKGGVKIVF

SEQ ID NO: 277 - protein, 15 aa, Homo sapiens
SSQMIGKKLFTKQTG

SEQ ID NO: 278 - protein, 15 aa, Homo sapiens
IGKKLFTKQTGEKGR

SEQ ID NO: 279 - protein, 15 aa, Homo sapiens
FTKQTGEKGRICNQV

SEQ ID NO: 280 - protein, 15 aa, Homo sapiens
IDIEEGIKKEQALQL

SEQ ID NO: 281 - protein, 17 aa, Homo sapiens
LGSKDVVIKAQVLAGGR

SEQ ID NO: 282 - protein, 13 aa, Homo sapiens
INFDSNSAYRQKK

SEQ ID NO: 283 - protein, 9 aa, Homo sapiens
KAVASVAKK

SEQ ID NO: 284 - protein, 8 aa, Homo sapiens
EALEAAKR

SEQ ID NO: 285 - protein, 15 aa, Homo sapiens
IDLEDAAKKAVASVA

SEQ ID NO: 286 - protein, 15 aa, Homo sapiens
DLEDAAKKAVASVAK

SEQ ID NO: 287 - protein, 6 aa, Homo sapiens
HQLLKK

SEQ ID NO: 288 - protein, 9 aa, Homo sapiens
KSSLKKGGR

SEQ ID NO: 289 - protein, 15 aa, Homo sapiens
DRQCAQVKRLENNHA

SEQ ID NO: 290 - protein, 10 aa, Homo sapiens
GTPGLSFGKK

SEQ ID NO: 291 - protein, 9 aa, Homo sapiens
REHIDKYKN

SEQ ID NO: 292 - protein, 15 aa, Homo sapiens
ARSDPDPKAPANKAF

SEQ ID NO: 293 - protein, 15 aa, Homo sapiens
GIKTKAEKKGDEYII

SEQ ID NO: 294 - protein, 15 aa, Homo sapiens
FEDVKVPKENVLIGD

SEQ ID NO: 295 - protein, 15 aa, Homo sapiens
DVAGIKTKAEKKGDE

SEQ ID NO: 296 - protein, 15 aa, Homo sapiens
IGDGAGFKVAMGAFD

SEQ ID NO: 297 - protein, 15 aa, Homo sapiens
IKTKAEKKGDEYIIN

SEQ ID NO: 298 - protein, 15 aa, Homo sapiens
PGIQIGRKELNMGQR

SEQ ID NO: 299 - protein, 8 aa, Homo sapiens
IHETNLKK

SEQ ID NO: 300 - protein, 15 aa, Homo sapiens
SVLCQRAKVAMSHFE

SEQ ID NO: 301 - protein, 24 aa, Homo sapiens
VAVPSTIHCDHLIEAQVGGEKDLR

SEQ ID NO: 302 - protein, 16 aa, Homo sapiens
DINQEVYNFLATAGAK

SEQ ID NO: 303 - protein, 8 aa, Homo sapiens
CPKVIGVK

SEQ ID NO: 304 - protein, 7 aa, Homo sapiens
MKKYLSK

SEQ ID NO: 305 - protein, 7 aa, Homo sapiens
MKKYLSK

SEQ ID NO: 306 - protein, 16 aa, Homo sapiens
SAAVAKQALAHGLKCK

SEQ ID NO: 307 - protein, 16 aa, Homo sapiens
SAAVAKQALAHGLKCK

SEQ ID NO: 308 - protein, 15 aa, Homo sapiens
CKSQFTITPGSEQIR

SEQ ID NO: 309 - protein, 15 aa, Homo sapiens
CIGQWDRKDIKKGEK

SEQ ID NO: 310 - protein, 13 aa, Homo sapiens
KGEKNTIVTSYNR

SEQ ID NO: 311 - protein, 13 aa, Homo sapiens
KGEKNTIVTSYNR

SEQ ID NO: 312 - protein, 16 aa, Homo sapiens
GKCTTDHISAAGPWLK

SEQ ID NO: 313 - protein, 16 aa, Homo sapiens
CTTDHISAAGPWLKFR

SEQ ID NO: 314 - protein, 26 aa, Homo sapiens
GHLDNISNNLLIGAINIENGKANSVR

SEQ ID NO: 315 - protein, 15 aa, Homo sapiens
KPLKCIIKHPNGTQE

SEQ ID NO: 316 - protein, 23 aa, Homo sapiens
LQLLEPFDKWDGKDLEDLQILIK

SEQ ID NO: 317 - protein, 16 aa, Homo sapiens
DIKKGEKNTIVTSYNR

SEQ ID NO: 318 - protein, 8 aa, Homo sapiens
YYKKHGIR

SEQ ID NO: 319 - protein, 8 aa, Homo sapiens
YYKKHGIR

SEQ ID NO: 320 - protein, 15 aa, Homo sapiens
PLEGPDQKSFIPVER

SEQ ID NO: 321 - protein, 15 aa, Homo sapiens
LLSHPEVKGPGVGLL

SEQ ID NO: 322 - protein, 15 aa, Homo sapiens
PKEVMGEKKESKPAA

SEQ ID NO: 323 - protein, 15 aa, Homo sapiens
GGGGGGGKRGGKKDD

SEQ ID NO: 324 - protein, 15 aa, Homo sapiens
PVLEYYQKKGVLETF

SEQ ID NO: 325 - protein, 17 aa, Homo sapiens
AVIMGAPGSGKGTVSSR

SEQ ID NO: 326 - protein, 15 aa, Homo sapiens
KAFIDQGKLIPDDVM

SEQ ID NO: 327 - protein, 26 aa, Homo sapiens
TVGIDDLTGEPLIQREDDKPETVIKR

SEQ ID NO: 328 - protein, 15 aa, Homo sapiens
VLEYYQKKGVLETFS

SEQ ID NO: 329 - protein, 36 aa, Homo sapiens
VYNIEFNPPKTVGIDDLTGEPLIQREDDKPETVIKR

SEQ ID NO: 330 - protein, 15 aa, Homo sapiens
EDGVAANKKNEARLR

SEQ ID NO: 331 - protein, 15 aa, Homo sapiens
DGVAANKKNEARLRI

SEQ ID NO: 332 - protein, 15 aa, Homo sapiens
KEHQQLGKGAFTFQG

SEQ ID NO: 333 - protein, 13 aa, Homo sapiens
RAVLYVPGNDEKK

SEQ ID NO: 334 - protein, 15 aa, Homo sapiens
VPGNDEKKIKKIPSL

SEQ ID NO: 335 - protein, 9 aa, Homo sapiens
VVRHQLLKT

SEQ ID NO: 336 - protein, 19 aa, Homo sapiens
GMKGLVYETSVLDPDEGIR

SEQ ID NO: 337 - protein, 15 aa, Homo sapiens
KLVAQLYKIVPNVLL

SEQ ID NO: 338 - protein, 15 aa, Homo sapiens
ILADLIPKEQARIKT

SEQ ID NO: 339 - protein, 15 aa, Homo sapiens
PKEQARIKTFRQQHG

SEQ ID NO: 340 - protein, 15 aa, Homo sapiens
FSIPECQKLLPKAKG

SEQ ID NO: 341 - protein, 12 aa, Homo sapiens
EVGKDVSDEKLR

SEQ ID NO: 342 - protein, 15 aa, Homo sapiens
CQREFALKHLPNDPM

SEQ ID NO: 343 - protein, 15 aa, Homo sapiens
NVLLEQGKAKNPWPN

SEQ ID NO: 344 - protein, 15 aa, Homo sapiens
GFPLERPKSMSTEGL

SEQ ID NO: 345 - protein, 15 aa, Homo sapiens
TFRQQHGKTVVGQIT

SEQ ID NO: 346 - protein, 10 aa, Homo sapiens
LMKFVDSKSG

SEQ ID NO: 347 - protein, 12 aa, Homo sapiens
FKLNTKVTGATK

SEQ ID NO: 348 - protein, 12 aa, Homo sapiens
FKLNTKVTGATK

SEQ ID NO: 349 - protein, 12 aa, Homo sapiens
NLAASFGKSINF

SEQ ID NO: 350 - protein, 15 aa, Homo sapiens
AHLFKQNKVVHVNGY

SEQ ID NO: 351 - protein, 20 aa, Homo sapiens
ALLNNSHYYHMAHGKDFASR

SEQ ID NO: 352 - protein, 15 aa, Homo sapiens
EQKSTAVKALTGGIA

SEQ ID NO: 353 - protein, 9 aa, Homo sapiens
LKTVTFSKL

SEQ ID NO: 354 - protein, 23 aa, Homo sapiens
SGENFKLLYDLADQLHAAVGASR

SEQ ID NO: 355 - protein, 15 aa, Homo sapiens
VAGTKCDKVAQDLCK

SEQ ID NO: 356 - protein, 15 aa, Homo sapiens
KVAQDLCKVAGIAKV

SEQ ID NO: 357 - protein, 15 aa, Homo sapiens
ISDIIAIKSPDTFVR

SEQ ID NO: 358 - protein, 9 aa, Homo sapiens
GLKSGENFK

SEQ ID NO: 359 - protein, 11 aa, Homo sapiens
VVPEMTEILKK

SEQ ID NO: 360 - protein, 12 aa, Homo sapiens
VVPEMTEILKKK

SEQ ID NO: 361 - protein, 15 aa, Homo sapiens
LKEKKLVKEVIAVSC

SEQ ID NO: 362 - protein, 15 aa, Homo sapiens
YAVKIRVKPDRTGVV

SEQ ID NO: 363 - protein, 15 aa, Homo sapiens
GVVTDGVKHSMNPFC

SEQ ID NO: 364 - protein, 15 aa, Homo sapiens
AKLAEKEKVDLVLLG

SEQ ID NO: 365 - protein, 15 aa, Homo sapiens
AVRLKEKKLVKEVIA

SEQ ID NO: 366 - protein, 15 aa, Homo sapiens
VAVGSGSKGKGGEIQ

SEQ ID NO: 367 - protein, 15 aa, Homo sapiens
VGSGSKGKGGEIQPV

SEQ ID NO: 368 - protein, 17 aa, Homo sapiens
AFGGQSLKFGKGGQAHR

SEQ ID NO: 369 - protein, 11 aa, Homo sapiens
GAHAREDYKVR

SEQ ID NO: 370 - protein, 15 aa, Homo sapiens
IDEYDYSKPIQGQQK

SEQ ID NO: 371 - protein, 15 aa, Homo sapiens
ELRLSMQKSMQNHAA

SEQ ID NO: 372 - protein, 15 aa, Homo sapiens
DPDKAGDKPHMQTYE

SEQ ID NO: 373 - protein, 15 aa, Homo sapiens
FTEERLAKLQDPFSL

SEQ ID NO: 374 - protein, 7 aa, Homo sapiens
AIAEIKK

SEQ ID NO: 375 - protein, 11 aa, Homo sapiens
IGIMPGHIHKK

SEQ ID NO: 376 - protein, 16 aa, Homo sapiens
MGHAGAIIAGGKGGAK

SEQ ID NO: 377 - protein, 16 aa, Homo sapiens
MGHAGAIIAGGKGGAK

SEQ ID NO: 378 - protein, 29 aa, Homo sapiens
ISALQSAGVVVSMSPAQLGTTIYKEFEKR

SEQ ID NO: 379 - protein, 15 aa, Homo sapiens
LYVDKNTKIICQGFT

SEQ ID NO: 380 - protein, 24 aa, Homo sapiens
IICQGFTGKQGTFHSQQALEYGTK

SEQ ID NO: 381 - protein, 15 aa, Homo sapiens
VGGTTPGKGGQTHLG

SEQ ID NO: 382 - protein, 11 aa, Homo sapiens
IGIMPGHIHKK

SEQ ID NO: 383 - protein, 15 aa, Homo sapiens
RQHLYVDKNTKIICQ

SEQ ID NO: 384 - protein, 29 aa, Homo sapiens
ISALQSAGVVVSMSPAQLGTTIYKEFEKR

SEQ ID NO: 385 - protein, 18 aa, Homo sapiens
MGHAGAIIAGGKGGAKEK

SEQ ID NO: 386 - protein, 15 aa, Homo sapiens
TSSVIRYKGVLGTIT

SEQ ID NO: 387 - protein, 15 aa, Homo sapiens
VKTEGRMKLYSGLPA

SEQ ID NO: 388 - protein, 15 aa, Homo sapiens
QSHLHGIKPRYTGTY

SEQ ID NO: 389 - protein, 15 aa, Homo sapiens
GGTHDGPKKKGKDLT

SEQ ID NO: 390 - protein, 15 aa, Homo sapiens
GTHDGPKKKGKDLTF

SEQ ID NO: 391 - protein, 15 aa, Homo sapiens
THDGPKKKGKDLTFV

SEQ ID NO: 392 - protein, 31 aa, Homo sapiens
SAPEVTAPGAPAYRVLMEQLTSVSEELDVRK

SEQ ID NO: 393 - protein, 15 aa, Homo sapiens
AGLESGGKDSCRGDS

SEQ ID NO: 394 - protein, 18 aa, Homo sapiens
QLLDDHKDVVTLLAEGLR

SEQ ID NO: 395 - protein, 16 aa, Homo sapiens
DIKPDNLLLDSKGHVK

SEQ ID NO: 396 - protein, 15 aa, Homo sapiens
KAGQRWIKCNKKTLT

SEQ ID NO: 397 - protein, 15 aa, Homo sapiens
LEIKSTDFATSLSVK

SEQ ID NO: 398 - protein, 15 aa, Homo sapiens
TKHSMVIKSAAFEDE

SEQ ID NO: 399 - protein, 15 aa, Homo sapiens
DLAELINKMRLAQQN

SEQ ID NO: 400 - protein, 15 aa, Homo sapiens
ANIKISGKWKAQKRF

SEQ ID NO: 401 - protein, 15 aa, Homo sapiens
SGKWKAQKRFLKMSG

SEQ ID NO: 402 - protein, 15 aa, Homo sapiens
KMKNAHLKKQNLERA

SEQ ID NO: 403 - protein, 8 aa, Homo sapiens
IYKETEKR

SEQ ID NO: 404 - protein, 15 aa, Homo sapiens
EKHYVFLKRLCQVLC

SEQ ID NO: 405 - protein, 12 aa, Homo sapiens
MKFGSWTYDKAK

SEQ ID NO: 406 - protein, 12 aa, Homo sapiens
MKFGSWTYDKAK

SEQ ID NO: 407 - protein, 16 aa, Homo sapiens
GLSSLLYGSIPKAAVR

SEQ ID NO: 408 - protein, 10 aa, Homo sapiens
MQGLEAHKYR

SEQ ID NO: 409 - protein, 17 aa, Homo sapiens
EQGLKGTYQGLTATVLK

SEQ ID NO: 410 - protein, 9 aa, Homo sapiens
AFYKGTVPR

SEQ ID NO: 411 - protein, 15 aa, Homo sapiens
TGTLDCAKKLYQEFG

SEQ ID NO: 412 - protein, 15 aa, Homo sapiens
FGIRGIYKGTVLTLM

SEQ ID NO: 413 - protein, 9 aa, Homo sapiens
MQVDPQKYK

SEQ ID NO: 414 - protein, 12 aa, Homo sapiens
VKLLLQVQHASK

SEQ ID NO: 415 - protein, 12 aa, Homo sapiens
IPKEQGFLSFWR

SEQ ID NO: 416 - protein, 15 aa, Homo sapiens
RLAADVGKGAAQREF

SEQ ID NO: 417 - protein, 14 aa, Homo sapiens
YFPTQALNFAFKDK

SEQ ID NO: 418 - protein, 12 aa, Homo sapiens
VKLLLQVQHASK

SEQ ID NO: 419 - protein, 12 aa, Homo sapiens
IPKEQGVLSFWR

SEQ ID NO: 420 - protein, 22 aa, Homo sapiens
TDAAVSFAKDFLAGGVAAAISK

SEQ ID NO: 421 - protein, 15 aa, Homo sapiens
RLAADVGKSGTEREF

SEQ ID NO: 422 - protein, 15 aa, Homo sapiens
GLGDCLVKITKSDGI

SEQ ID NO: 423 - protein, 12 aa, Homo sapiens
VKLLLQVQHASK

SEQ ID NO: 424 - protein, 12 aa, Homo sapiens
IPKEQGVLSFWR

SEQ ID NO: 425 - protein, 15 aa, Homo sapiens
IFLGGVDKHTQFWRY

SEQ ID NO: 426 - protein, 10 aa, Homo sapiens
AVDALKSDKK

SEQ ID NO: 427 - protein, 12 aa, Homo sapiens
DRLEGLLAFKEK

SEQ ID NO: 428 - protein, 15 aa, Homo sapiens
YNLLKLYKINGDWQR

SEQ ID NO: 429 - protein, 15 aa, Homo sapiens
NQLPRSNKQLFELLC

SEQ ID NO: 430 - protein, 15 aa, Homo sapiens
TVQVPLSKRAKFVGP

SEQ ID NO: 431 - protein, 6 aa, Homo sapiens
LNEYKR

SEQ ID NO: 432 - protein, 27 aa, Homo sapiens
SQIVMLEASAENILQQDFCHAIKVGVK

SEQ ID NO: 433 - protein, 15 aa, Homo sapiens
LRRQKRFKLEEKVKK

SEQ ID NO: 434 - protein, 15 aa, Homo sapiens
KRFKLEEKVKKGGSG

SEQ ID NO: 435 - protein, 15 aa, Homo sapiens
KLEEKVKKGGSGAAT

SEQ ID NO: 436 - protein, 15 aa, Homo sapiens
PVVTSASKLSPPLLS

SEQ ID NO: 437 - protein, 19 aa, Homo sapiens
EKQFQKVITEYLNAQESAK

SEQ ID NO: 438 - protein, 15 aa, Homo sapiens
LDAEVVEKPAKEETV

SEQ ID NO: 439 - protein, 15 aa, Homo sapiens
GINLVQAKKLVESLP

SEQ ID NO: 440 - protein, 15 aa, Homo sapiens
VKLIKEIKNYIQGIN

SEQ ID NO: 441 - protein, 9 aa, Homo sapiens
HITGLCGKK

SEQ ID NO: 442 - protein, 15 aa, Homo sapiens
PDTAEIIKTLPQKYR

SEQ ID NO: 443 - protein, 15 aa, Homo sapiens
TEAEREKKRLERAKR

SEQ ID NO: 444 - protein, 15 aa, Homo sapiens
QMLWADAKKARRIKT

SEQ ID NO: 445 - protein, 15 aa, Homo sapiens
RIKTNMWKHNIKFHQ

SEQ ID NO: 446 - protein, 15 aa, Homo sapiens
IRHFWTPKQQTDFLD

SEQ ID NO: 447 - protein, 15 aa, Homo sapiens
RSKRENVKTGVKPDA

SEQ ID NO: 448 - protein, 15 aa, Homo sapiens
HVYTNAKKQMLISLA

SEQ ID NO: 449 - protein, 15 aa, Homo sapiens
PTVVKNYKDLEKAVQ

SEQ ID NO: 450 - protein, 15 aa, Homo sapiens
RYGTEAQKARWLIPL

SEQ ID NO: 451 - protein, 15 aa, Homo sapiens
VHRATVAKLELKHRI

SEQ ID NO: 452 - protein, 11 aa, Homo sapiens
TVAKLELKHRI

SEQ ID NO: 453 - protein, 15 aa, Homo sapiens
KEGFRVFKEMPFTNP

SEQ ID NO: 454 - protein, 15 aa, Homo sapiens
KNMNLSKKPKKAVKI

SEQ ID NO: 455 - protein, 15 aa, Homo sapiens
MGHPQFWKYEFGACT

SEQ ID NO: 456 - protein, 15 aa, Homo sapiens
VEAHVDQKNKVVTTP

SEQ ID NO: 457 - protein, 15 aa, Homo sapiens
LGAKHCVKEVVEAHV

SEQ ID NO: 458 - protein, 26 aa, Homo sapiens
GVEVTVGHEQEEGGKWPYAGTAEAIK

SEQ ID NO: 459 - protein, 13 aa, Homo sapiens
NLSTFAVDGKDCK

SEQ ID NO: 460 - protein, 7 aa, Homo sapiens
VNKEVER

SEQ ID NO: 461 - protein, 15 aa, Homo sapiens
DLKEEHLKKNIPLGR

SEQ ID NO: 462 - protein, 15 aa, Homo sapiens
QSVYSASKGGLVGFS

SEQ ID NO: 463 - protein, 15 aa, Homo sapiens
TDMTKDLKEEHLKKN

SEQ ID NO: 464 - protein, 15 aa, Homo sapiens
NTFEEMEKHLGRVNF

SEQ ID NO: 465 - protein, 15 aa, Homo sapiens
LKEEHLKKNIPLGRF

SEQ ID NO: 466 - protein, 15 aa, Homo sapiens
MSQIANVKKDMIILE

SEQ ID NO: 467 - protein, 11 aa, Homo sapiens
VKELYSLNEKK

SEQ ID NO: 468 - protein, 10 aa, Homo sapiens
LVQLGQAEKR

SEQ ID NO: 469 - protein, 15 aa, Homo sapiens
LRSSDEQKQQPPNSF

SEQ ID NO: 470 - protein, 15 aa, Homo sapiens
RGASVMIKKAINNVR

SEQ ID NO: 471 - protein, 14 aa, Homo sapiens
ANLEAIAKKGKFNK

SEQ ID NO: 472 - protein, 15 aa, Homo sapiens
GGSIDLQKEDNGIGI

SEQ ID NO: 473 - protein, 15 aa, Homo sapiens
RDVQDECKKKGLPWT

SEQ ID NO: 474 - protein, 15 aa, Homo sapiens
DVQDECKKKGLPWTL

SEQ ID NO: 475 - protein, 7 aa, Homo sapiens
VAEAKAR

SEQ ID NO: 476 - protein, 13 aa, Homo sapiens
AAKDGANIVIAAK

SEQ ID NO: 477 - protein, 26 aa, Homo sapiens
FGGIDILVNNASAISLTNTLDTPTKR

SEQ ID NO: 478 - protein, 9 aa, Homo sapiens
MAVEGVLKR

SEQ ID NO: 479 - protein, 15 aa, Homo sapiens
DAVPEEVKRAAREMG

SEQ ID NO: 480 - protein, 15 aa, Homo sapiens
SFVAMDTKDIPQILQ

SEQ ID NO: 481 - protein, 15 aa, Homo sapiens
CKELGLEKEDAALLP

SEQ ID NO: 482 - protein, 15 aa, Homo sapiens
GPRTTIKAKQLETLK

SEQ ID NO: 483 - protein, 15 aa, Homo sapiens
GPRTTIKAKQLETLK

SEQ ID NO: 484 - protein, 15 aa, Homo sapiens
HDCGELTKKDVKLAK

SEQ ID NO: 485 - protein, 20 aa, Homo sapiens
VALQAEKMNHHPEWFNVYNK

SEQ ID NO: 486 - protein, 14 aa, Homo sapiens
QQQAGARKKELLER

SEQ ID NO: 487 - protein, 15 aa, Homo sapiens
QSLTEDQKLIDEGLE

SEQ ID NO: 488 - protein, 15 aa, Homo sapiens
WLKVNGLKAKKLSLY

SEQ ID NO: 489 - protein, 15 aa, Homo sapiens
AIPQAMQKLVVTRLS

SEQ ID NO: 490 - protein, 9 aa, Homo sapiens
LPHSVNSKL

SEQ ID NO: 491 - protein, 15 aa, Homo sapiens
RASLRDEKGALFQAH

SEQ ID NO: 492 - protein, 15 aa, Homo sapiens
RAATGFLKLLADKNS

SEQ ID NO: 493 - protein, 15 aa, Homo sapiens
FLKLLADKNSELFRK

SEQ ID NO: 494 - protein, 15 aa, Homo sapiens
KNSELFRKYALFSPS

SEQ ID NO: 495 - protein, 15 aa, Homo sapiens
QFGEEMMKRALQYSP

SEQ ID NO: 496 - protein, 15 aa, Homo sapiens
EDAKNPQKNTPKFLY

SEQ ID NO: 497 - protein, 15 aa, Homo sapiens
ACLILGVKRPPEEKL

SEQ ID NO: 498 - protein, 15 aa, Homo sapiens
NRRAIHDKDYVKAGG

SEQ ID NO: 499 - protein, 15 aa, Homo sapiens
DVKAERLKVKELMGC

SEQ ID NO: 500 - protein, 15 aa, Homo sapiens
TIQVKNMKNKDKRLK

SEQ ID NO: 501 - protein, 15 aa, Homo sapiens
ITLLNELKRRGKRAY

SEQ ID NO: 502 - protein, 15 aa, Homo sapiens
VHDDKGTKRSITVTQ

SEQ ID NO: 503 - protein, 15 aa, Homo sapiens
RATLSEDKKHYILNG

SEQ ID NO: 504 - protein, 15 aa, Homo sapiens
AAQLALDKSDSHPSD

SEQ ID NO: 505 - protein, 15 aa, Homo sapiens
GILLFGTKAQKEKYL

SEQ ID NO: 506 - protein, 15 aa, Homo sapiens
SGLGSALKNPFGNAG

SEQ ID NO: 507 - protein, 15 aa, Homo sapiens
KLTIQGLKDFTPGKP

SEQ ID NO: 508 - protein, 15 aa, Homo sapiens
RASLRDEKGALFQAH

SEQ ID NO: 509 - protein, 15 aa, Homo sapiens
RASLRDEKGALFRAH

SEQ ID NO: 510 - protein, 9 aa, Homo sapiens
KPQVSESKL

SEQ ID NO: 511 - protein, 15 aa, Homo sapiens
QFVGAYMKETYHRIN

SEQ ID NO: 512 - protein, 15 aa, Homo sapiens
ENLFEWAKNSPLNKA

SEQ ID NO: 513 - protein, 9 aa, Homo sapiens
HLKSLQSKL

SEQ ID NO: 514 - protein, 15 aa, Homo sapiens
LMKYAQVKPDGTYVK

SEQ ID NO: 515 - protein, 15 aa, Homo sapiens
IREIGTHKPLPGITV

SEQ ID NO: 516 - protein, 14 aa, Homo sapiens
HESYKHLKSLQSKL

SEQ ID NO: 517 - protein, 15 aa, Homo sapiens
VVKLFSEKLLKIQDK

SEQ ID NO: 518 - protein, 15 aa, Homo sapiens
RKSAIMVKKMREFGI

SEQ ID NO: 519 - protein, 15 aa, Homo sapiens
CGGHGYSKLSGLPSL

SEQ ID NO: 520 - protein, 15 aa, Homo sapiens
LVFPKQFKTQQYYNV

SEQ ID NO: 521 - protein, 15 aa, Homo sapiens
CKIVGRSKDMIIRGG

SEQ ID NO: 522 - protein, 15 aa, Homo sapiens
PSFPGSRKLRSLKTS

SEQ ID NO: 523 - protein, 15 aa, Homo sapiens
LDFRSLVKSASPEHT

SEQ ID NO: 524 - protein, 15 aa, Homo sapiens
PKDQEEWKRRTGLLL

SEQ ID NO: 525 - protein, 15 aa, Homo sapiens
KELQQHVKSVTAPYK

SEQ ID NO: 526 - protein, 15 aa, Homo sapiens
AQKEKEGKRGPNPAF

SEQ ID NO: 527 - protein, 15 aa, Homo sapiens
KEIQEHVKKTTAPYK

SEQ ID NO: 528 - protein, 15 aa, Homo sapiens
TNILPFEKINEGFDL

SEQ ID NO: 529 - protein, 15 aa, Homo sapiens
STAGKVIKCKAAVLW

SEQ ID NO: 530 - protein, 15 aa, Homo sapiens
VADFMAKKFSLDALI

SEQ ID NO: 531 - protein, 15 aa, Homo sapiens
PLTNLCGKISNLKSP

SEQ ID NO: 532 - protein, 15 aa, Homo sapiens
KDKGKKGKKDKGPKV

SEQ ID NO: 533 - protein, 15 aa, Homo sapiens
DKGKKGKKDKGPKVP

SEQ ID NO: 534 - protein, 15 aa, Homo sapiens
GKKGKKDKGPKVPKE

SEQ ID NO: 535 - protein, 15 aa, Homo sapiens
LKRDHAVKYSKALID

SEQ ID NO: 536 - protein, 15 aa, Homo sapiens
LGLQLFVKDPALRLP

SEQ ID NO: 537 - protein, 15 aa, Homo sapiens
NRVLEIHKEHLSPVV

SEQ ID NO: 538 - protein, 15 aa, Homo sapiens
ASGSELGKKLKATMD

SEQ ID NO: 539 - protein, 15 aa, Homo sapiens
EWQDAVSKKTFPTVN

SEQ ID NO: 540 - protein, 15 aa, Homo sapiens
GERGFFIKPTVFGGV

SEQ ID NO: 541 - protein, 15 aa, Homo sapiens
LQNQIVWKIKETVKE

SEQ ID NO: 542 - protein, 15 aa, Homo sapiens
RASGTNDKPGGPHYI

SEQ ID NO: 543 - protein, 15 aa, Homo sapiens
QVIKETHKPLGDWSY

SEQ ID NO: 544 - protein, 15 aa, Homo sapiens
HSLWPQIKGRLLEEH

SEQ ID NO: 545 - protein, 15 aa, Homo sapiens
ARKEWDLKPIADRAQ

SEQ ID NO: 546 - protein, 15 aa, Homo sapiens
NHGHKVAKFCYADKS

SEQ ID NO: 547 - protein, 15 aa, Homo sapiens
KAFAEAMKKNLRVGN

SEQ ID NO: 548 - protein, 15 aa, Homo sapiens
GGKFVESKSDKWIDI

SEQ ID NO: 549 - protein, 15 aa, Homo sapiens
DAAIASCKRAFPAWA

SEQ ID NO: 550 - protein, 15 aa, Homo sapiens
AVLVGEAKKWLPELV

SEQ ID NO: 551 - protein, 15 aa, Homo sapiens
ELPFGGYKKSGFGRE

SEQ ID NO: 552 - protein, 15 aa, Homo sapiens
SILAKQQKKFGEEIA

SEQ ID NO: 553 - protein, 15 aa, Homo sapiens
ILAKQQKKFGEEIAR

SEQ ID NO: 554 - protein, 15 aa, Homo sapiens
RSLVLDLKQPRGAAV

SEQ ID NO: 555 - protein, 15 aa, Homo sapiens
KFADVFAKKTKAEWC

SEQ ID NO: 556 - protein, 15 aa, Homo sapiens
YYPKRPNKPLFTALV

SEQ ID NO: 557 - protein, 15 aa, Homo sapiens
GTTLAMTKNARVLGG

SEQ ID NO: 558 - protein, 15 aa, Homo sapiens
EKKDPSRKKEWWENA

SEQ ID NO: 559 - protein, 15 aa, Homo sapiens
LKYIGMAKREINKLL

SEQ ID NO: 560 - protein, 15 aa, Homo sapiens
KSKSGAGKGKLTRSL

SEQ ID NO: 561 - protein, 15 aa, Homo sapiens
KSGAGKGKLTRSLAV

SEQ ID NO: 562 - protein, 15 aa, Homo sapiens
NQERRKSKSGAGKGK

SEQ ID NO: 563 - protein, 15 aa, Homo sapiens
DILEIEFKKGVPVKV

SEQ ID NO: 564 - protein, 15 aa, Homo sapiens
AQFDYGRKCSEVARL

SEQ ID NO: 565 - protein, 15 aa, Homo sapiens
ATLKKTGKTVSYLGL

SEQ ID NO: 566 - protein, 15 aa, Homo sapiens
ARVAPKQKEFVITSL

SEQ ID NO: 567 - protein, 15 aa, Homo sapiens
VFARVAPKQKEFVIT

SEQ ID NO: 568 - protein, 15 aa, Homo sapiens
ELSSGSVKLMRERNK

SEQ ID NO: 569 - protein, 15 aa, Homo sapiens
LGNAIDGKGPIGSKT

SEQ ID NO: 570 - protein, 15 aa, Homo sapiens
DGSDEKKKLYCIYVA

SEQ ID NO: 571 - protein, 15 aa, Homo sapiens
EGLVRGQKVLDSGAP

SEQ ID NO: 572 - protein, 15 aa, Homo sapiens
IKGPEDKKKHLLIGV

SEQ ID NO: 573 - protein, 15 aa, Homo sapiens
ADLKLLAKKAQAQPV

SEQ ID NO: 574 - protein, 15 aa, Homo sapiens
PETKLDKKKYPYWPH

SEQ ID NO: 575 - protein, 15 aa, Homo sapiens
IQKLFVDKIREYKSK

SEQ ID NO: 576 - protein, 15 aa, Homo sapiens
ERELFKLKQMFGNAD

SEQ ID NO: 577 - protein, 15 aa, Homo sapiens
DKKLLEVKLGELPSW

SEQ ID NO: 578 - protein, 15 aa, Homo sapiens
PVPVKDKKLLEVKLG

SEQ ID NO: 579 - protein, 15 aa, Homo sapiens
SVLNPYVKRSIKVKS

SEQ ID NO: 580 - protein, 15 aa, Homo sapiens
VKRSIKVKSLNDITA

SEQ ID NO: 581 - protein, 15 aa, Homo sapiens
EAGGAFGKREQAEEE

SEQ ID NO: 582 - protein, 15 aa, Homo sapiens
LNAAAFLKLSQEREL

SEQ ID NO: 583 - protein, 15 aa, Homo sapiens
GAAEGPPKRPGGSSP

SEQ ID NO: 584 - protein, 15 aa, Homo sapiens
DLDREPKKEAVKSFI

SEQ ID NO: 585 - protein, 15 aa, Homo sapiens
DEVNYWDKQDHPISR

SEQ ID NO: 586 - protein, 8 aa, Homo sapiens
YPLDHFDK

SEQ ID NO: 587 - protein, 15 aa, Homo sapiens
VGAAYAAKRANANRV

SEQ ID NO: 588 - protein, 15 aa, Homo sapiens
KDAVDLMKALKFKKV

SEQ ID NO: 589 - protein, 15 aa, Homo sapiens
PQLKNLNKKLFTVVA

SEQ ID NO: 590 - protein, 15 aa, Homo sapiens
DFIHKHVKGSRLHLM

SEQ ID NO: 591 - protein, 15 aa, Homo sapiens
EGIRDVSKWSERTRK

SEQ ID NO: 592 - protein, 15 aa, Homo sapiens
VFASPGCKKWDTCAP

SEQ ID NO: 593 - protein, 15 aa, Homo sapiens
AAINDMKKSPEIISG

SEQ ID NO: 594 - protein, 15 aa, Homo sapiens
IAAINDMKKSPEIIS

SEQ ID NO: 595 - protein, 15 aa, Homo sapiens
IYTDLELKKHGLRLH

SEQ ID NO: 596 - protein, 15 aa, Homo sapiens
VDPTVPQKLITINQQ

SEQ ID NO: 597 - protein, 15 aa, Homo sapiens
TDVILDRKKNPEPWE

SEQ ID NO: 598 - protein, 15 aa, Homo sapiens
RAQSTAAKLGKTRLK

SEQ ID NO: 599 - protein, 15 aa, Homo sapiens
PLQPQKIKLAQKSEI

SEQ ID NO: 600 - protein, 15 aa, Homo sapiens
PESYLSNKRNVLNVY

SEQ ID NO: 601 - protein, 15 aa, Homo sapiens
KKQVRLLKENQMLRR

SEQ ID NO: 602 - protein, 15 aa, Homo sapiens
PTLEHTTKSFLRNWR

SEQ ID NO: 603 - protein, 15 aa, Homo sapiens
AETCQEPKEFRLPKD

SEQ ID NO: 604 - protein, 15 aa, Homo sapiens
DICGPGTKKVHVIFN

SEQ ID NO: 605 - protein, 15 aa, Homo sapiens
LKEEEEDKKRKEEEE

SEQ ID NO: 606 - protein, 15 aa, Homo sapiens
KEEEEDKKRKEEEEA

SEQ ID NO: 607 - protein, 15 aa, Homo sapiens
DAQIFIQKKAVKNFT

SEQ ID NO: 608 - protein, 15 aa, Homo sapiens
AQIFIQKKAVKNFTE

SEQ ID NO: 609 - protein, 15 aa, Homo sapiens
KVFEHIGKKTPIAVR

SEQ ID NO: 610 - protein, 15 aa, Homo sapiens
LTKVWPHKDYPLIPV

SEQ ID NO: 611 - protein, 15 aa, Homo sapiens
PSFIHSQKRNPQTHL

SEQ ID NO: 612 - protein, 15 aa, Homo sapiens
VLNEEQRKRLCENIA

SEQ ID NO: 613 - protein, 15 aa, Homo sapiens
CIIVMPEKMSSEKVD

SEQ ID NO: 614 - protein, 15 aa, Homo sapiens
KELEKSEKEEDEDDD

SEQ ID NO: 615 - protein, 15 aa, Homo sapiens
FGHPSLVKALSYLYE

SEQ ID NO: 616 - protein, 15 aa, Homo sapiens
DMLKEEEKDVKEVDV

SEQ ID NO: 617 - protein, 15 aa, Homo sapiens
HRMLQEEKRLRTAYL

SEQ ID NO: 618 - protein, 15 aa, Homo sapiens
LENLQELKDLNQRQA

SEQ ID NO: 619 - protein, 15 aa, Homo sapiens
KPSPLSVKNTKRRLE

SEQ ID NO: 620 - protein, 15 aa, Homo sapiens
MGTISDVKTNVQLLK

SEQ ID NO: 621 - protein, 15 aa, Homo sapiens
EDAKLDAKAVERLKS

SEQ ID NO: 622 - protein, 15 aa, Homo sapiens
SRTWAGEKSKREMYS

SEQ ID NO: 623 - protein, 15 aa, Homo sapiens
DTEIQLLKEKLTLSE

SEQ ID NO: 624 - protein, 15 aa, Homo sapiens
SVSDEELKRRVAEEL

SEQ ID NO: 625 - protein, 15 aa, Homo sapiens
DMTIHEGKRWSAACA

SEQ ID NO: 626 - protein, 15 aa, Homo sapiens
DQQNCKMKFGSWTYD

SEQ ID NO: 627 - protein, 15 aa, Homo sapiens
FGSWTYDKAKIDLEQ

SEQ ID NO: 628 - protein, 15 aa, Homo sapiens
DNRESLEKRIQTYLQ

SEQ ID NO: 629 - protein, 15 aa, Homo sapiens
GPIAGKSKRRISCKD

SEQ ID NO: 630 - protein, 9 aa, Homo sapiens
PEELGLDKV

SEQ ID NO: 631 - protein, 15 aa, Homo sapiens
LVPSISNKRIVGCIC

SEQ ID NO: 632 - protein, 15 aa, Homo sapiens
PRCGAHYKLVPQQLA

SEQ ID NO: 633 - protein, 8 aa, Homo sapiens
AGIFQSVK

SEQ ID NO: 634 - protein, 15 aa, Homo sapiens
KLNEINEKIAPSFAV

SEQ ID NO: 635 - protein, 15 aa, Homo sapiens
TGLAAGAKTYKMSMA

SEQ ID NO: 636 - protein, 15 aa, Homo sapiens
VEMDAVGKDGRVISH

SEQ ID NO: 637 - protein, 15 aa, Homo sapiens
AAGAKTYKMSMANRG

SEQ ID NO: 638 - protein, 15 aa, Homo sapiens
NLAVPLYKNGVKIMG

SEQ ID NO: 639 - protein, 15 aa, Homo sapiens
LFAEAVQKSRKVDSK

SEQ ID NO: 640 - protein, 15 aa, Homo sapiens
LEDGTKMKGYSFGHP

SEQ ID NO: 641 - protein, 15 aa, Homo sapiens
GQWLQEEKVPAIYGV

SEQ ID NO: 642 - protein, 15 aa, Homo sapiens
VAVDCGIKNNVIRLL

SEQ ID NO: 643 - protein, 15 aa, Homo sapiens
TITSVLPKPALVASR

SEQ ID NO: 644 - protein, 15 aa, Homo sapiens
TPRLPMNKEWPSNLD

SEQ ID NO: 645 - protein, 15 aa, Homo sapiens
PLIQNVRKILESDRK

SEQ ID NO: 646 - protein, 15 aa, Homo sapiens
VLKEYGVKVLGTSVE

SEQ ID NO: 647 - protein, 15 aa, Homo sapiens
NCGVELFKRGVLKEY

SEQ ID NO: 648 - protein, 15 aa, Homo sapiens
QLHNEGFKLFATEAT

SEQ ID NO: 649 - protein, 15 aa, Homo sapiens
PLYKNGVKIMGTSPL

SEQ ID NO: 650 - protein, 15 aa, Homo sapiens
AMKEENVKTVLMNPN

SEQ ID NO: 651 - protein, 15 aa, Homo sapiens
GIPLPEIKNVVSGKT

SEQ ID NO: 652 - protein, 15 aa, Homo sapiens
EKLTYIDKWFLYKMR

SEQ ID NO: 653 - protein, 15 aa, Homo sapiens
KSRKVDSKSLFHYRQ

SEQ ID NO: 654 - protein, 15 aa, Homo sapiens
FSLIKKGKATTITSV

SEQ ID NO: 655 - protein, 15 aa, Homo sapiens
KDVKVYGKGNPTKVV

SEQ ID NO: 656 - protein, 15 aa, Homo sapiens
KEIGFSDKQISKCLG

SEQ ID NO: 657 - protein, 15 aa, Homo sapiens
TRELRLKKNIHPWVK

SEQ ID NO: 658 - protein, 15 aa, Homo sapiens
KVKDATRKIAKAFAI

SEQ ID NO: 659 - protein, 15 aa, Homo sapiens
TRIYAIAKAIDDNMS

SEQ ID NO: 660 - protein, 15 aa, Homo sapiens
SQAVKAMKEENVKTV

SEQ ID NO: 661 - protein, 15 aa, Homo sapiens
FSDKQISKCLGLTEA

SEQ ID NO: 662 - protein, 15 aa, Homo sapiens
DATRKIAKAFAISGP

SEQ ID NO: 663 - protein, 15 aa, Homo sapiens
TGFKIPQKGILIGIQ

SEQ ID NO: 664 - protein, 15 aa, Homo sapiens
RDILNMEKTLKGLNS

SEQ ID NO: 665 - protein, 15 aa, Homo sapiens
YAFTSFGKKLTKNKM

SEQ ID NO: 666 - protein, 15 aa, Homo sapiens
MKDCSAGKGFDRHLL

SEQ ID NO: 667 - protein, 15 aa, Homo sapiens
AFTSFGKKLTKNKML

SEQ ID NO: 668 - protein, 15 aa, Homo sapiens
EQTPEHGKKKRGRGK

SEQ ID NO: 669 - protein, 15 aa, Homo sapiens
KCEDGSWKRLPSYKR

SEQ ID NO: 670 - protein, 15 aa, Homo sapiens
YRLEEVAKRNSLKEL

SEQ ID NO: 671 - protein, 8 aa, Homo sapiens
KLAYRPPK

SEQ ID NO: 672 - protein, 15 aa, Homo sapiens
MMLFGMGKRRCIGEV

SEQ ID NO: 673 - protein, 15 aa, Homo sapiens
IVLVPNKKVGLQFLQ

SEQ ID NO: 674 - protein, 15 aa, Homo sapiens
PATVEDLKKLRYLEC

SEQ ID NO: 675 - protein, 15 aa, Homo sapiens
SLEHSWIKAIRRRNV

SEQ ID NO: 676 - protein, 15 aa, Homo sapiens
TLQTQTAKLQVENST

SEQ ID NO: 677 - protein, 15 aa, Homo sapiens
ISKDSIQKSLQMYLE

SEQ ID NO: 678 - protein, 15 aa, Homo sapiens
QPVLVAVKMLRADAN

SEQ ID NO: 679 - protein, 15 aa, Homo sapiens
MLRADANKNARNDFL

SEQ ID NO: 680 - protein, 15 aa, Homo sapiens
LESWQIGKIGTVMTF

SEQ ID NO: 681 - protein, 15 aa, Homo sapiens
EPAAAGQKEKDEALE

SEQ ID NO: 682 - protein, 15 aa, Homo sapiens
KDEALEEKLRNLTFR

SEQ ID NO: 683 - protein, 15 aa, Homo sapiens
RLVATAVKLHGGIDI

SEQ ID NO: 684 - protein, 15 aa, Homo sapiens
KHFYSLVKQRESLGA

SEQ ID NO: 685 - protein, 15 aa, Homo sapiens
QLALDHFKKTKKMGG

SEQ ID NO: 686 - protein, 15 aa, Homo sapiens
QEMRARHKEAFLKKH

SEQ ID NO: 687 - protein, 15 aa, Homo sapiens
FLKKHNLKLGFMSAF

SEQ ID NO: 688 - protein, 15 aa, Homo sapiens
HKEAFLKKHNLKLGF

SEQ ID NO: 689 - protein, 15 aa, Homo sapiens
KQALKQIKAKGLKRR

SEQ ID NO: 690 - protein, 15 aa, Homo sapiens
RITMPLSKNDRKKYN

SEQ ID NO: 691 - protein, 15 aa, Homo sapiens
VSTRSRSKLPAGKNV

SEQ ID NO: 692 - protein, 15 aa, Homo sapiens
FKEADATKNKEKLEK

SEQ ID NO: 693 - protein, 15 aa, Homo sapiens
EADATKNKEKLEKLK

SEQ ID NO: 694 - protein, 15 aa, Homo sapiens
KNKEKLEKLKSQADQ

SEQ ID NO: 695 - protein, 15 aa, Homo sapiens
KDKSERIKGLRERSS

SEQ ID NO: 696 - protein, 15 aa, Homo sapiens
ETQALLRKKEKEFEE

SEQ ID NO: 697 - protein, 15 aa, Homo sapiens
ATVMNLSKKIKKIGV

SEQ ID NO: 698 - protein, 15 aa, Homo sapiens
AGLDVGWKSRKGQGL

SEQ ID NO: 699 - protein, 9 aa, Homo sapiens
LAGSPSSKL

SEQ ID NO: 700 - protein, 15 aa, Homo sapiens
NQLATANKMITSVLE

SEQ ID NO: 701 - protein, 15 aa, Homo sapiens
KPLGRIHKPDPWLST

SEQ ID NO: 702 - protein, 15 aa, Homo sapiens
KNENESEKRELVFKE

SEQ ID NO: 703 - protein, 15 aa, Homo sapiens
WDWKKGEKLSIARGS

SEQ ID NO: 704 - protein, 15 aa, Homo sapiens
VNSNLVCKVSDFGMS

SEQ ID NO: 705 - protein, 15 aa, Homo sapiens
GFNSNYLKKVISYWR

SEQ ID NO: 706 - protein, 15 aa, Homo sapiens
FNSNYLKKVISYWRN

SEQ ID NO: 707 - protein, 15 aa, Homo sapiens
FYVQQFKKSGFRGPL

SEQ ID NO: 708 - protein, 15 aa, Homo sapiens
QFYVQQFKKSGFRGP

SEQ ID NO: 709 - protein, 15 aa, Homo sapiens
DDIGANLKPARDLGM

SEQ ID NO: 710 - protein, 15 aa, Homo sapiens
RNMERNWKWACKSLG

SEQ ID NO: 711 - protein, 15 aa, Homo sapiens
TGEVVAIKKIFDAFR

SEQ ID NO: 712 - protein, 15 aa, Homo sapiens
VAQHDVYKGLLPEEL

SEQ ID NO: 713 - protein, 15 aa, Homo sapiens
CLDPGAFKELFPDWK

SEQ ID NO: 714 - protein, 15 aa, Homo sapiens
ELWVIDEKNWKPGRV

SEQ ID NO: 715 - protein, 15 aa, Homo sapiens
RPTLEGGKRIAYGAR

SEQ ID NO: 716 - protein, 15 aa, Homo sapiens
KELFPDWKEKGAPLN

SEQ ID NO: 717 - protein, 15 aa, Homo sapiens
ARLWGIQKNRPAMNY

SEQ ID NO: 718 - protein, 15 aa, Homo sapiens
KDFFEVAKIKMTGTT

SEQ ID NO: 719 - protein, 15 aa, Homo sapiens
EQNVPVPKEPIIFSK

SEQ ID NO: 720 - protein, 15 aa, Homo sapiens
LPPQEDSKVTKQDKN

SEQ ID NO: 721 - protein, 15 aa, Homo sapiens
TNYSHHSKSQRNKDI

SEQ ID NO: 722 - protein, 15 aa, Homo sapiens
RAKSQTEKLAKVYQP

SEQ ID NO: 723 - protein, 15 aa, Homo sapiens
AKGKETEKAKERYDK

SEQ ID NO: 724 - protein, 15 aa, Homo sapiens
DALDAKSKEFAQIIK

SEQ ID NO: 725 - protein, 15 aa, Homo sapiens
RFGQHLIKPSVVFLK

SEQ ID NO: 726 - protein, 15 aa, Homo sapiens
KLIEESKKLLKLKSE

SEQ ID NO: 727 - protein, 15 aa, Homo sapiens
EESKKLLKLKSEMEE

SEQ ID NO: 728 - protein, 15 aa, Homo sapiens
ESGSSLGKKVAGALQ

SEQ ID NO: 729 - protein, 15 aa, Homo sapiens
LEIAKSEKEPLHVVA

SEQ ID NO: 730 - protein, 15 aa, Homo sapiens
LGVQLDDKRKEMLKR

SEQ ID NO: 731 - protein, 15 aa, Homo sapiens
VQLDDKRKEMLKRHP

SEQ ID NO: 732 - protein, 15 aa, Homo sapiens
EASHLAEKVFLSTTG

SEQ ID NO: 733 - protein, 15 aa, Homo sapiens
NPEGFTGKRVIIIGI

SEQ ID NO: 734 - protein, 15 aa, Homo sapiens
MALGICAKGVIVYEV

SEQ ID NO: 735 - protein, 15 aa, Homo sapiens
YCPDIKEKGLSGSPR

SEQ ID NO: 736 - protein, 15 aa, Homo sapiens
GLIRARLKGAAVSKG

SEQ ID NO: 737 - protein, 15 aa, Homo sapiens
ARRDDFCKQNSKASS

SEQ ID NO: 738 - protein, 15 aa, Homo sapiens
FSYPVVPKGKARIRV

SEQ ID NO: 739 - protein, 15 aa, Homo sapiens
RMADDMLKRGIFVIG

SEQ ID NO: 740 - protein, 8 aa, Homo sapiens
IQAFTASK

SEQ ID NO: 741 - protein, 15 aa, Homo sapiens
PEMVSLLKRNNCGKA

SEQ ID NO: 742 - protein, 15 aa, Homo sapiens
KALEIAEKKLLGPLG

SEQ ID NO: 743 - protein, 15 aa, Homo sapiens
IINLPGSKKGSQECF

SEQ ID NO: 744 - protein, 15 aa, Homo sapiens
GEMFLEEKIPSISDL

SEQ ID NO: 745 - protein, 15 aa, Homo sapiens
ESQRVNKKVNNDASL

SEQ ID NO: 746 - protein, 15 aa, Homo sapiens
GIPGSVFKRTSPFLT

SEQ ID NO: 747 - protein, 15 aa, Homo sapiens
IRAYLNQKGEGHRTV

SEQ ID NO: 748 - protein, 15 aa, Homo sapiens
RHIGPGDKDQREMLQ

SEQ ID NO: 749 - protein, 15 aa, Homo sapiens
ILDTRPFKKSANIEA

SEQ ID NO: 750 - protein, 8 aa, Homo sapiens
EKKDQDSK

SEQ ID NO: 751 - protein, 15 aa, Homo sapiens
ILGFPKAKPYEGSIL

SEQ ID NO: 752 - protein, 15 aa, Homo sapiens
GGAKAGVKINPKNYT

SEQ ID NO: 753 - protein, 15 aa, Homo sapiens
DVPFGGAKAGVKINP

SEQ ID NO: 754 - protein, 15 aa, Homo sapiens
SHAQKLGKLGFPVYS

SEQ ID NO: 755 - protein, 15 aa, Homo sapiens
YMAAETAKELTPFLL

SEQ ID NO: 756 - protein, 15 aa, Homo sapiens
LEAMQAGKVHLARFV

SEQ ID NO: 757 - protein, 8 aa, Homo sapiens
HAIHQVTK

SEQ ID NO: 758 - protein, 15 aa, Homo sapiens
CKDADEAKRVESQLK

SEQ ID NO: 759 - protein, 15 aa, Homo sapiens
AYRDDNGKPYVLPSV

SEQ ID NO: 760 - protein, 15 aa, Homo sapiens
KRVESQLKILIRPMY

SEQ ID NO: 761 - protein, 15 aa, Homo sapiens
EIATVVKKRNLFAFF

SEQ ID NO: 762 - protein, 15 aa, Homo sapiens
YVLPSVRKAEAQIAA

SEQ ID NO: 763 - protein, 15 aa, Homo sapiens
SVLGNLAKKAKLTED

SEQ ID NO: 764 - protein, 15 aa, Homo sapiens
DGIVSHLKKQAGPAS

SEQ ID NO: 765 - protein, 15 aa, Homo sapiens
LRTEEEFKKFISDKD

SEQ ID NO: 766 - protein, 15 aa, Homo sapiens
IYFSPANKKLNPKKY

SEQ ID NO: 767 - protein, 15 aa, Homo sapiens
ILNYIASKYNLYGKD

SEQ ID NO: 768 - protein, 15 aa, Homo sapiens
ISNLPTVKKFLQPGS

SEQ ID NO: 769 - protein, 15 aa, Homo sapiens
FLSVMLEKGSLSAMR

SEQ ID NO: 770 - protein, 15 aa, Homo sapiens
KIATPKVKNQLKETT

SEQ ID NO: 771 - protein, 15 aa, Homo sapiens
QIPIHFPKDFLSVML

SEQ ID NO: 772 - protein, 15 aa, Homo sapiens
ISRFEGLKKISAYMK

SEQ ID NO: 773 - protein, 11 aa, Homo sapiens
HANSPNKKFYQ

SEQ ID NO: 774 - protein, 15 aa, Homo sapiens
LLTQMVSKGFLGRKS

SEQ ID NO: 775 - protein, 15 aa, Homo sapiens
HGQVLCKKNKFGAPQ

SEQ ID NO: 776 - protein, 15 aa, Homo sapiens
GGNEKLVKLESGLKV

SEQ ID NO: 777 - protein, 15 aa, Homo sapiens
LIEEEVAKLLKLKAQ

SEQ ID NO: 778 - protein, 15 aa, Homo sapiens
EEVAKLLKLKAQLGP

SEQ ID NO: 779 - protein, 15 aa, Homo sapiens
AVASTEVKMKLQEFV

SEQ ID NO: 780 - protein, 15 aa, Homo sapiens
DLADRMQKCVNSCKS

SEQ ID NO: 781 - protein, 15 aa, Homo sapiens
NPDAPEGKRSSGLTA

SEQ ID NO: 782 - protein, 15 aa, Homo sapiens
NLKNEITKKVQVPNC

SEQ ID NO: 783 - protein, 15 aa, Homo sapiens
KETLEQQKRICEMAA

SEQ ID NO: 784 - protein, 15 aa, Homo sapiens
DSWSRMVKSLNVSSS

SEQ ID NO: 785 - protein, 15 aa, Homo sapiens
SGANGILKKVKKGSL

SEQ ID NO: 786 - protein, 15 aa, Homo sapiens
GANGILKKVKKGSLL

SEQ ID NO: 787 - protein, 15 aa, Homo sapiens
GHLLLVAKQTAKAEG

SEQ ID NO: 788 - protein, 15 aa, Homo sapiens
PVLTPNLKGFEAAVA

SEQ ID NO: 789 - protein, 15 aa, Homo sapiens
SSTSDLPKRLASRKC

SEQ ID NO: 790 - protein, 15 aa, Homo sapiens
QREQFYHKVNFSPPG

SEQ ID NO: 791 - protein, 15 aa, Homo sapiens
DTQTSLYKGLEAFGG

SEQ ID NO: 792 - protein, 15 aa, Homo sapiens
ENVYDFYKPNLASEY

SEQ ID NO: 793 - protein, 15 aa, Homo sapiens
IFHTPFCKMVQKSLA

SEQ ID NO: 794 - protein, 15 aa, Homo sapiens
LEDTYTNKDLDKALL

SEQ ID NO: 795 - protein, 15 aa, Homo sapiens
YTNKDLDKALLKASQ

SEQ ID NO: 796 - protein, 15 aa, Homo sapiens
PFCKMVQKSLARLMF

SEQ ID NO: 797 - protein, 15 aa, Homo sapiens
KAMEERYKMYLEKAR

SEQ ID NO: 798 - protein, 10 aa, Homo sapiens
AVIGYKMKAQ

SEQ ID NO: 799 - protein, 15 aa, Homo sapiens
SYAEELAKHGMKVVL

SEQ ID NO: 800 - protein, 15 aa, Homo sapiens
DQVSSEIKEKFKVET

SEQ ID NO: 801 - protein, 15 aa, Homo sapiens
ASEDIYDKIKTGLAG

SEQ ID NO: 802 - protein, 15 aa, Homo sapiens
EDIYDKIKTGLAGLE

SEQ ID NO: 803 - protein, 15 aa, Homo sapiens
LVLWDINKRGVEETA

SEQ ID NO: 804 - protein, 15 aa, Homo sapiens
NISNTVMKVKQILGR

SEQ ID NO: 805 - protein, 15 aa, Homo sapiens
TAEAYLGKKVTHAVV

SEQ ID NO: 806 - protein, 15 aa, Homo sapiens
KGVGDSFKKYPDYES

SEQ ID NO: 807 - protein, 15 aa, Homo sapiens
FKLKQMWKSPNGTIR

SEQ ID NO: 808 - protein, 15 aa, Homo sapiens
STTTDFIKSVIGHLQ

SEQ ID NO: 809 - protein, 15 aa, Homo sapiens
RMNSVNFKNILQWES

SEQ ID NO: 810 - protein, 15 aa, Homo sapiens
IARTDLKKNRDYPLA

SEQ ID NO: 811 - protein, 15 aa, Homo sapiens
PLASKDAKKQLLCGA

SEQ ID NO: 812 - protein, 15 aa, Homo sapiens
IGGFKMPKDKVDSKD

SEQ ID NO: 813 - protein, 15 aa, Homo sapiens
ASAGLEAKPRIWSLA

SEQ ID NO: 814 - protein, 15 aa, Homo sapiens
MPGFSTSKKLDKLGM

SEQ ID NO: 815 - protein, 15 aa, Homo sapiens
EAKITKKKSLVSFKA

SEQ ID NO: 816 - protein, 15 aa, Homo sapiens
DSDELTIKRFIEGKE

SEQ ID NO: 817 - protein, 15 aa, Homo sapiens
IKRFIEGKEKPGALW

SEQ ID NO: 818 - protein, 15 aa, Homo sapiens
SKGKWVYKGEWTHGF

SEQ ID NO: 819 - protein, 15 aa, Homo sapiens
ASLAILQKQLSEMAN

SEQ ID NO: 820 - protein, 15 aa, Homo sapiens
TVMRQNVKRATFDEV

SEQ ID NO: 821 - protein, 15 aa, Homo sapiens
RGGSGFLKSGGEMLK

SEQ ID NO: 822 - protein, 15 aa, Homo sapiens
KSGGEMLKVGGSVNR

SEQ ID NO: 823 - protein, 15 aa, Homo sapiens
PPAPPTRKSSLEQRS

SEQ ID NO: 824 - protein, 15 aa, Homo sapiens
AKIDEERKALETKLD

SEQ ID NO: 825 - protein, 15 aa, Homo sapiens
QKEIEELKKKLEEGE

SEQ ID NO: 826 - protein, 15 aa, Homo sapiens
KEIEELKKKLEEGEE

SEQ ID NO: 827 - protein, 15 aa, Homo sapiens
TPRHILVKFWNSSDK

SEQ ID NO: 828 - protein, 15 aa, Homo sapiens
PNAVLAFKREGYRPF

SEQ ID NO: 829 - protein, 15 aa, Homo sapiens
RHYEKDIKKVKEEKA

SEQ ID NO: 830 - protein, 15 aa, Homo sapiens
ARFYVYNKKKRLVNT

SEQ ID NO: 831 - protein, 15 aa, Homo sapiens
HYEKDIKKVKEEKAY

SEQ ID NO: 832 - protein, 15 aa, Homo sapiens
VVLVGLGKKAAGIDE

SEQ ID NO: 833 - protein, 15 aa, Homo sapiens
HREKELQKRSEVAKD

SEQ ID NO: 834 - protein, 15 aa, Homo sapiens
DGPWEKQKSSGLNLC

SEQ ID NO: 835 - protein, 10 aa, Homo sapiens
SNKCDVVVVG

SEQ ID NO: 836 - protein, 8 aa, Homo sapiens
EDFVKTLK

SEQ ID NO: 837 - protein, 15 aa, Homo sapiens
AVLVSPSKMKPPAPE

SEQ ID NO: 838 - protein, 15 aa, Homo sapiens
LEKEGVPKAEATRKI

SEQ ID NO: 839 - protein, 15 aa, Homo sapiens
QGQPSRQKRSVENTG

SEQ ID NO: 840 - protein, 15 aa, Homo sapiens
FGKGENAKKYLRTDD

SEQ ID NO: 841 - protein, 15 aa, Homo sapiens
KQCCVYRKCDKIEAR

SEQ ID NO: 842 - protein, 15 aa, Homo sapiens
CDKIEARKMERLAKK

SEQ ID NO: 843 - protein, 15 aa, Homo sapiens
TDNIFAIKSWAKRKF

SEQ ID NO: 844 - protein, 15 aa, Homo sapiens
FAIKSWAKRKFGFEE

SEQ ID NO: 845 - protein, 15 aa, Homo sapiens
LVQQNQLKKGDALVV

SEQ ID NO: 846 - protein, 15 aa, Homo sapiens
VQFETNMKGRTSRKL

SEQ ID NO: 847 - protein, 15 aa, Homo sapiens
RIFGLDIKGRDCGNE

SEQ ID NO: 848 - protein, 15 aa, Homo sapiens
GVRSVMLKKSLTATD

SEQ ID NO: 849 - protein, 15 aa, Homo sapiens
HSEYQKSKRISIFLS

SEQ ID NO: 850 - protein, 15 aa, Homo sapiens
PEELPGVKPFTRGPY

SEQ ID NO: 851 - protein, 15 aa, Homo sapiens
SAIKRVHKFMEREGR

SEQ ID NO: 852 - protein, 15 aa, Homo sapiens
KWMDLASKAGKHLQL

SEQ ID NO: 853 - protein, 15 aa, Homo sapiens
DLASKAGKHLQLKET

SEQ ID NO: 854 - protein, 15 aa, Homo sapiens
METVSKAKGNLEKMC

SEQ ID NO: 855 - protein, 15 aa, Homo sapiens
KEEYAKGKIKSSQDG

SEQ ID NO: 856 - protein, 15 aa, Homo sapiens
IKSSQDGKVTVETED

SEQ ID NO: 857 - protein, 15 aa, Homo sapiens
CIRSNAEKKELCFDD

SEQ ID NO: 858 - protein, 15 aa, Homo sapiens
IRSNAEKKELCFDDE

SEQ ID NO: 859 - protein, 15 aa, Homo sapiens
KKDHGEYKATLKDDR

SEQ ID NO: 860 - protein, 15 aa, Homo sapiens
IRMYNIKKTCSDPKS

SEQ ID NO: 861 - protein, 15 aa, Homo sapiens
KSMEPSLKYINKKFP

SEQ ID NO: 862 - protein, 15 aa, Homo sapiens
VDHYNEVKSVTICDY

SEQ ID NO: 863 - protein, 15 aa, Homo sapiens
RQIIGQAKKHPSLIP

SEQ ID NO: 864 - protein, 15 aa, Homo sapiens
DVLEEIPKNAAYRKY

SEQ ID NO: 865 - protein, 15 aa, Homo sapiens
QSGVNNTKKWKMEFD

SEQ ID NO: 866 - protein, 15 aa, Homo sapiens
KARAVAPKPSSRGEY

SEQ ID NO: 867 - protein, 15 aa, Homo sapiens
LSRGDWYKTKEILLK

SEQ ID NO: 868 - protein, 15 aa, Homo sapiens
LKAGKIPKPGPRSGR

SEQ ID NO: 869 - protein, 15 aa, Homo sapiens
VADRPDIKKATQAAK

SEQ ID NO: 870 - protein, 15 aa, Homo sapiens
QSDVAYRKDAKENLH

SEQ ID NO: 871 - protein, 15 aa, Homo sapiens
ELDRPDIKMATQISK

SEQ ID NO: 872 - protein, 15 aa, Homo sapiens
STVPTPAKRRSSALW

SEQ ID NO: 873 - protein, 15 aa, Homo sapiens
PPTSPMEKFPKSRLS

SEQ ID NO: 874 - protein, 15 aa, Homo sapiens
SPMEKFPKSRLSFDL

SEQ ID NO: 875 - protein, 15 aa, Homo sapiens
IYELRTYKLKPGTMI

SEQ ID NO: 876 - protein, 15 aa, Homo sapiens
KIQFHNVKPEYLDAY

SEQ ID NO: 877 - protein, 13 aa, Homo sapiens
SRIMIPLKISPLQ

SEQ ID NO: 878 - protein, 15 aa, Homo sapiens
ELRTYKLKPGTMIEW

SEQ ID NO: 879 - protein, 15 aa, Homo sapiens
YSDIDLKKLAEIRQQ

SEQ ID NO: 880 - protein, 15 aa, Homo sapiens
VYSDIDLKKLAEIRQ

SEQ ID NO: 881 - protein, 15 aa, Homo sapiens
AMLLGDAKKTCDALQ

SEQ ID NO: 882 - protein, 15 aa, Homo sapiens
AGVQNLVKQGFNVVV

SEQ ID NO: 883 - protein, 15 aa, Homo sapiens
NSLLDDAKARLRKYD

SEQ ID NO: 884 - protein, 15 aa, Homo sapiens
SASASSEKGDLLDSP

SEQ ID NO: 885 - protein, 15 aa, Homo sapiens
YCGDTEGKKVMSILL

SEQ ID NO: 886 - protein, 15 aa, Homo sapiens
GNSVHEDKKFLDKYM

SEQ ID NO: 887 - protein, 15 aa, Homo sapiens
HLQAATPKGYEPDAS

SEQ ID NO: 888 - protein, 15 aa, Homo sapiens
QVTMKTAKVAASDWT

SEQ ID NO: 889 - protein, 15 aa, Homo sapiens
MGQEEEKKKRLQAFQ

SEQ ID NO: 890 - protein, 15 aa, Homo sapiens
SMGQEEEKKKRLQAF

SEQ ID NO: 891 - protein, 15 aa, Homo sapiens
LKFRIKQKGEYLPLL

SEQ ID NO: 892 - protein, 15 aa, Homo sapiens
AVLARVYKQSDLDTL

SEQ ID NO: 893 - protein, 15 aa, Homo sapiens
EDGRSCKKVTIRMTI

SEQ ID NO: 894 - protein, 15 aa, Homo sapiens
VEPEGIEKERTQSFL

SEQ ID NO: 895 - protein, 15 aa, Homo sapiens
VKVIAHGKDHPTAAT

SEQ ID NO: 896 - protein, 15 aa, Homo sapiens
HFHPKALKDVKGQIG

SEQ ID NO: 897 - protein, 15 aa, Homo sapiens
PKALKDVKGQIGAPM

SEQ ID NO: 898 - protein, 15 aa, Homo sapiens
YAPKPSGKASKGNKS

SEQ ID NO: 899 - protein, 15 aa, Homo sapiens
GKASKGNKSKGKKSK

SEQ ID NO: 900 - protein, 15 aa, Homo sapiens
RFQNQVDKMKEKVKN

SEQ ID NO: 901 - protein, 15 aa, Homo sapiens
QNQVDKMKEKVKNVE

SEQ ID NO: 902 - protein, 15 aa, Homo sapiens
QVDKMKEKVKNVEER

SEQ ID NO: 903 - protein, 15 aa, Homo sapiens
MSLLADLKTMVETKK

SEQ ID NO: 904 - protein, 15 aa, Homo sapiens
LKTMVETKKVTSSGV

SEQ ID NO: 905 - protein, 15 aa, Homo sapiens
LKADQLYKQKIIRGF

SEQ ID NO: 906 - protein, 15 aa, Homo sapiens
LKADQLYKQKFIRGF

SEQ ID NO: 907 - protein, 15 aa, Homo sapiens
ERTLDGFKKFLESGG

SEQ ID NO: 908 - protein, 15 aa, Homo sapiens
KTAAESFKGKILFIF

SEQ ID NO: 909 - protein, 15 aa, Homo sapiens
EDVAFDEKKNVFVEF

SEQ ID NO: 910 - protein, 15 aa, Homo sapiens
TAETDLAKRFDVSGY

SEQ ID NO: 911 - protein, 15 aa, Homo sapiens
VEKAEQQKKAAIISA

SEQ ID NO: 912 - protein, 15 aa, Homo sapiens
ERAGLQRKGGIKKRA

SEQ ID NO: 913 - protein, 15 aa, Homo sapiens
PDGREDQKRKGISDL

SEQ ID NO: 914 - protein, 15 aa, Homo sapiens
IAFYNGNKREKQTVH

SEQ ID NO: 915 - protein, 15 aa, Homo sapiens
QNNEKEGKKERAVVD

SEQ ID NO: 916 - protein, 15 aa, Homo sapiens
NVKPEDVKRVASKML

SEQ ID NO: 917 - protein, 15 aa, Homo sapiens
PKTIGCSKNFPGKTA

SEQ ID NO: 918 - protein, 15 aa, Homo sapiens
VYVQHLLKQDREHLW

SEQ ID NO: 919 - protein, 15 aa, Homo sapiens
AQAVDYIKKLMTKGR

SEQ ID NO: 920 - protein, 15 aa, Homo sapiens
VPRWSNAKMEIATKD

SEQ ID NO: 921 - protein, 15 aa, Homo sapiens
FHDIDDVKKFKPGYL

SEQ ID NO: 922 - protein, 15 aa, Homo sapiens
EKTQKRVKEGNGTSN

SEQ ID NO: 923 - protein, 9 aa, Homo sapiens
EKPFAIAKE

SEQ ID NO: 924 - protein, 15 aa, Homo sapiens
GEKGFGYKNSKFHRV

SEQ ID NO: 925 - protein, 15 aa, Homo sapiens
RPEGYNLKDEEGKLK

SEQ ID NO: 926 - protein, 15 aa, Homo sapiens
NLKDEEGKLKDLSTV

SEQ ID NO: 927 - protein, 15 aa, Homo sapiens
QRMDVLAKKATEMGV

SEQ ID NO: 928 - protein, 15 aa, Homo sapiens
RDGSGTNKRDKQYQA

SEQ ID NO: 929 - protein, 15 aa, Homo sapiens
SCHPLTRKNNFGNGR

SEQ ID NO: 930 - protein, 15 aa, Homo sapiens
LEETNIPKRLYKALS

SEQ ID NO: 931 - protein, 15 aa, Homo sapiens
CKYSEEMKKIQEKQR

SEQ ID NO: 932 - protein, 9 aa, Homo sapiens
AKPLTDQEK

SEQ ID NO: 933 - protein, 15 aa, Homo sapiens
SSSMNSIKTFWSPEL

SEQ ID NO: 934 - protein, 15 aa, Homo sapiens
PSKMKRSKGIHVFKK

SEQ ID NO: 935 - protein, 15 aa, Homo sapiens
SKGIHVFKKPSFSKK

SEQ ID NO: 936 - protein, 15 aa, Homo sapiens
FKKPSFSKKKEKDFK

SEQ ID NO: 937 - protein, 15 aa, Homo sapiens
TFPKMKSKFHDKAAK

SEQ ID NO: 938 - protein, 15 aa, Homo sapiens
KFHDKAAKDKGFAKW

SEQ ID NO: 939 - protein, 15 aa, Homo sapiens
LTREKMRKENIINDL

SEQ ID NO: 940 - protein, 15 aa, Homo sapiens
IINDLSDKLKSTMQQ

SEQ ID NO: 941 - protein, 15 aa, Homo sapiens
HIKSLIEKIPTAKPE

SEQ ID NO: 942 - protein, 15 aa, Homo sapiens
IEKIPTAKPELFAYP

SEQ ID NO: 943 - protein, 15 aa, Homo sapiens
ARCLPGVKQQLGTVR

SEQ ID NO: 944 - protein, 15 aa, Homo sapiens
PQKNVQGKRKISPSK

SEQ ID NO: 945 - protein, 15 aa, Homo sapiens
AHCASVYKHHYMDGQ

SEQ ID NO: 946 - protein, 15 aa, Homo sapiens
FNFVSTPKCSCGQLA

SEQ ID NO: 947 - protein, 15 aa, Homo sapiens
NYDPQKDKRFSGTVR

SEQ ID NO: 948 - protein, 15 aa, Homo sapiens
HMDIEALKKLNKNKK

SEQ ID NO: 949 - protein, 15 aa, Homo sapiens
MDIEALKKLNKNKKL

SEQ ID NO: 950 - protein, 15 aa, Homo sapiens
FGIRRNEKIAVHCTV

SEQ ID NO: 951 - protein, 15 aa, Homo sapiens
AKATGDWKGLRITVK

SEQ ID NO: 952 - protein, 15 aa, Homo sapiens
KSRDSFLKRVKENDQ

SEQ ID NO: 953 - protein, 15 aa, Homo sapiens
CESAFLSKRNPRQIN

SEQ ID NO: 954 - protein, 15 aa, Homo sapiens
GRKAVIVKNIDDGTS

SEQ ID NO: 955 - protein, 12 aa, Homo sapiens
GKFMKPGKVVLV

SEQ ID NO: 956 - protein, 15 aa, Homo sapiens
KVTAAMGKKKIAKRS

SEQ ID NO: 957 - protein, 15 aa, Homo sapiens
IGQGYLIKDGKLIKN

SEQ ID NO: 958 - protein, 15 aa, Homo sapiens
KWQDEDGKKQLEKDF

SEQ ID NO: 959 - protein, 15 aa, Homo sapiens
LKAWNDIKKVYASQR

SEQ ID NO: 960 - protein, 15 aa, Homo sapiens
LSIPHSTKRFPGYDS

SEQ ID NO: 961 - protein, 15 aa, Homo sapiens
GKGDVPTKRPPVLRA

SEQ ID NO: 962 - protein, 15 aa, Homo sapiens
ISHNPETKKTRVKLP

SEQ ID NO: 963 - protein, 15 aa, Homo sapiens
SHNPETKKTRVKLPS

SEQ ID NO: 964 - protein, 15 aa, Homo sapiens
GQFVYCGKKAQLNIG

SEQ ID NO: 965 - protein, 15 aa, Homo sapiens
RKQHSLLKRLRKAKK

SEQ ID NO: 966 - protein, 15 aa, Homo sapiens
KYVDEASKKEIKDIL

SEQ ID NO: 967 - protein, 15 aa, Homo sapiens
ATYDKLCKEVPNYKL

SEQ ID NO: 968 - protein, 15 aa, Homo sapiens
DKMCSMVKKWQTMIE

SEQ ID NO: 969 - protein, 15 aa, Homo sapiens
KIASDGLKGRVFEVS

SEQ ID NO: 970 - protein, 15 aa, Homo sapiens
LFCVGFTKKRNNQIR

SEQ ID NO: 971 - protein, 15 aa, Homo sapiens
SNSYAIKKKDELERV

SEQ ID NO: 972 - protein, 15 aa, Homo sapiens
SSNSYAIKKKDELER

SEQ ID NO: 973 - protein, 15 aa, Homo sapiens
IVKPNGEKPDEFESG

SEQ ID NO: 974 - protein, 15 aa, Homo sapiens
IGGHAIYKVEDTNMI

SEQ ID NO: 975 - protein, 15 aa, Homo sapiens
DFDVLSYKKTMLHLT

SEQ ID NO: 976 - protein, 14 aa, Homo sapiens
SPFDPNNKRVKGIY

SEQ ID NO: 977 - protein, 15 aa, Homo sapiens
LAFTCKLKSPVPFLG

SEQ ID NO: 978 - protein, 15 aa, Homo sapiens
RQRLDEYKRLMSLGK

SEQ ID NO: 979 - protein, 15 aa, Homo sapiens
VVDVKNGKGSVLPNS

SEQ ID NO: 980 - protein, 15 aa, Homo sapiens
IGGIFAFKVKDGPGG

SEQ ID NO: 981 - protein, 15 aa, Homo sapiens
SSASDGFKANLVFKE

SEQ ID NO: 982 - protein, 15 aa, Homo sapiens
VLPNSDKKADCTITM

SEQ ID NO: 983 - protein, 15 aa, Homo sapiens
VGMTKFVKPGAENSR

SEQ ID NO: 984 - protein, 15 aa, Homo sapiens
YGDLKHLKTFDRGMV

SEQ ID NO: 985 - protein, 15 aa, Homo sapiens
ATAATTTKFIVRSKD

SEQ ID NO: 986 - protein, 15 aa, Homo sapiens
NIRLIEDKPRTSHRR

SEQ ID NO: 987 - protein, 15 aa, Homo sapiens
RRTSGRDKYGPPVRT

SEQ ID NO: 988 - protein, 15 aa, Homo sapiens
TLAKGEYKMALKNLE

SEQ ID NO: 989 - protein, 15 aa, Homo sapiens
AKAPASEKGDGTLAQ

SEQ ID NO: 990 - protein, 15 aa, Homo sapiens
KNSFDCFKKVLRYEG

SEQ ID NO: 991 - protein, 15 aa, Homo sapiens
KNSFDCFKKVLRYEG

SEQ ID NO: 992 - protein, 15 aa, Homo sapiens
GLYLPLFKPSVSTSK

SEQ ID NO: 993 - protein, 15 aa, Homo sapiens
EGPKALWKGAGARVF

SEQ ID NO: 994 - protein, 15 aa, Homo sapiens
VTPEKAIKLAANDFF

SEQ ID NO: 995 - protein, 15 aa, Homo sapiens
QIPYTMMKFACFERT

SEQ ID NO: 996 - protein, 15 aa, Homo sapiens
KKLRDVFKKGDLYFN

SEQ ID NO: 997 - protein, 15 aa, Homo sapiens
ENHEFDGKKLFQHIA

SEQ ID NO: 998 - protein, 15 aa, Homo sapiens
AINVAEGKKQAQILA

SEQ ID NO: 999 - protein, 15 aa, Homo sapiens
INVAEGKKQAQILAS

SEQ ID NO: 1000 - protein, 15 aa, Homo sapiens
AEIMESIKRLYPGSV

SEQ ID NO: 1001 - protein, 15 aa, Homo sapiens
EAKLKEIKTILEEEI

SEQ ID NO: 1002 - protein, 15 aa, Homo sapiens
EITPTIQKLKEERSS

SEQ ID NO: 1003 - protein, 15 aa, Homo sapiens
TPTIQKLKEERSSYL

SEQ ID NO: 1004 - protein, 15 aa, Homo sapiens
TQEPSLGKQKVFIIR

SEQ ID NO: 1005 - protein, 15 aa, Homo sapiens
PNGGGEPKGELLEAI

SEQ ID NO: 1006 - protein, 8 aa, Homo sapiens
ERYMACKK

SEQ ID NO: 1007 - protein, 15 aa, Homo sapiens
LEAFETFKKGLGLKI

SEQ ID NO: 1008 - protein, 15 aa, Homo sapiens
AGAAGRAKGESRIRR

SEQ ID NO: 1009 - protein, 15 aa, Homo sapiens
KGRTETLKPDPHLRK

SEQ ID NO: 1010 - protein, 15 aa, Homo sapiens
LWENNKLKLQQKLVI

SEQ ID NO: 1011 - protein, 15 aa, Homo sapiens
FRKSVASKSIRRFLE

SEQ ID NO: 1012 - protein, 15 aa, Homo sapiens
QTLDNLLKLKASFNL

SEQ ID NO: 1013 - protein, 15 aa, Homo sapiens
HNKFVKCKKTSAVLG

SEQ ID NO: 1014 - protein, 30 aa, Homo sapiens
LEKDLSDKQTAYRIDLEKDLSDKQTAYRID

SEQ ID NO: 1015 - protein, 15 aa, Homo sapiens
CWEKCMDKPGPKLDS

SEQ ID NO: 1016 - protein, 15 aa, Homo sapiens
GSKHKLTKAASLPGK

SEQ ID NO: 1017 - protein, 15 aa, Homo sapiens
KAASLPGKNGNPTFA

SEQ ID NO: 1018 - protein, 15 aa, Homo sapiens
AVTAGYDKSPGGNGF

SEQ ID NO: 1019 - protein, 15 aa, Homo sapiens
VKDKLCSKRTDLCDH

SEQ ID NO: 1020 - protein, 15 aa, Homo sapiens
KNSYEGQKQKVIQPR

SEQ ID NO: 1021 - protein, 15 aa, Homo sapiens
ISTKELGKVMRMLGQ

SEQ ID NO: 1022 - protein, 15 aa, Homo sapiens
LMQMLLLKVSAHITE

SEQ ID NO: 1023 - protein, 15 aa, Homo sapiens
KRKSARKKEFIMAEL

SEQ ID NO: 1024 - protein, 15 aa, Homo sapiens
AELLQTEKAYVRDLH

SEQ ID NO: 1025 - protein, 15 aa, Homo sapiens
IGKCCLNKLSSDDAV

SEQ ID NO: 1026 - protein, 15 aa, Homo sapiens
RRVLAGEKGEVKGRA

SEQ ID NO: 1027 - protein, 15 aa, Homo sapiens
REKEKPEKKATHKEK

SEQ ID NO: 1028 - protein, 15 aa, Homo sapiens
PEKKATHKEKIEKKE

SEQ ID NO: 1029 - protein, 15 aa, Homo sapiens
THKEKIEKKEKPETK

SEQ ID NO: 1030 - protein, 15 aa, Homo sapiens
ERLRKLNKVQYSTRP

SEQ ID NO: 1031 - protein, 15 aa, Homo sapiens
RIKATNNKGKEKIYS

SEQ ID NO: 1032 - protein, 15 aa, Homo sapiens
TNNKGKEKIYSAESF

SEQ ID NO: 1033 - protein, 15 aa, Homo sapiens
GYSFVNCKKALETCG

SEQ ID NO: 1034 - protein, 12 aa, Homo sapiens
MPRRKQQAPRRS

SEQ ID NO: 1035 - protein, 12 aa, Homo sapiens
MPRRKQQAPRRA

SEQ ID NO: 1036 - protein, 15 aa, Homo sapiens
VLENLESKRFQLVDS

SEQ ID NO: 1037 - protein, 15 aa, Homo sapiens
YRALVSTKWLAESIR

SEQ ID NO: 1038 - protein, 15 aa, Homo sapiens
RRGLVMVKPGSIKPH

SEQ ID NO: 1039 - protein, 15 aa, Homo sapiens
QCYLTRDKHGVDKGL

SEQ ID NO: 1040 - protein, 15 aa, Homo sapiens
RFLLAGRKRRRSKTS

SEQ ID NO: 1041 - protein, 15 aa, Homo sapiens
EAVQNHVKSLNWGHR

SEQ ID NO: 1042 - protein, 15 aa, Homo sapiens
HSRKETGKTARAIRD

SEQ ID NO: 1043 - protein, 15 aa, Homo sapiens
QKSGELGKKITELTL

SEQ ID NO: 1044 - protein, 15 aa, Homo sapiens
ANNTILVKWLPQNDL

SEQ ID NO: 1045 - protein, 15 aa, Homo sapiens
ANNTILVKWLPQNDL

SEQ ID NO: 1046 - protein, 15 aa, Homo sapiens
PSLIDYRKPSALKFE

SEQ ID NO: 1047 - protein, 15 aa, Homo sapiens
GSNTRLYKWLPQNDL

SEQ ID NO: 1048 - protein, 15 aa, Homo sapiens
LWRFDGNKPDTLGLN

SEQ ID NO: 1049 - protein, 15 aa, Homo sapiens
KKNTAFVKKLKTITE

SEQ ID NO: 1050 - protein, 8 aa, Homo sapiens
EREEWAKK

SEQ ID NO: 1051 - protein, 15 aa, Homo sapiens
LKHQILPKEQWTKYE

SEQ ID NO: 1052 - protein, 15 aa, Homo sapiens
LSDIPEGKNMAFKWR

SEQ ID NO: 1053 - protein, 8 aa, Homo sapiens
PAAYENDK

SEQ ID NO: 1054 - protein, 15 aa, Homo sapiens
IFVPNTGKKSGKLKA

SEQ ID NO: 1055 - protein, 15 aa, Homo sapiens
PQGVQQAKRELLELA

SEQ ID NO: 1056 - protein, 15 aa, Homo sapiens
RHKGVSEKYRKLVSA

SEQ ID NO: 1057 - protein, 15 aa, Homo sapiens
EEAKKGSKPLRVKKL

SEQ ID NO: 1058 - protein, 15 aa, Homo sapiens
PSSSASKKKCEKGGR

SEQ ID NO: 1059 - protein, 15 aa, Homo sapiens
REGLDSAKRCTQALY

SEQ ID NO: 1060 - protein, 15 aa, Homo sapiens
YKCMVCKKIFMLAAS

SEQ ID NO: 1061 - protein, 15 aa, Homo sapiens
DMEDLGDKAVYCRCW

SEQ ID NO: 1062 - protein, 15 aa, Homo sapiens
DVVPQYYKDPKKLCE

SEQ ID NO: 1063 - protein, 15 aa, Homo sapiens
KVCKKKLKEKATTEA

SEQ ID NO: 1064 - protein, 15 aa, Homo sapiens
CKKKLKEKATTEADS

SEQ ID NO: 1065 - protein, 15 aa, Homo sapiens
TTEADSTKKEEDASS

SEQ ID NO: 1066 - protein, 15 aa, Homo sapiens
LVTPSISKEEILESS

SEQ ID NO: 1067 - protein, 15 aa, Homo sapiens
TLSGTNSKREKGPNN

SEQ ID NO: 1068 - protein, 15 aa, Homo sapiens
PLAPKHIKGITNLGY

SEQ ID NO: 1069 - protein, 15 aa, Homo sapiens
KSKDNRIKLMNEILN

SEQ ID NO: 1070 - protein, 15 aa, Homo sapiens
NEILNGIKVLKLYAW

SEQ ID NO: 1071 - protein, 15 aa, Homo sapiens
AKLKPAFKKDGSTTA

SEQ ID NO: 1072 - protein, 15 aa, Homo sapiens
GIGAGYCKRQVSTWT

SEQ ID NO: 1073 - protein, 15 aa, Homo sapiens
GGRAIITKSFARIHE

SEQ ID NO: 1074 - protein, 15 aa, Homo sapiens
VRHQSEIKPGEPEPQ

SEQ ID NO: 1075 - protein, 15 aa, Homo sapiens
LNDSTYDKVGKTHIK

SEQ ID NO: 1076 - protein, 15 aa, Homo sapiens
TYRNRFWKEHHFEAI

SEQ ID NO: 1077 - protein, 15 aa, Homo sapiens
EAIALVEKALQAAYG

SEQ ID NO: 1078 - protein, 15 aa, Homo sapiens
ATVVTGGKRHQLGKN

SEQ ID NO: 1079 - protein, 15 aa, Homo sapiens
TFVLPPQKFTGAVCW

SEQ ID NO: 1080 - protein, 15 aa, Homo sapiens
RLNAENEKDSRRRQA

SEQ ID NO: 1081 - protein, 15 aa, Homo sapiens
TSTICSDKTGTLTQN

SEQ ID NO: 1082 - protein, 15 aa, Homo sapiens
TSTICSDKTGTLTQN

SEQ ID NO: 1083 - protein, 15 aa, Homo sapiens
TSTICSDKTGTLTQN

SEQ ID NO: 1084 - protein, 15 aa, Homo sapiens
TSTICSDKTGTLTQN

SEQ ID NO: 1085 - protein, 15 aa, Homo sapiens
KAGGGGGKRKEKLEN

SEQ ID NO: 1086 - protein, 15 aa, Homo sapiens
EQSDAKLKEIVTNFL

SEQ ID NO: 1087 - protein, 15 aa, Homo sapiens
DIKGPEDKKKHLLIG

SEQ ID NO: 1088 - protein, 15 aa, Homo sapiens
DQFLVAFKEVGRKPP

SEQ ID NO: 1089 - protein, 11 aa, Homo sapiens
PKFEVIEKPQA

SEQ ID NO: 1090 - protein, 15 aa, Homo sapiens
VRDTVLLKSGPRKTS

SEQ ID NO: 1091 - protein, 15 aa, Homo sapiens
NFPMKGRKGMVDWAR

SEQ ID NO: 1092 - protein, 15 aa, Homo sapiens
SDVLKRLKLSSRIFQ

SEQ ID NO: 1093 - protein, 15 aa, Homo sapiens
GFGFSLAKHLHSKGF

SEQ ID NO: 1094 - protein, 15 aa, Homo sapiens
NDKTVSEKNNKCQLI

SEQ ID NO: 1095 - protein, 15 aa, Homo sapiens
FIKKNKAKENKNKKV

SEQ ID NO: 1096 - protein, 15 aa, Homo sapiens
LEKQMQEKSDQLEMH

SEQ ID NO: 1097 - protein, 15 aa, Homo sapiens
ERRKMTSKSSESNIY

SEQ ID NO: 1098 - protein, 15 aa, Homo sapiens
EKVTMVSKIVDAIYR

SEQ ID NO: 1099 - protein, 15 aa, Homo sapiens
LCDNNCMKDVMLQVI

SEQ ID NO: 1100 - protein, 15 aa, Homo sapiens
EQHVVDGKERTLLVH

SEQ ID NO: 1101 - protein, 15 aa, Homo sapiens
PDFLDLLKKQVATYS

SEQ ID NO: 1102 - protein, 15 aa, Homo sapiens
MGENSRPKSGLIVRG

SEQ ID NO: 1103 - protein, 15 aa, Homo sapiens
NRLTLTEKVEKKRCE

SEQ ID NO: 1104 - protein, 15 aa, Homo sapiens
RRLTAEGKDIDDLKR

SEQ ID NO: 1105 - protein, 15 aa, Homo sapiens
GKDIDDLKRINNMIV

SEQ ID NO: 1106 - protein, 15 aa, Homo sapiens
EEQIAEFKEAFSLFD

SEQ ID NO: 1107 - protein, 15 aa, Homo sapiens
QTSGMEYKKTDAPQP

SEQ ID NO: 1108 - protein, 15 aa, Homo sapiens
TSGMEYKKTDAPQPD

SEQ ID NO: 1109 - protein, 10 aa, Homo sapiens
MDKVCAVFGG

SEQ ID NO: 1110 - protein, 15 aa, Homo sapiens
KWVQDSMKYLDQKSP

SEQ ID NO: 1111 - protein, 14 aa, Homo sapiens
SMKYLDQKSPTPKP

SEQ ID NO: 1112 - protein, 15 aa, Homo sapiens
QAEHVESKQLVGVDL

SEQ ID NO: 1113 - protein, 15 aa, Homo sapiens
VAGVAFKKTFSYAGF

SEQ ID NO: 1114 - protein, 15 aa, Homo sapiens
PPSPMVKKQRRKKLT

SEQ ID NO: 1115 - protein, 15 aa, Homo sapiens
MVKKQRRKKLTTPSK

SEQ ID NO: 1116 - protein, 15 aa, Homo sapiens
STPTPIRKQSKRRSN

SEQ ID NO: 1117 - protein, 15 aa, Homo sapiens
TPIRKQSKRRSNIFT

SEQ ID NO: 1118 - protein, 15 aa, Homo sapiens
RGMGYMPKRGLDVNK

SEQ ID NO: 1119 - protein, 15 aa, Homo sapiens
CCACPETKKARDACI

SEQ ID NO: 1120 - protein, 15 aa, Homo sapiens
PSVNWKHKDAAIYLV

SEQ ID NO: 1121 - protein, 15 aa, Homo sapiens
HFDYNDEKFLRLMYL

SEQ ID NO: 1122 - protein, 15 aa, Homo sapiens
LAEDEEWKRIRSLLS

SEQ ID NO: 1123 - protein, 15 aa, Homo sapiens
RPKEGWQKGPRSDNS

SEQ ID NO: 1124 - protein, 15 aa, Homo sapiens
KKSDGIRKVWQKRKC

SEQ ID NO: 1125 - protein, 15 aa, Homo sapiens
GIRKVWQKRKCSVKN

SEQ ID NO: 1126 - protein, 15 aa, Homo sapiens
GYTNKGMKIACTQPR

SEQ ID NO: 1127 - protein, 15 aa, Homo sapiens
IDHIAKCKLELATKA

SEQ ID NO: 1128 - protein, 15 aa, Homo sapiens
KDSQLEFKRVSATTQ

SEQ ID NO: 1129 - protein, 15 aa, Homo sapiens
LRSEHREKMNRMRQR

SEQ ID NO: 1130 - protein, 15 aa, Homo sapiens
LSAETQWKDRAETVI

SEQ ID NO: 1131 - protein, 15 aa, Homo sapiens
VFTDLGSKDLKREKI

SEQ ID NO: 1132 - protein, 15 aa, Homo sapiens
DLGSKDLKREKISFV

SEQ ID NO: 1133 - protein, 15 aa, Homo sapiens
NLLLSVSKDHALRLW

SEQ ID NO: 1134 - protein, 15 aa, Homo sapiens
LWKFETSKYYVTIID

SEQ ID NO: 1135 - protein, 15 aa, Homo sapiens
TTGHLIYKCGGIDKR

SEQ ID NO: 1136 - protein, 15 aa, Homo sapiens
VLEKEASKMQQSGHP

SEQ ID NO: 1137 - protein, 15 aa, Homo sapiens
RFGQKTGKGWYQYDK

SEQ ID NO: 1138 - protein, 15 aa, Homo sapiens
DGIDPNFKMEHQNKR

SEQ ID NO: 1139 - protein, 15 aa, Homo sapiens
QHEELLMKLWNLLMP

SEQ ID NO: 1140 - protein, 15 aa, Homo sapiens
WNLLMPTKKLNARIS

SEQ ID NO: 1141 - protein, 15 aa, Homo sapiens
GGGLIGRKGYIGTLG

SEQ ID NO: 1142 - protein, 15 aa, Homo sapiens
LLLLCNIKPRRYLTV

SEQ ID NO: 1143 - protein, 15 aa, Homo sapiens
KRKVLEVKSDSKEDE

SEQ ID NO: 1144 - protein, 15 aa, Homo sapiens
NEVINSPKGKKRKVE

SEQ ID NO: 1145 - protein, 15 aa, Homo sapiens
SQALNDAKRELRFLL

SEQ ID NO: 1146 - protein, 15 aa, Homo sapiens
YVRKQVEKIKNSMDG

SEQ ID NO: 1147 - protein, 15 aa, Homo sapiens
RKQVEKIKNSMDGKN

SEQ ID NO: 1148 - protein, 15 aa, Homo sapiens
VTVVHKDKAHSIGKA

SEQ ID NO: 1149 - protein, 15 aa, Homo sapiens
RKPRKFSKLVLLTAS

SEQ ID NO: 1150 - protein, 15 aa, Homo sapiens
TASKDSTKVAGAKRK

SEQ ID NO: 1151 - protein, 15 aa, Homo sapiens
STKVAGAKRKGVHCV

SEQ ID NO: 1152 - protein, 15 aa, Homo sapiens
FLKVPRSKDDPGKGS

SEQ ID NO: 1153 - protein, 15 aa, Homo sapiens
RSKDDPGKGSYWAID

SEQ ID NO: 1154 - protein, 15 aa, Homo sapiens
KIKQLLVKTKKELAD

SEQ ID NO: 1155 - protein, 15 aa, Homo sapiens
QLLVKTKKELADSKQ

SEQ ID NO: 1156 - protein, 15 aa, Homo sapiens
KKELADSKQAETDHL

SEQ ID NO: 1157 - protein, 15 aa, Homo sapiens
SFLQRFFKFSRDVFL

SEQ ID NO: 1158 - protein, 15 aa, Homo sapiens
EKTPVKKKAKKAGAT

SEQ ID NO: 1159 - protein, 15 aa, Homo sapiens
PVKKKAKKAGATAGK

SEQ ID NO: 1160 - protein, 15 aa, Homo sapiens
AEAYRKTKAESKSSK

SEQ ID NO: 1161 - protein, 15 aa, Homo sapiens
KTLREKMKSNEAASK

SEQ ID NO: 1162 - protein, 9 aa, Homo sapiens
KNPLAVSKI

SEQ ID NO: 1163 - protein, 9 aa, Homo sapiens
KVAQATCKL

SEQ ID NO: 1164 - protein, 15 aa, Homo sapiens
QNRRMKEKKINRDRL

SEQ ID NO: 1165 - protein, 15 aa, Homo sapiens
KKDISENKRAVRRLR

SEQ ID NO: 1166 - protein, 15 aa, Homo sapiens
RAAWEHMKKQKYGRI

SEQ ID NO: 1167 - protein, 15 aa, Homo sapiens
EQYLELYKPLPRAGK

SEQ ID NO: 1168 - protein, 15 aa, Homo sapiens
QKLLELEKHIRVTIQ

SEQ ID NO: 1169 - protein, 15 aa, Homo sapiens
LPFKVVEKKTKPYIQ

SEQ ID NO: 1170 - protein, 15 aa, Homo sapiens
VLEDSDLKKSDIDEI

SEQ ID NO: 1171 - protein, 15 aa, Homo sapiens
QMQVRNSKASFDKLK

SEQ ID NO: 1172 - protein, 15 aa, Homo sapiens
CFNTLRTKEQLGYIV

SEQ ID NO: 1173 - protein, 15 aa, Homo sapiens
IFQEIYDKQYKSQFE

SEQ ID NO: 1174 - protein, 15 aa, Homo sapiens
NLSELDTKIQEKAMK

SEQ ID NO: 1175 - protein, 15 aa, Homo sapiens
NSEIAGEKKSAQWRT

SEQ ID NO: 1176 - protein, 15 aa, Homo sapiens
AEAIHSGKKKEAMIQ

SEQ ID NO: 1177 - protein, 15 aa, Homo sapiens
EAIHSGKKKEAMIQC

SEQ ID NO: 1178 - protein, 15 aa, Homo sapiens
AKEEDKEKTKLKEPG

SEQ ID NO: 1179 - protein, 15 aa, Homo sapiens
EEDKEKTKLKEPGLL

SEQ ID NO: 1180 - protein, 15 aa, Homo sapiens
MKDQGPDKENSGAIE

SEQ ID NO: 1181 - protein, 15 aa, Homo sapiens
ANEKMNAKLEELRQH

SEQ ID NO: 1182 - protein, 15 aa, Homo sapiens
KKLNEQSKLLKLKES

SEQ ID NO: 1183 - protein, 15 aa, Homo sapiens
NEQSKLLKLKESTER

SEQ ID NO: 1184 - protein, 15 aa, Homo sapiens
CARAYSEKVTEFMLC

SEQ ID NO: 1185 - protein, 15 aa, Homo sapiens
KGQITSDKKSREKIT

SEQ ID NO: 1186 - protein, 15 aa, Homo sapiens
RFYVYNKKKRLVNTP

SEQ ID NO: 1187 - protein, 15 aa, Homo sapiens
EQIVRVIKQTSPGKG

SEQ ID NO: 1188 - protein, 15 aa, Homo sapiens
IKQTSPGKGLMPTPV

SEQ ID NO: 1189 - protein, 15 aa, Homo sapiens
QMGKIDKKALIRHFH

SEQ ID NO: 1190 - protein, 15 aa, Homo sapiens
HLKGAHSKNLFLKDK

SEQ ID NO: 1191 - protein, 15 aa, Homo sapiens
KNLFLKDKKKKNYWL

SEQ ID NO: 1192 - protein, 15 aa, Homo sapiens
RVRNFGGKGQGRWEF

SEQ ID NO: 1193 - protein, 15 aa, Homo sapiens
GTETTLEKQKPVSKP

SEQ ID NO: 1194 - protein, 15 aa, Homo sapiens
CGSKANIKHKPGGGD

SEQ ID NO: 1195 - protein, 11 aa, Homo sapiens
MGLKAAQKTLF

SEQ ID NO: 1196 - protein, 15 aa, Homo sapiens
MGLKAAQKTLFPLRS

SEQ ID NO: 1197 - protein, 15 aa, Homo sapiens
DSFWKNKKIYLDIIH

SEQ ID NO: 1198 - protein, 15 aa, Homo sapiens
KAMKQLVKQEELKRL

SEQ ID NO: 1199 - protein, 15 aa, Homo sapiens
VKNWMDMKRRVGPYR

SEQ ID NO: 1200 - protein, 15 aa, Homo sapiens
YVKTVFAKGAASEDG

SEQ ID NO: 1201 - protein, 15 aa, Homo sapiens
RLTQAIQKARDHGLL

SEQ ID NO: 1202 - protein, 15 aa, Homo sapiens
PKAQKGTKVRMSVQR

SEQ ID NO: 1203 - protein, 15 aa, Homo sapiens
QRKLSWIKMTKLEYE

SEQ ID NO: 1204 - protein, 15 aa, Homo sapiens
KEITSAIKRVHKFME

SEQ ID NO: 1205 - protein, 15 aa, Homo sapiens
KRKELEEKMVTLLKE

SEQ ID NO: 1206 - protein, 15 aa, Homo sapiens
EKMVTLLKEKNDLQL

SEQ ID NO: 1207 - protein, 15 aa, Homo sapiens
TVKRKRGKSQPNCAL

SEQ ID NO: 1208 - protein, 15 aa, Homo sapiens
TAACIPQKAKNVASE

SEQ ID NO: 1209 - protein, 15 aa, Homo sapiens
SQSENKKKSTKKDFE

SEQ ID NO: 1210 - protein, 15 aa, Homo sapiens
DFDVYFRKTKAATIL

SEQ ID NO: 1211 - protein, 15 aa, Homo sapiens
KSSVAQIKPSSGHDR

SEQ ID NO: 1212 - protein, 15 aa, Homo sapiens
AVSSIAQKIRMTSGV

SEQ ID NO: 1213 - protein, 15 aa, Homo sapiens
LAAKANTKNTSDVMY

SEQ ID NO: 1214 - protein, 15 aa, Homo sapiens
NTSDVMYKKDYEKNK

SEQ ID NO: 1215 - protein, 15 aa, Homo sapiens
TSDVMYKKDYEKNKG

SEQ ID NO: 1216 - protein, 15 aa, Homo sapiens
NTSDVMYKKDYEKSK

SEQ ID NO: 1217 - protein, 15 aa, Homo sapiens
TSDVMYKKDYEKSKG

SEQ ID NO: 1218 - protein, 15 aa, Homo sapiens
AHSTLLSKKETSNLY

SEQ ID NO: 1219 - protein, 15 aa, Homo sapiens
GRGKGKGKKKERGSG

SEQ ID NO: 1220 - protein, 15 aa, Homo sapiens
SHLNANIKSSSRYLR

SEQ ID NO: 1221 - protein, 15 aa, Homo sapiens
TTQYGKMKGMSRHSQ

SEQ ID NO: 1222 - protein, 15 aa, Homo sapiens
DVNKALRKVMGSKIH

SEQ ID NO: 1223 - protein, 11 aa, Homo sapiens
LRKVMGSKIHS

SEQ ID NO: 1224 - protein, 15 aa, Homo sapiens
GPSNNDEKSEMSFPG

SEQ ID NO: 1225 - protein, 15 aa, Homo sapiens
RLTSTATKDPEGFVG

SEQ ID NO: 1226 - protein, 15 aa, Homo sapiens
VNAFKLMKRLNTEWS

SEQ ID NO: 1227 - protein, 15 aa, Homo sapiens
MLALLKRKDLANLEV

SEQ ID NO: 1228 - protein, 15 aa, Homo sapiens
KQDGSGVKGYEEKLN

SEQ ID NO: 1229 - protein, 15 aa, Homo sapiens
MGIGSEEKPGTGRDE

SEQ ID NO: 1230 - protein, 15 aa, Homo sapiens
MQAIGGQKVNMPSLS

SEQ ID NO: 1231 - protein, 15 aa, Homo sapiens
RWYQDLDKIDQKDLM

SEQ ID NO: 1232 - protein, 15 aa, Homo sapiens
CRTWELSKQWSEKVT

SEQ ID NO: 1233 - protein, 15 aa, Homo sapiens
LSKQWSEKVTEEFFH

SEQ ID NO: 1234 - protein, 15 aa, Homo sapiens
FHQGDIEKKYHLGVS

SEQ ID NO: 1235 - protein, 15 aa, Homo sapiens
DSVKALSKKARDGKL

SEQ ID NO: 1236 - protein, 15 aa, Homo sapiens
FIEEHATKLSRTKEE

SEQ ID NO: 1237 - protein, 15 aa, Homo sapiens
EVVGIAHKFFGAENS

SEQ ID NO: 1238 - protein, 15 aa, Homo sapiens
AQQPGHEKSQPGPAK

SEQ ID NO: 1239 - protein, 15 aa, Homo sapiens
SKNMENAKALADSGG

SEQ ID NO: 1240 - protein, 15 aa, Homo sapiens
SPAKNFFKKAAAALF

SEQ ID NO: 1241 - protein, 15 aa, Homo sapiens
GRGNYEFKQITEDTV

SEQ ID NO: 1242 - protein, 15 aa, Homo sapiens
RALCTGEKGFGYKGS

SEQ ID NO: 1243 - protein, 15 aa, Homo sapiens
RSLSPPHKRWRRSSC

SEQ ID NO: 1244 - protein, 15 aa, Homo sapiens
PEPPAPEKPSLLRPV

SEQ ID NO: 1245 - protein, 15 aa, Homo sapiens
VENVAELKKSFNRHL

SEQ ID NO: 1246 - protein, 15 aa, Homo sapiens
QLSESSTKDSLKSMT

SEQ ID NO: 1247 - protein, 15 aa, Homo sapiens
MVREANMKQAASEKQ

SEQ ID NO: 1248 - protein, 15 aa, Homo sapiens
GLKRSMDKLKEKERD

SEQ ID NO: 1249 - protein, 15 aa, Homo sapiens
KRSMDKLKEKERDKV

SEQ ID NO: 1250 - protein, 15 aa, Homo sapiens
KKEKHKTKTAQQIAK

SEQ ID NO: 1251 - protein, 15 aa, Homo sapiens
EARAETEKPANQKEP

SEQ ID NO: 1252 - protein, 15 aa, Homo sapiens
TEKPANQKEPTTCHT

SEQ ID NO: 1253 - protein, 15 aa, Homo sapiens
QVTTRTRKSVTEVKE

SEQ ID NO: 1254 - protein, 15 aa, Homo sapiens
CMKKSTKKKTLKNKS

SEQ ID NO: 1255 - protein, 15 aa, Homo sapiens
QGQDTEVKASDPHSM

SEQ ID NO: 1256 - protein, 15 aa, Homo sapiens
VKFGRMSKKQRDSLY

SEQ ID NO: 1257 - protein, 15 aa, Homo sapiens
RPVIVAYKKNLVEQH

SEQ ID NO: 1258 - protein, 15 aa, Homo sapiens
PVNKSGGKAKKKKWS

SEQ ID NO: 1259 - protein, 15 aa, Homo sapiens
VLDEGKMKLDYILGL

SEQ ID NO: 1260 - protein, 15 aa, Homo sapiens
DFRVVSLKLLRTHFK

SEQ ID NO: 1261 - protein, 15 aa, Homo sapiens
KLLRTHFKKSLDDGK

SEQ ID NO: 1262 - protein, 15 aa, Homo sapiens
GKGKVFPKLRKRSSI

SEQ ID NO: 1263 - protein, 15 aa, Homo sapiens
KVFPKLRKRSSIRSV

SEQ ID NO: 1264 - protein, 15 aa, Homo sapiens
SPRLLSVKGSPKVDE

SEQ ID NO: 1265 - protein, 15 aa, Homo sapiens
LSVKGSPKVDEKVLP

SEQ ID NO: 1266 - protein, 15 aa, Homo sapiens
YSEGYPGKRYYGGAE

SEQ ID NO: 1267 - protein, 15 aa, Homo sapiens
VMYEFVGKSAPFLIL

SEQ ID NO: 1268 - protein, 15 aa, Homo sapiens
SESPLMEKKNSLKED

SEQ ID NO: 1269 - protein, 15 aa, Homo sapiens
QALNFAFKDKYKQLF

SEQ ID NO: 1270 - protein, 15 aa, Homo sapiens
EGAKAFFKGAWSNVL

SEQ ID NO: 1271 - protein, 15 aa, Homo sapiens
DCLVKITKSDGIRGL

SEQ ID NO: 1272 - protein, 15 aa, Homo sapiens
VLAPPKMKSTKTAME

SEQ ID NO: 1273 - protein, 11 aa, Homo sapiens
MGCKVLLNIGQ

SEQ ID NO: 1274 - protein, 10 aa, Homo sapiens
GRGNIFQKRR

SEQ ID NO: 1275 - protein, 9 aa, Homo sapiens
TERYMACKK

SEQ ID NO: 1276 - protein, 15 aa, Homo sapiens
TGVQNLRKKHKRLEA

SEQ ID NO: 1277 - protein, 15 aa, Homo sapiens
TDQAQDVKKIEKFHS

SEQ ID NO: 1278 - protein, 15 aa, Homo sapiens
DVAYQYVKKGSRIYL

SEQ ID NO: 1279 - protein, 15 aa, Homo sapiens
RKKPVEVKKGKDPNA

SEQ ID NO: 1280 - protein, 15 aa, Homo sapiens
GKDPNAPKRPMSAYM

SEQ ID NO: 1281 - protein, 15 aa, Homo sapiens
KVPEVPKKPEEKVPV

SEQ ID NO: 1282 - protein, 15 aa, Homo sapiens
VPKKPEEKVPVLIPK

SEQ ID NO: 1283 - protein, 15 aa, Homo sapiens
NKPHDGGKPITNYIL

SEQ ID NO: 1284 - protein, 15 aa, Homo sapiens
ITNYILEKRETMSKR

SEQ ID NO: 1285 - protein, 15 aa, Homo sapiens
NHKKWKVKYYKGLGT

SEQ ID NO: 1286 - protein, 15 aa, Homo sapiens
GLGTSTSKEAKEYFA

SEQ ID NO: 1287 - protein, 15 aa, Homo sapiens
AAAWHGQKPGTHPPS

SEQ ID NO: 1288 - protein, 15 aa, Homo sapiens
VTTQVAHKKSSLQTS

SEQ ID NO: 1289 - protein, 15 aa, Homo sapiens
KKSEKKDKDGKREGK

SEQ ID NO: 1290 - protein, 15 aa, Homo sapiens
FNIIPSPKTICYMIS

SEQ ID NO: 1291 - protein, 15 aa, Homo sapiens
NGGNQNLKVADEALK

SEQ ID NO: 1292 - protein, 15 aa, Homo sapiens
RGRLAEVKGNLEEAK

SEQ ID NO: 1293 - protein, 15 aa, Homo sapiens
KWGFTGQKTEEVKQD

SEQ ID NO: 1294 - protein, 15 aa, Homo sapiens
TEEVKQDKDDIINIF

SEQ ID NO: 1295 - protein, 9 aa, Homo sapiens
KEREEWAKK

SEQ ID NO: 1296 - protein, 15 aa, Homo sapiens
PRQALGDKIVRDIRP

SEQ ID NO: 1297 - protein, 15 aa, Homo sapiens
GKIKSSYKRECINLG

SEQ ID NO: 1298 - protein, 15 aa, Homo sapiens
NNFALGYKAADFQLH

SEQ ID NO: 1299 - protein, 15 aa, Homo sapiens
VCFRQVTKTTSEKMP

SEQ ID NO: 1300 - protein, 15 aa, Homo sapiens
HKEKGANKMHKCKFC

SEQ ID NO: 1301 - protein, 15 aa, Homo sapiens
YKCSLCEKTFINTSS

SEQ ID NO: 1302 - protein, 15 aa, Homo sapiens
KGKVGGLKARCSSCN